(19) United States
(12) Patent Application Publication
(10) Pub. No.: US 2022/0328773 A1
(43) Pub. Date: Oct. 13, 2022

(54) ELECTROLUMINESCENT DEVICE

(71) Applicant: BEIJING SUMMER SPROUT TECHNOLOGY CO., LTD., Beijing (CN)

(72) Inventors: Yang WANG, Beijing (CN); Feng LI, Beijing (CN); Jianfei YAO, Beijing (CN); Gang YANG, Beijing (CN); Junfei WANG, Beijing (CN); Shang DING, Beijing (CN); Ming SANG, Beijing (CN); Hongbo LI, Beijing (CN); Zhen WANG, Beijing (CN); Wei CAI, Beijing (CN); Chi Yuen Raymond Kwong, Beijing (CN); Chuanjun Xia, Beijing (CN)

(21) Appl. No.: 17/705,696

(22) Filed: Mar. 28, 2022

(30) Foreign Application Priority Data
Mar. 30, 2021 (CN) 202110338405.6

(51) Int. Cl. H01L 51/00; C07D 209/86; C09K 11/06; C07D 405/14; C07F 15/00

(52) U.S. Cl. CPC H01L 51/0085; C07D 209/86; C09K 11/06; H01L 51/0072; C07D 405/14; H01L 51/0073; H01L 51/0067; C07F 15/0033; H01L 51/5012

(57) ABSTRACT

Provided is an electroluminescent device. The organic electroluminescent device comprises a first metal complex comprising a ligand Lₐ having a structure of Formula 1, a first compound having a structure of Formula 2 and a second compound having a structure of Formula 3. Compared to the related art, such a compound composition can significantly improve the performance of the organic electroluminescent device, especially significantly extend a device lifetime, and finally significantly improve the overall performance of the device. Further provided are an electronic device comprising the electroluminescent device and a compound composition comprising the first metal complex, the first compound and the second compound.

### CROSS-REFERENCE TO RELATED APPLICATION(S)

This present disclosure claims priority to Chinese Patent Application No. CN 202110338405.6 filed on Mar. 30, 2021, the disclosure of which is incorporated herein by reference in its entirety.

### TECHNICAL FIELD

The present disclosure relates to an electroluminescent device. More particularly, the present disclosure relates to an electroluminescent device which comprises a first metal complex comprising a ligand Lₐ having a structure of Formula 1, a first compound having a structure of Formula 2 and a second compound having a structure of Formula 3, an electronic device comprising the electroluminescent device and a compound composition comprising the first metal complex, the first compound and the second compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which includes an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may include multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may include one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

US20200203631A1 has disclosed an organic electroluminescent device containing a first host compound having a structure of Formula 1

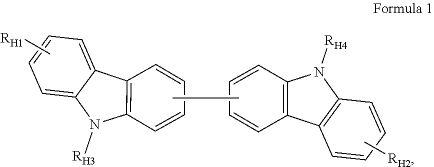

a second host compound having a structure of Formula 2

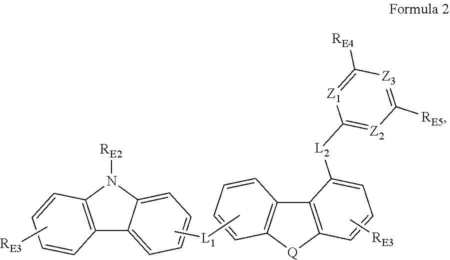

and a metal complex having a structure of Formula 3

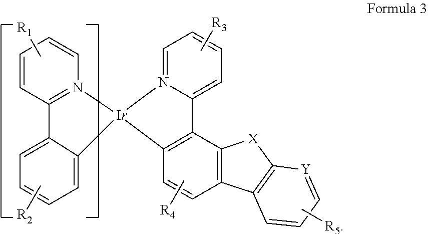

The second host compound in this application is a compound containing a dibenzo five-membered heterocyclic group joined to substituted or unsubstituted carbazolyl, and does not disclose the second compound in present application with a metal complex containing a particular fluorine/cyano-substituted ligand can achieve better device performance when used in the device.

US2020091442A1 has disclosed a metal complex containing a fluorine-substituted ligand. The fluorine-substituted ligand has the following structure:

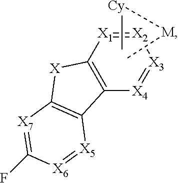

where X₁ to X₇ are selected from C, CR or N. This application has disclosed only devices in which such metal complexes containing fluorine-substituted ligands are used with host materials

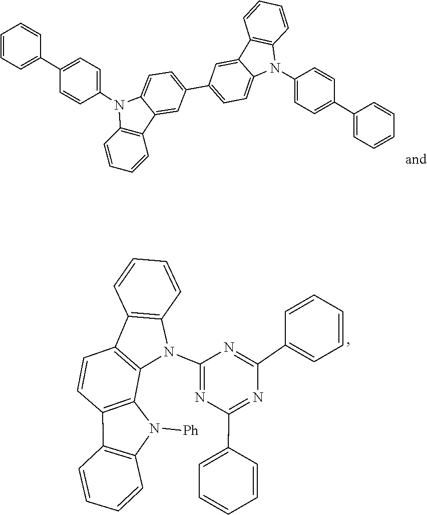 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2nc(-c3ccccc3)nc(-n3c4ccccc4c4ccc5c6ccccc6n(-c6ccccc6)c5c43)n2)cc1]

and has not studied the device performance of such metal complexes containing fluorine-substituted ligands with other host materials.

US2020251666A1 has disclosed a metal complex containing a cyano-substituted ligand. The cyano-substituted ligand has the following structure:

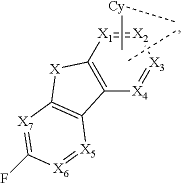

where X₁ to X₄ are selected from C, CRₓ₁ or N, X₅ to X₈ are selected from CRₓ₂ or N, and at least one of Rₓ₁ and Rₓ₂ is cyano. This application has disclosed only devices in which such metal complexes containing cyano-substituted ligands are used with host materials

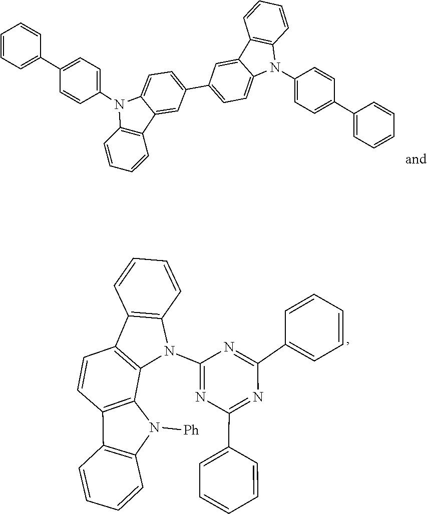 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2nc(-c3ccccc3)nc(-n3c4ccccc4c4ccc5c6ccccc6n(-c6ccccc6)c5c43)n2)cc1]

and has not studied the device performance of such metal complexes containing cyano-substituted ligands with other host materials.

US2019363261A1 has disclosed a compound formed through

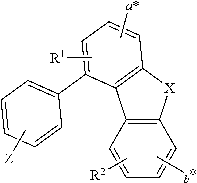

bonded to

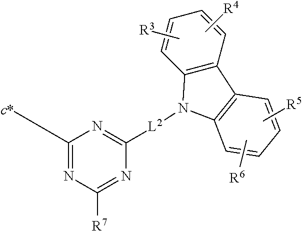

and an electroluminescent device containing the compound. In an embodiment, the following metal complex

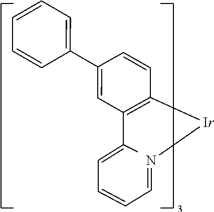

and the use of the metal complex in the host material

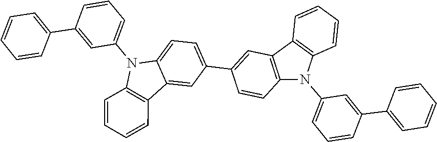 [SMILES: c1ccc(-c2cccc(-n3c4ccccc4c4cc(-c5ccc6c(c5)c5ccccc5n6-c5cccc(-c6ccccc6)c5)ccc43)c2)cc1]

have been disclosed. This application has not focused on that device in which a metal complex containing a particular fluorine/cyano-substituted ligand is used in such host materials can achieve better performance.

### SUMMARY

The present disclosure provides a series of electroluminescent devices each comprising a first metal complex comprising a ligand Lₐ having a structure of Formula 1, a first compound having a structure of Formula 2 and a second compound having a structure of Formula 3 to solve at least part of the above problems.

According to an embodiment of the present disclosure, disclosed is an electroluminescent device comprising:

an anode,

a cathode, and

an organic layer disposed between the anode and the cathode, wherein the organic layer comprises at least a first metal complex, a first compound and a second compound;

wherein the first metal complex comprises a metal M and a ligand Lₐ coordinated to the metal M, wherein the ligand Lₐ has a structure represented by Formula 1:

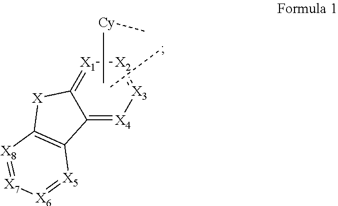

wherein

the metal M is selected from a metal with a relative atomic mass greater than 40;

Cy is, at each occurrence identically or differently, selected from substituted or unsubstituted aryl having 5 to 24 ring atoms or substituted or unsubstituted heteroaryl having 5 to 24 ring atoms; and Cy is joined to the metal M by a metal-carbon bond or a metal-nitrogen bond;

X is, at each occurrence identically or differently, selected from the group consisting of O, S, Se, NR′, CR′R′ and SiR′R′; wherein when two R′ are present, the two R′ are identical or different;

X₁ to X₈ are, at each occurrence identically or differently, selected from C, CRₓ or N, wherein at least one of X₁ to X₄ is C and joined to Cy;

X₁, X₂, X₃ or X₄ is joined to the metal M by a metal-carbon bond or a metal-nitrogen bond; at least one of X₁ to X₈ is CRₓ, wherein the Rₓ is cyano or fluorine; and adjacent substituents R′, Rₓ can be optionally joined to form a ring;

wherein the first compound has a structure represented by Formula 2:

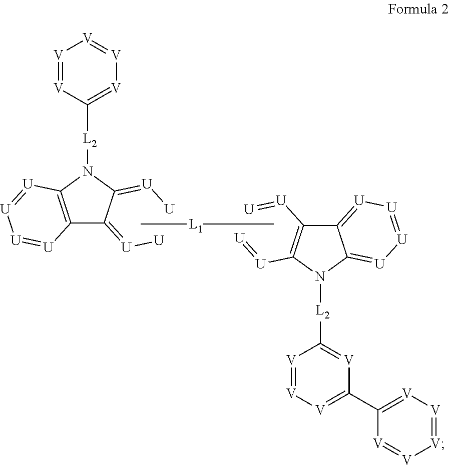

wherein

U is, at each occurrence identically or differently, selected from C, CRᵤ or N;

V is, at each occurrence identically or differently, selected from CRᵥ or N;

L₁ and L₂ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and

adjacent substituents Rᵤ, and Rᵥ, can be optionally joined to form a ring;

wherein the second compound has a structure represented by Formula 3:

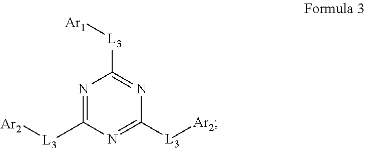

wherein

Ar₁ has a structure represented by Formula A:

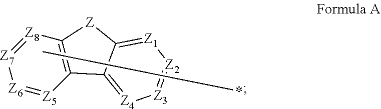

wherein

Z is, at each occurrence identically or differently, selected from the group consisting of O, S and Se;

L₃ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;

Z₁ to Z₈ are, at each occurrence identically or differently, selected from C, CR_{z} or N, and at least one of Z₁ to Z₈ is C and joined to L₃;

at least one of Z₁ to Z₈ is CR_{z}, wherein the R_{z} is substituted or unsubstituted aryl having 6 to 30 carbon atoms;

Ar₂ is, at each occurrence identically or differently, selected from substituted or unsubstituted aryl having 6 to 30 carbon atoms, substituted or unsubstituted heteroaryl having 3 to 30 carbon atoms or a combination thereof;

“*” represents a position where Ar₁ is joined to L₃;

adjacent substituents R_{z} can be optionally joined to form a ring;

R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and

R′, Rₓ, Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof.

According to an embodiment of the present disclosure, disclosed is an electronic device comprising the electroluminescent device in the preceding embodiment.

According to another embodiment of the present disclosure, disclosed is a compound composition comprising the first metal complex, the first compound and the second compound in the preceding embodiment.

The present disclosure provides the series of electroluminescent devices each comprising the first metal complex comprising the ligand Lₐ having the structure of Formula 1, the first compound having the structure of Formula 2 and the second compound having the structure of Formula 3. Compared to the related art, such a compound composition can significantly improve the performance of an organic electroluminescent device, especially significantly extend a device lifetime, and finally significantly improve the overall performance of the device.

### BRIEF DESCRIPTION OF DRAWINGS

FIG. 1 is a schematic diagram of an electroluminescent device 100 disclosed herein.

FIG. 2 is a schematic diagram of another electroluminescent device 200 disclosed herein.

### DETAILED DESCRIPTION

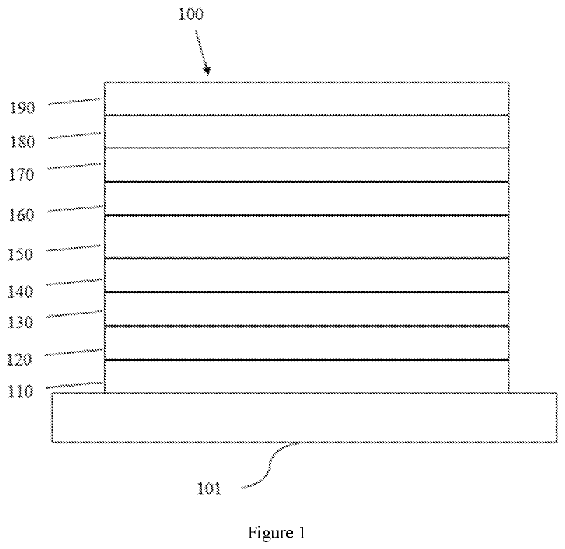

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

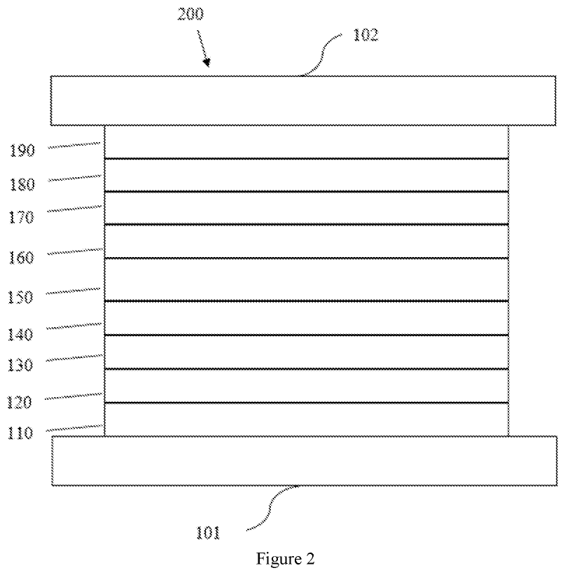

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (AEs-T). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small AES-T. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups includes saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenyl ethyl silyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyl dimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldii sopropylgermanyl, diphenyli sopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitution refers to a range that includes a di-substitution, up to the maximum available substitution. When substitution in the compounds mentioned in the present disclosure represents multiple substitution (including di-, tri-, and tetra-substitutions etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

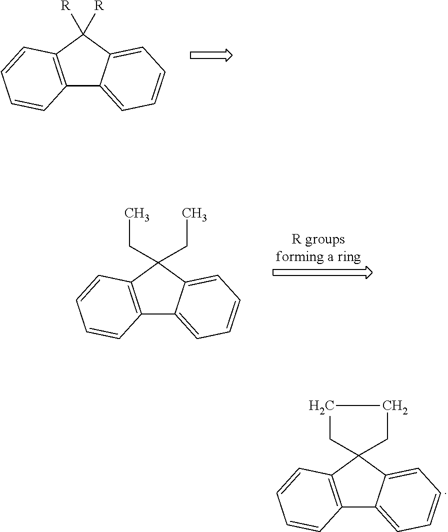

The expression that adjacent substituents can be optionally joined to form a ring is also intended to mean that two substituents bonded to carbon atoms which are directly bonded to each other are joined to each other via a chemical bond to form a ring, which can be exemplified by the following formula:

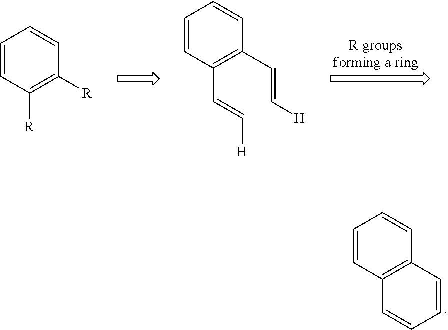

The expression that adjacent substituents can be optionally joined to form a ring is also intended to mean that two substituents bonded to a further distant carbon atom are joined to each other via a chemical bond to form a ring, which can be exemplified by the following formula:

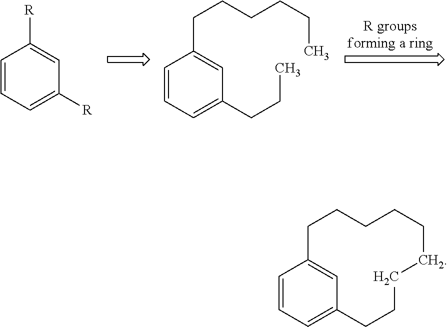

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

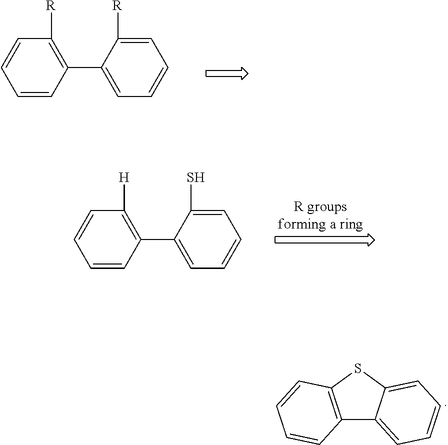

According to an embodiment of the present disclosure, disclosed is an electroluminescent device comprising:

an anode,

a cathode, and

an organic layer disposed between the anode and the cathode, wherein the organic layer comprises at least a first metal complex, a first compound and a second compound;

wherein the first metal complex comprises a metal M and a ligand Lₐ coordinated to the metal M, wherein the ligand Lₐ has a structure represented by Formula 1:

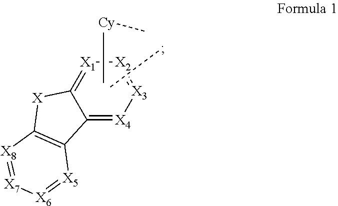

wherein

the metal M is selected from a metal with a relative atomic mass greater than 40;

Cy is, at each occurrence identically or differently, selected from substituted or unsubstituted aryl having 5 to 24 ring atoms or substituted or unsubstituted heteroaryl having 5 to 24 ring atoms; and Cy is joined to the metal M by a metal-carbon bond or a metal-nitrogen bond;

X is, at each occurrence identically or differently, selected from the group consisting of O, S, Se, NR′, CR′R′ and SiR′R′; wherein when two R′ are present, the two R′ are identical or different;

X₁ to X₈ are, at each occurrence identically or differently, selected from C, CRₓ or N, wherein at least one of X₁ to X₄ is C and joined to Cy;

X₁, X₂, X₃ or X₄ is joined to the metal M by a metal-carbon bond or a metal-nitrogen bond;

at least one of X₁ to X₈ is CRₓ, wherein the Rₓ is cyano or fluorine; and

adjacent substituents R′, Rₓ can be optionally joined to form a ring;

wherein the first compound has a structure represented by Formula 2:

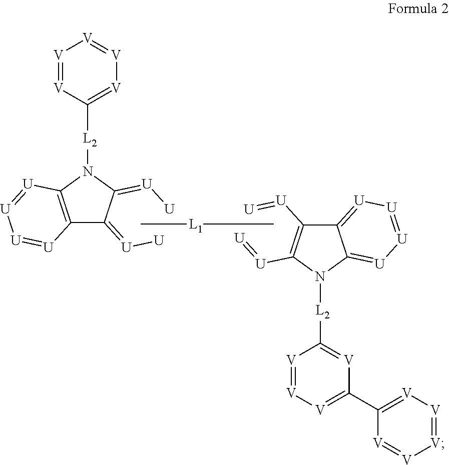

wherein

U is, at each occurrence identically or differently, selected from C, CRᵤ or N;

V is, at each occurrence identically or differently, selected from CRₓ or N;

L₁ and L₂ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and

adjacent substituents Rᵤ and Rᵥ can be optionally joined to form a ring;

wherein the second compound has a structure represented by Formula 3:

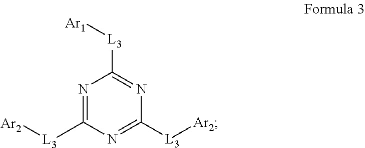

wherein

Ar₁ has a structure represented by Formula A:

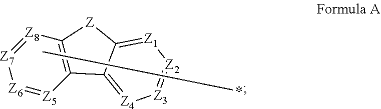

wherein

Z is, at each occurrence identically or differently, selected from the group consisting of O, S and Se;

L₃ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;

Z₁ to Z₈ are, at each occurrence identically or differently, selected from C, CR_{z} or N, and at least one of Z₁ to Z₈ is C and joined to L₃;

at least one of Z₁ to Z₈ is CR_{z}, wherein the R_{z} is substituted or unsubstituted aryl having 6 to 30 carbon atoms;

Ar₂ is, at each occurrence identically or differently, selected from substituted or unsubstituted aryl having 6 to 30 carbon atoms, substituted or unsubstituted heteroaryl having 3 to 30 carbon atoms or a combination thereof;

“*” represents a position where Ar₁ is joined to L₃;

adjacent substituents R_{z} can be optionally joined to form a ring;

R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and

R′, Rₓ, Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof.

In the present disclosure, the expression that “adjacent substituents R′, Rₓ can be optionally joined to form a ring” is intended to mean that any one or more of groups of adjacent substituents, such as two substituents R′, two substituents Rₓ, and substituents R′ and Rₓ, can be joined to form a ring. Obviously, it is possible that none of these substituents are joined to form a ring.

In the present disclosure, the expression that “adjacent substituents Rᵤ, Rᵥ can be optionally joined to form a ring” is intended to mean that any one or more of groups of adjacent substituents, such as two substituents Rᵤ, two substituents Rᵥ, and substituents Rᵤ and Rᵥ, can be joined to form a ring. Obviously, it is possible that none of these substituents are joined to form a ring.

In the present disclosure, the expression that “adjacent substituents R_{z} can be optionally joined to form a ring” is intended to mean that any one or more of groups of adjacent substituents, such as any two adjacent substituents R_{z}, can be joined to form a ring. Obviously, it is possible that none of these substituents are joined to form a ring.

According to an embodiment of the present disclosure, Ar₁ does not contain a substituted or unsubstituted carbazolyl group.

According to an embodiment of the present disclosure, Cy is selected from any structure in the group consisting of:

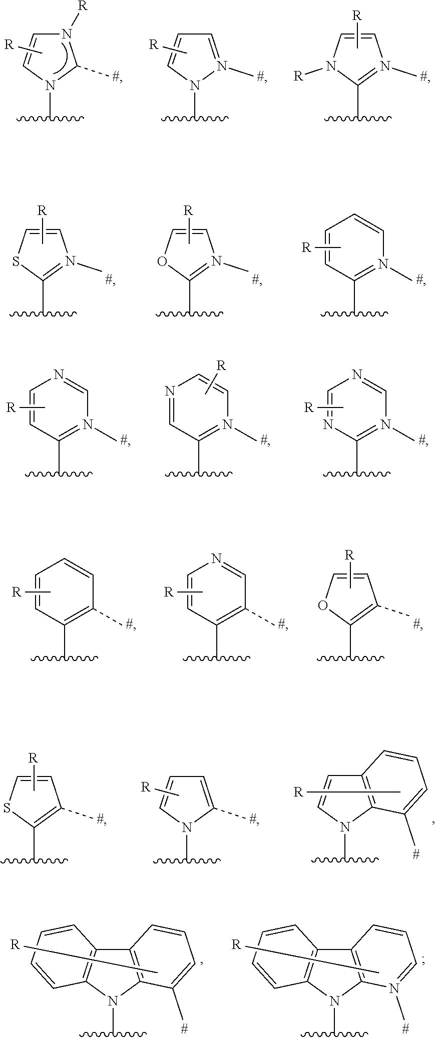

wherein

R represents, at each occurrence identically or differently, mono-substitution, multiple substitutions or non-substitution; and when a plurality of R are present in any structure, the plurality of R may be identical or different;

R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;

two adjacent substituents R can be optionally joined to form a ring; and

“#” represents a position where Cy is joined to the metal M, and

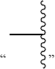

represents a position where Cy is joined to X₁, X₂, X₃ or X₄.

In this embodiment, the expression that “adjacent substituents R can be optionally joined to form a ring” is intended to mean that any one or more of groups of adjacent substituents, such as any two adjacent substituents R, can be joined to form a ring. Obviously, it is possible that none of these substituents are joined to form a ring.

According to an embodiment of the present disclosure, the first metal complex has a general formula of M(Lₐ)ₘ(L_{b})ₙ(L_{c})_{q};

wherein

M is, at each occurrence identically or differently, selected from the group consisting of Cu, Ag, Au, Ru, Rh, Pd, Os, Ir and Pt;

Lₐ, L_{b} and L_{c} are a first ligand, a second ligand and a third ligand coordinated to the metal M, respectively, and L_{c} is identical to or different from Lₐ or L_{b}; wherein Lₐ, L_{b} and L_{c} can be optionally joined to form a multidentate ligand;

m is selected from 1, 2 or 3, n is selected from 0, 1 or 2, q is selected from 0, 1 or 2, and m+n+q equals an oxidation state of the metal M; wherein when m is greater than or equal to 2, a plurality of Lₐ are identical or different; when n is equal to 2, two L_{b} are identical or different; when q is equal to 2, two L_{c} are identical or different;

Lₐ is, at each occurrence identically or differently, selected from the group consisting of:

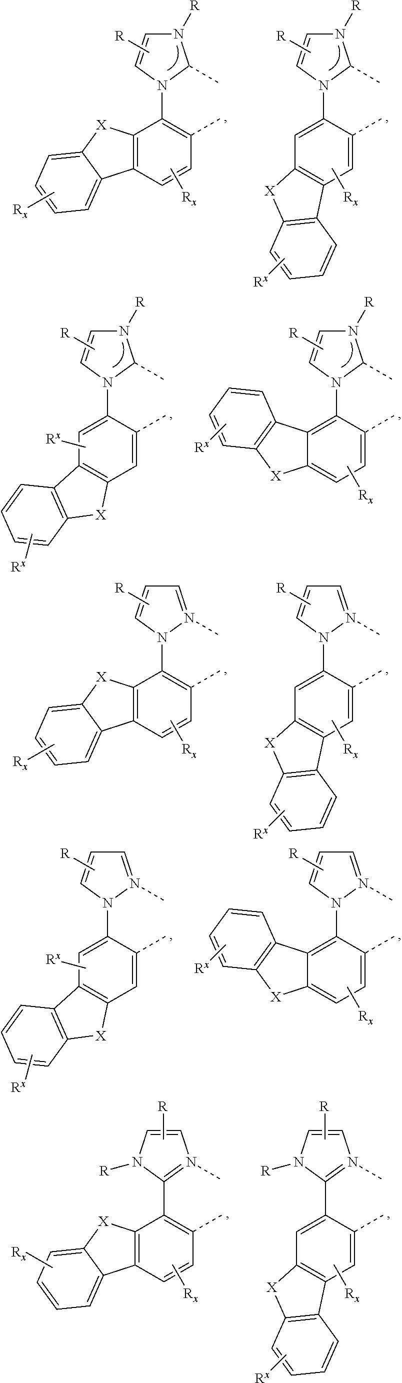

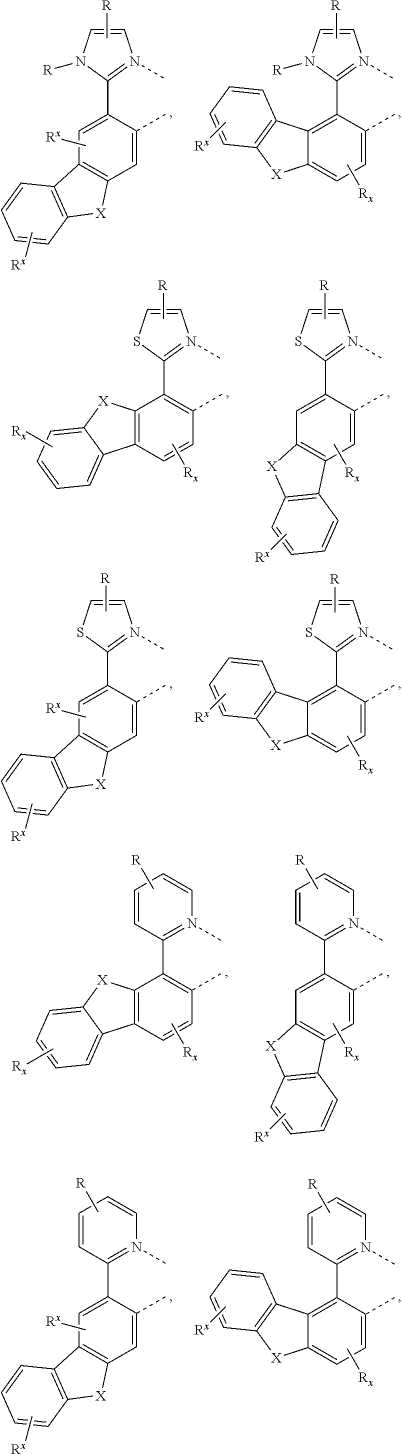

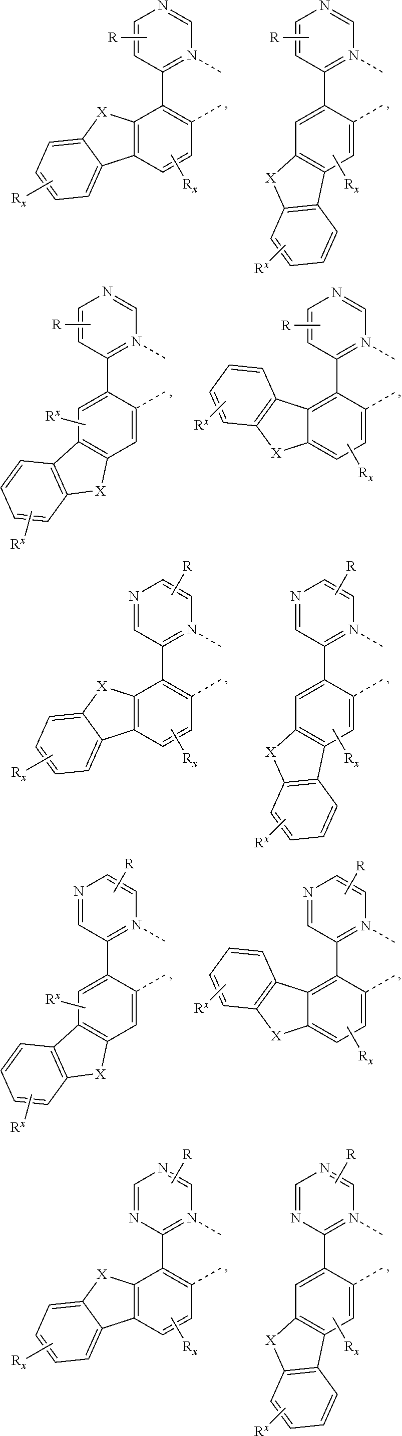

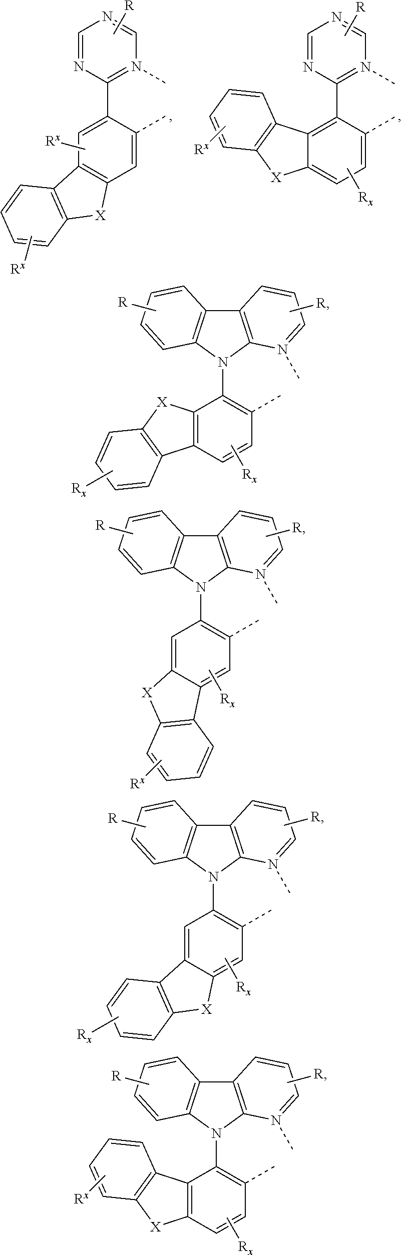

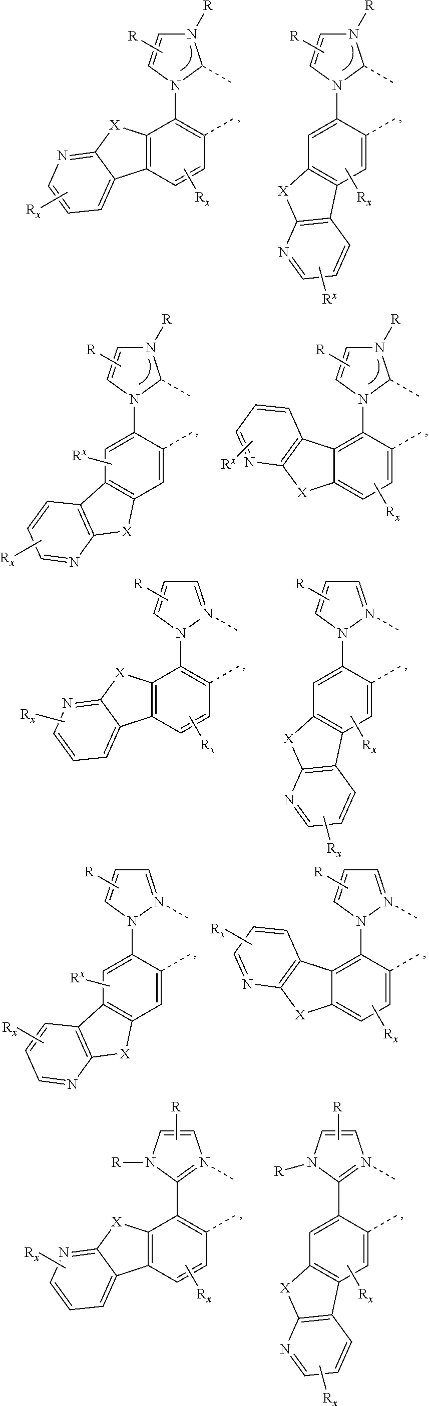

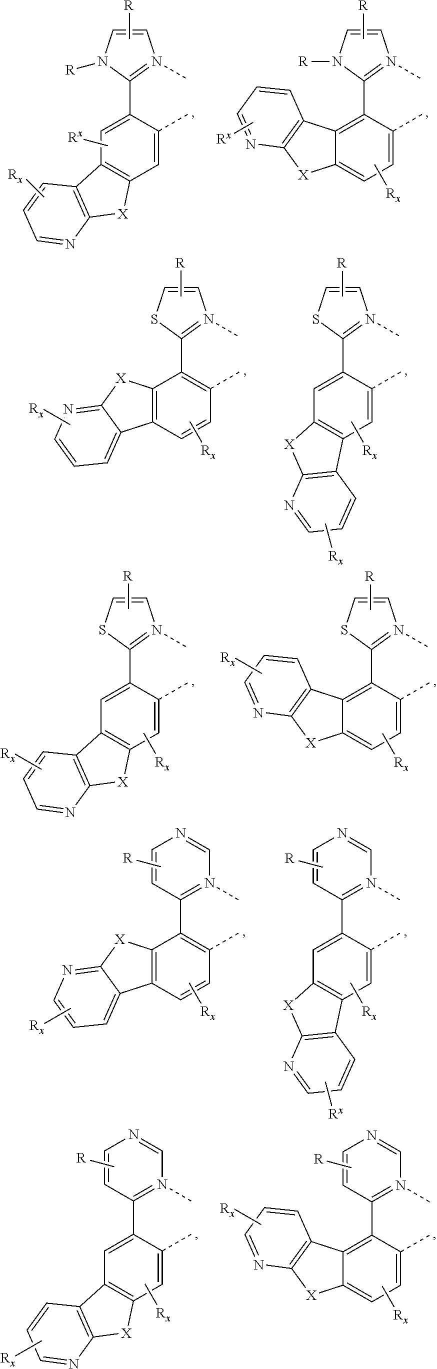

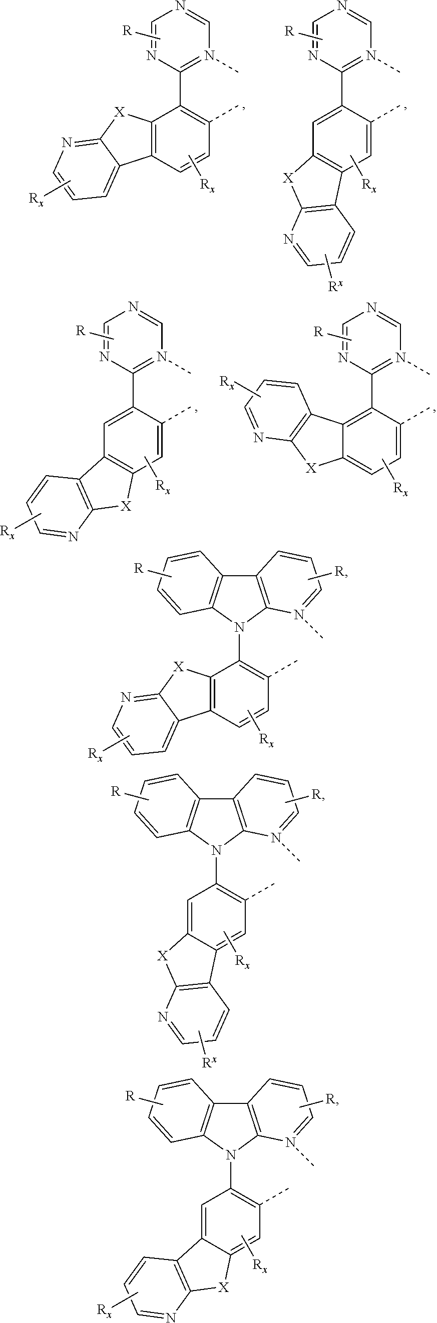

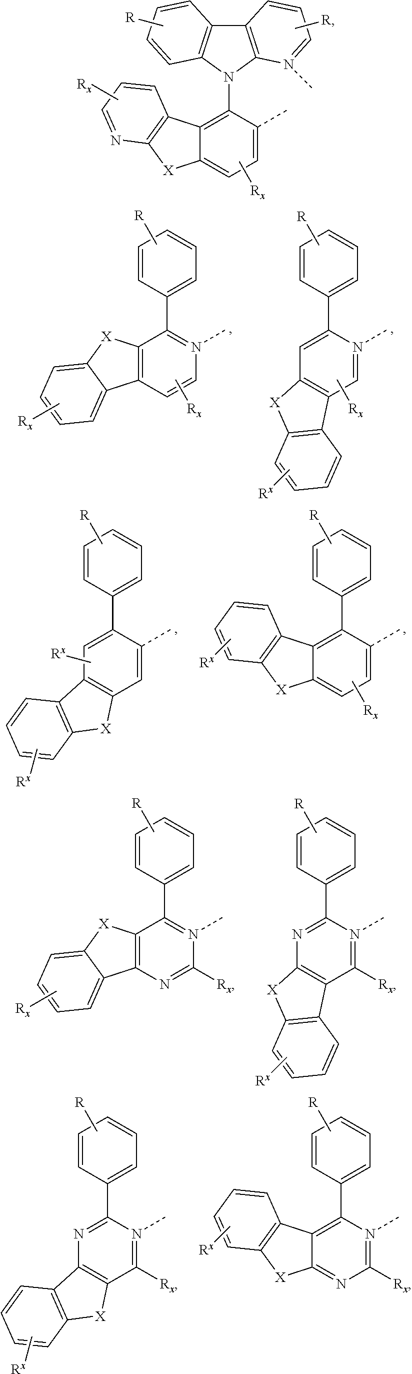

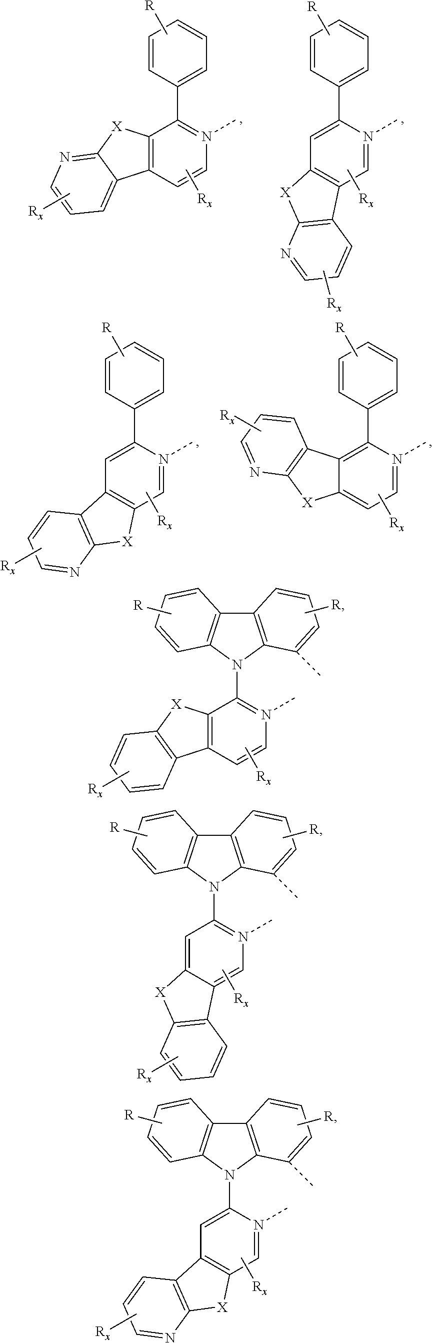

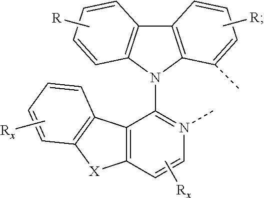

X is selected from the group consisting of O, S, Se, NR′, CR′R′ and SiR′R; wherein when two R′ are present, the two R′ are identical or different;

R and Rₓ represent, at each occurrence identically or differently, mono-substitution, multiple substitutions or non-substitution;

at least one Rₓ is selected from cyano or fluorine;

adjacent substituents R, R′, Rₓ can be optionally joined to form a ring;

L_{b} and L_{c} are, at each occurrence identically or differently, selected from a structure represented by any one of the group consisting of:

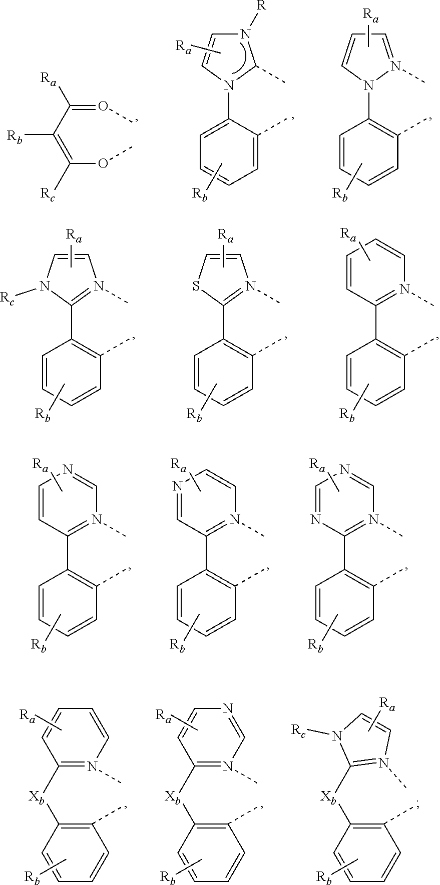

wherein

X_{b} is, at each occurrence identically or differently, selected from the group consisting of: O, S, Se, NR_{N1} and CR_{C1}R_{C2};

Rₐ and R_{b} represent, at each occurrence identically or differently, mono-substitution, multiple substitutions or non-substitution;

adjacent substituents Rₐ, R_{b}, R_{c}, R_{N1}, R_{C1}, R_{C2} can be optionally joined to form a ring; and R, R′, Rₓ, Rₐ, R_{b}, R_{c}, R_{N1}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof.

In the present application, the expression that “adjacent substituents R, R′, Rₓ can be optionally joined to form a ring” is intended to mean that any one or more of groups of adjacent substituents, such as two substituents R′, two substituents R, two substituents Rₓ, substituents R′ and Rₓ, and substituents R and Rₓ, can be joined to form a ring. Obviously, it is possible that none of these substituents are joined to form a ring.

In this embodiment, the expression that “adjacent substituents Rₐ, R_{b}, R_{c}, R_{N1}, R_{C1}, R_{C2} can be optionally joined to form a ring” is intended to mean that any one or more of groups of adjacent substituents, such as two substituents Rₐ, two substituents R_{b}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{c2}, substituents R_{b} and R_{C1}, substituents R_{b} and R_{C2}, and substituents R_{C1} and R_{c2}, can be joined to form a ring. Obviously, it is possible that none of these substituents are joined to form a ring.

According to an embodiment of the present disclosure, the metal M is, at each occurrence identically or differently, selected from Pt or Ir.

According to an embodiment of the present disclosure, the first metal complex Ir(Lₐ)ₘ(L_{b})₃₋ₘ has a structure represented by Formula 5:

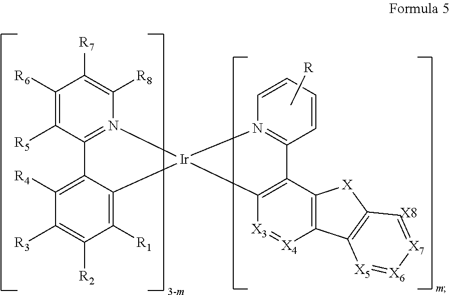

wherein

m is 1, 2 or 3; when m is 2 or 3, a plurality of Lₐ are identical or different; when m is 1, two L_{b} are identical or different;

X is selected from the group consisting of O, S, Se, NR′, CR′R′ and SiR′R; wherein when two R′ are present, the two R′ are identical or different;

X₃ to X₈ are, at each occurrence identically or differently, selected from CRₓ or N;

at least one of X₃ to X₈ is CRₓ, wherein the Rₓ is cyano or fluorine; and

R represents, at each occurrence identically or differently, mono-substitution, multiple substitutions or non-substitution;

R₁ to R₈, R′, Rₓ and R are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;

adjacent substituents R₁ to R₈ can be optionally joined to form a ring; and

adjacent substituents R′, R and Rₓ can be optionally joined to form a ring.

In this embodiment, the expression that “adjacent substituents R₁ to R₈ can be optionally joined to form a ring” is intended to mean that any one or more of groups of adjacent substituents, such as any two adjacent substituents of R₁ to R₈, can be joined to form a ring. Obviously, it is possible that none of these substituents are joined to form a ring.

According to an embodiment of the present disclosure, X is selected from 0 or S.

According to an embodiment of the present disclosure, X is selected from 0.

According to an embodiment of the present disclosure, at least one of X₃ to X₈ is selected from N, for example, one of X₃ to X₈ is selected from N, or two of X₃ to X₈ are selected from N.

According to an embodiment of the present disclosure, X₃ to X₈ are selected from CRₓ, and at least one Rₓ is cyano or fluorine.

According to an embodiment of the present disclosure, at least one of X₅ to X₈ is selected from CRₓ, wherein the Rₓ is cyano or fluorine; preferably, X₇ or X₈ is CRₓ, wherein the Rₓ is cyano or fluorine

According to an embodiment of the present disclosure, at least one of X₅ to X₈ is selected from CRₓ, wherein the Rₓ is cyano or fluorine; and at least one of X₅ to X₈ is selected from CRₓ, wherein the Rₓ is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms.

According to an embodiment of the present disclosure, X₇ is selected from CRₓ, wherein the Rₓ is cyano or fluorine; and X₈ is also selected from CRₓ, wherein the Rₓ is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms.

According to an embodiment of the present disclosure, X₈ is selected from CRₓ, wherein the Rₓ is cyano; and X₇ is also selected from CRₓ, wherein the Rₓ is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms.

According to an embodiment of the present disclosure, at least one, two, three or all of R₂, R₃, R₆ and R₇ is(are) selected from the group consisting of: deuterium, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, at least one, two, three or all of R₂, R₃, R₆ and R₇ is(are) selected from the group consisting of: deuterium, substituted or unsubstituted alkyl having 1 to 20 carbon atoms, substituted or unsubstituted cycloalkyl having 3 to 20 ring carbon atoms and combinations thereof.

According to an embodiment of the present disclosure, at least one, two, three or all of R₂, R₃, R₆ and R₇ is(are) selected from the group consisting of: deuterium, methyl, ethyl, propyl, isopropyl, n-butyl, isobutyl, t-butyl, cyclopentyl, cyclohexyl, neopentyl, t-pentyl and combinations thereof; optionally, hydrogen in the above groups can be partially or fully deuterated.

According to an embodiment of the present disclosure, at least one or more of R₅ to R₈ is(are) selected from substituted or unsubstituted alkyl having 1 to 20 carbon atoms, substituted or unsubstituted cycloalkyl having 3 to 20 ring carbon atoms or a combination thereof; and the total number of carbon atoms in R₅ to R₈ is at least four.

According to an embodiment of the present disclosure, at least two of X₃ to X₈ are selected from CRₓ, wherein one Rₓ is selected from fluorine or cyano, and at least another Rₓ is selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, cyano and combinations thereof.

According to an embodiment of the present disclosure, at least two of X₃ to X₈ are selected from CRₓ, wherein one Rₓ is selected from fluorine or cyano, and at least another Rₓ is selected from the group consisting of: hydrogen, deuterium, fluorine, substituted or unsubstituted alkyl having 1 to 6 carbon atoms, substituted or unsubstituted cycloalkyl having 3 to 6 ring carbon atoms, substituted or unsubstituted aryl having 6 to 12 carbon atoms, substituted or unsubstituted heteroaryl having 3 to 12 carbon atoms, substituted or unsubstituted alkylsilyl having 3 to 6 carbon atoms, cyano and combinations thereof.

According to an embodiment of the present disclosure, R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms and combinations thereof.

According to an embodiment of the present disclosure, at least one of R is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, R′ is selected from the group consisting of: substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, R′ is selected from methyl or deuterated methyl.

According to an embodiment of the present disclosure, Lₐ is, at each occurrence identically or differently, selected from the group consisting of Lₐ₁ to Lₐ₁₈₈, wherein the specific structures of Lₐ₁ to Lₐ₁₈₈ are referred to claim 10.

According to an embodiment of the present disclosure, L_{b} is, at each occurrence identically or differently, selected from the group consisting of L_{b1} to L_{b128}, wherein the specific structures of L_{b1} to L_{b128} are referred to claim 11.

According to an embodiment of the present disclosure, L_{c} is, at each occurrence identically or differently, selected from the group consisting of L_{c1} to L_{c360}, wherein the specific structures of L_{c1} to L_{c360} are referred to claim 12.

According to an embodiment of the present disclosure, the first metal complex has a general formula of Ir(Lₐ)(L_{b})(L_{c}), Ir(Lₐ)₂(L_{b}), Ir(Lₐ)₂(L_{c}) or Ir(Lₐ)₃, wherein Lₐ is, at each occurrence identically or differently, selected from the group consisting of Lₐ₁ to Lₐ₁₈₈, L_{b} is, at each occurrence identically or differently, selected from the group consisting of L_{b1} to L_{b128}, and L_{c} is, at each occurrence identically or differently, selected from the group consisting of L_{c1} to L_{c360}, wherein the specific structures of Lₐ₁ to Lₐ₁₈₈ are referred to claim 10, the specific structures of L_{b1} to L_{b128} are referred to claim 11, and the specific structures of L_{c1} to L_{c360} are referred to claim 12.

According to an embodiment of the present disclosure, the first metal complex is selected from the group consisting of GD1 to GD178, wherein the specific structures of GD1 to GD178 are referred to claim 13.

According to an embodiment of the present disclosure, the first compound has a structure represented by Formula 2a:

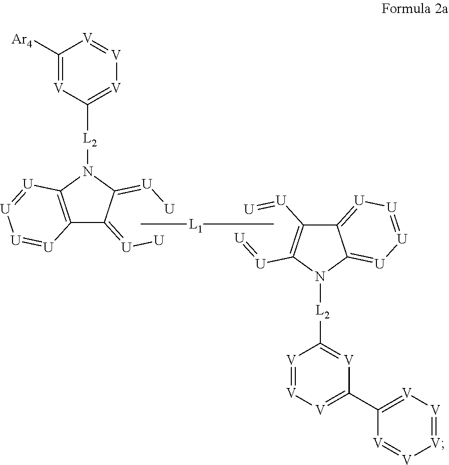

wherein in Formula 2a,

U is, at each occurrence identically or differently, selected from C, CRᵤ or N;

V is, at each occurrence identically or differently, selected from CRᵥ or N;

Ar₄ is, at each occurrence identically or differently, selected from substituted or unsubstituted aryl having 6 to 30 carbon atoms, substituted or unsubstituted heteroaryl having 3 to 30 carbon atoms or a combination thereof;

L₁ and L₂ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;

Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and

adjacent substituents Ar₄, Rᵤ and Rᵥ can be optionally joined to form a ring.

In the present disclosure, the expression that “adjacent substituents Ar₄, Rᵤ and Rᵥ can be optionally joined to form a ring” is intended to mean that any one or more of groups of adjacent substituents, such as two substituents Rᵤ, two substituents Rᵥ, and substituents Rᵥ and Ar₄, can be joined to form a ring. Obviously, it is possible that none of these substituents are joined to form a ring.

According to an embodiment of the present disclosure, the first compound has a structure represented by Formula 2b:

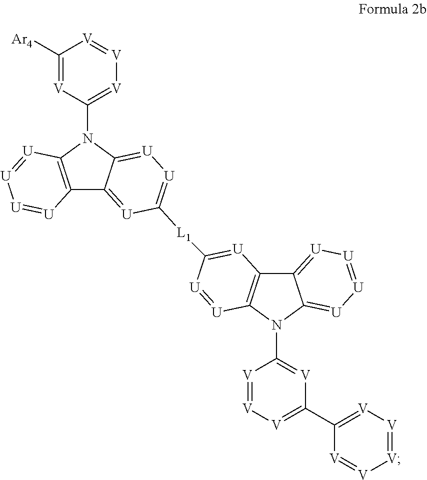

wherein in Formula 2b,

U is, at each occurrence identically or differently, selected from CRᵤ or N;

V is, at each occurrence identically or differently, selected from CRᵥ or N;

Ar₄ is, at each occurrence identically or differently, selected from substituted or unsubstituted aryl having 6 to 30 carbon atoms, substituted or unsubstituted heteroaryl having 3 to 30 carbon atoms or a combination thereof;

L₁ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;

Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and

adjacent substituents Ar₄, Rᵤ and Rᵥ can be optionally joined to form a ring.

According to an embodiment of the present disclosure, U is, at each occurrence identically or differently, selected from C or CRᵤ.

According to an embodiment of the present disclosure, V is, at each occurrence identically or differently, selected from CR_{Y}.

According to an embodiment of the present disclosure, at least one of U is selected from N, for example, at least one of U is N, or at least two of U are N.

According to an embodiment of the present disclosure, at least one of V is selected from N, for example, at least one of V is N, or at least two of V are N.

According to an embodiment of the present disclosure, Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, cyano, isocyano, hydroxyl, a sulfanyl group and combinations thereof.

According to an embodiment of the present disclosure, Rᵤ and Rᵥ are, at each occurrence identically or differently, selected from the group consisting of: hydrogen, deuterium, fluorine, substituted or unsubstituted aryl having 6 to 20 carbon atoms, substituted or unsubstituted heteroaryl having 3 to 20 carbon atoms and combinations thereof.

According to an embodiment of the present disclosure, Rᵤ and Rᵥ are, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbozolyl, substituted or unsubstituted dibenzofuranyl, substituted or unsubstituted dibenzothienyl and combinations thereof.

According to an embodiment of the present disclosure, Z₁ to Z₈ are, at each occurrence identically or differently, selected from C or CR_{z}.

According to an embodiment of the present disclosure, at least one of Z₁ to Z₈ is selected from N, for example, one of Z₁ to Z₈ is selected from N, or two of Z₁ to Z₈ are selected from N.

According to an embodiment of the present disclosure, at least two of Z₁ to Z₈ are CR_{z}, wherein at least one of R_{z} is selected from substituted or unsubstituted aryl having 6 to 30 carbon atoms; and at least another one of R_{z} is selected from hydrogen, deuterium, halogen, cyano, substituted or unsubstituted aryl having 6 to 30 carbon atoms or a combination thereof.

According to an embodiment of the present disclosure, at least one, two or three of Z₁ to Z₈ is(are) selected from CR_{z}, wherein the R_{z} is selected from substituted or unsubstituted aryl having 6 to 30 carbon atoms.

According to an embodiment of the present disclosure, at least one, two or three of Z₁ to Z₈ is(are) selected from CR_{z}, wherein the R_{z} is selected from any one of the following groups that are unsubstituted or substituted with one or more of deuterium, halogen and cyano: phenyl, naphthyl, biphenyl and terphenyl.

According to an embodiment of the present disclosure, at least one of Z₁ to Z₄ is selected from C and joined to L₃; Z₅ and/or Z₈ are(is) selected from CR_{z}, wherein the R_{z} is substituted or unsubstituted aryl having 6 to 30 carbon atoms.

According to an embodiment of the present disclosure, at least one of Z₁ to Z₄ is selected from C and joined to L₃; and at least one of Z₁ to Z₄ is selected from CR_{z}, wherein the R_{z} is substituted or unsubstituted aryl having 6 to 30 carbon atoms.

According to an embodiment of the present disclosure, the second compound has a structure represented by one of Formula 3-1 to Formula 3-4:

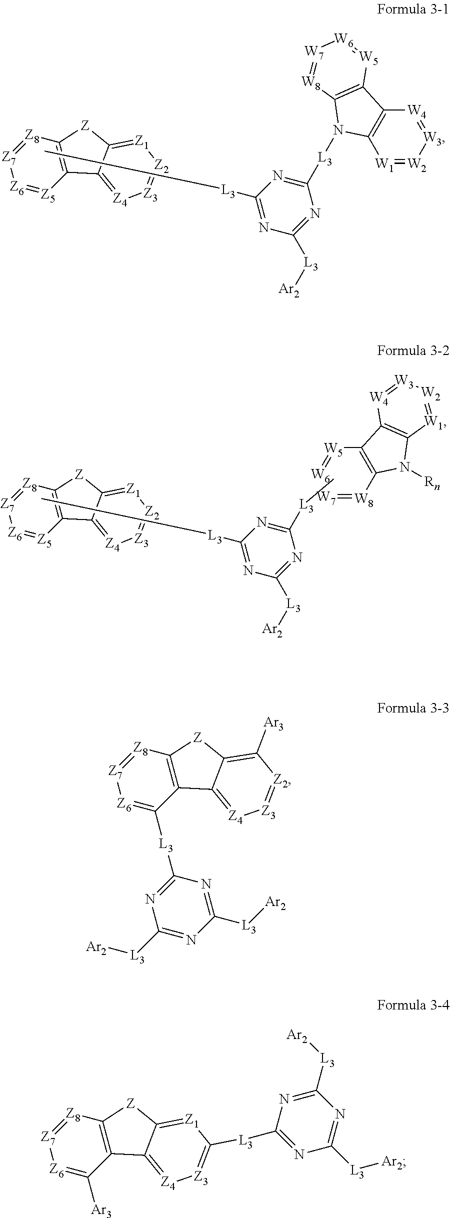

wherein

Z is, at each occurrence identically or differently, selected from the group consisting of O, S and Se;

L₃ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 20 carbon atoms or a combination thereof;

Z₁ to Z₈ are, at each occurrence identically or differently, selected from C, CR_{z} or N;

in Formula 3-1 and Formula 3-2, at least one of Z₁ to Z₈ is C and joined to L₃, and at least one of Z₁ to Z₈ is selected from CR_{z}, wherein the R_{z} is substituted or unsubstituted aryl having 6 to 30 carbon atoms;

W₁ to W₈ are, at each occurrence identically or differently, selected from C, CR_{L} or N;

Ar₂ and Ar₃ are, at each occurrence identically or differently, selected from substituted or unsubstituted aryl having 6 to 30 carbon atoms, substituted or unsubstituted heteroaryl having 3 to 30 carbon atoms or a combination thereof;

R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;

R_{w} and Rₙ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and adjacent substituents R_{z}, R_{w}, Rₙ can be optionally joined to form a ring.

In this embodiment, the expression that “adjacent substituents R_{z}, R_{w}, Rₙ can be optionally joined to form a ring” is intended to mean that any one or more of groups of adjacent substituents, such as two substituents R_{z}, two substituents R_{w}, two substituents Rₙ, and substituents R_{w} and Rₙ, can be joined to form a ring. Obviously, it is possible that none of these substituents are joined to form a ring.

According to an embodiment of the present disclosure, L₁ to L₃ are, at each occurrence identically or differently, selected from a single bond, substituted or unsubstituted arylene having 6 to 20 carbon atoms, substituted or unsubstituted heteroarylene having 3 to 20 carbon atoms or a combination thereof.

According to an embodiment of the present disclosure, L₁ to L₂ are, at each occurrence identically or differently, selected from a single bond, substituted or unsubstituted phenylene, substituted or unsubstituted biphenylene or a combination thereof.

According to an embodiment of the present disclosure, L₁ to L₂ are, at each occurrence identically or differently, selected from a single bond, phenylene or biphenylene.

According to an embodiment of the present disclosure, L₁ to L₂ are, at each occurrence identically or differently, selected from a single bond.

According to an embodiment of the present disclosure, L₃ is, at each occurrence identically or differently, selected from a single bond, substituted or unsubstituted phenylene, substituted or unsubstituted biphenylene or a combination thereof.

According to an embodiment of the present disclosure, L₃ is, at each occurrence identically or differently, selected from a single bond, phenylene or biphenylene.

According to an embodiment of the present disclosure, L₃ is, at each occurrence identically or differently, selected from a single bond.

According to an embodiment of the present disclosure, Ar₂ to Ar₃ are, at each occurrence identically or differently, selected from substituted or unsubstituted aryl having 6 to 20 carbon atoms, substituted or unsubstituted heteroaryl having 3 to 20 carbon atoms or a combination thereof.

According to an embodiment of the present disclosure, Ar₂ to Ar₃ are, at each occurrence identically of differently, selected from any one of the following groups that are unsubstituted or substituted with one or more of deuterium, halogen and cyano: phenyl, naphthyl, biphenyl, terphenyl, phenanthryl, fluorenyl, carbazolyl, dibenzofuranyl, dibenzothienyl, pyridyl, pyrimidinyl, pyrazinyl, azafluorenyl, azacarbazolyl, azadibenzofuranyl, azadibenzothienyl, diazafluorenyl, diazacarbazolyl, diazadibenzofuranyl, diazadibenzothienyl and combinations thereof.

According to an embodiment of the present disclosure, Ar₂ to Ar₃ are, at each occurrence identically or differently, selected from any one of the following groups that are unsubstituted or substituted with one or more of deuterium, halogen and cyano: phenyl, naphthyl, biphenyl, terphenyl, phenanthryl, fluorenyl, carbazolyl, dibenzofuranyl, dibenzothienyl and combinations thereof.

According to an embodiment of the present disclosure, Ar₄ is, at each occurrence identically or differently, selected from substituted or unsubstituted aryl having 6 to 20 carbon atoms, substituted or unsubstituted heteroaryl having 3 to 20 carbon atoms or a combination thereof.

According to an embodiment of the present disclosure, Ar₄ is, at each occurrence identically of differently, selected from any one of the following groups that are unsubstituted or substituted with one or more of deuterium, halogen and cyano: phenyl, naphthyl, biphenyl, terphenyl, phenanthryl, fluorenyl, carbazolyl, dibenzofuranyl, dibenzothienyl, pyridyl, pyrimidinyl, pyrazinyl, azafluorenyl, azacarbazolyl, azadibenzofuranyl, azadibenzothienyl, diazafluorenyl, diazacarbazolyl, diazadibenzofuranyl, diazadibenzothienyl and combinations thereof.

According to an embodiment of the present disclosure, Ar₄ is, at each occurrence identically or differently, selected from any one of the following groups that are unsubstituted or substituted with one or more of deuterium, halogen and cyano: phenyl, naphthyl, biphenyl, terphenyl, phenanthryl, fluorenyl, carbazolyl, dibenzofuranyl, dibenzothienyl and combinations thereof.

According to an embodiment of the present disclosure, the first compound is selected from the group consisting of Compound 2-1 to Compound 2-241, wherein the specific structures of Compound 2-1 to Compound 2-241 are referred to claim 26.

According to an embodiment of the present disclosure, the second compound is selected from the group consisting of Compound H-1 to Compound H-178, wherein the specific structures of Compound H-1 to Compound H-178 are referred to claim 27.

According to an embodiment of the present disclosure, the second compound is selected from the group consisting of Compound H-1 to Compound H-178, Compound H-179 and Compound H-180, wherein the specific structures of Compound H-1 to Compound H-178 are referred to claim 27.

According to an embodiment of the present disclosure, the organic layer is a light-emitting layer, the first metal complex is doped in the first compound and the second compound, and the weight of the first metal complex accounts for 1% to 30% of the total weight of the light-emitting layer.

According to an embodiment of the present disclosure, the organic layer is a light-emitting layer, the first metal complex is doped in the first compound and the second compound, and the weight of the first metal complex accounts for 3% to 13% of the total weight of the light-emitting layer.

According to an embodiment of the present disclosure, further disclosed is an electronic device comprising the electroluminescent device in any one of the preceding embodiments.

According to an embodiment of the present disclosure, further disclosed is a compound composition comprising the first metal complex, the first compound and the second compound in any one of the preceding embodiments.

Combination with Other Materials

The materials described in the present disclosure for a particular layer in an organic light-emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light-emitting device may be used in combination with a variety of other materials present in the device. For example, compounds disclosed herein may be used in combination with a wide variety of hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### Device Example

The method for preparing an electroluminescent device is not limited. The preparation methods in the following examples are merely examples and not to be construed as limitations. Those skilled in the art can make reasonable improvements on the preparation methods in the following examples based on the related art. Exemplarily, the proportions of various materials in a light-emitting layer are not particularly limited. Those skilled in the art can reasonably select the proportions within a certain range based on the related art. For example, taking the total weight of the materials in the light-emitting layer as reference, a host material may account for 70% to 99% and a light-emitting material may account for 1% to 30%; the host material may account for 90% to 98% and the light-emitting material may account for 2% to 10%; or the host material may account for 87% to 98% and the light-emitting material may account for 2% to 13%. Further, the host material may include one material or two materials, where a ratio of two host materials may be 99:1 to 1:99; or the ratio may be 80:20 to 20:80; or the ratio may be 60:40 to 40:60. Characteristics of light-emitting devices prepared in examples are tested using conventional devices in the art by a method well-known to those skilled in the art. As the persons skilled in the art are aware of the above-mentioned equipment use, test methods and other related contents, the inherent data of the sample can be obtained with certainty and without influence, so the above related contents are not further described in this patent. Compounds used in the present disclosure, such as a first metal complex, a first compound and a second compound, are easily obtained by those skilled in the art. For example, the compounds are commercially available or may be obtained with reference to the preparation method in the related art or may be obtained with reference to the preparation methods in U.S. Patent Publication Nos. US20200251666A1 and US2019363261A1, which are not repeated here.

### Device Example 1a

First, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and a vacuum degree of about 10⁻⁸ torr. Compound HI was used as a hole injection layer (HIL). Compound HT was used as a hole transporting layer (HTL). Compound C-1 was used as an electron blocking layer (EBL). Compound GD121 was doped in Compound 2-1 and Compound H-1, all of which were deposited for use as an emissive layer (EML). Compound C-3 was used as a hole blocking layer (HBL). On the HBL, Compound ET and 8-hydroxyquinolinolato-lithium (Liq) were co-deposited for use as an electron transporting layer (ETL). Finally, 8-hydroxyquinolinolato-lithium (Liq) was deposited as an electron injection layer with a thickness of 1 nm and Al was deposited as a cathode with a thickness of 120 nm. The device was transferred back to the glovebox and encapsulated with a glass cover plate to complete the device.

### Device Example 1b

Device Example 1b was prepared by the same method as Device Example 1a, except that in the EML, Compound 2-1 was replaced with Compound 2-171.

### Device Example 1c

Device Example 1c was prepared by the same method as Device Example 1a, except that in the EML, Compound 2-1 was replaced with Compound 2-167.

### Device Example 1d

Device Example 1d was prepared by the same method as Device Example 1a, except that in the EML, Compound 2-1 was replaced with Compound 2-170.

### Device Comparative Example 1a

Device Comparative Example 1a was prepared by the same method as Device Example 1a, except that in the EML, Compound 2-1 was replaced with Compound C-1.

### Device Comparative Example 1b

Device Comparative Example 1b was prepared by the same method as Device Example 1a, except that in the EML, Compound H-1 was replaced with Compound C-2.

Detailed structures and thicknesses of layers of the devices are shown in the following table. A layer using more than one material is obtained by doping different compounds at their weight ratio as recorded.

TABLE 1
Device structures in Device Examples 1a to 1d and
Device Comparative Examples 1a and 1b

| Device ID | HIL | HTL | EBL | EML | HBL | ETL |
|---|---|---|---|---|---|---|
| Example 1a | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-1:Compound | (50 Å) | (350 Å) |
| | | | | GD121 (64:28:8) | | |
| | | | | (400 Å) | | |
| Example 1b | Compound | Compound | Compound | Compound | Compound | Compound |
| | HI (100 Å) | HT | C-1 | 2-171:Compound | C-3 | ET:Liq (40:60) |
| | | (350 Å) | (50 Å) | H-1:Compound | (50 Å) | (350 Å) |
| | | | | GD121 (64:28:8) | | |
| | | | | (400 Å) | | |
| Example 1c | Compound | Compound | Compound | Compound | Compound | Compound |
| | HI (100 Å) | HT | C-1 | 2-167:Compound | C-3 | ET:Liq (40:60) |
| | | (350 Å) | (50 Å) | H-1:Compound | (50 Å) | (350 Å) |
| | | | | GD121 (64:28:8) | | |
| | | | | (400 Å) | | |
| Example 1d | Compound | Compound | Compound | Compound | Compound | Compound |
| | HI (100 Å) | HT | C-1 | 2-170:Compound | C-3 | ET:Liq (40:60) |
| | | (350 Å) | (50 Å) | H-1:Compound | (50 Å) | (350 Å) |
| | | | | GD121 (64:28:8) | | |
| | | | | (400 Å) | | |
| Comparative | Compound | Compound | Compound | Compound | Compound | Compound |
| Example 1a | HI (100 Å) | HT | C-1 | C-1:Compound | C-3 | ET:Liq (40:60) |
| | | (350 AÅ) | (50 Å) | H-1:Compound | (50 Å) | (350 Å) |
| | | | | GD121 (64:28:8) | | |
| | | | | (400 Å) | | |
| Comparative | Compound | Compound | Compound | Compound | Compound | Compound |
| Example 1b | HI (100 Å) | HT | C-1 | 2-1:Compound | C-3 | ET:Liq (40:60) |
| | | (350 Å) | (50 Å) | C-2:Compound | (50 Å) | (350 Å) |
| | | | | GD121 (64:28:8) | | |
| | | | | (400 Å) | | |

The structures of the materials used in the devices are shown as follows:

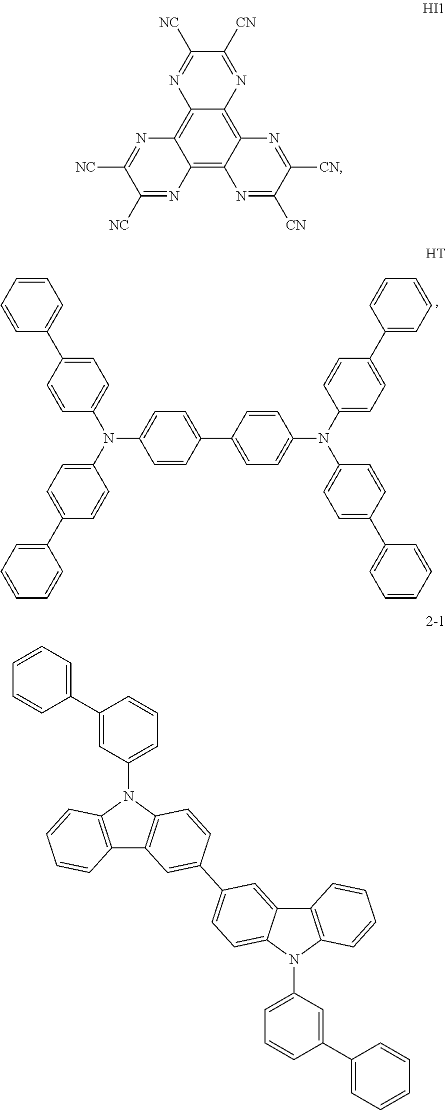 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

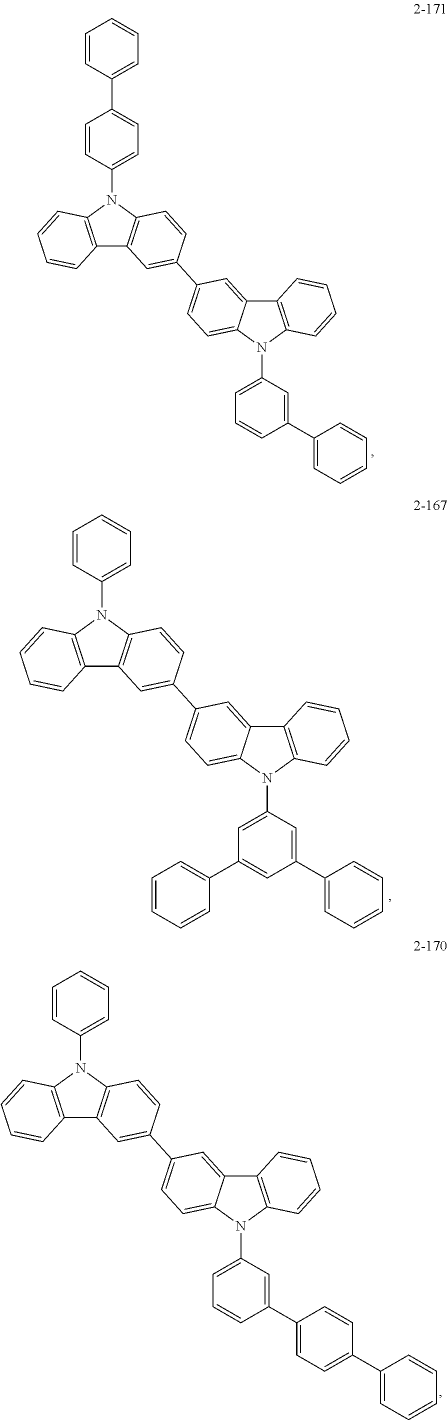 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

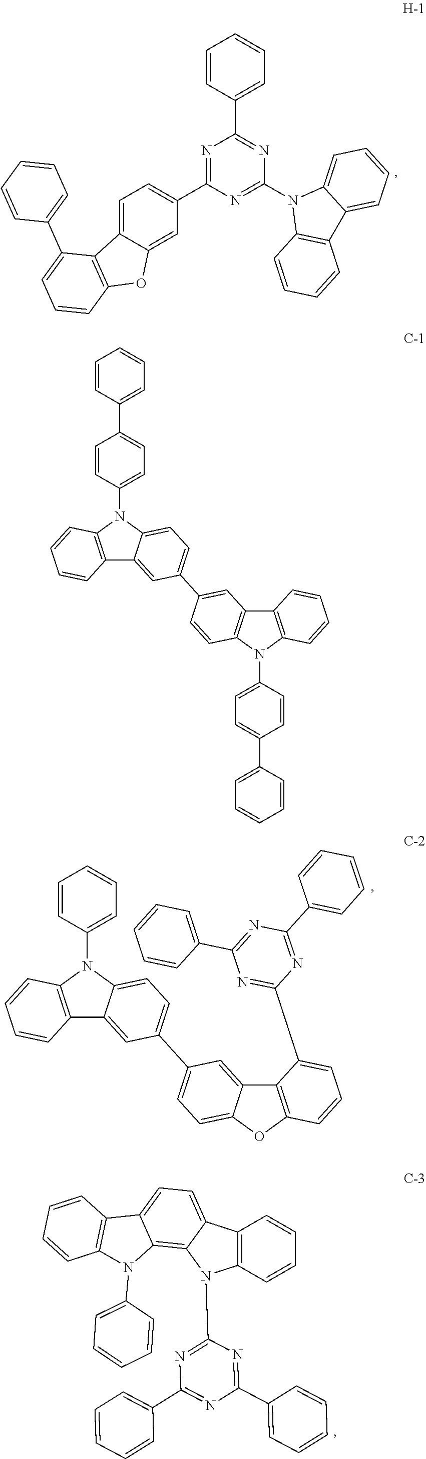

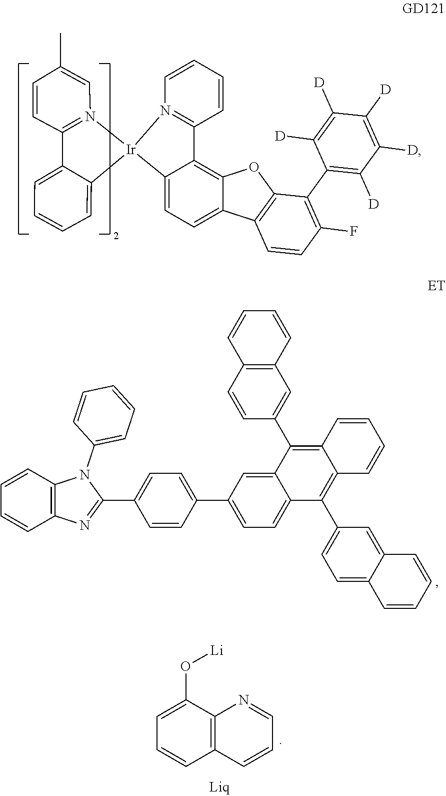

Table 2 shows CIE data and current efficiency measured at a constant current density of 15 mA/cm² and a device lifetime (LT95) measured at a constant current density of 80 mA/cm².

TABLE 2
Device data in Device Examples 1a to 1d and Device
Comparative Examples 1a and 1b

| | | | Current | |
|---|---|---|---|---|
| | | | Efficiency | Lifetime |
| Device ID | EML | CIE (x, y) | (cd/A) | LT95 (h) |
| Example 1a | 2-l:H-l:GD121 | (0.350, 0.624) | 85 | 105.0 |
| Example 1b | 2-171:H-l:GD121 | (0.349, 0.624) | 85 | 79.0 |
| Example 1c | 2-167:H-l:GD121 | (0.350, 0.624) | 85 | 71.9 |
| Example 1d | 2-170:H-l:GD121 | (0.351, 0.623) | 85 | 88.2 |
| Comparative | C-1:H-1:GD121 | (0.358, 0.618) | 84 | 49.7 |
| Example 1a | | | | |
| Comparative | 2-l:C-2:GD121 | (0.351, 0.624) | 84 | 54.3 |
| Example 1b | | | | |

### Discussion

In Examples 1a to 1d and Comparative Example 1a, Metal Complex GD121 of the present disclosure and the second compound H-1 of the present disclosure are used in the organic light-emitting layer, and the first compounds 2-1, 2-171, 2-167 and 2-170 of the present disclosure and non-inventive Compound C-1 are also used, respectively. As can be seen from the test results in Table 2, Examples 1a to 1d have substantially the same current efficiency as Comparative Example 1a. However, compared to that in Comparative Example 1a, the device lifetimes in Examples 1a to 1d are improved by 111.3%, 58.9%, 44.7% and 77.5%, respectively.

In Example 1a and Comparative Example 1b, Metal Complex GD121 of the present disclosure and the first compound 2-1 of the present disclosure are used in the organic light-emitting layer, and the second compound H-1 of the present disclosure and non-inventive Compound C-2 are also used, respectively. Compared to Comparative Example 1b, Example 1a has substantially the same current efficiency and a device lifetime improved by 93.4%. It is to be noted that Compound C-2 is a commercial host material. When an electroluminescent device comprising the first metal complex and the first compound of the present disclosure further comprises the second compound of the present application, its performance is significantly superior to that of an electroluminescent device comprising the commercially available Compound C-2, especially a significant improved device lifetime, showing the superiority of the performance of the electroluminescent device of the present disclosure.

As can be seen from the above discussion, compared to a non-inventive electroluminescent device, the electroluminescent device comprising the first compound, the second compound and the first metal complex of the present disclosure has a significantly extended device lifetime.

### Device Example 2

Device Example 2 was prepared by the same method as Device Example 1a, except that in the EML, Compound H-1 was replaced with Compound H-82.

### Device Comparative Example 2

Device Comparative Example 2 was prepared by the same method as Device Example 2, except that in the EML, Compound 2-1 was replaced with Compound C-1.

### Device Example 3

Device Example 3 was prepared by the same method as Device Example 1a, except that in the EML, Compound H-1 was replaced with Compound H-96.

### Device Comparative Example 3

Device Comparative Example 3 was prepared by the same method as Device Example 3, except that in the EML, Compound 2-1 was replaced with Compound C-1.

### Device Example 4

Device Example 4 was prepared by the same method as Device Example 1a, except that in the EML, Compound GD121 was replaced with Compound GD120.

### Device Comparative Example 4a

Device Comparative Example 4a was prepared by the same method as Device Example 4, except that in the EML, Compound 2-1 was replaced with Compound C-1.

### Device Comparative Example 4b

Device Comparative Example 4b was prepared by the same method as Device Example 4, except that in the EML, Compound H-1 was replaced with Compound C-2.

Detailed structures and thicknesses of layers of the devices are shown in the following table. A layer using more than one material is obtained by doping different compounds at their weight ratio as recorded.

TABLE 3
Device structures in Device Examples 2 to 4 and
Device Comparative Examples 2, 3, 4a and 4b

| Device ID | HIL | HTL | EBL | EML | HBL | ETL |
|---|---|---|---|---|---|---|
| Example 2 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-82:Compound | (50 Å) | (350 Å) |
| | | | | GD121 (64:28:8) | | |
| | | | | (400 Å) | | |
| Comparative | Compound | Compound | Compound | Compound | Compound | Compound |
| Example 2 | HI (100 Å) | HT | C-1 | C-1:Compound | C-3 | ET:Liq (40:60) |
| | | (350 Å) | (50 Å) | H-82:Compound | (50 Å) | (350 Å) |
| | | | | GD121 (64:28:8) | | |
| | | | | (400 Å) | | |
| Example 3 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-96:Compound | (50 Å) | (350 Å) |
| | | | | GD121 (64:28:8) | | |
| | | | | (400 Å) | | |
| Comparative | Compound | Compound | Compound | Compound | Compound | Compound |
| Example 3 | HI (100 Å) | HT | C-1 | C-1:Compound | C-3 | ET:Liq (40:60) |
| | | (350 Å) | (50 Å) | H-96:Compound | (50 Å) | (350 Å) |
| | | | | GD121 (64:28:8) | | |
| | | | | (400 Å) | | |
| Example 4 | Compound | Compound | Compound | Compound | | |
| | HI (100 Å) | HT | C-1 | 2-1:Compound | Compound | Compound |
| | | (350 Å) | (50 Å) | H-1:Compound | C-3 | ET:Liq (40:60) |
| | | | | GD120 (64:28:8) | (50 Å) | (350 Å) |
| | | | | (400 Å) | | |
| Comparative | Compound | Compound | Compound | Compound | Compound | Compound |
| Example 4a | HI (100 Å) | HT | C-1 | C-1:Compound | C-3 | ET:Liq (40:60) |
| | | (350 Å) | (50 Å) | H-1:Compound | (50 Å) | (350 Å) |
| | | | | GD120 (64:28:8) | | |
| | | | | (400 Å) | | |
| Comparative | Compound | Compound | Compound | Compound | Compound | Compound |
| Example 4b | HI (100 Å) | HT | C-1 | 2-1:Compound | C-3 | ET:Liq (40:60) |
| | | (350 Å) | (50 Å) | C-2:Compound | (50 Å) | (350 Å) |
| | | | | GD120 (64:28:8) | | |
| | | | | (400 Å) | | |

The structures of the materials used in the devices are shown as follows:

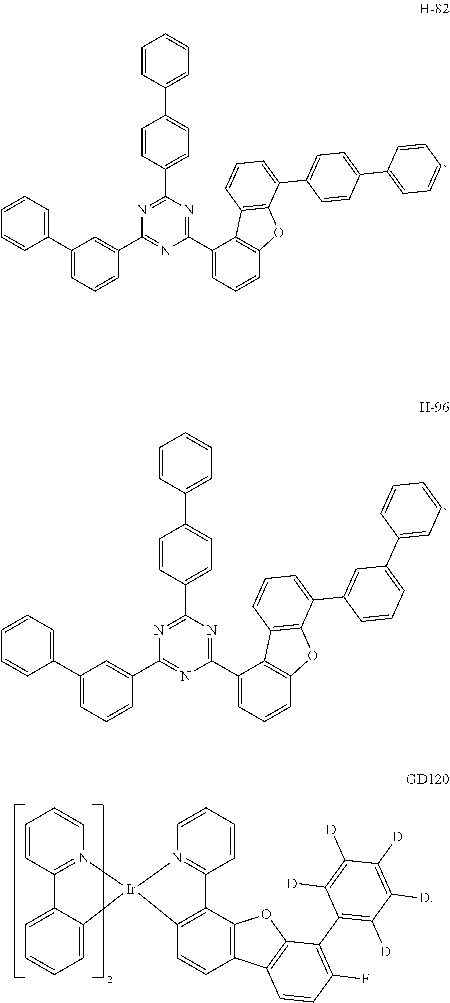

Table 4 shows CIE data and current efficiency measured at a constant current density of 15 mA/cm² and a device lifetime (LT95) measured at a constant current density of 80 mA/cm².

TABLE 4
Device data in Device Examples 2 to 4 and Device Comparative Examples 2, 3, 4a and 4b

| | | | Current | |
|---|---|---|---|---|
| | | | Efficiency | Lifetime |
| Device ID | EML | CIE (x, y) | (cd/A) | LT95 (h) |
| Example 2 | 2-l:H-82:GD121 | (0.355, 0.620) | 85 | 59.0 |
| Comparative | C-1:H-82:GD121 | (0.355, 0.620) | 86 | 48.9 |
| Example 2 | | | | |
| Example 3 | 2-l:H-96:GD121 | (0.354, 0.621) | 85 | 62.2 |
| Comparative | C-1:H-96:GD121 | (0.354, 0.621) | 86 | 50.0 |
| Example 3 | | | | |
| Example 4 | 2-l:H-l:GD120 | (0.337, 0.631) | 85 | 74.7 |
| Comparative | C-l:H-l:GD120 | (0.339, 0.630) | 83 | 52.2 |
| Example 4a | | | | |
| Comparative | 2-l:C-2:GD120 | (0.337, 0.631) | 85 | 45.3 |

Example 4b

### Discussion

In Example 2 and Comparative Example 2, Metal Complex GD121 of the present disclosure and the second host compound H-82 of the present disclosure are used in the organic light-emitting layer, and Compound 2-1 of the present disclosure and non-inventive Compound C-1 are also used, respectively. Compared to Comparative Example 2, Example 2 has substantially the same current efficiency and a device lifetime improved by 20.7%.

In Example 3 and Comparative Example 3, Metal Complex GD121 of the present disclosure and the second host compound H-96 of the present disclosure are used in the organic light-emitting layer, and Compound 2-1 of the present disclosure and non-inventive Compound C-1 are also used, respectively. Compared to Comparative Example 3, Example 3 has substantially the same current efficiency and a device lifetime improved by 24.4%.

In Example 4 and Comparative Example 4a, Metal Complex GD120 of the present disclosure and the second compound H-1 of the present disclosure are used in the organic light-emitting layer, and the first compound 2-1 of the present disclosure and non-inventive Compound C-1 are also used, respectively. Compared to Comparative Example 4a, Example 4 has slightly improved current efficiency and a device lifetime improved by 43.1%.

In Example 4 and Comparative Example 4b, Metal Complex GD120 of the present disclosure and the first compound 2-1 of the present disclosure are used in the organic light-emitting layer, and the second compound H-1 of the present disclosure and non-inventive Compound C-2 are also used, respectively. Compared to Comparative Example 4b, Example 4 has substantially the same current efficiency and a device lifetime improved by 64.9%. It is to be noted that Compound C-2 is a commercially available host material. When an electroluminescent device comprising the first metal complex and the first compound of the present disclosure further comprises the second compound of the present application, its performance is significantly superior to that of an electroluminescent device comprising the commercially available Compound C-2, especially a significant extended device lifetime, showing the superiority of the performance of the electroluminescent device of the present disclosure.

As can be seen from the above, compared to an electroluminescent device not provided in the present disclosure, the electroluminescent device comprising the first compound, the second compound and the first metal complex of the present disclosure has a significantly extended device lifetime.

### Device Example 5

Device Example 5 was prepared by the same method as Device Example 1a, except that in the EML, Compound H-1 was replaced with Compound H-107 and 2-1:H-107:GD121=69:23:8.

### Device Comparative Example 5a

Device Comparative Example 5a was prepared by the same method as Device Example 5, except that in the EML, Compound 2-1 was replaced with Compound C-1.

### Device Comparative Example 5b

Device Comparative Example 5b was prepared by the same method as Device Example 5, except that in the EML, Compound H-107 was replaced with Compound C-3.

### Device Example 6

Device Example 6 was prepared by the same method as Device Example 5, except that in the EML, Compound H-107 was replaced with Compound H-119.

### Device Comparative Example 6a

Device Comparative Example 6a was prepared by the same method as Device Example 6, except that in the EML, Compound 2-1 was replaced with Compound C-1.

### Device Comparative Example 6b

Device Comparative Example 6b was prepared by the same method as Device Example 6, except that in the EML, Compound GD121 was replaced with Compound C-4.

Detailed structures and thicknesses of layers of the devices are shown in the following table. A layer using more than one material is obtained by doping different compounds at their weight ratio as recorded.

TABLE 5
Device structures in Device Examples 5 and 6 and
Device Comparative Examples 5a, 5b, 6a and 6b

| Device ID | HIL | HTL | EBL | EML | HBL | ETL |
|---|---|---|---|---|---|---|
| Example 5 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-107:Compound | (50 Å) | (350 Å) |
| | | | | GD121 (69:23:8) | | |
| | | | | (400 Å) | | |
| Comparative | Compound | Compound | Compound | Compound | Compound | Compound |
| Example 5a | HI (100 Å) | HT | C-1 | C-1:Compound | C-3 | ET:Liq (40:60) |
| | | (350 Å) | (50 Å) | H-107:Compound | (50 Å) | (350 Å) |
| | | | | GD121 (69:23:8) | | |
| | | | | (400 Å) | | |
| Comparative | Compound | Compound | Compound | Compound | Compound | Compound |
| Example 5b | HI (100 Å) | HT | C-1 | 2-1:Compound | C-3 | ET:Liq (40:60) |
| | | (350 Å) | (50 Å) | C-3:Compound | (50 Å) | (350 Å) |
| | | | | GD121 (69:23:8) | | |
| | | | | (400 Å) | | |
| Example 6 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-119:Compound | (50 Å) | (350 Å) |
| | | | | GD121 (69:23:8) | | |
| | | | | (400 Å) | | |
| Comparative | Compound | Compound | Compound | Compound | Compound | Compound |
| Example 6a | HI (100 Å) | HT | C-1 | C-1:Compound | C-3 | ET:Liq (40:60) |
| | | (350 Å) | (50 Å) | H-119:Compound | (50 Å) | (350 Å) |
| | | | | GD121 (69:23:8) | | |
| | | | | (400 Å) | | |
| Comparative | Compound | Compound | Compound | Compound | Compound | Compound |
| Example 6b | HI (100 Å) | HT | C-1 | 2-1:Compound | C-3 | ET:Liq (40:60) |
| | | (350 Å) | (50 Å) | H-119:Compound | (50 Å) | (350 Å) |
| | | | | C-4 (69:23:8) | | |
| | | | | (400 Å) | | |

The structures of the new materials used in the devices are shown as follows:

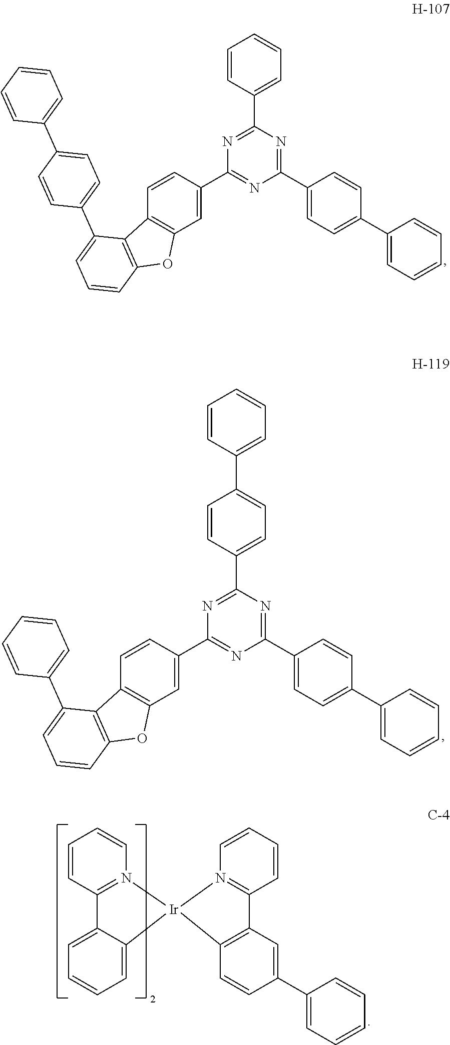

Table 6 shows CIE data and current efficiency measured at a constant current density of 15 mA/cm² and a device lifetime (LT95) measured at a constant current density of 80 mA/cm².

TABLE 6
Device data in Device Examples 5 and 6 and Device Comparative Examples 5a, 5b, 6a and 6b

| | | | Current | |
|---|---|---|---|---|
| | | | Efficiency | Lifetime |
| Device ID | EML | CIE (x, y) | (cd/A) | LT95 (h) |
| Example 5 | 2-l:H-107:GD121 | (0.354, 0.620) | 85 | 67.9 |
| Comparative | C-l:H-107:GD121 | (0.357, 0.619) | 85 | 54.5 |
| Example 5a | | | | |
| Comparative | 2-l:C-3:GD121 | (0.345, 0.627) | 81 | 41.0 |
| Example 5b | | | | |
| Example 6 | 2-l:H-119:GD121 | (0.355, 0.620) | 83 | 66.5 |
| Comparative | C-1:H-119:GD121 | (0.358, 0.618) | 83 | 59.3 |
| Example 6a | | | | |
| Comparative | 2-l:H-119:C-4 | (0.320, 0.630) | 67 | 42.0 |
| Example 6b | | | | |

### Discussion

In Example 5 and Comparative Example 5a, Metal Complex GD121 of the present disclosure and the second compound H-107 of the present disclosure are used in the organic light-emitting layer, and the first compound 2-1 of the present disclosure and non-inventive Compound C-1 are also used, respectively. Compared to Comparative Example 5, Example 5 has substantially the same current efficiency and a device lifetime improved by 24.6%.

Similarly, in Example 6 and Comparative Example 6a, Metal Complex GD121 of the present disclosure and the second compound H-119 of the present disclosure are used in the organic light-emitting layer, and the first compound 2-1 of the present disclosure and non-inventive Compound C-1 are also used, respectively. Compared to Comparative Example 6a, Example 6 has substantially the same current efficiency and a device lifetime improved by 12.1%.

In Example 5 and Comparative Example 5b, Metal Complex GD121 of the present disclosure and the first compound 2-1 of the present disclosure are used in the organic light-emitting layer, and the second compound H-107 of the present disclosure and non-inventive Compound C-3 are also used, respectively. Compared to Comparative Example 5b, Example 5 has slightly improved current efficiency and a device lifetime improved by 65.6%.

In Example 6 and Comparative Example 6b, the first compound 2-1 of the present disclosure and the host compound H-119 of the present disclosure are used in the organic light-emitting layer, and Metal Complex GD121 of the present disclosure and non-inventive Metal Complex C-4 are also used, respectively. Compared to Comparative Example 6b, Example 6 has current efficiency and a device lifetime significantly improved by 23.8% and 58.3%, respectively.

As can be seen from the above results, compared to an electroluminescent device not provided in the present disclosure, the electroluminescent device comprising the first compound, the second compound and the first metal complex of the present disclosure has significantly improved device performance, especially an extended device lifetime.

### Device Example 7

Device Example 7 was prepared by the same method as Device Example 1a, except that in the EML, Compound GD121 was replaced with Compound GD30 and 2-1:H-1:GD30=47:47:6.

### Device Comparative Example 7a

Device Comparative Example 7a was prepared by the same method as Device Example 7, except that in the EML, Compound 2-1 was replaced with Compound C-1.

### Device Comparative Example 7b

Device Comparative Example 7b was prepared by the same method as Device Example 7, except that in the EML, Compound H-1 was replaced with Compound C-2.

### Device Example 8

Device Example 8 was prepared by the same method as Device Example 7, except that in the EML, Compound GD30 was replaced with Compound GD43.

### Device Comparative Example 8a

Device Comparative Example 8a was prepared by the same method as Device Example 8, except that in the EML, Compound H-1 was replaced with Compound C-2.

### Device Comparative Example 8b

Device Comparative Example 8b was prepared by the same method as Device Example 8, except that in the EML, Compound GD43 was replaced with Compound C-5.

Detailed structures and thicknesses of layers of the devices are shown in the following table. A layer using more than one material is obtained by doping different compounds at their weight ratio as recorded.

TABLE 7
Device structures in Device Examples 7 and 8 and
Device Comparative Examples 7a, 7b, 8a and 8b

| Device ID | HIL | HTL | EBL | EML | HBL | ETL |
|---|---|---|---|---|---|---|
| Example 7 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-1:Compound | (50 Å) | (350 Å) |
| | | | | GD30 (47:47:6) | | |
| | | | | (400 Å) | | |
| Comparative | Compound | Compound | Compound | Compound | Compound | Compound |
| Example 7a | HI (100 Å) | HT | C-1 | C-1:Compound | C-3 | ET:Liq (40:60) |
| | | (350 Å) | (50 Å) | H-1:Compound | (50 Å) | (350 Å) |
| | | | | GD30 (47:47:6) | | |
| | | | | (400 Å) | | |
| Comparative | Compound | Compound | Compound | Compound | Compound | Compound |
| Example 7b | HI (100 Å) | HT | C-1 | 2-1:Compound | C-3 | ET:Liq (40:60) |
| | | (350 Å) | (50 Å) | C-2:Compound | (50 Å) | (350 Å) |
| | | | | GD30 (47:47:6) | | |
| | | | | (400 Å) | | |
| Example 8 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-1:Compound | (50 Å) | (350 Å) |
| | | | | GD43 (47:47:6) | | |
| | | | | (400 Å) | | |
| Comparative | Compound | Compound | Compound | Compound | Compound | Compound |
| Example 8a | HI (100 Å) | HT | C-1 | 2-1:Compound | C-3 | ET:Liq (40:60) |
| | | (350 Å) | (50 Å) | C-2:Compound | (50 Å) | (350 Å) |
| | | | | GD43 (47:47:6) | | |
| | | | | (400 Å) | | |
| Comparative | Compound | Compound | Compound | Compound | Compound | Compound |
| Example 8b | HI (100 Å) | HT | C-1 | 2-1:Compound | C-3 | ET:Liq (40:60) |
| | | (350 Å) | (50 Å) | H-1:Compound | (50 Å) | (350 Å) |
| | | | | C-5 (47:47:6) | | |
| | | | | (400 Å) | | |

The structures of the new materials used in the devices are shown as follows:

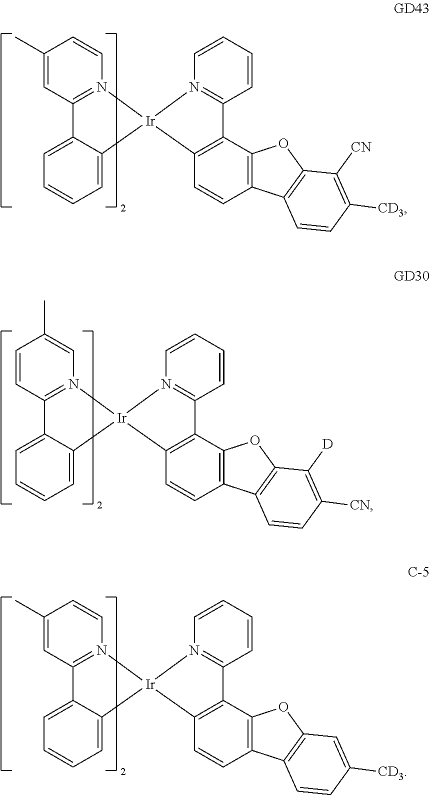

Table 8 shows CIE data and current efficiency measured at a constant current density of 15 mA/cm² and a device lifetime (LT95) measured at a constant current density of 80 mA/cm².

TABLE 8
Device data in Device Examples 7 and 8 and Device Comparative Examples 7a, 7b, 8a and 8b

| | | | Current | |
|---|---|---|---|---|
| | | | Efficiency | Lifetime |
| Device ID | EML | CIE (x, y) | (cd/A) | LT95 (h) |
| Example 7 | 2-l:H-l:GD30 | (0.339, 0.635) | 91 | 78.7 |
| Comparative | C-l:H-l:GD30 | (0.340, 0.634) | 90 | 65.5 |
| Example 7a | | | | |
| Comparative | 2-l:C-2:GD30 | (0.341, 0.634) | 89 | 42.7 |
| Example 7b | | | | |
| Example 8 | 2-l:H-l:GD43 | (0.343, 0.629) | 85 | 52.0 |
| Comparative | 2-l:C-2:GD43 | (0.343, 0.629) | 83 | 31.5 |
| Example 8a | | | | |
| Comparative | 2-l:H-l:C-5 | (0.366, 0.612) | 76 | 43.5 |
| Example 8b | | | | |

### Discussion

In Example 7 and Comparative Example 7a, Metal Complex GD30 of the present disclosure and the second compound H-1 of the present disclosure are used in the organic light-emitting layer, and the first compound 2-1 of the present disclosure and non-inventive Compound C-1 are also used, respectively. Compared to Comparative Example 7a, Example 7 has improved current efficiency and a device lifetime improved by 20.1%. Similarly, when Metal Complex GD43 of the present disclosure is used, compared to Comparative Example 8a, Example 8 also has improved current efficiency and a device lifetime improved by 65.1%.

In Example 7 and Comparative Example 7b, Metal Complex GD30 of the present disclosure and the first compound 2-1 of the present disclosure are used in the organic light-emitting layer, and the second compound H-1 of the present disclosure and non-inventive Compound C-2 are also used, respectively. Compared to Comparative Example 7b, Example 7 has improved current efficiency and a device lifetime improved by 84.3%.

In Examples 7 and 8 and Comparative Example 8b, the first compound 2-1 of the present disclosure and the second compound H-1 of the present disclosure are used in the organic light-emitting layer, and Metal Complexes GD30 and GD43 of the present disclosure and non-inventive Metal Complex C-5 are also used, respectively. Compared to Comparative Example 8b, Examples 7 and 8 have current efficiency improved by 19.7% and 11.8%, respectively and device lifetimes improved by 80.9% and 19.5%, respectively.

As can be seen from the above results, compared to an electroluminescent device not provided in the present disclosure, the electroluminescent device comprising the first compound, the second compound and the first metal complex of the present disclosure has significantly improved device performance, especially an extended device lifetime.

### Device Example 9

Device Example 9 was prepared by the same method as Device Example 1a, except that in the EML, Compound H-1 was replaced with Compound H-120 and 2-1:H-120:GD121=69:23:8.

### Device Example 10

Device Example 10 was prepared by the same method as Device Example 9, except that in the EML, Compound H-120 was replaced with Compound H-179.

### Device Example 11

Device Example 11 was prepared by the same method as Device Example 9, except that in the EML, Compound H-120 was replaced with Compound H-180.

Detailed structures and thicknesses of layers of the devices are shown in the following table. A layer using more than one material is obtained by doping different compounds at their weight ratio as recorded.

TABLE 9
Device structures in Device Examples 9-11

| Device ID | HIL | HTL | EBL | EML | HBL | ETL |
|---|---|---|---|---|---|---|
| Example 9 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-120:Compound | (50 Å) | (350 Å) |
| | | | | GD121 (69:23:8) | | |
| | | | | (400 Å) | | |
| Example 10 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-179:Compound | (50 Å) | (350 Å) |
| | | | | GD121 (69:23:8) | | |
| | | | | (400 Å) | | |
| Example 11 | Compound | Compound | Compound | Compound | Compound | Compound |
| | HI (100 Å) | HT | C-1 | 2-1:Compound | C-3 | ET:Liq (40:60) |
| | | (350 Å) | (50 Å) | H-180:Compound | (50 Å) | (350 Å) |
| | | | | GD121 (69:23:8) | | |
| | | | | (400 Å) | | |

The structures of the new materials used in the devices are shown as follows:

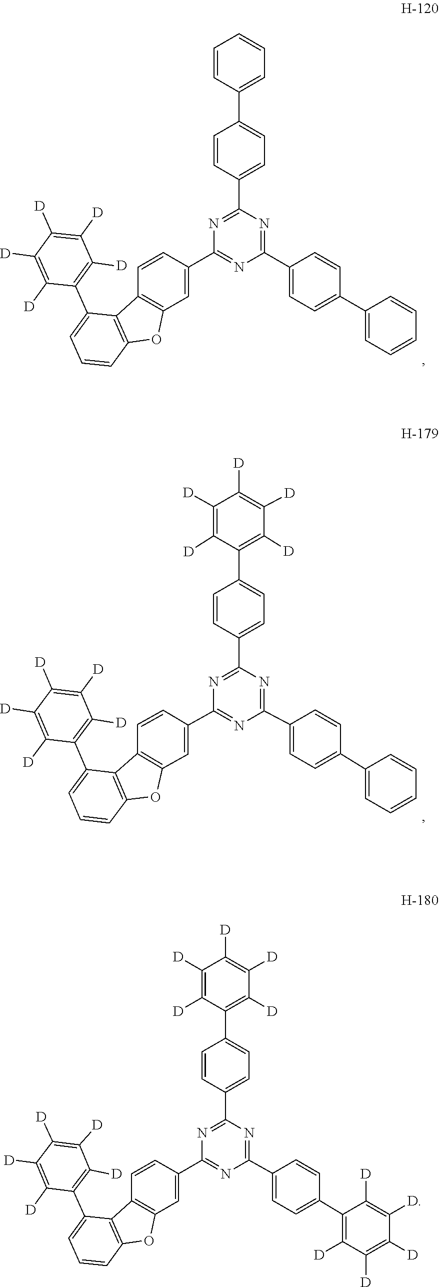

Table 10 shows CIE data and current efficiency measured at a constant current density of 15 mA/cm² and a device lifetime (LT95) measured at a constant current density of 80 mA/cm².

TABLE 10
Device data in Device Examples 9-11

| | | | Current | |
|---|---|---|---|---|
| | | | Efficiency | Lifetime |
| Device ID | EML | CIE (x, y) | (cd/A) | LT95 (h) |
| Example 9 | 2-l:H-120:GD121 | (0.354, 0.621) | 85 | 93.5 |
| Example 10 | 2-l:H-179:GD121 | (0.356, 0.619) | 83 | 85.2 |
| Example 11 | 2-l:H-180:GD121 | (0.357, 0.619) | 83 | 78.7 |

### Discussion

In Example 9 to Example 11, Metal Complex GD121 of the present disclosure and the first compound 2-1 of the present disclosure are used in the organic light-emitting layer, and the second compound H-120, H-179 and H-180 of the present disclosure are also used, those devices achieved a high current efficiency and a long device lifetime. In particular, the performance of Examples 9 to 11 has been further improved on the basis of Example 6, and the only difference between Examples 9 to 11 and Example 6 are that the second compound used are deuterium substituted for different aryl groups in compound H-119, and its performance was greatly improved, and the device lifetime was increased by 40.6%, 28.1% and 18.3%, respectively.

To conclude, compared to an electroluminescent device not provided in the present disclosure, the electroluminescent device comprising the first compound, the second compound and the first metal complex of the present disclosure has significantly improved device performance, especially a significantly extended device lifetime. Therefore, an excellent material combination of the light-emitting layer is provided for the industry.

It should be understood that various embodiments described herein are merely examples and not intended to limit the scope of the present disclosure. Therefore, it is apparent to the persons skilled in the art that the present disclosure as claimed may include variations from specific embodiments and preferred embodiments described herein. Many of materials and structures described herein may be substituted with other materials and structures without departing from the spirit of the present disclosure. It should be understood that various theories as to why the present disclosure works are not intended to be limitative.

What is claimed is:

1. An electroluminescent device, comprising:
an anode,
a cathode, and
an organic layer disposed between the anode and the cathode, wherein the organic layer comprises at least a first metal complex, a first compound and a second compound;
wherein the first metal complex comprises a metal M and a ligand Lₐ coordinated to the metal M, wherein the ligand Lₐ has a structure represented by Formula 1: 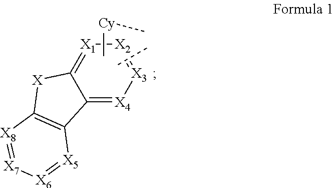
wherein
the metal M is selected from a metal with a relative atomic mass greater than 40;
Cy is, at each occurrence identically or differently, selected from substituted or unsubstituted aryl having 5 to 24 ring atoms or substituted or unsubstituted heteroaryl having 5 to 24 ring atoms; and Cy is joined to the metal M by a metal-carbon bond or a metal-nitrogen bond;
X is, at each occurrence identically or differently, selected from the group consisting of O, S, Se, NR′, CR′R′ and SiR′R′; wherein when two R′ are present, the two R′ are identical or different;
X₁ to X₈ are, at each occurrence identically or differently, selected from C, CRₓ or N, wherein at least one of X₁ to X₄ is C and joined to Cy;
X₁, X₂, X₃ or X₄ is joined to the metal M by a metal-carbon bond or a metal-nitrogen bond;
at least one of X₁ to X₈ is CRₓ, wherein the Rₓ is cyano or fluorine; and
adjacent substituents R′, Rₓ can be optionally joined to form a ring;
wherein the first compound has a structure represented by Formula 2: 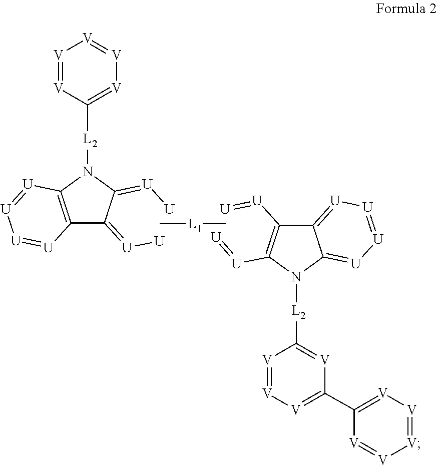
wherein
U is, at each occurrence identically or differently, selected from C, CRᵤ or N;
V is, at each occurrence identically or differently, selected from CRᵥ or N;
L₁ and L₂ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and
adjacent substituents Rᵤ and Rᵥ can be optionally joined to form a ring;
wherein the second compound has a structure represented by Formula 3: 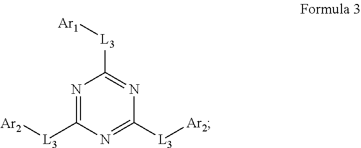
wherein
Ar₁ has a structure represented by Formula A: 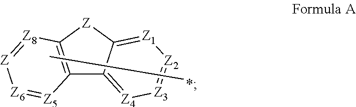
wherein
Z is, at each occurrence identically or differently, selected from the group consisting of O, S and Se;
L₃ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;
Z₁ to Z₈ are, at each occurrence identically or differently, selected from C, CR_{z} or N, and at least one of Z₁ to Z₈ is C and joined to L₃;
at least one of Z₁ to Z₈ is CR_{z}, wherein the R_{z} is substituted or unsubstituted aryl having 6 to 30 carbon atoms;
Ar₂ is, at each occurrence identically or differently, selected from substituted or unsubstituted aryl having 6 to 30 carbon atoms, substituted or unsubstituted heteroaryl having 3 to 30 carbon atoms or a combination thereof;
“*” represents a position where Ar₁ is joined to L₃;
adjacent substituents R_{z} can be optionally joined to form a ring;
R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and
R′, Rₓ, Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof.

2. The electroluminescent device according to claim 1, wherein Cy is selected from any structure in the group consisting of: 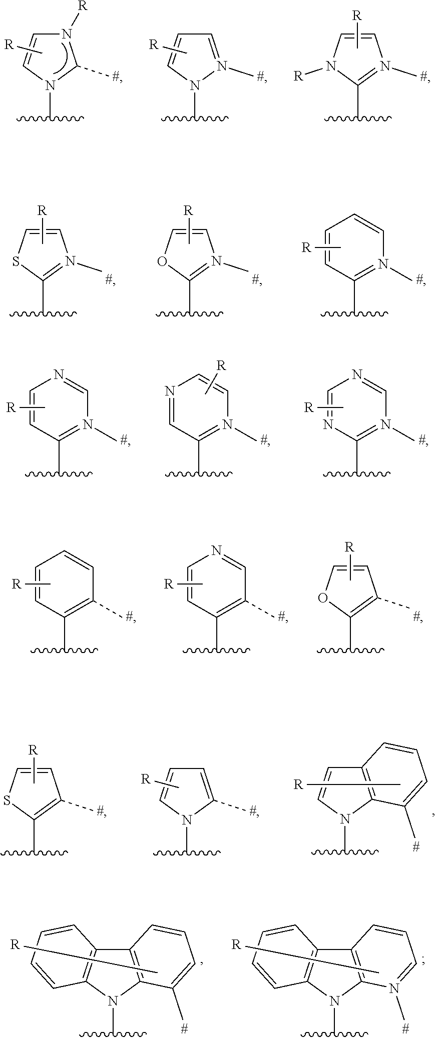
wherein R represents, at each occurrence identically or differently, mono-substitution, multiple substitutions or non-substitution; and when a plurality of R are present in any structure, the plurality of R may be identical or different;
R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;
two adjacent substituents R can be optionally joined to form a ring; and
“#” represents a position where Cy is joined to the metal M, and 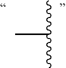
represents a position where Cy is joined to X₁, X₂, X₃ or X₄.

3. The electroluminescent device according to claim 1, wherein the first metal complex has a general formula of M(Lₐ)ₘ(L_{b})ₙ(L_{c})_{q};
wherein
M is, at each occurrence identically or differently, selected from the group consisting of Cu, Ag, Au, Ru, Rh, Pd, Os, Ir and Pt; preferably, M is, at each occurrence identically or differently, selected from Pt or Ir;
Lₐ, L_{b} and L_{c} are a first ligand, a second ligand and a third ligand coordinated to the metal M, respectively, and L_{c} is identical to or different from Lₐ or L_{b}; wherein Lₐ, L_{b} and L_{c} can be optionally joined to form a multidentate ligand;
m is selected from 1, 2 or 3, n is selected from 0, 1 or 2, q is selected from 0, 1 or 2, and m+n+q equals an oxidation state of the metal M; wherein when m is greater than or equal to 2, a plurality of Lₐ are identical or different; when n is equal to 2, two L_{b} are identical or different; when q is equal to 2, two L_{c} are identical or different;
Lₐ is, at each occurrence identically or differently, selected from the group consisting of: 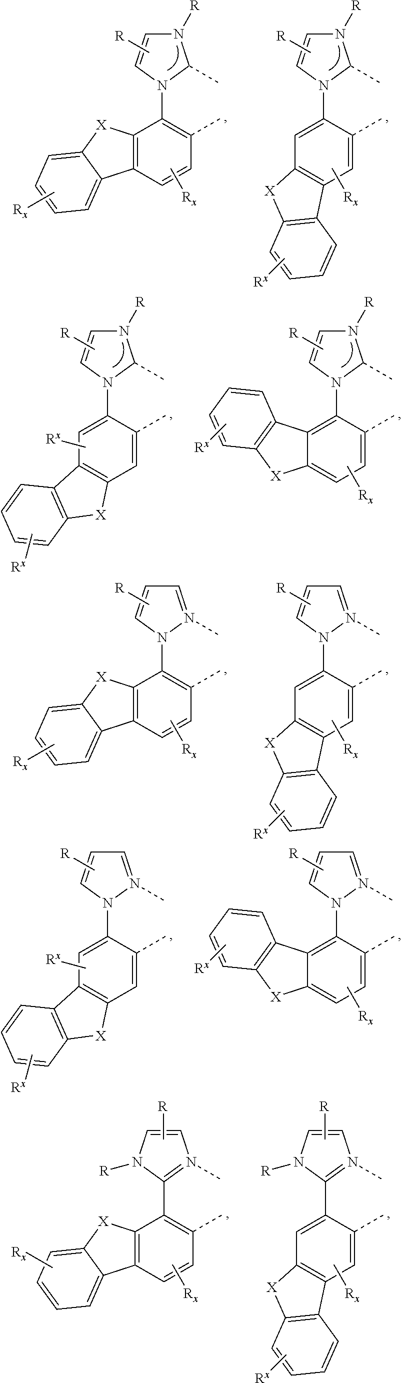 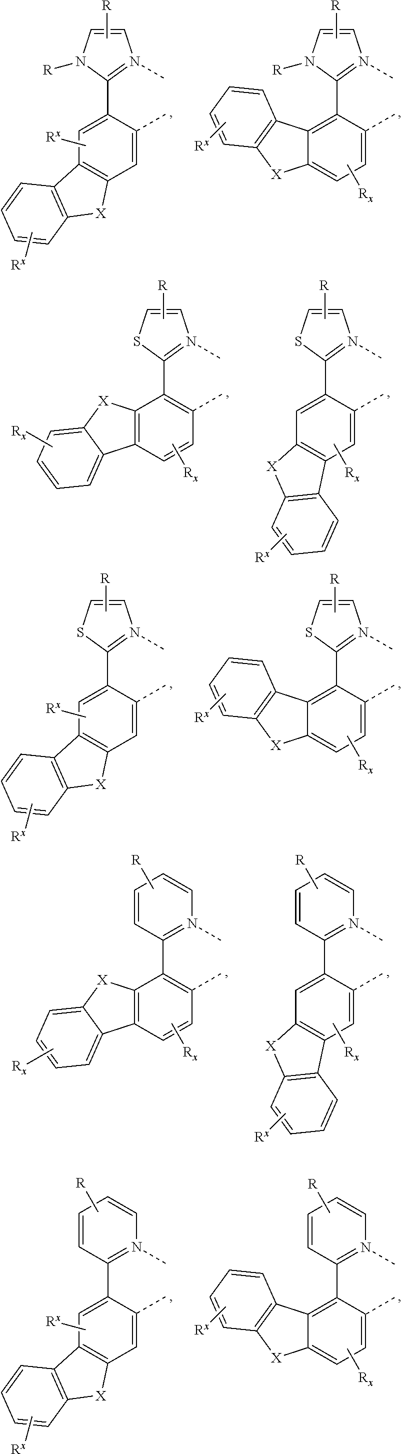 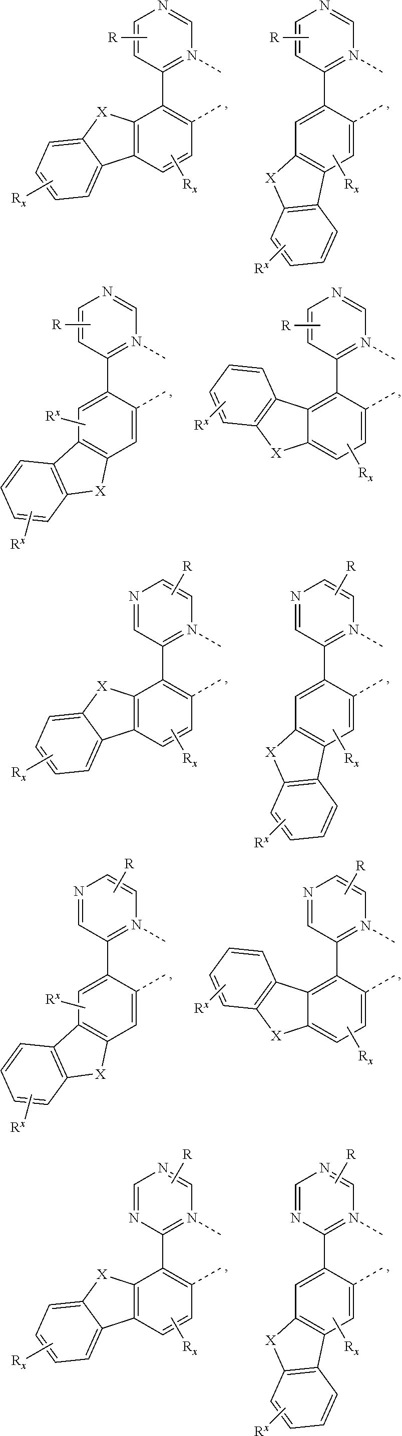 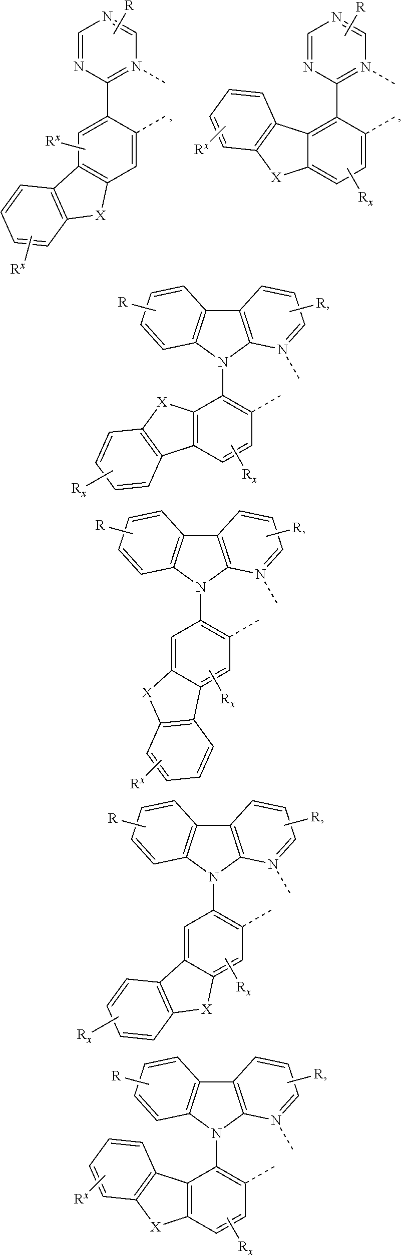 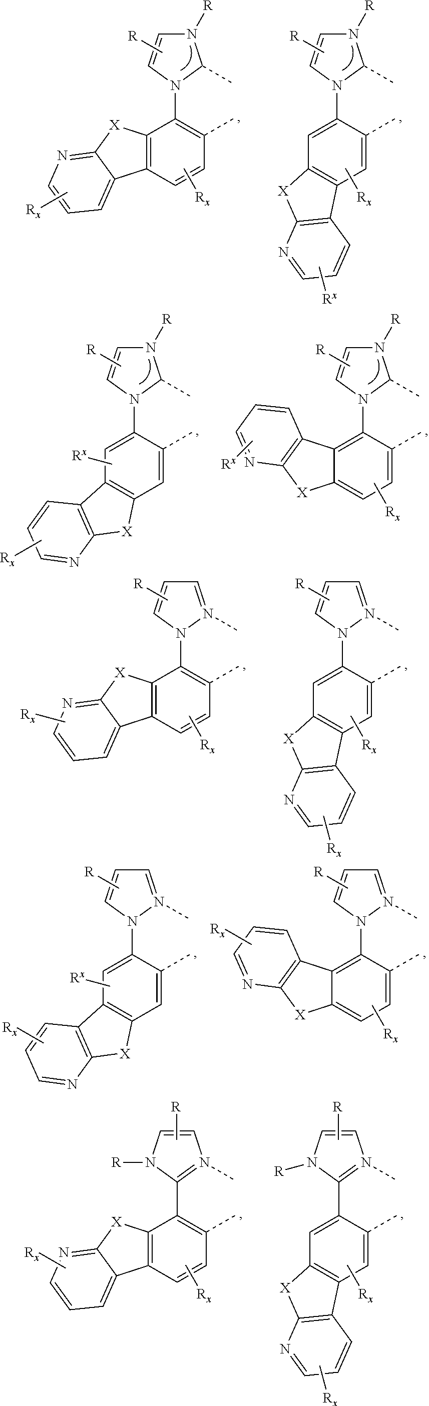 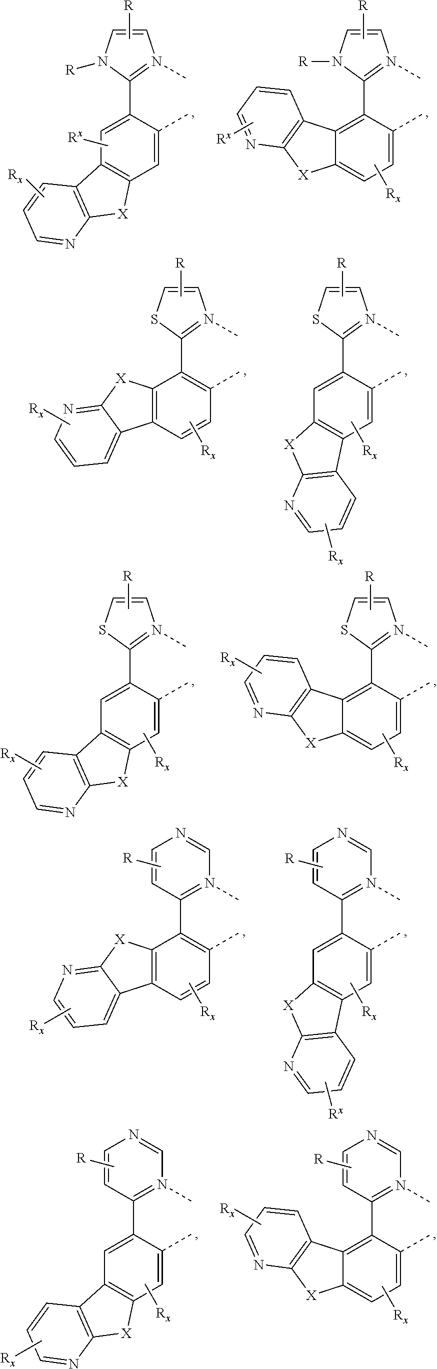 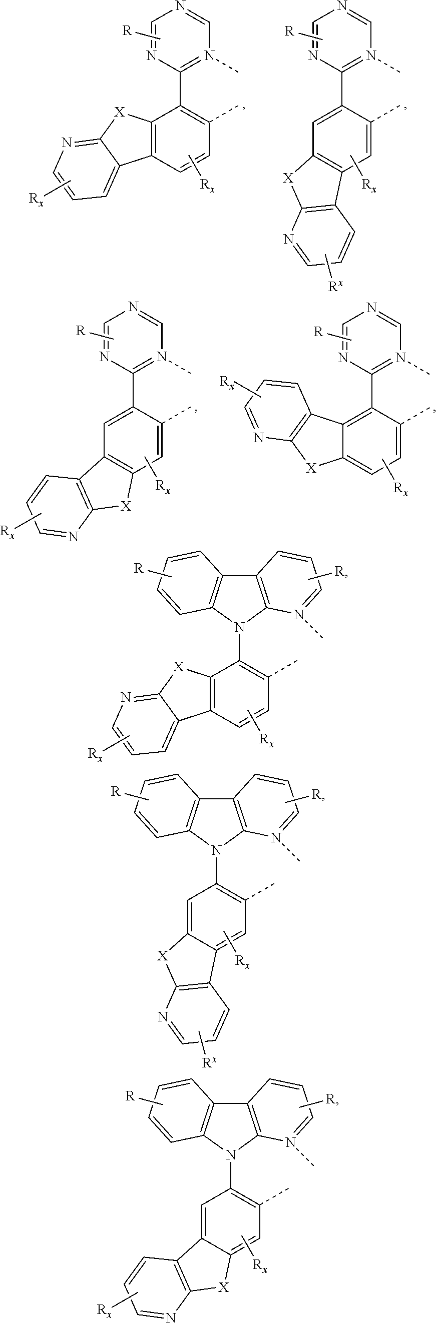 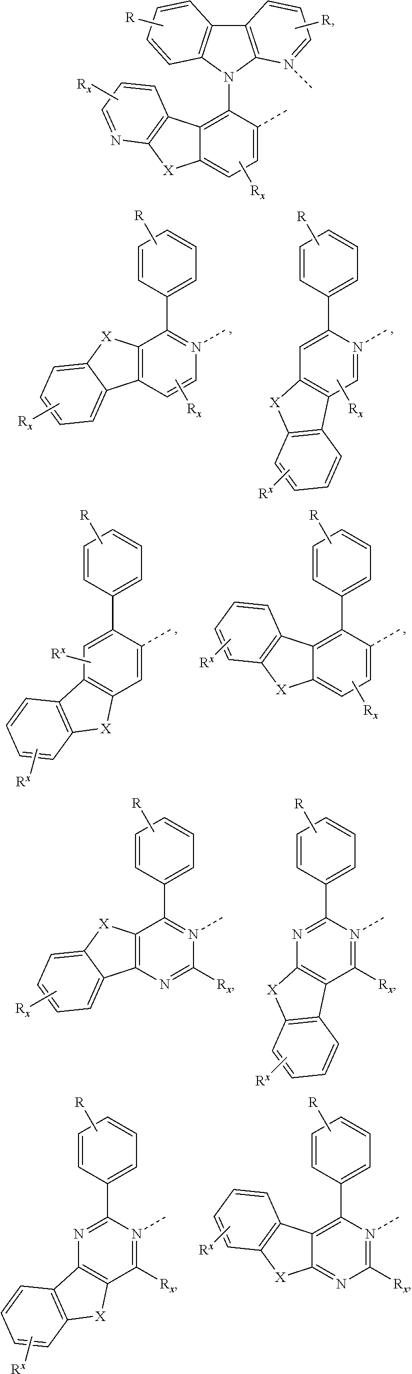 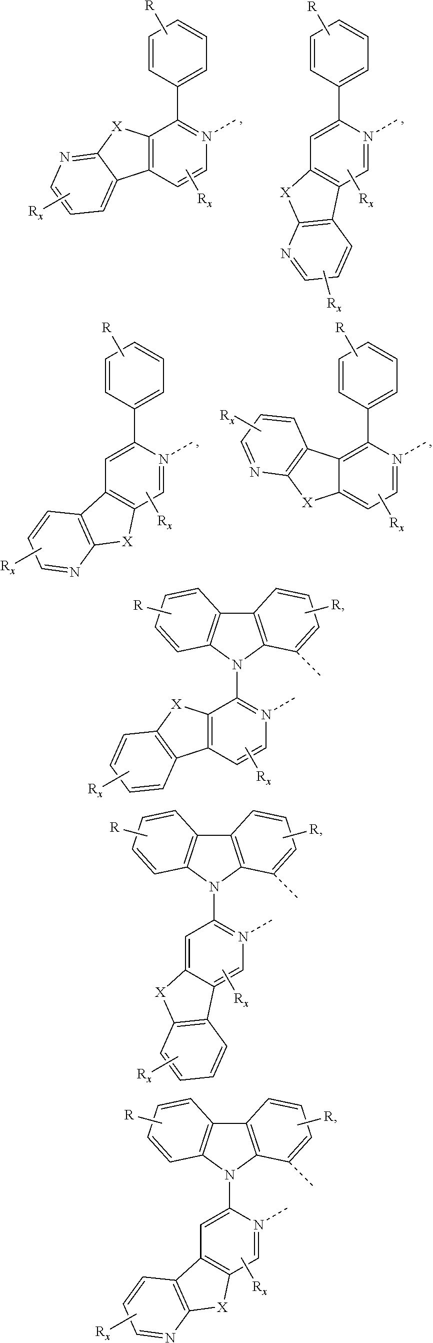 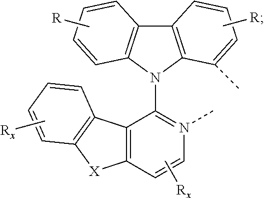
X is selected from the group consisting of O, S, Se, NR′, CR′R′ and SiR′R; wherein when two R′ are present, the two R′ are identical or different;
R and Rₓ represent, at each occurrence identically or differently, mono-substitution, multiple substitutions or non-substitution;
at least one Rₓ is selected from cyano or fluorine;
adjacent substituents R, R′, Rₓ can be optionally joined to form a ring;
L_{b} and L_{c} are, at each occurrence identically or differently, selected from a structure represented by any one of the group consisting of: 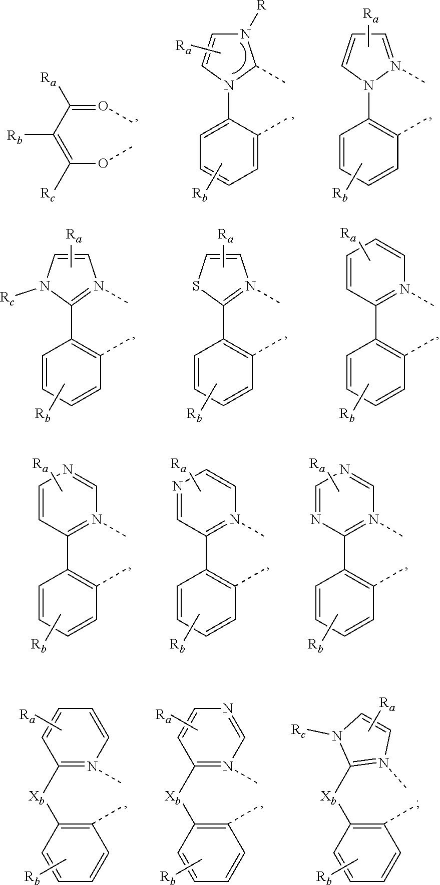
wherein
X_{b} is, at each occurrence identically or differently, selected from the group consisting of: O, S, Se, NR_{N1} and CR_{C1}R_{C2};
Rₐ and R_{b} represent, at each occurrence identically or differently, mono-substitution, multiple substitutions or non-substitution;
adjacent substituents Rₐ, R_{b}, R_{N1}, R_{C1}, R_{C2} can be optionally joined to form a ring; and
R, R′, Rₓ, Rₐ, R_{b}, R_{c}, R_{N1}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof.

4. The electroluminescent device according to claim 1, wherein the first metal complex Ir(Lₐ)ₘ(L_{b})₃₋ₘ has a structure represented by Formula 5: 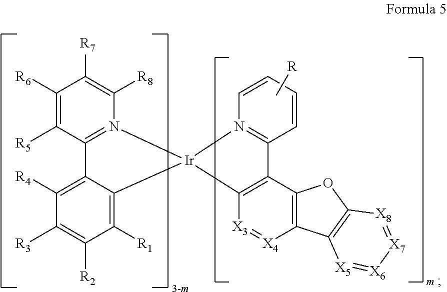
wherein
m is 1, 2 or 3; when m is 2 or 3, a plurality of Lₐ are identical or different; when m is 1, two L_{b} are identical or different;
X is selected from the group consisting of O, S, Se, NR′, CR′R′ and SiR′R; wherein when two R′ are present, the two R′ are identical or different;
X₃ to X₈ are, at each occurrence identically or differently, selected from CRₓ or N;
at least one of X₃ to X₈ is CRₓ, wherein the Rₓ is cyano or fluorine;
R represents, at each occurrence identically or differently, mono-substitution, multiple substitutions or non-substitution;
R₁ to R₈, R′, Rₓ and R are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;
adjacent substituents R₁ to R₈ can be optionally joined to form a ring; and
adjacent substituents R′, R and Rₓ can be optionally joined to form a ring.

5. The electroluminescent device according to claim 1, wherein X is selected from O or S.

6. The electroluminescent device according to claim 4, wherein at least one of X₅ to X₈ is selected from CRₓ, wherein the Rₓ is cyano or fluorine; preferably, X₇ or X₈ is CRₓ, wherein the Rₓ is cyano or fluorine.

7. The electroluminescent device according to claim 4, wherein at least one, two, three or all of R₂, R₃, R₆ and R₇ is(are) selected from the group consisting of: deuterium, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof;
preferably, at least one, two, three or all of R₂, R₃, R₆ and R₇ is(are) selected from the group consisting of: deuterium, substituted or unsubstituted alkyl having 1 to 20 carbon atoms, substituted or unsubstituted cycloalkyl having 3 to 20 ring carbon atoms and combinations thereof; and
more preferably, at least one, two, three or all of R₂, R₃, R₆ and R₇ is(are) selected from the group consisting of: deuterium, methyl, ethyl, propyl, isopropyl, n-butyl, isobutyl, t-butyl, cyclopentyl, cyclohexyl, neopentyl, t-pentyl and combinations thereof; optionally, hydrogen in the above groups can be partially or fully deuterated.

8. The electroluminescent device according to claim 4, wherein at least two of X₃ to X₈ are selected from CRₓ, wherein one Rₓ is selected from fluorine or cyano, and at least another Rₓ is selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, cyano and combinations thereof; and
preferably, at least two of X₃ to X₈ are selected from CRₓ, wherein one Rₓ is selected from fluorine or cyano, and at least another Rₓ is selected from the group consisting of: hydrogen, deuterium, fluorine, substituted or unsubstituted alkyl having 1 to 6 carbon atoms, substituted or unsubstituted cycloalkyl having 3 to 6 ring carbon atoms, substituted or unsubstituted aryl having 6 to 12 carbon atoms, substituted or unsubstituted heteroaryl having 3 to 12 carbon atoms, substituted or unsubstituted alkylsilyl having 3 to 6 carbon atoms, cyano and combinations thereof.

9. The electroluminescent device according to claim 2, wherein R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms and combinations thereof; and
preferably, at least one R is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

10. The electroluminescent device according to claim 1, wherein Lₐ is, at each occurrence identically or differently, selected from any one of the group consisting of:

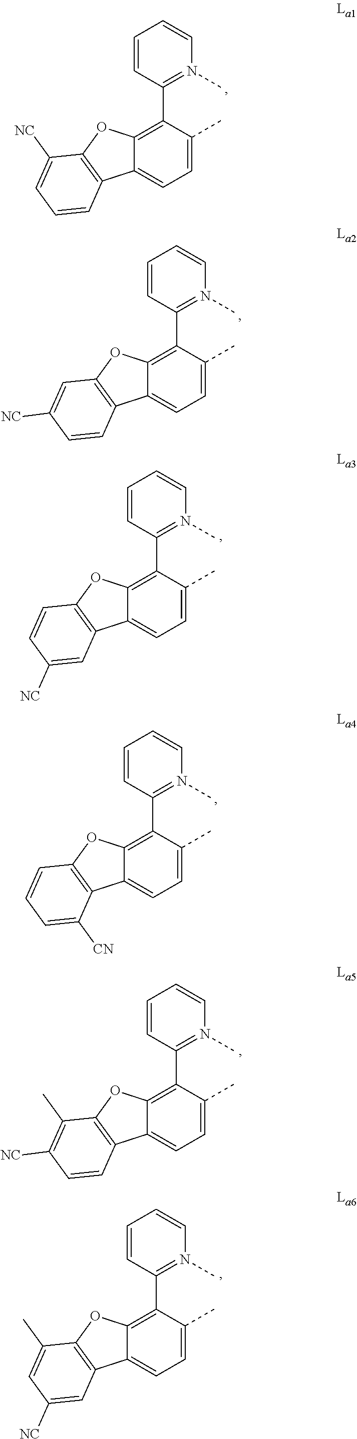

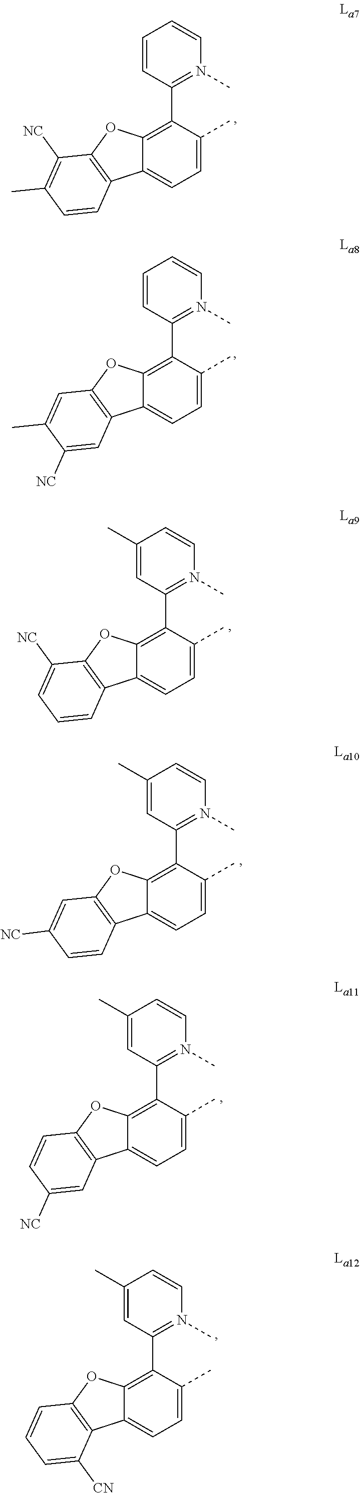

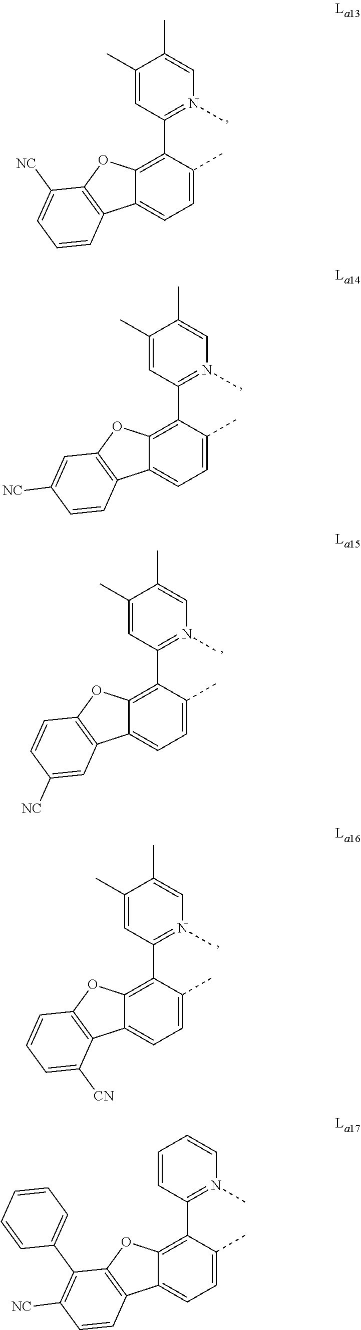

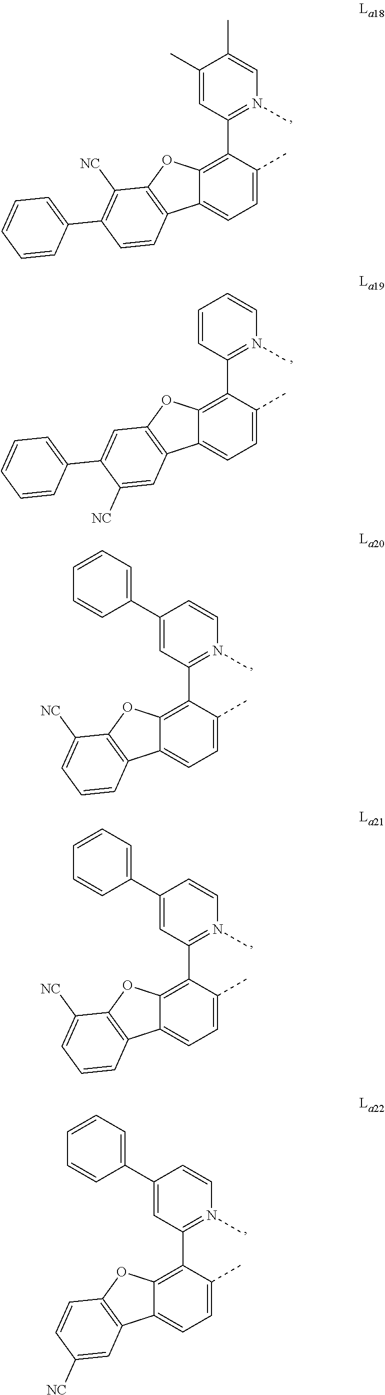

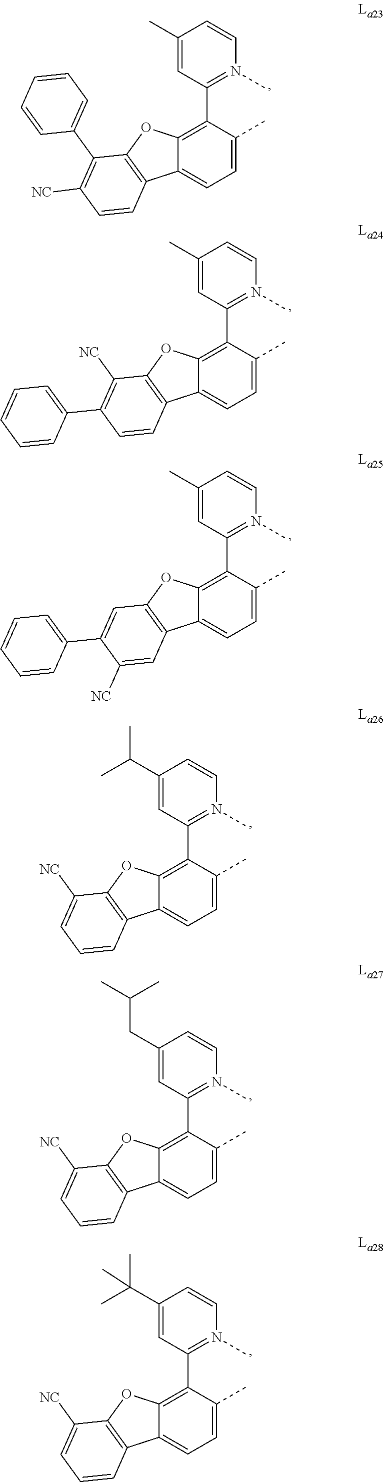

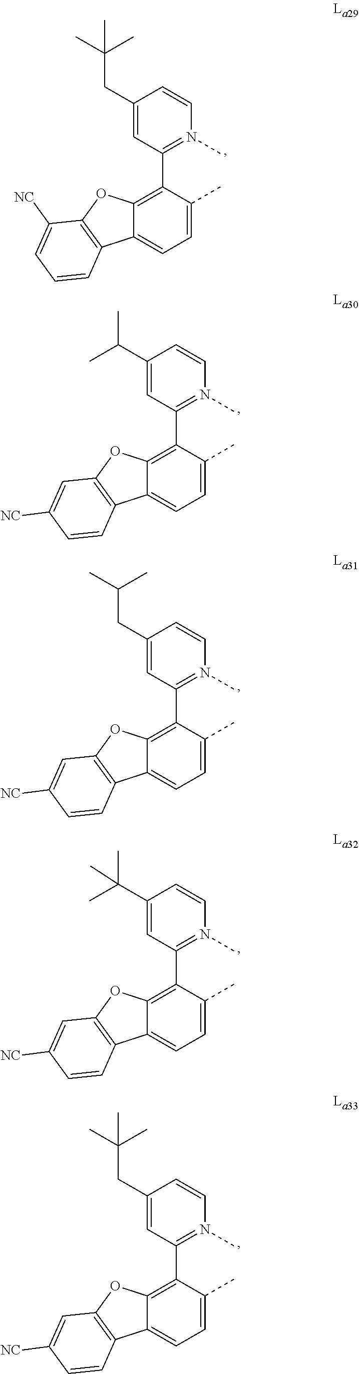

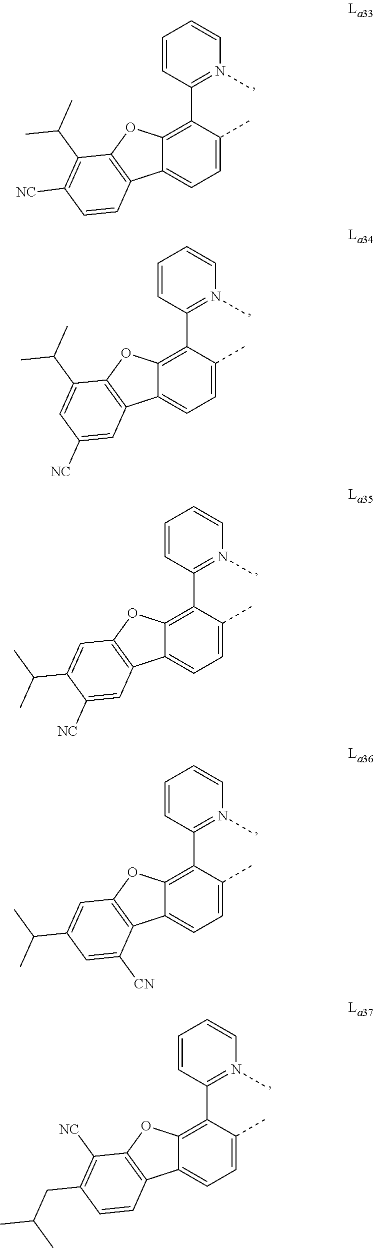

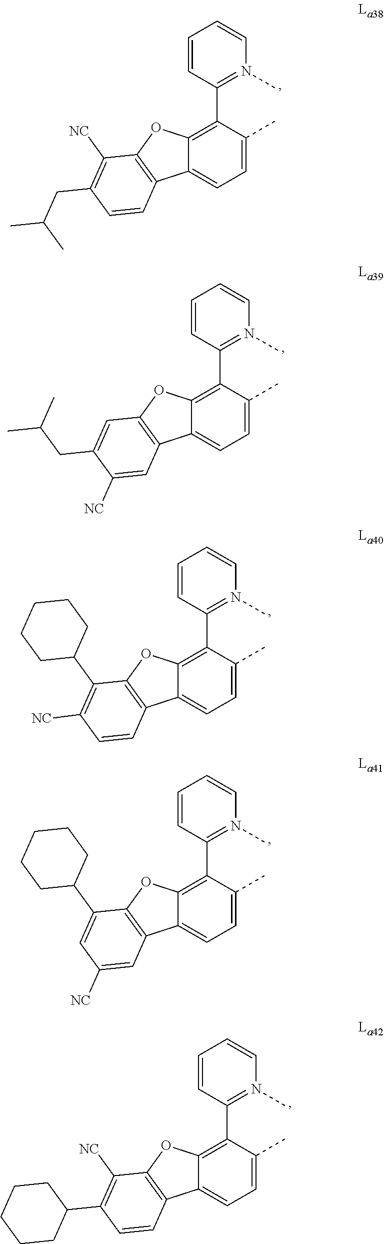

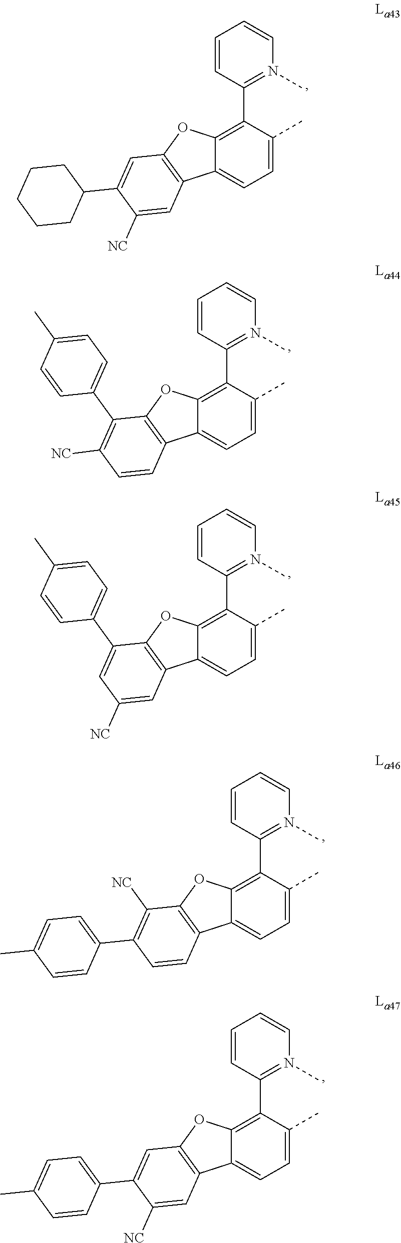

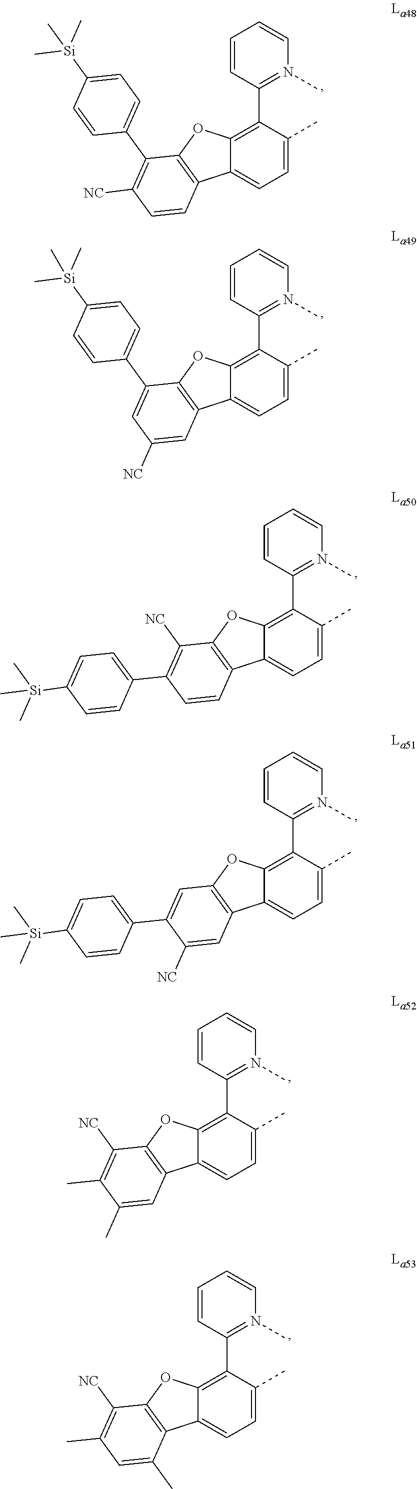

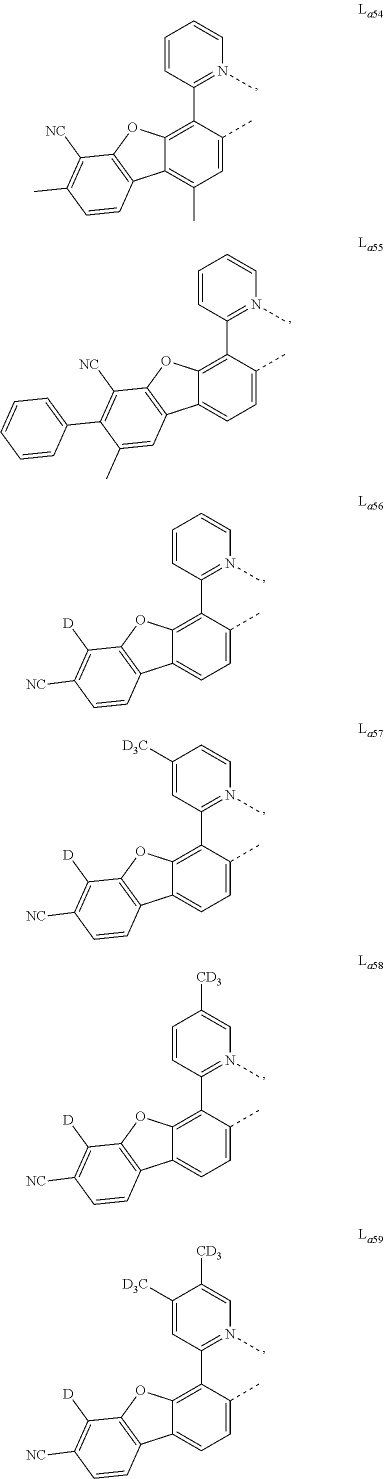

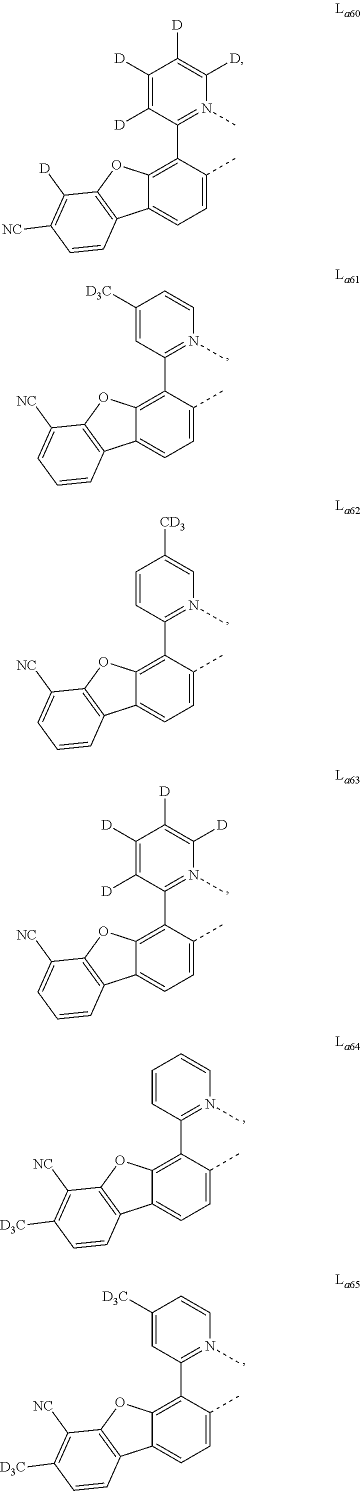

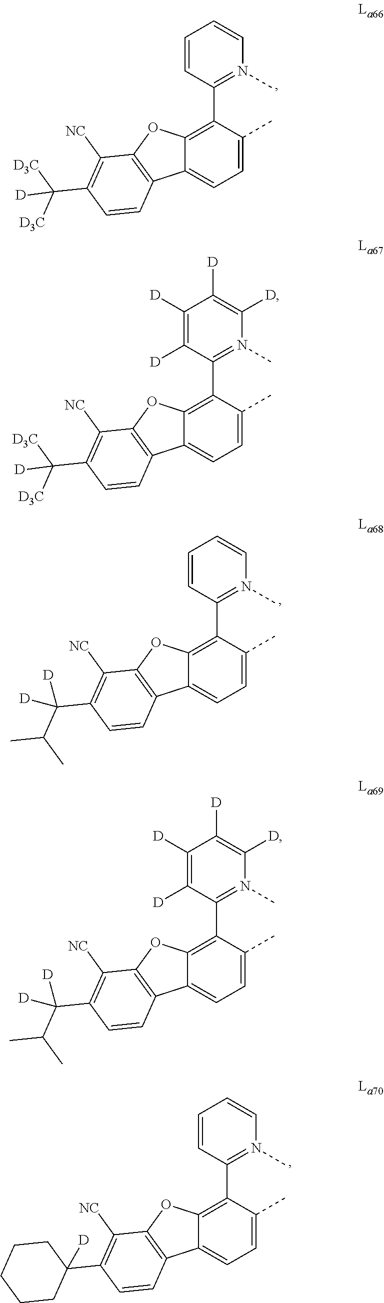

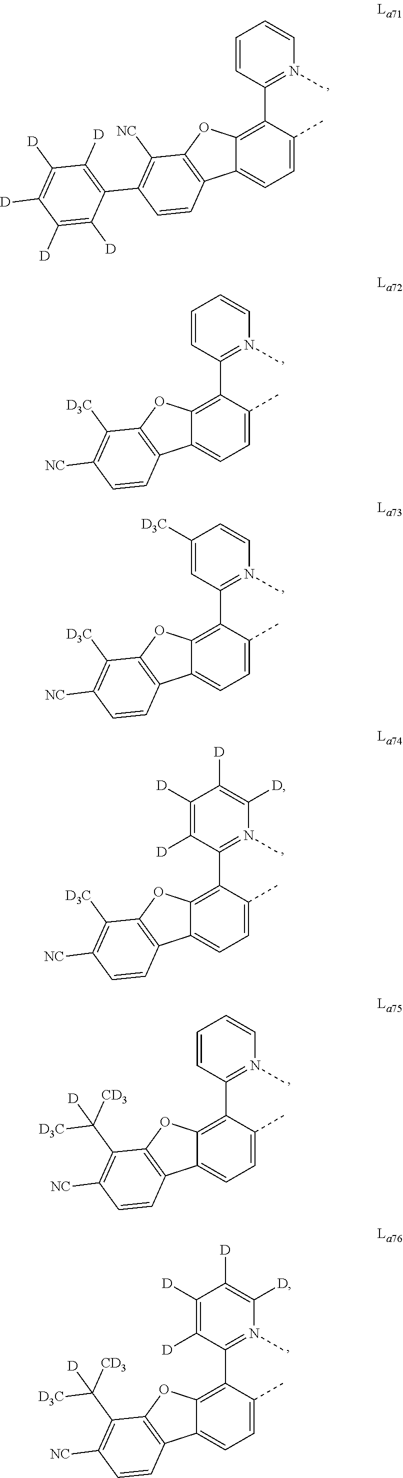

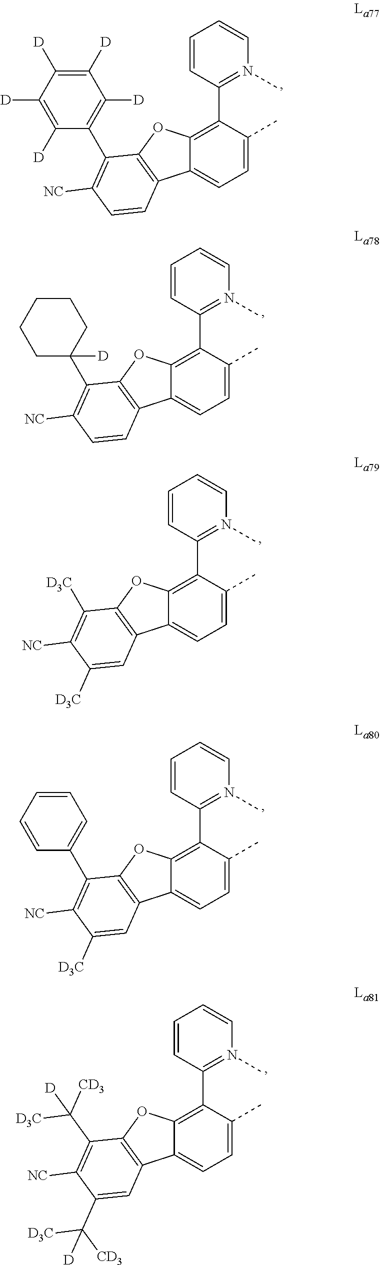

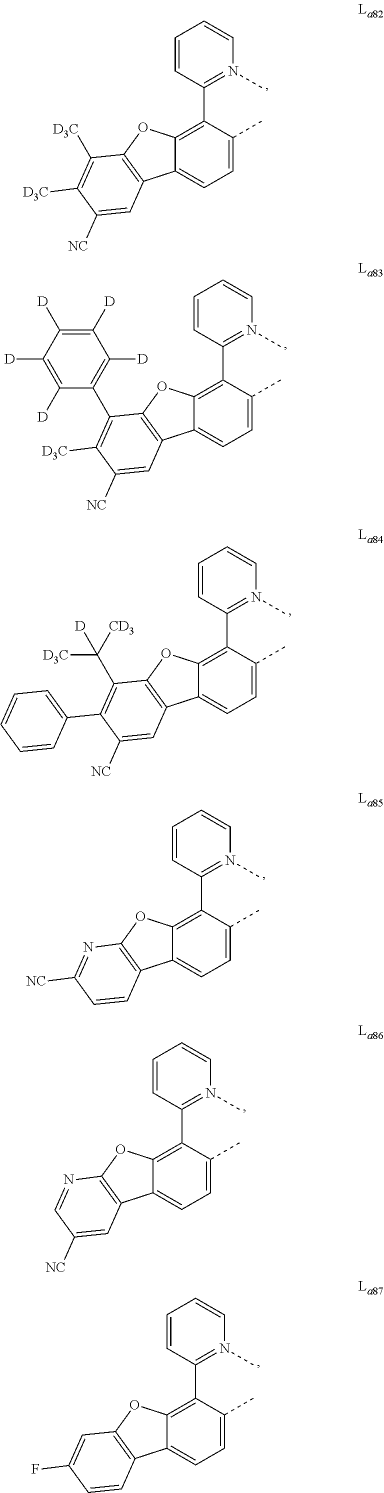

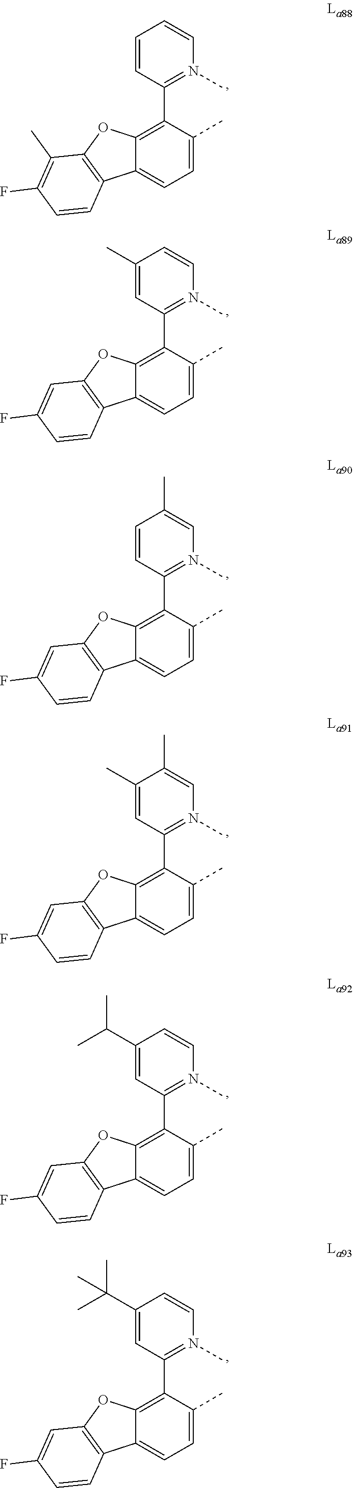

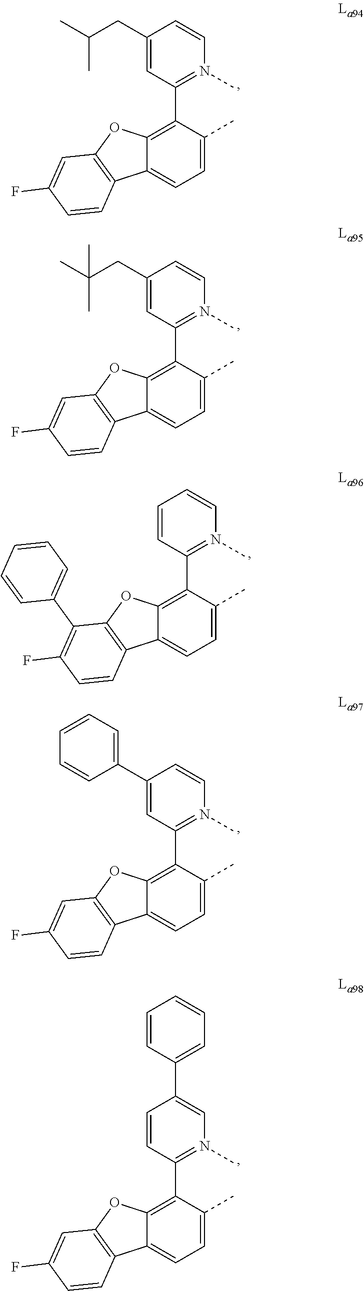

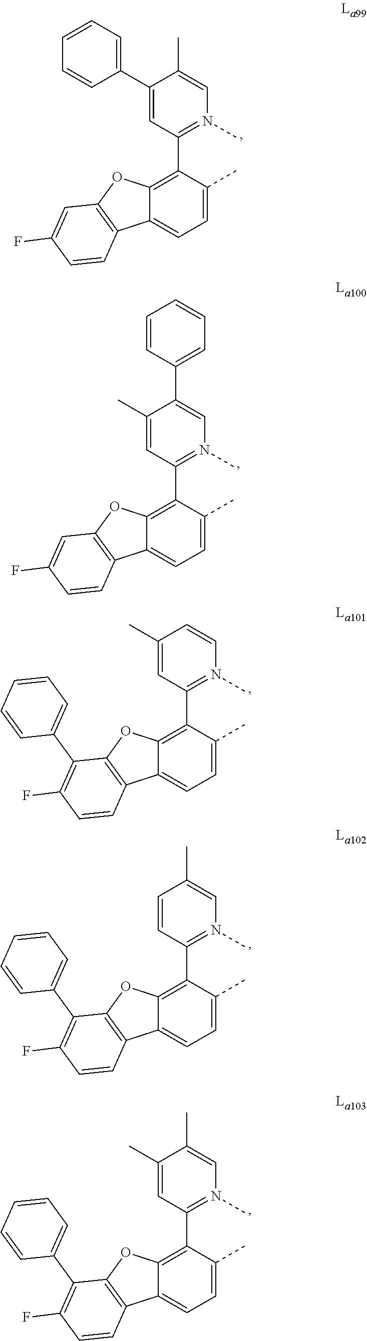

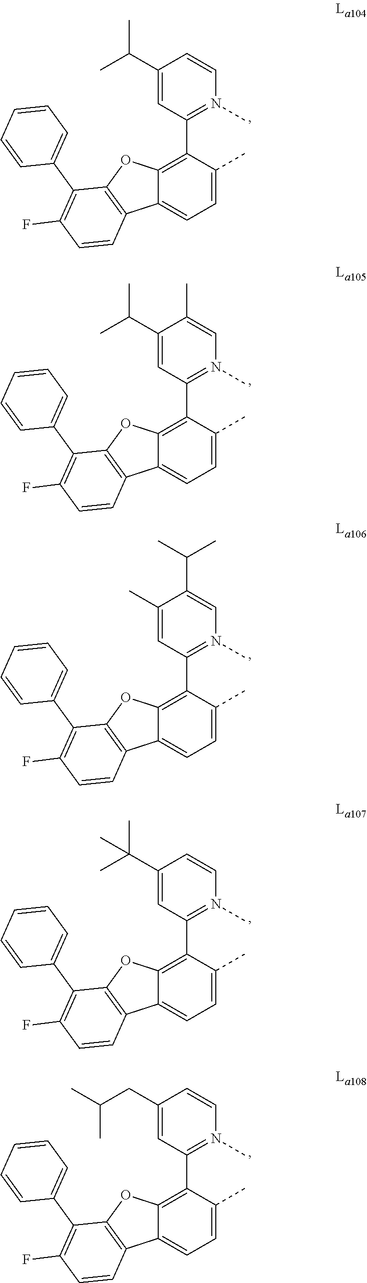

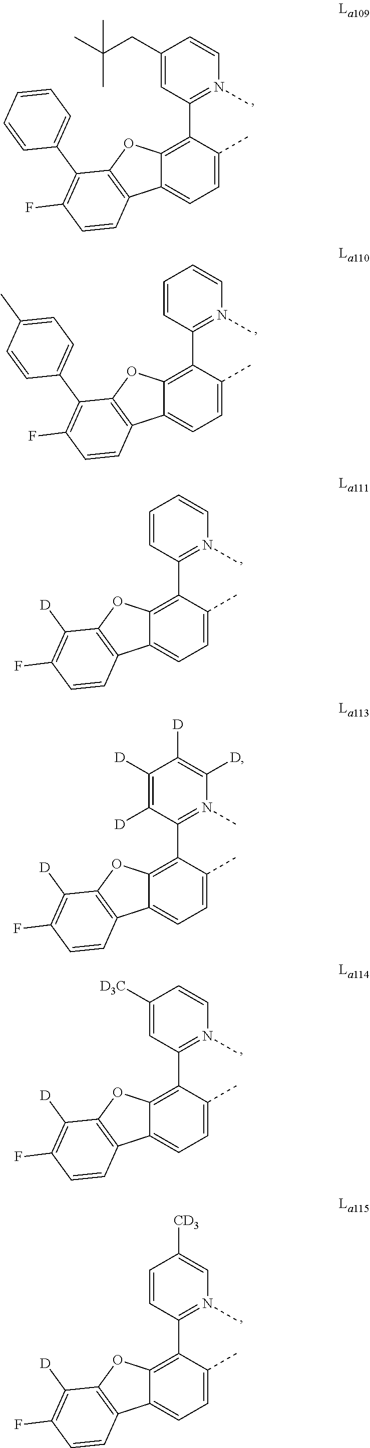

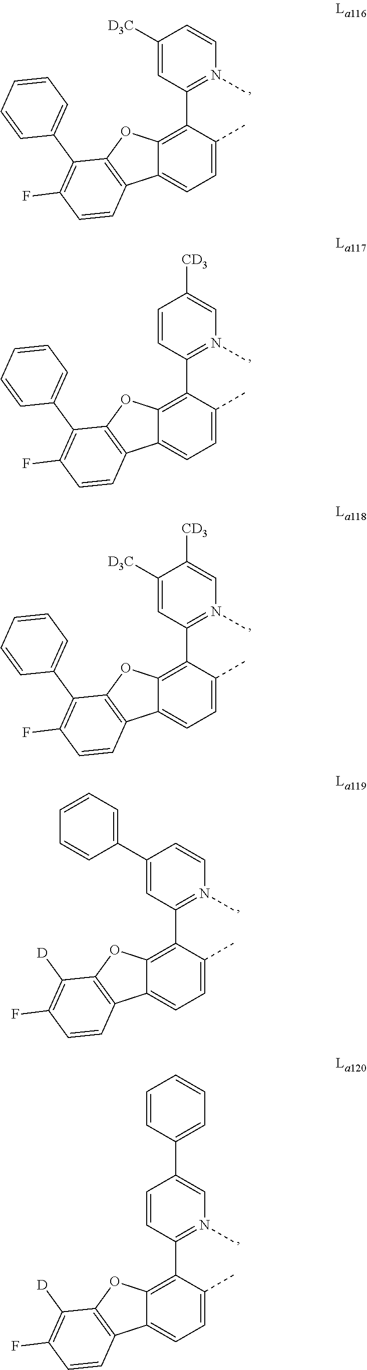

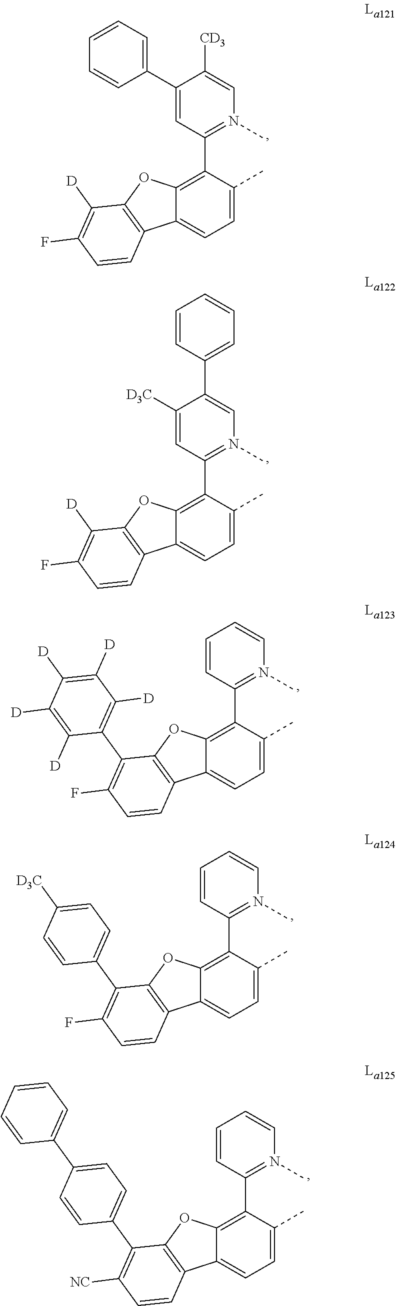

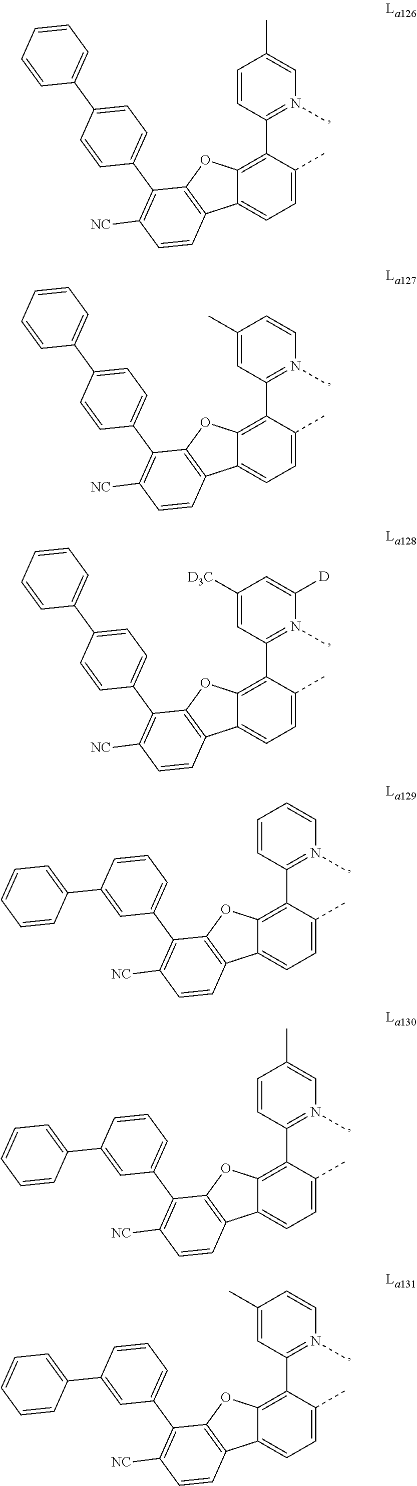

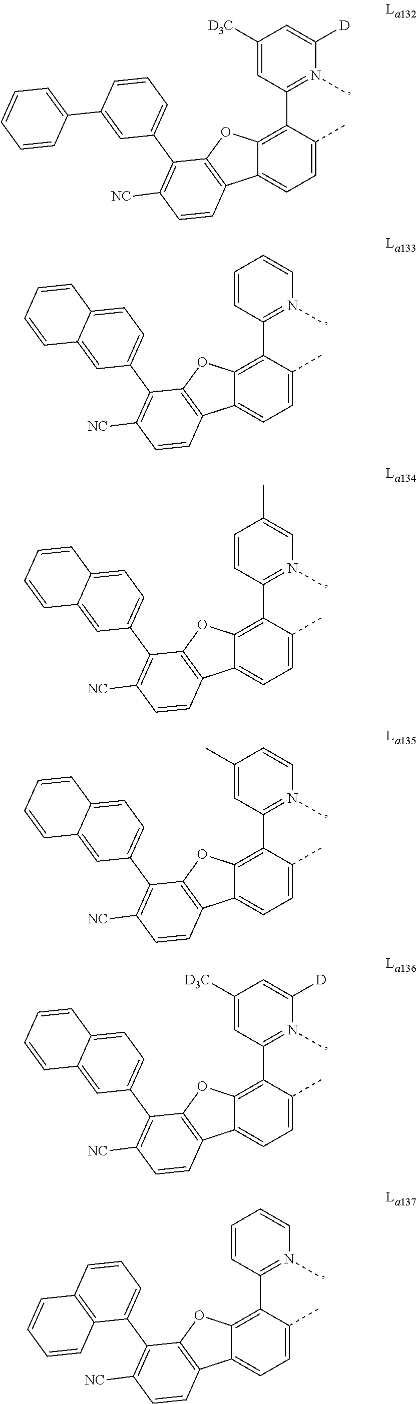

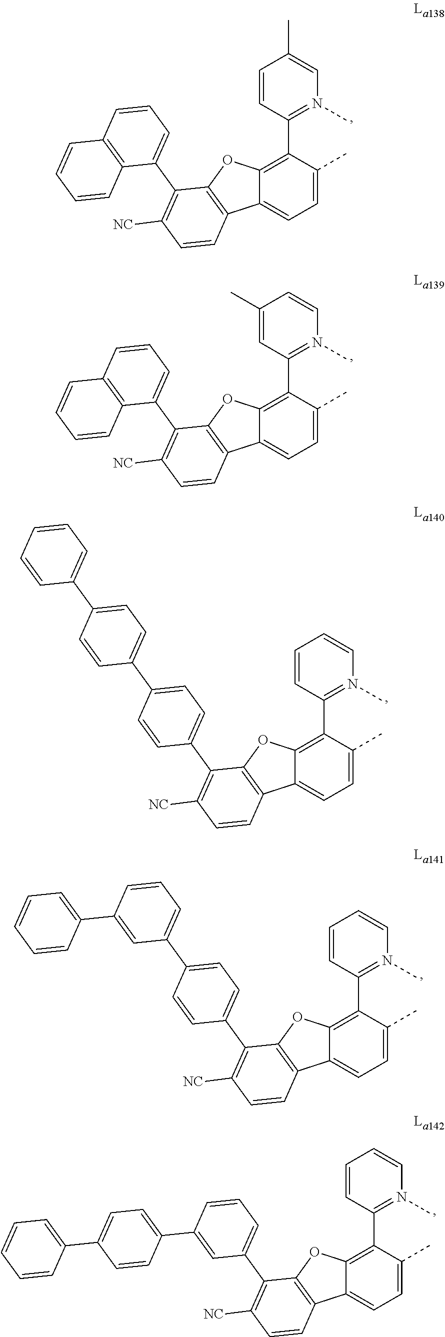

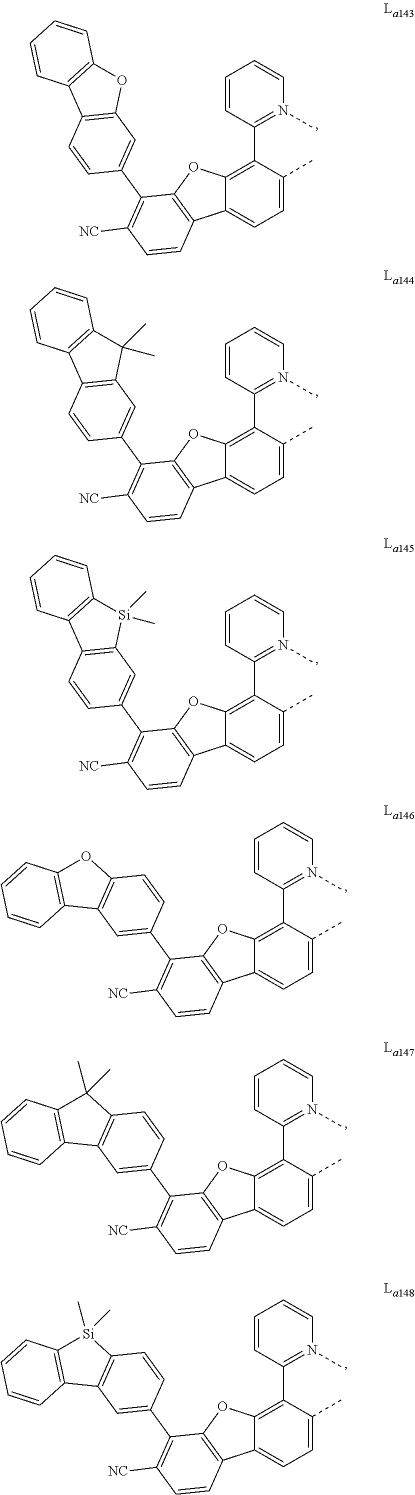

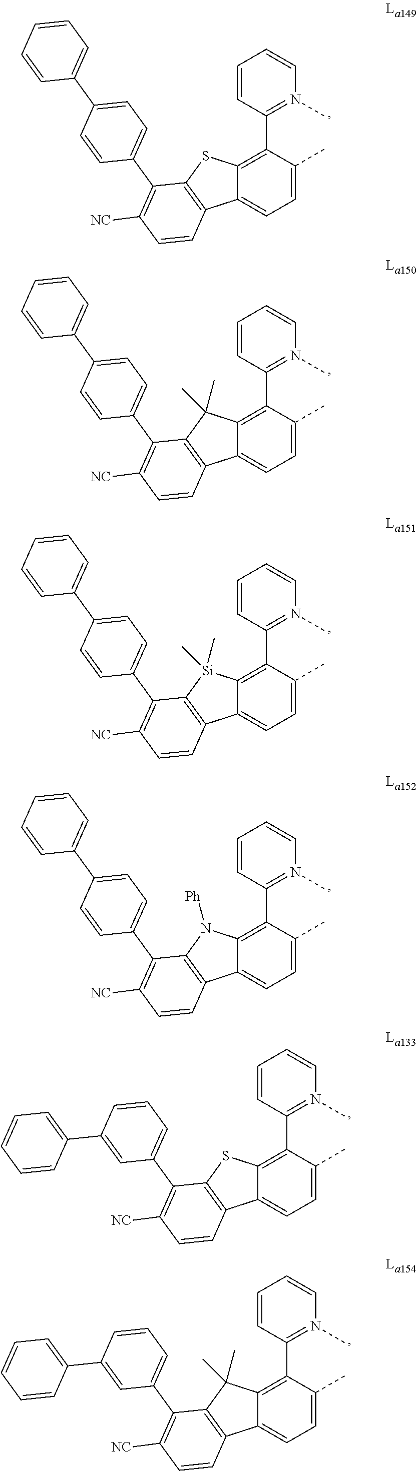

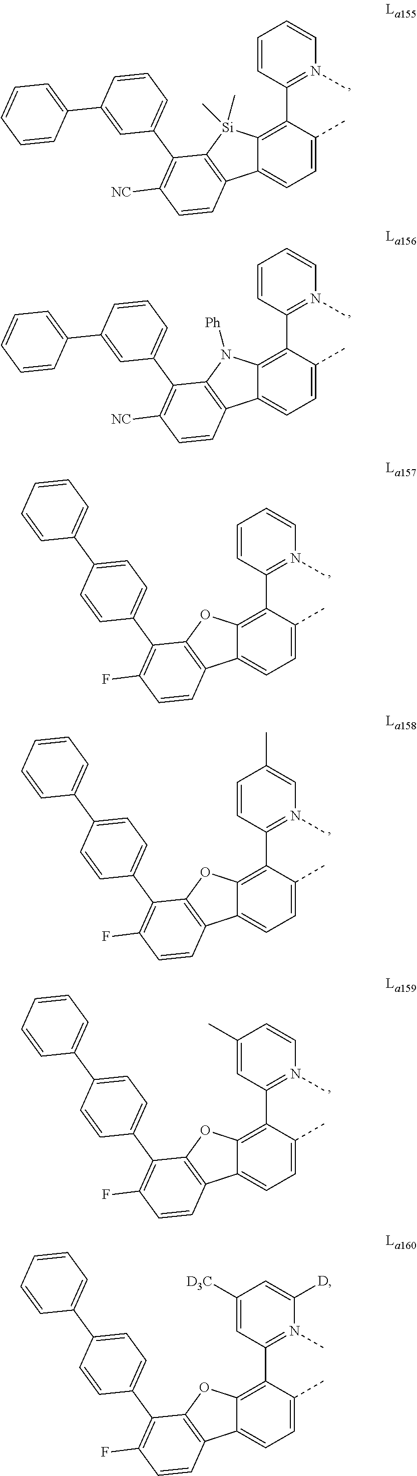

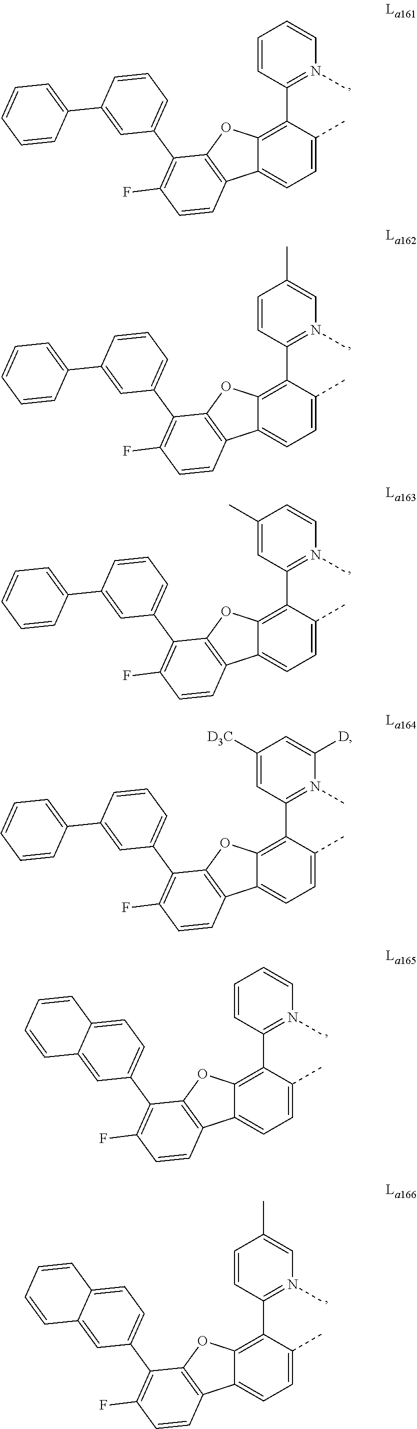

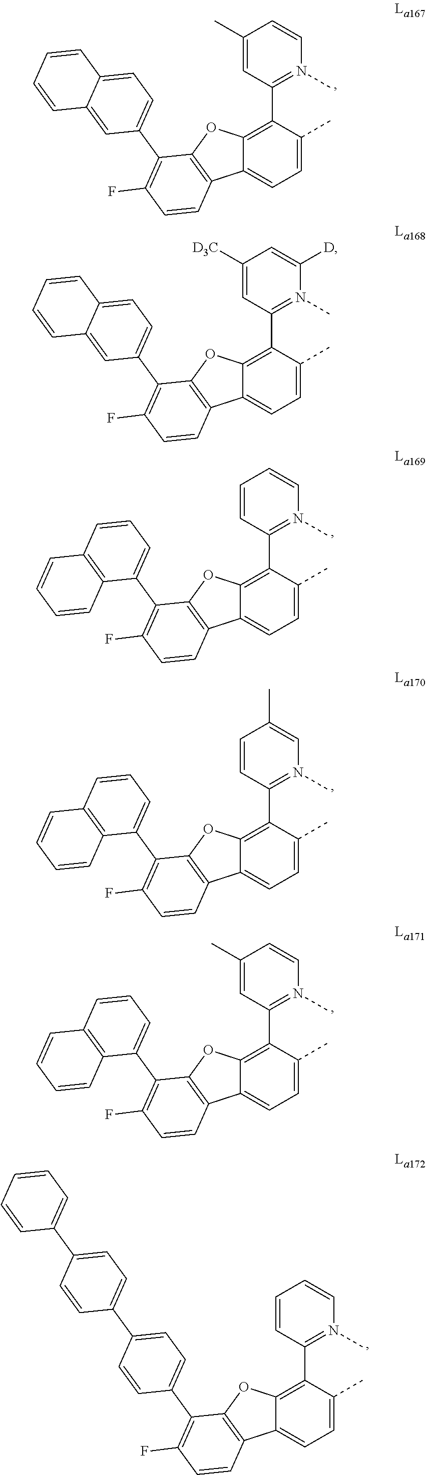

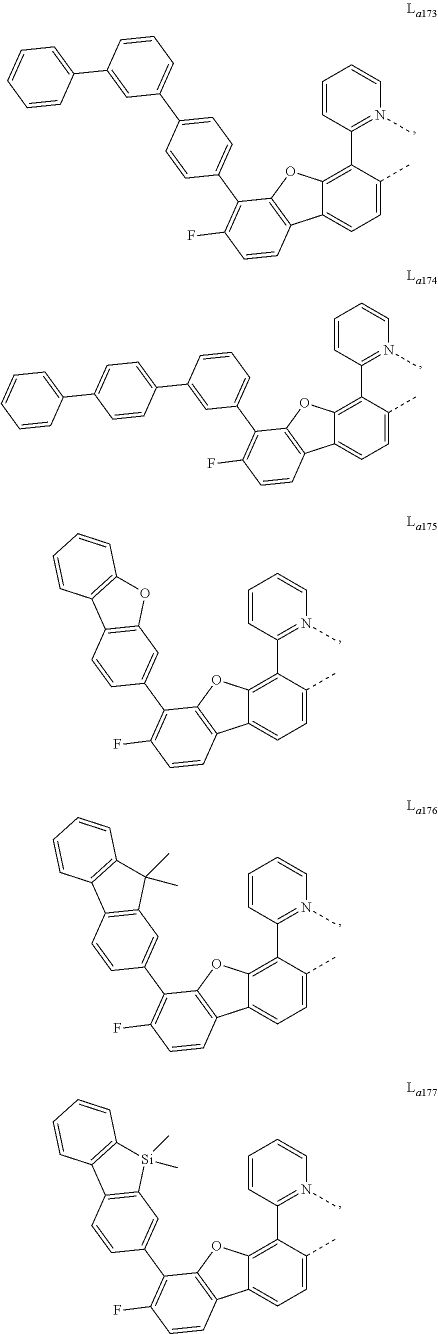

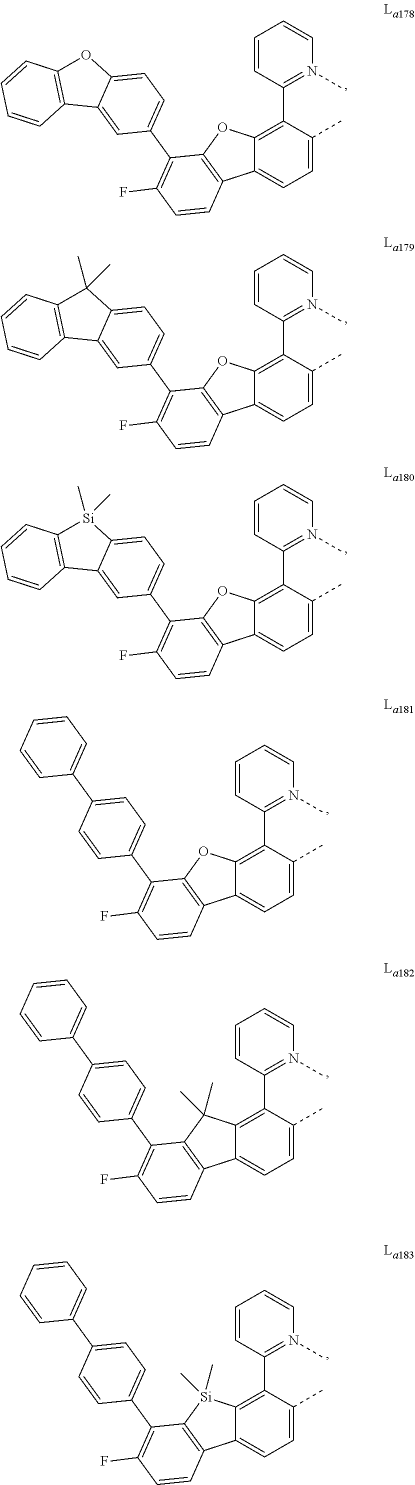

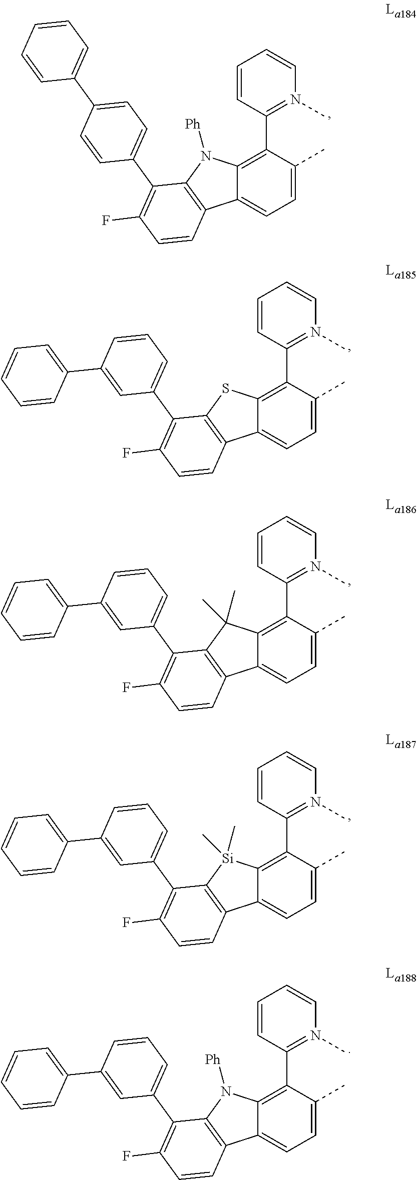

11. The electroluminescent device according to claim 3, wherein L_{b} is, at each occurrence identically or differently, selected from the group consisting of:

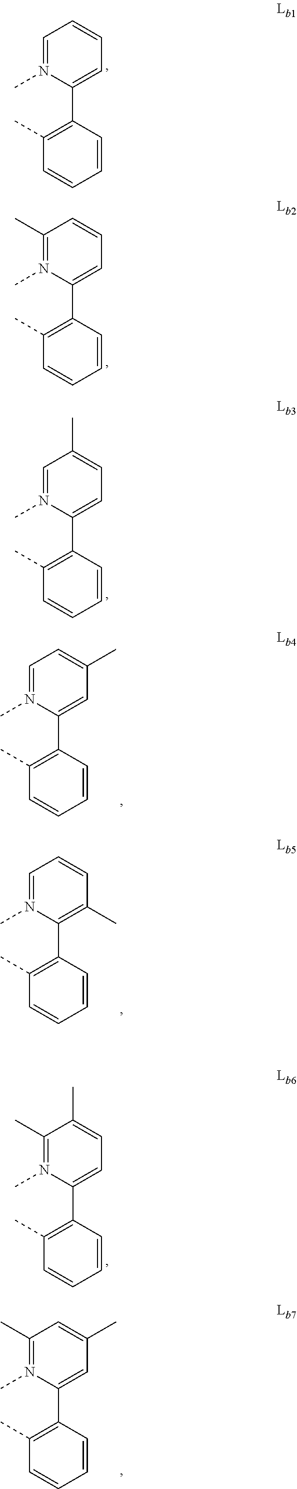

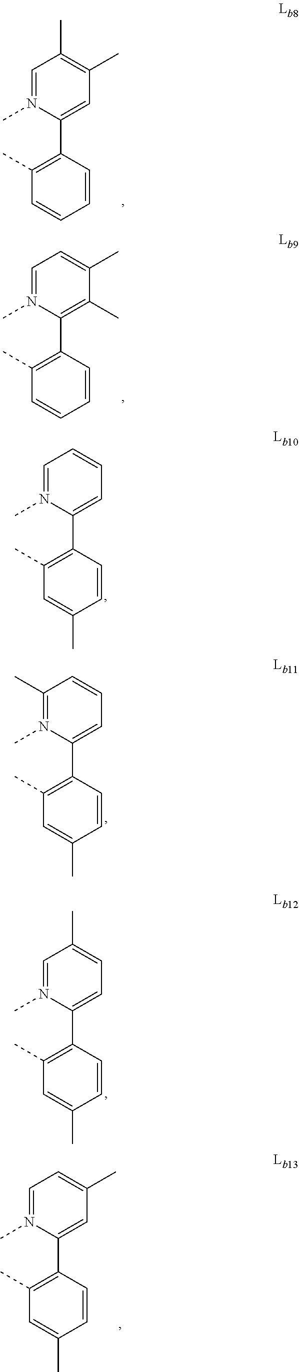

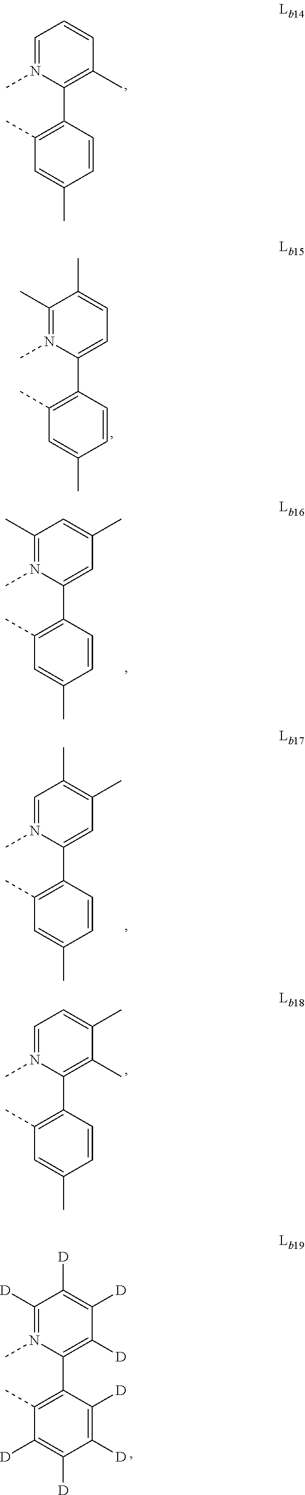

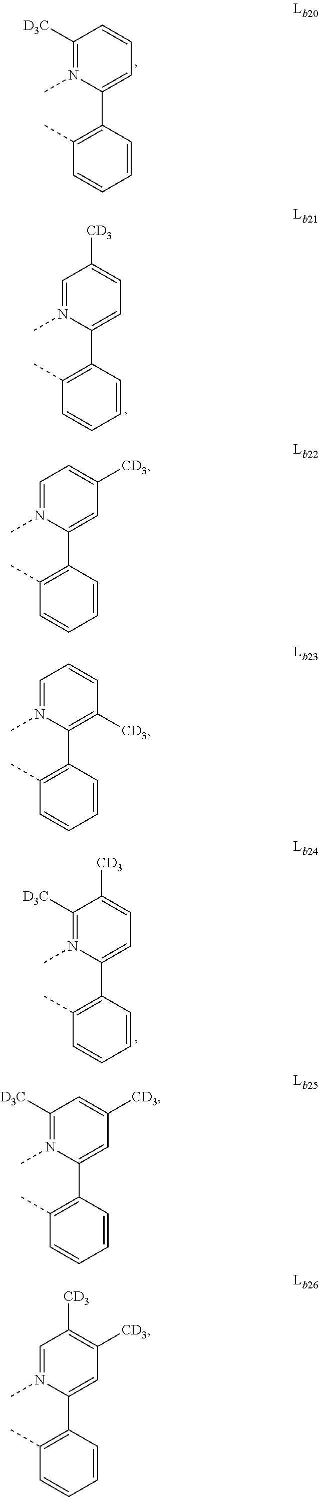

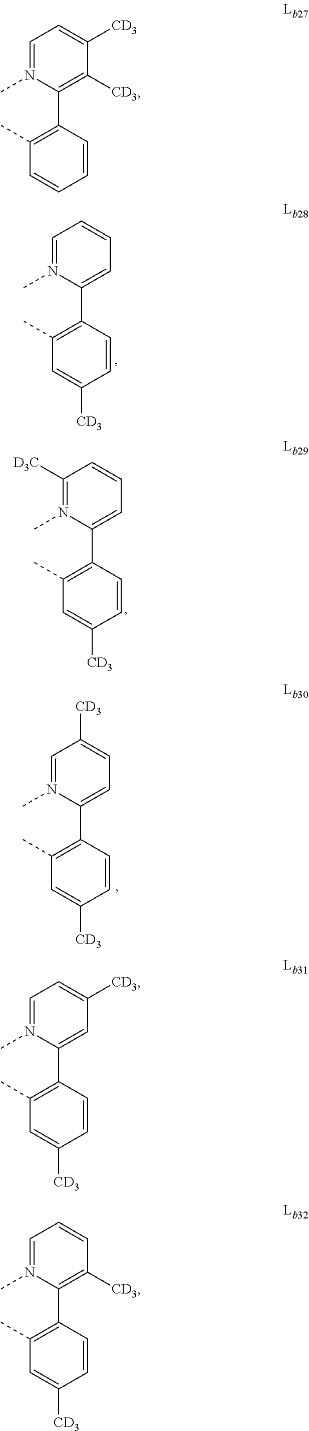

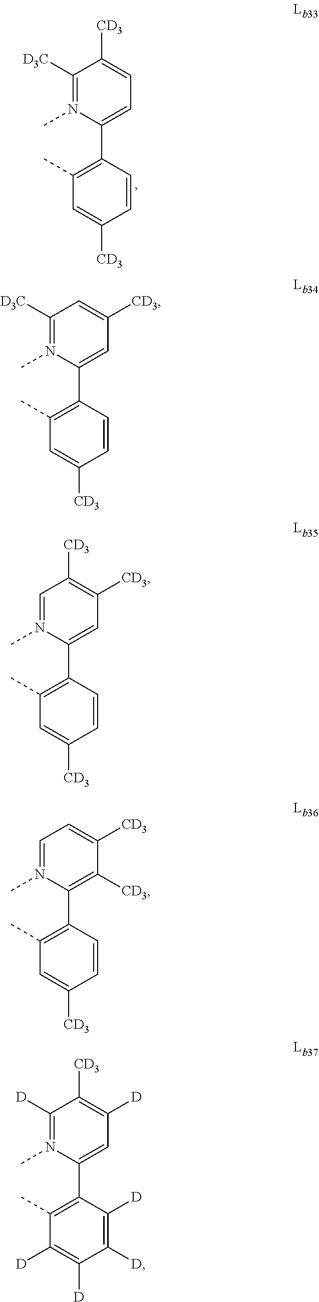

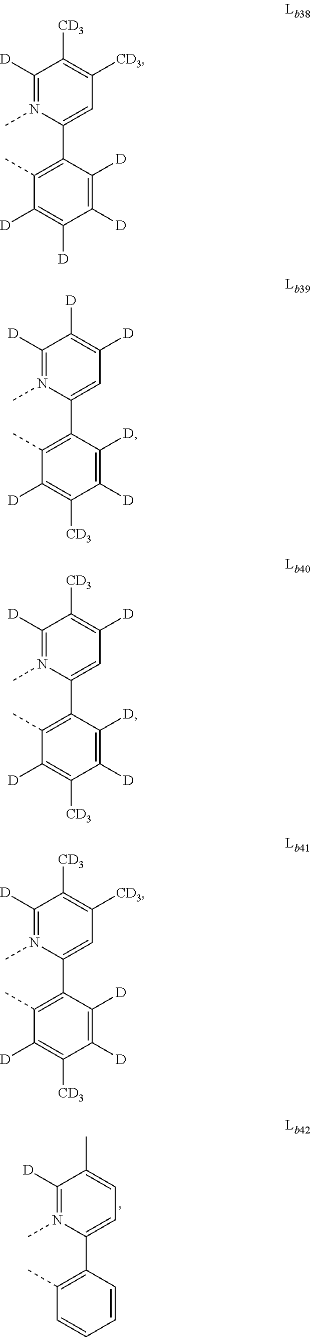

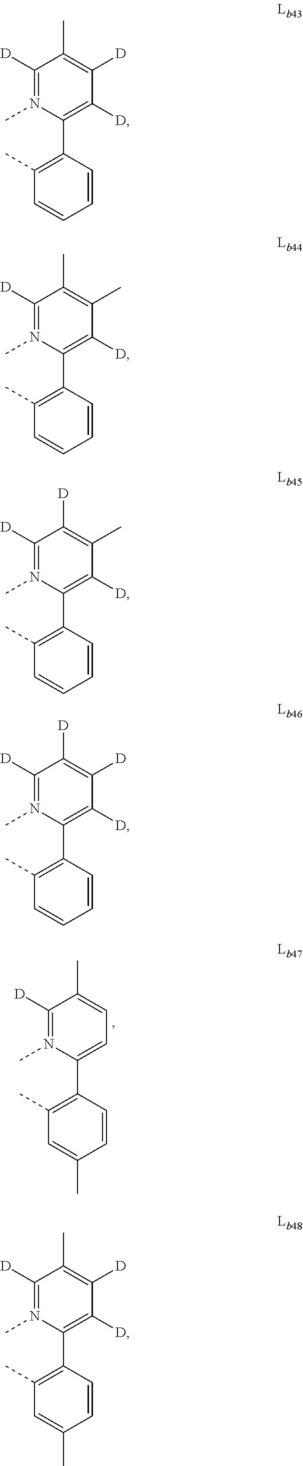

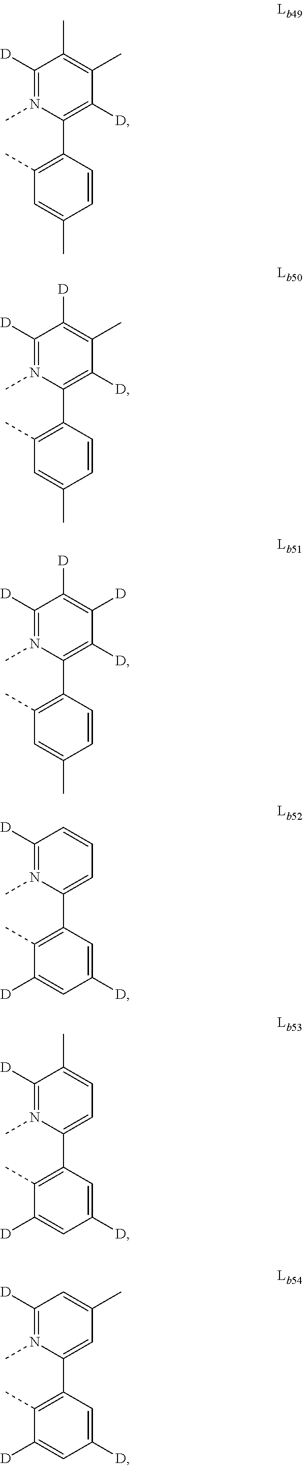

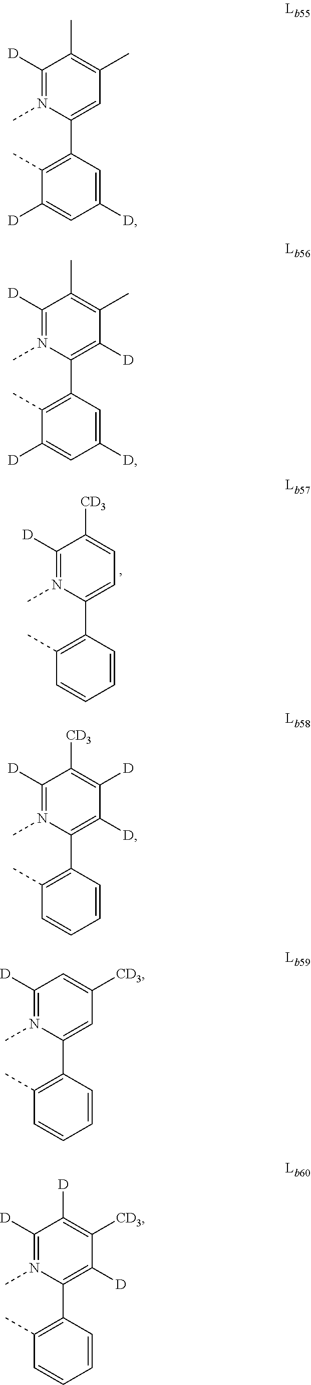

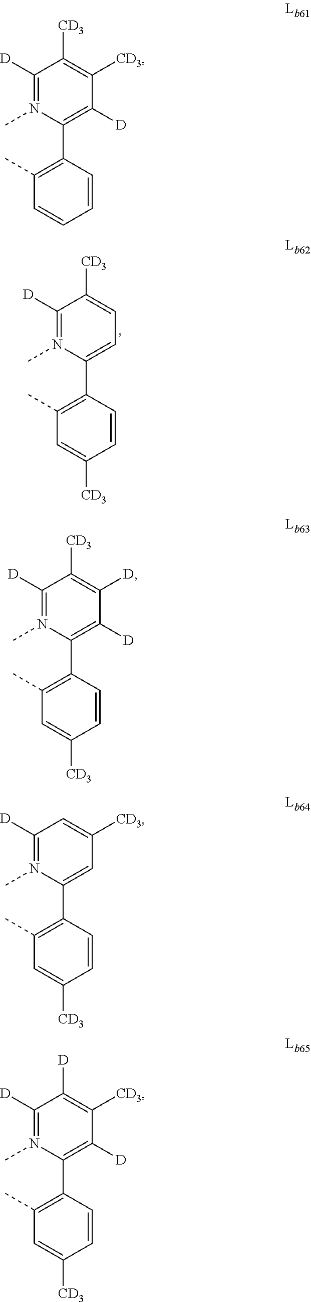

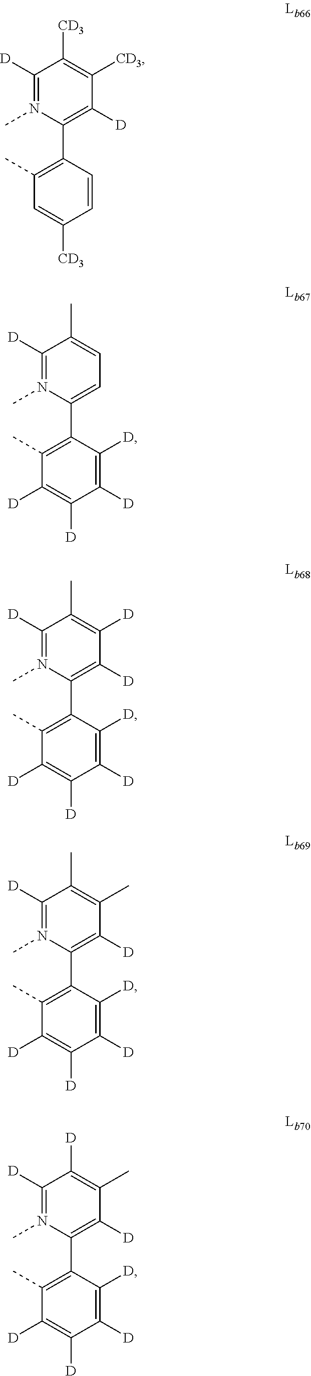

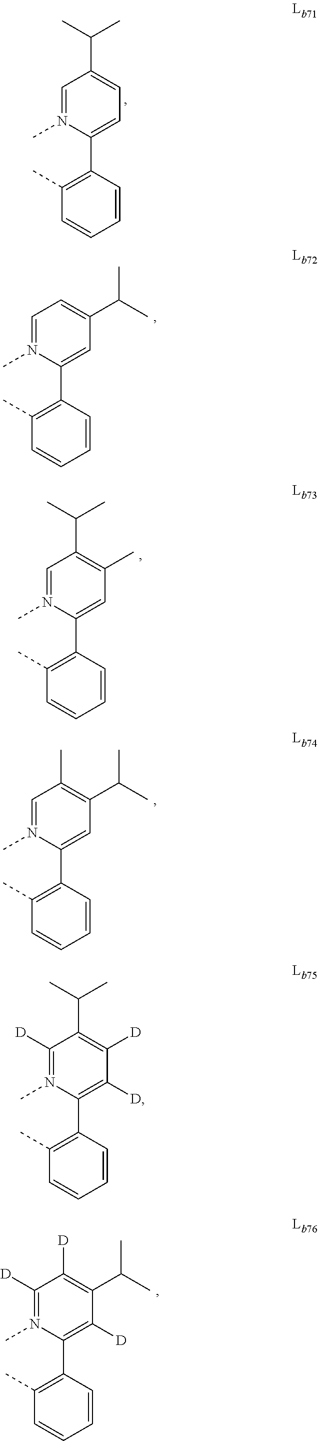

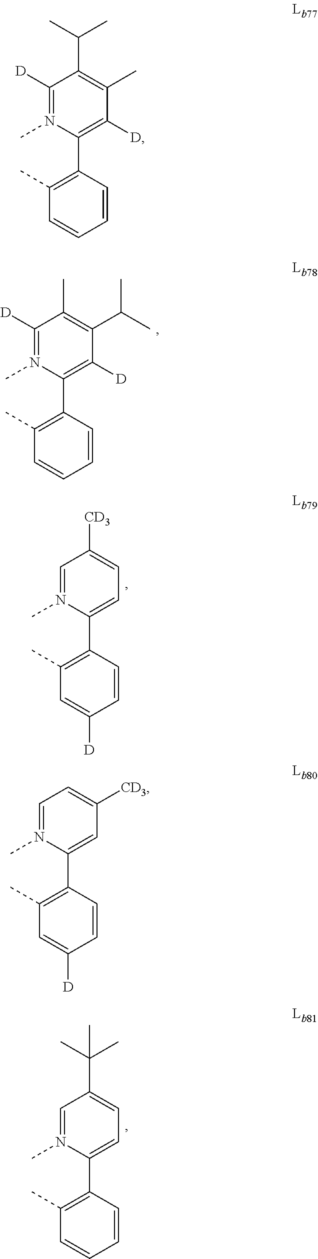

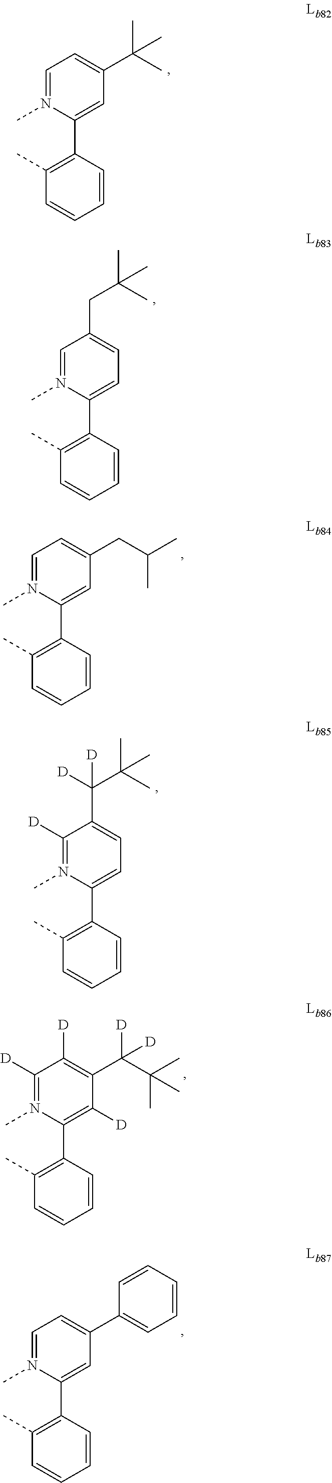

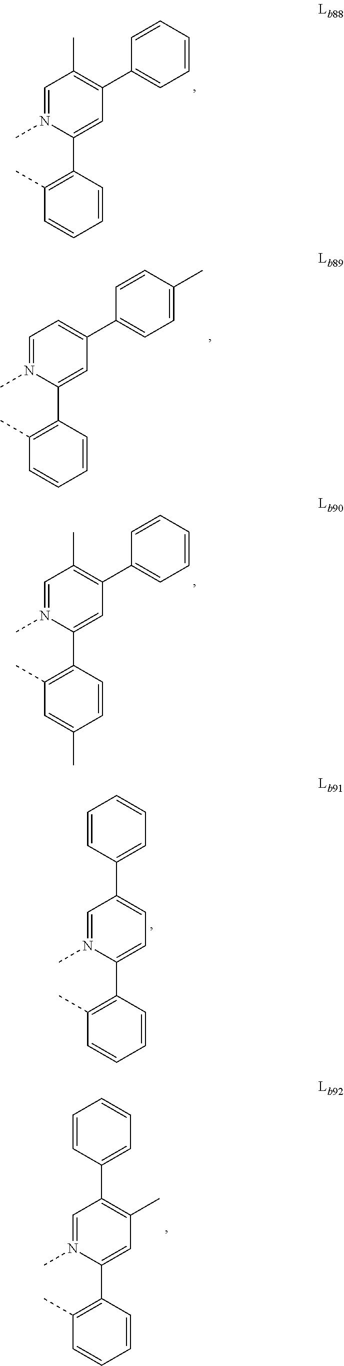

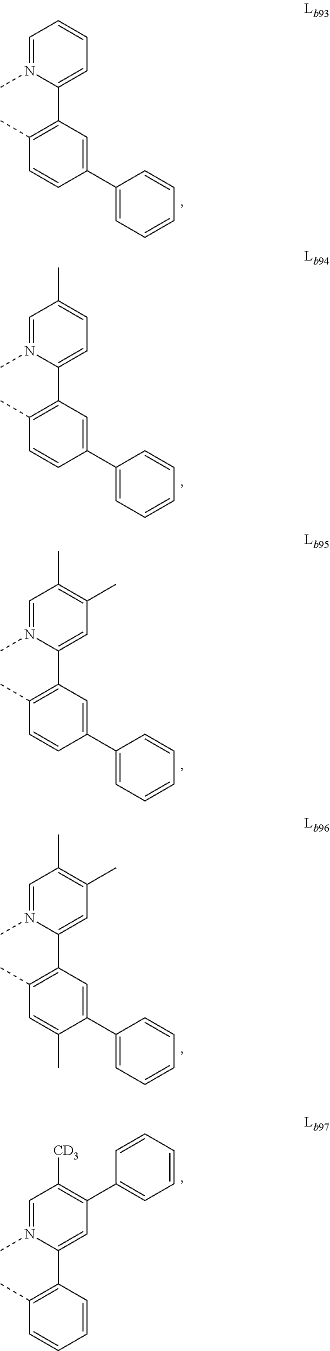

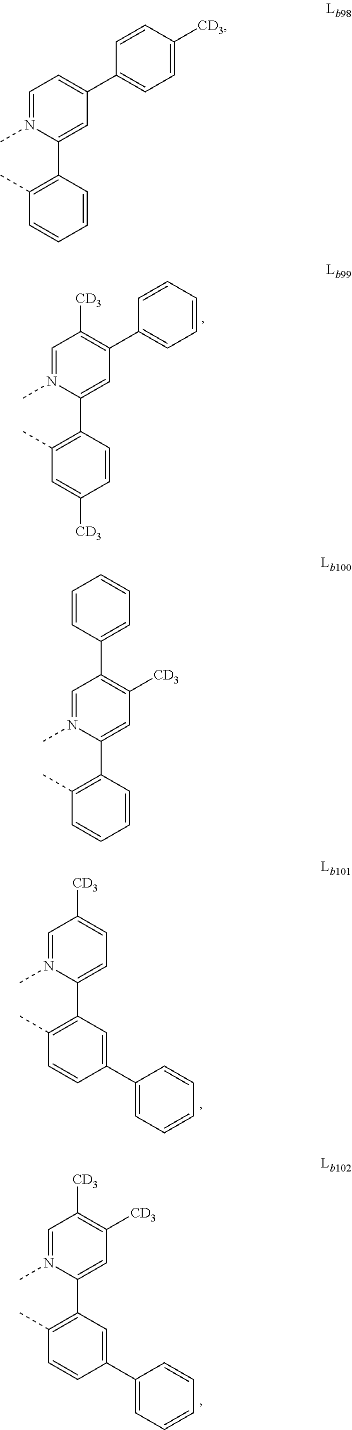

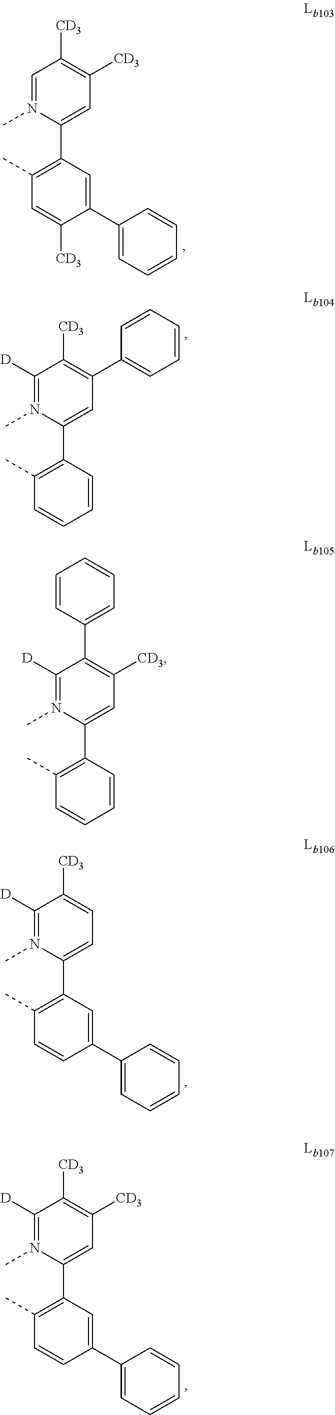

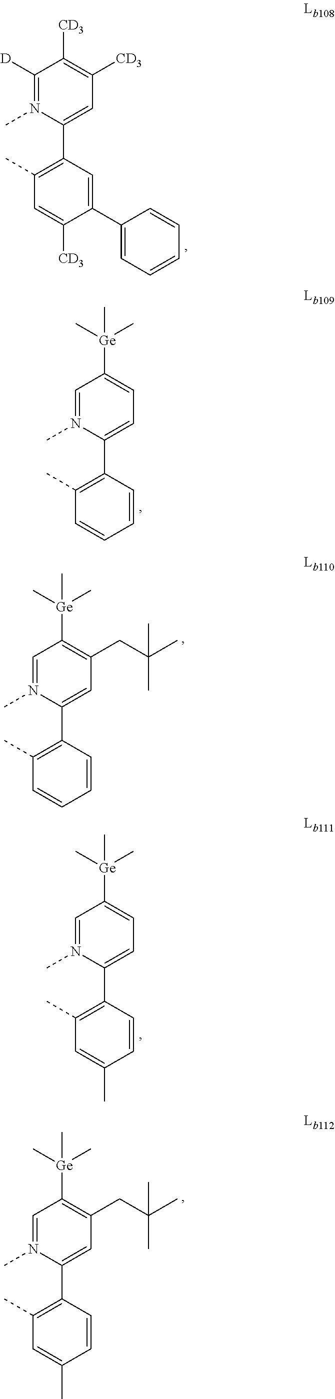

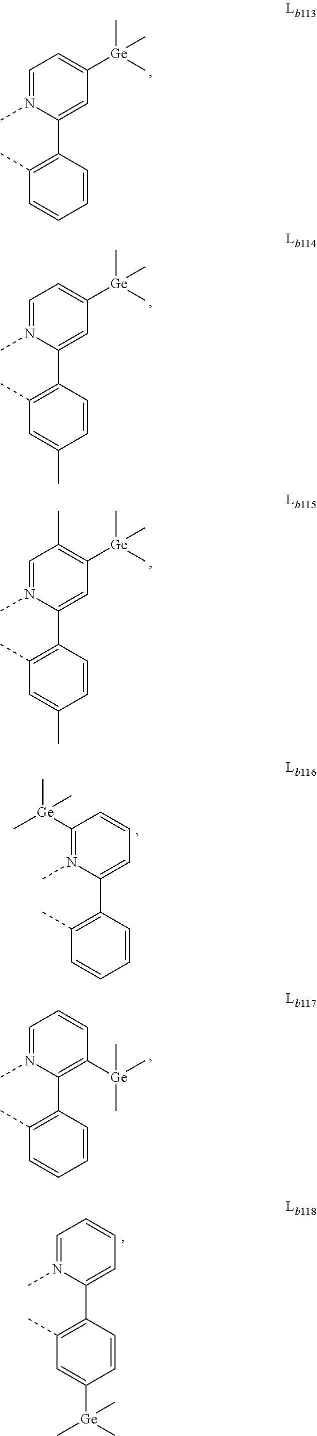

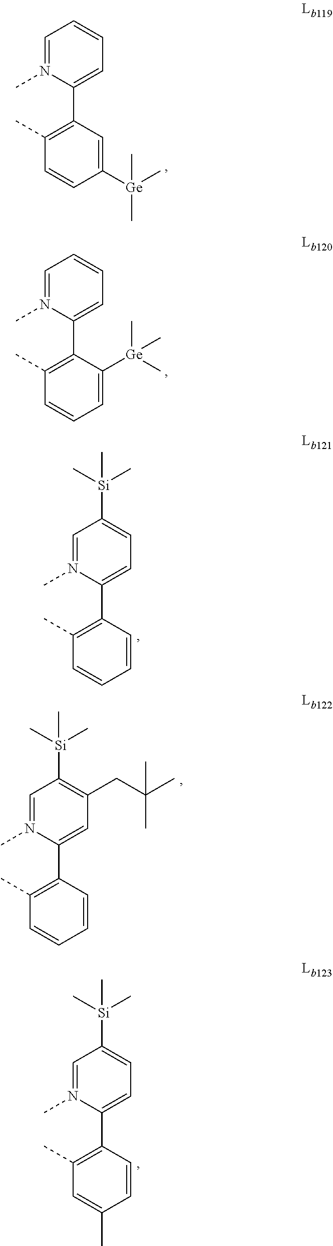

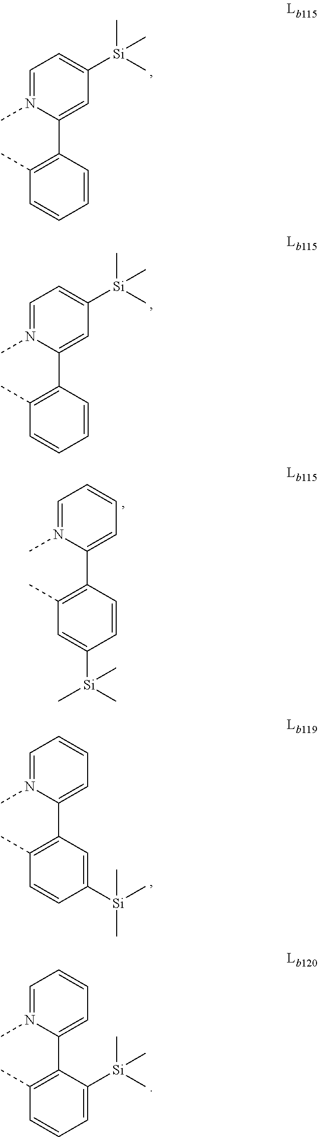

12. The electroluminescent device according to claim 3, wherein L_{c} is, at each occurrence identically or differently, selected from the group consisting of:

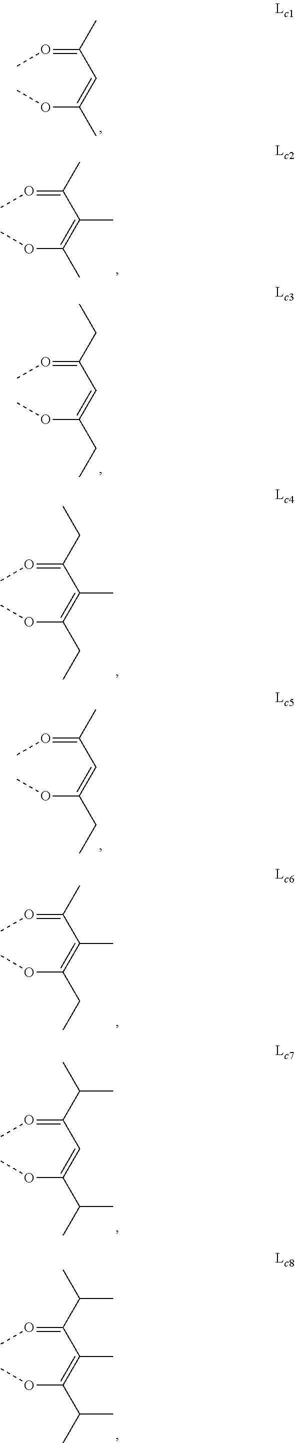

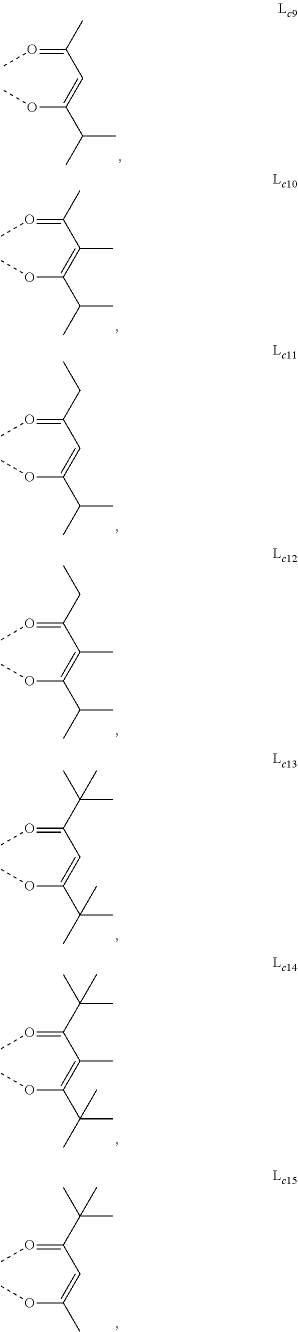

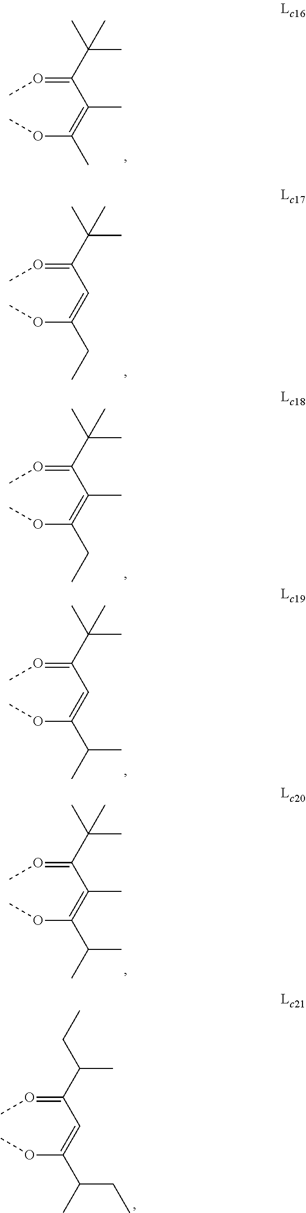

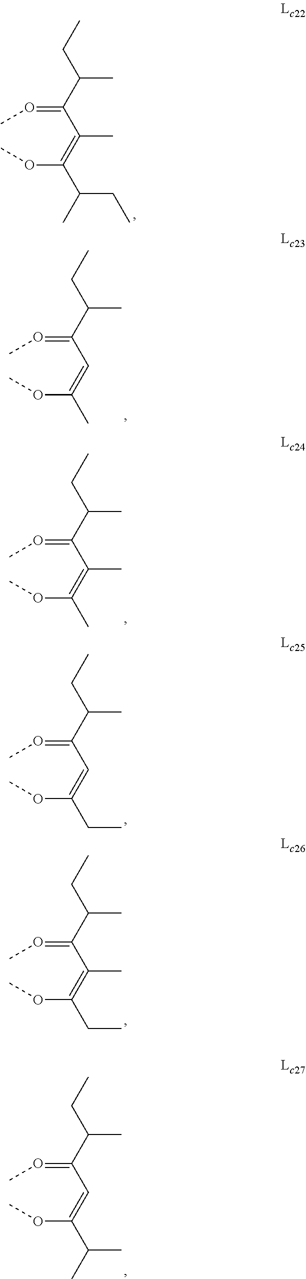

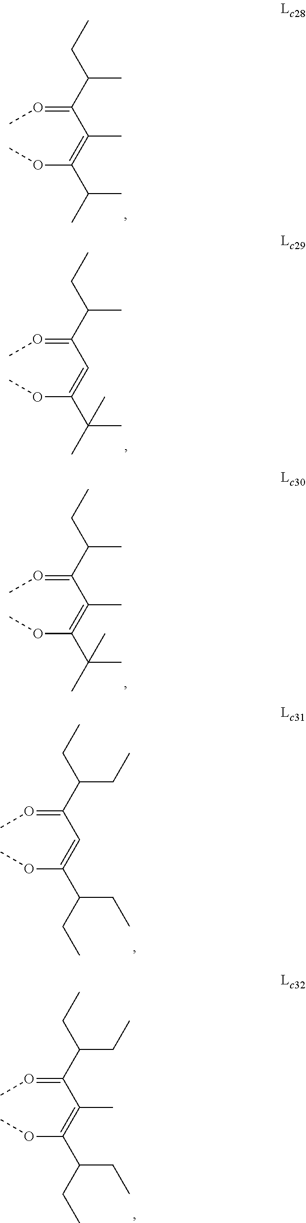

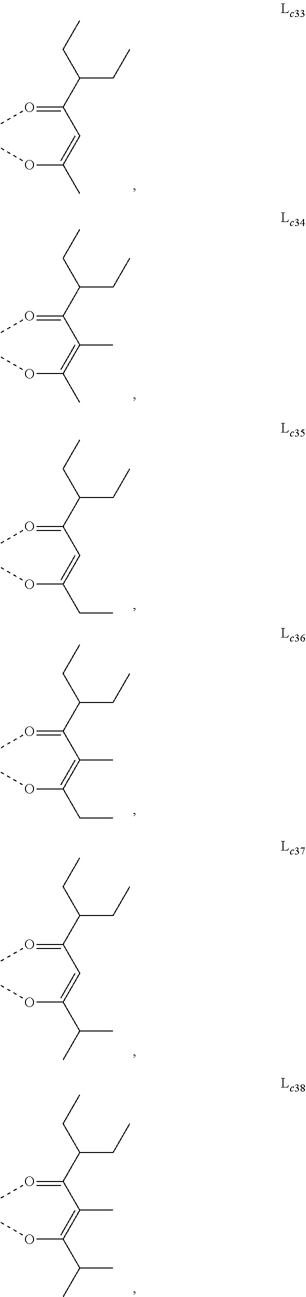

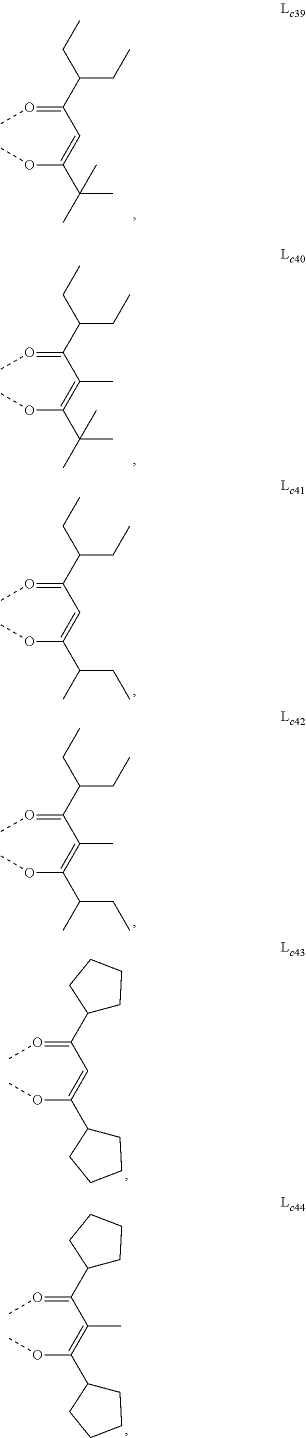

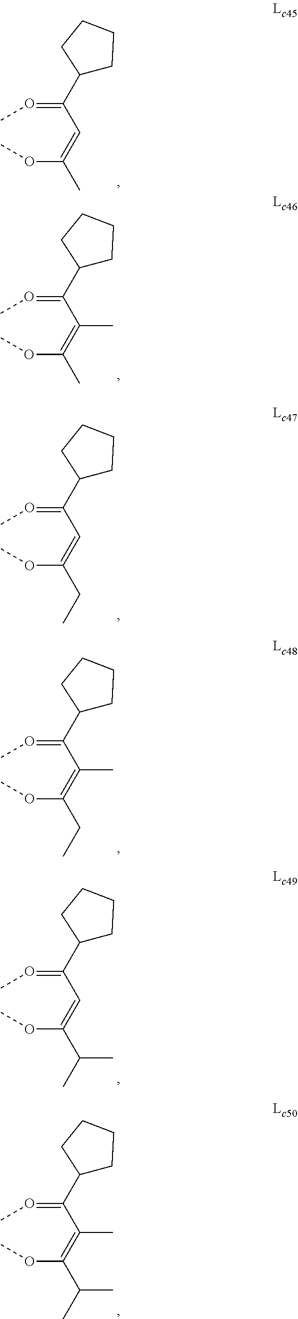

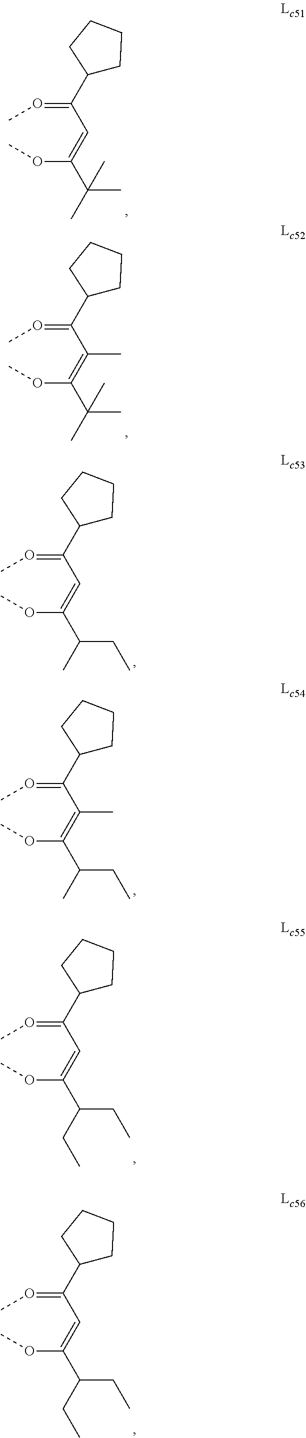

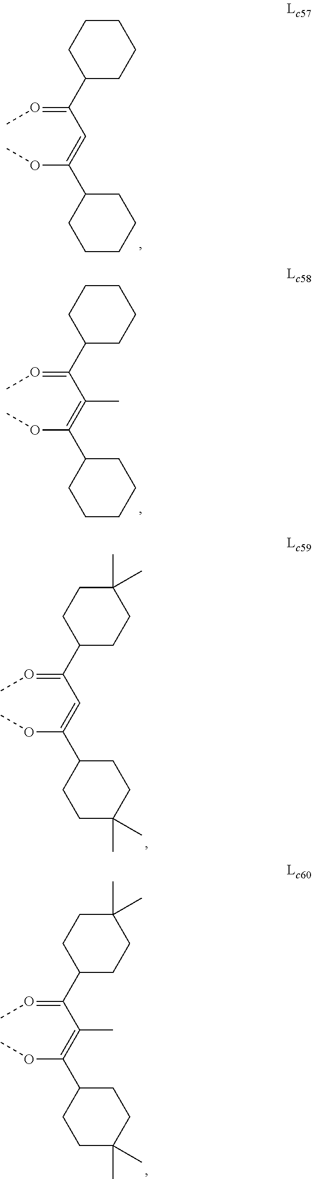

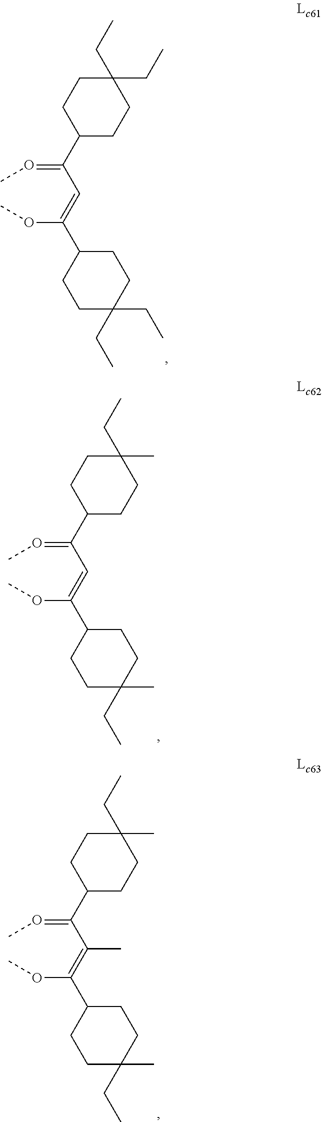

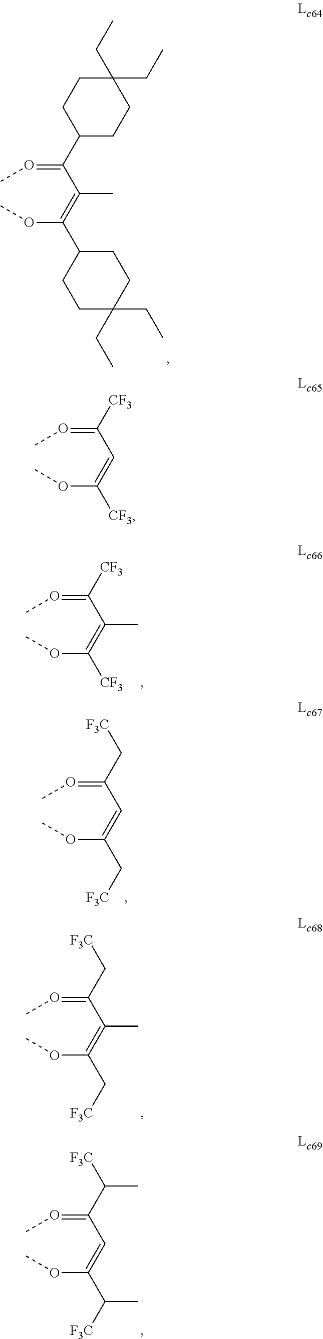

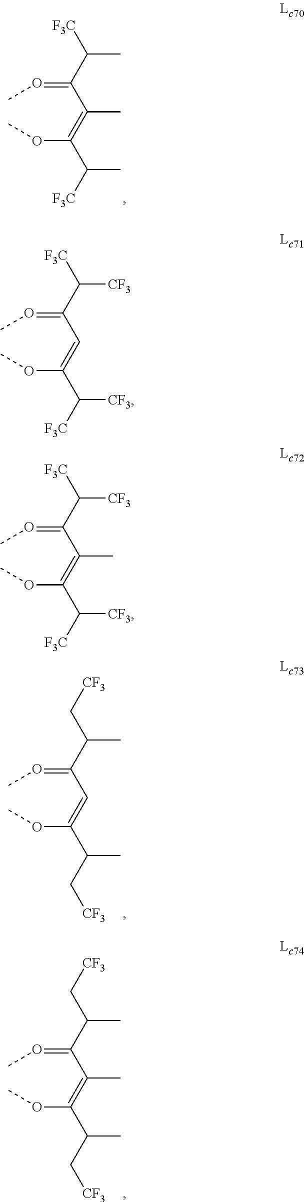

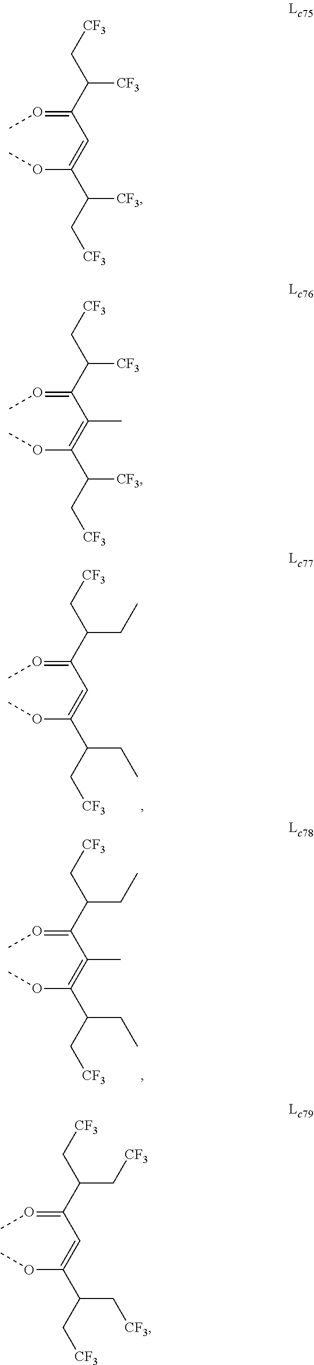

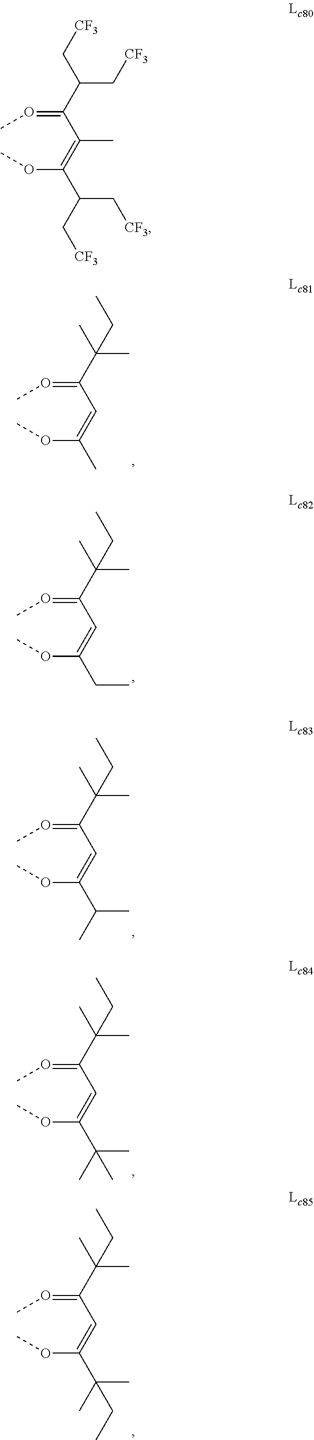

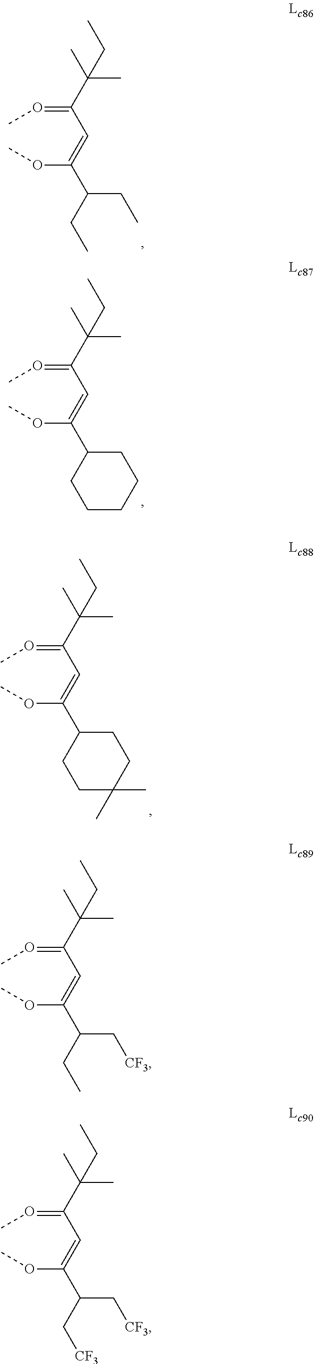

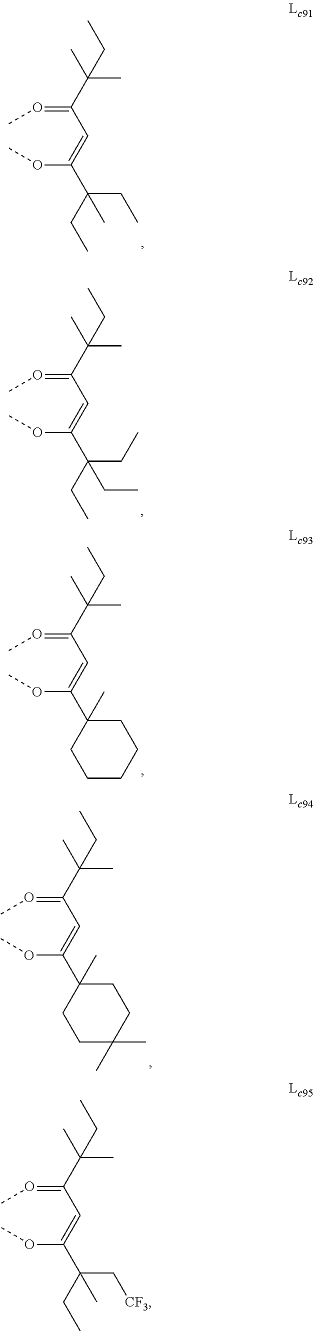

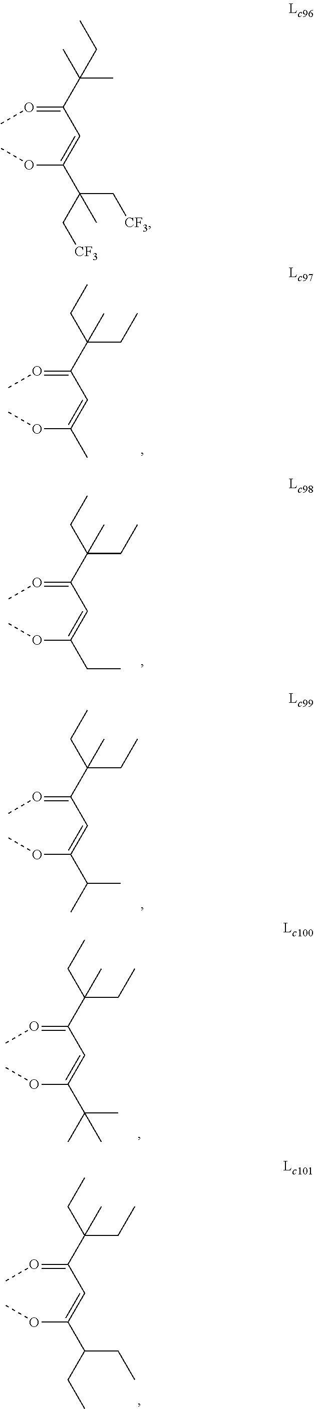

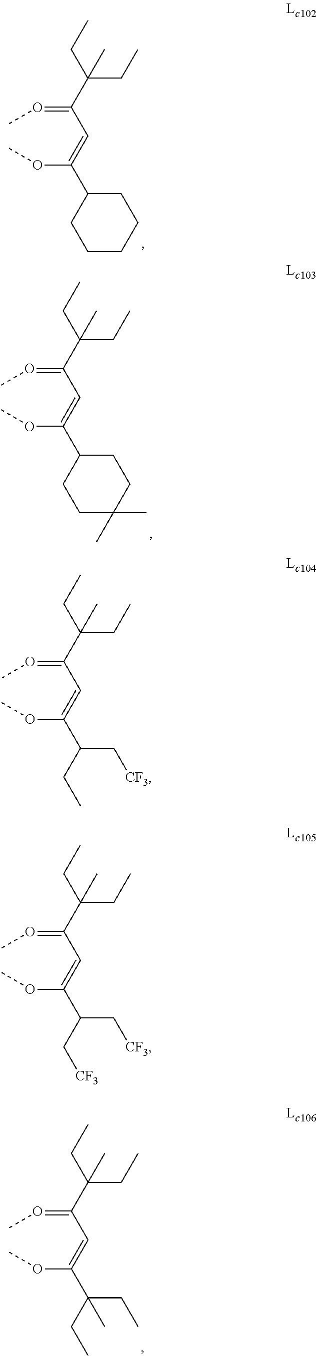

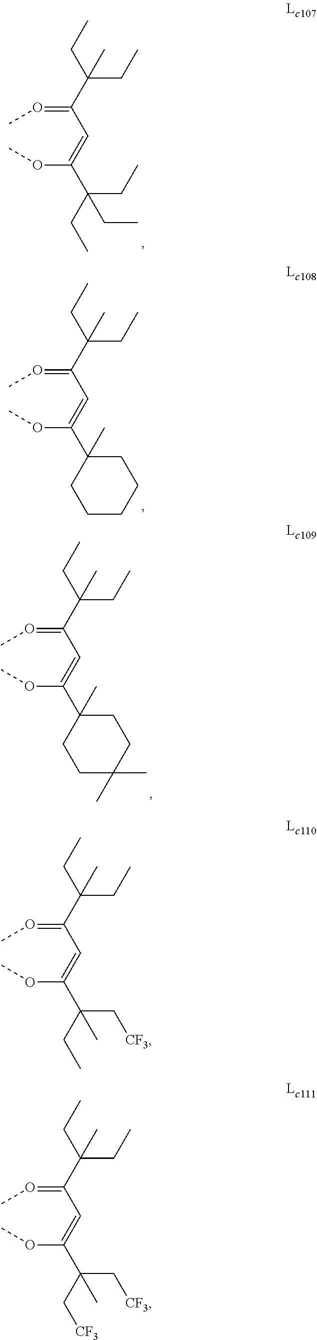

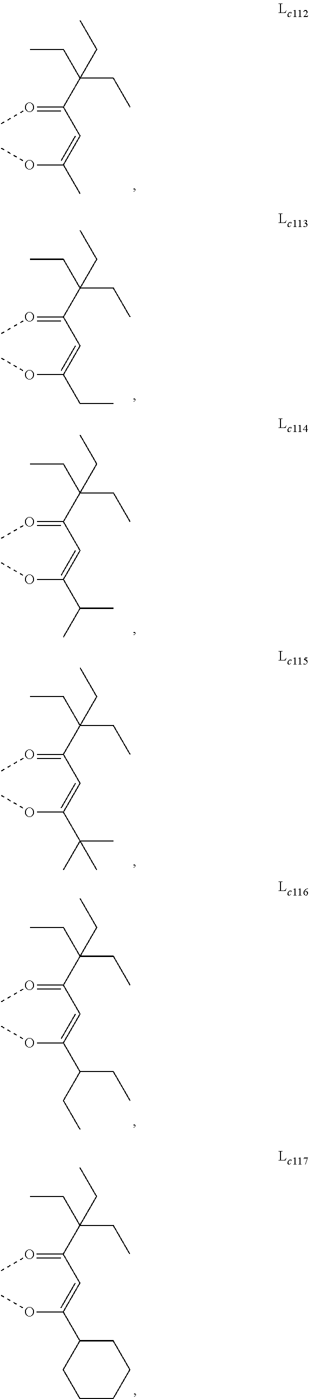

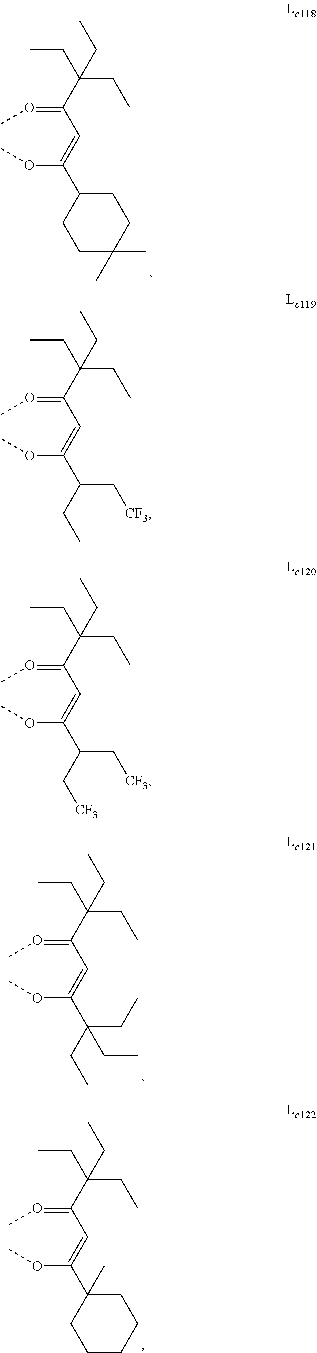

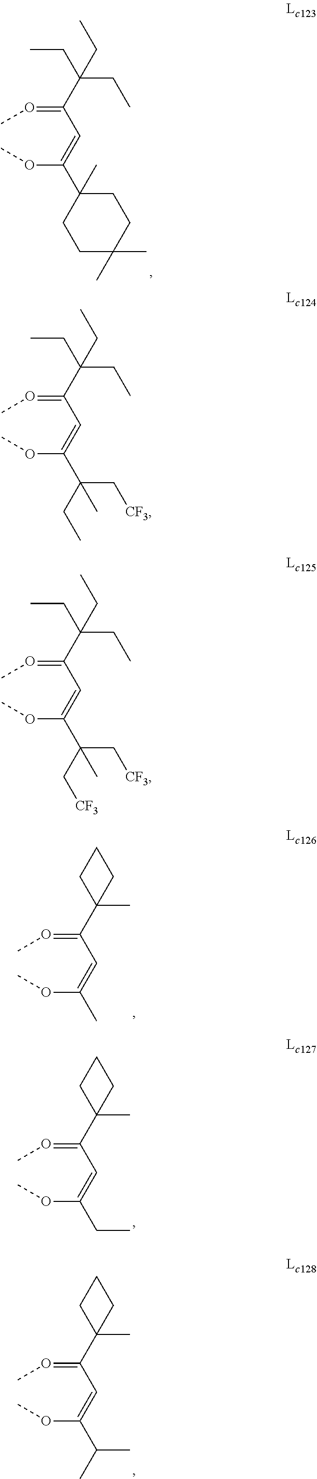

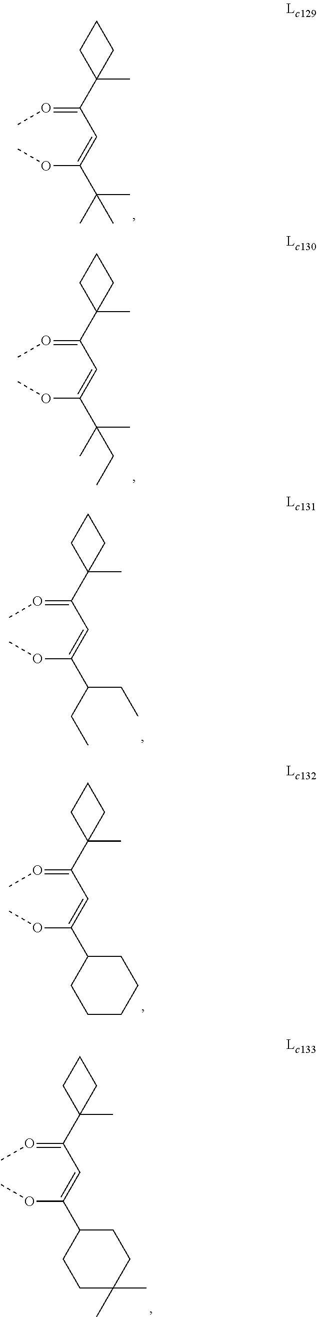

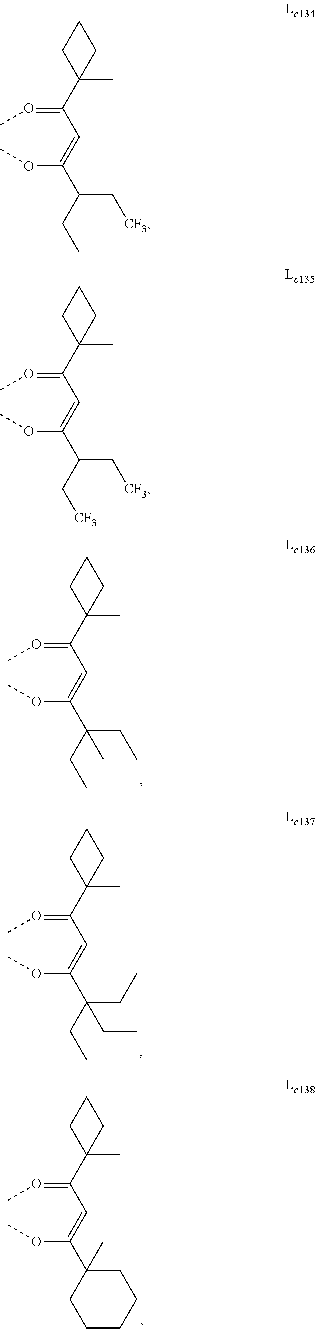

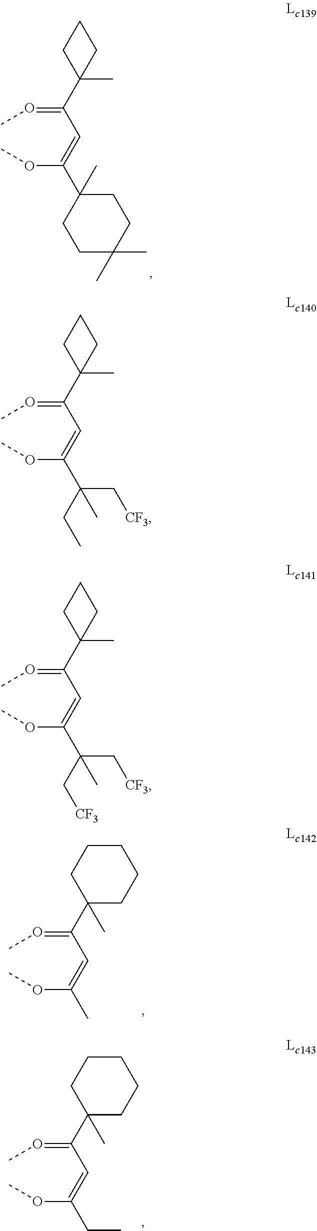

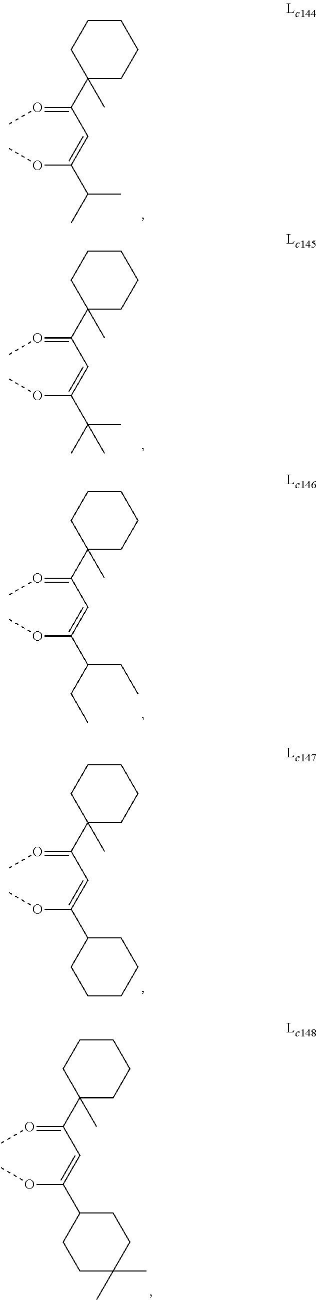

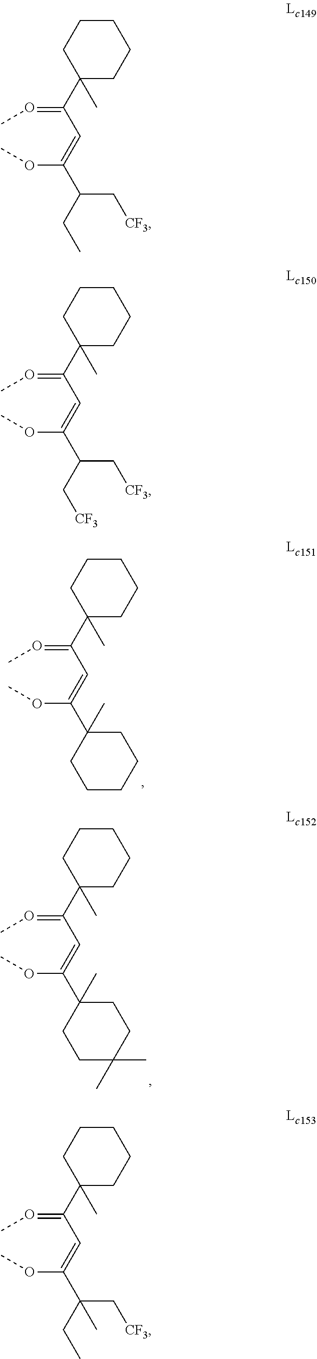

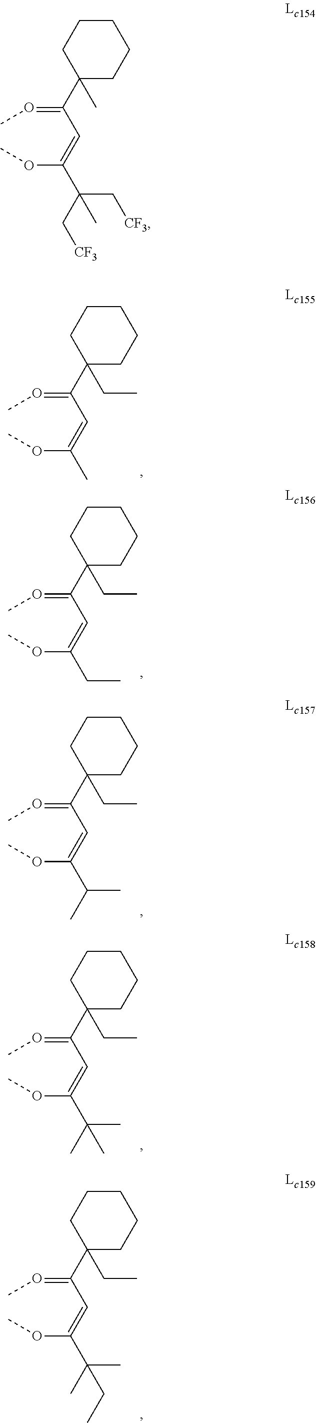

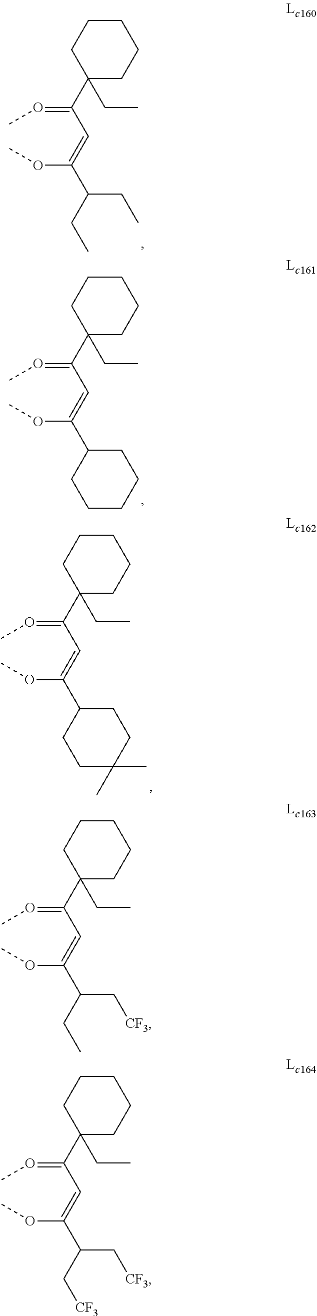

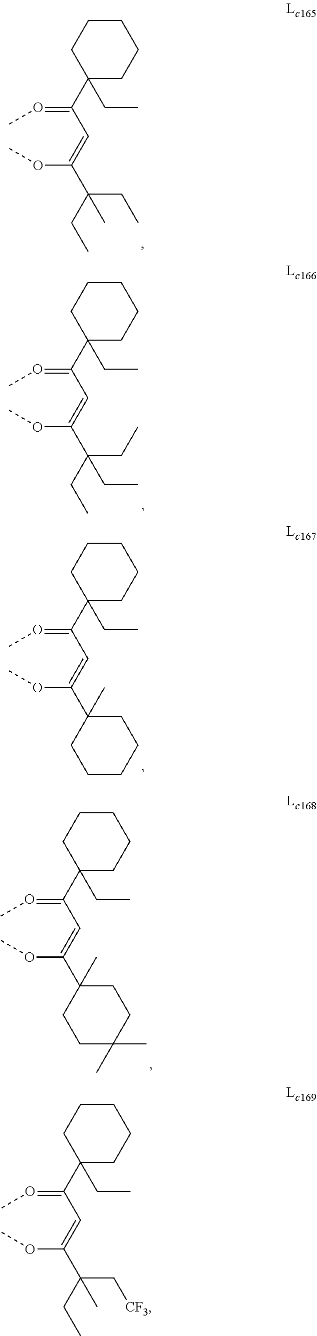

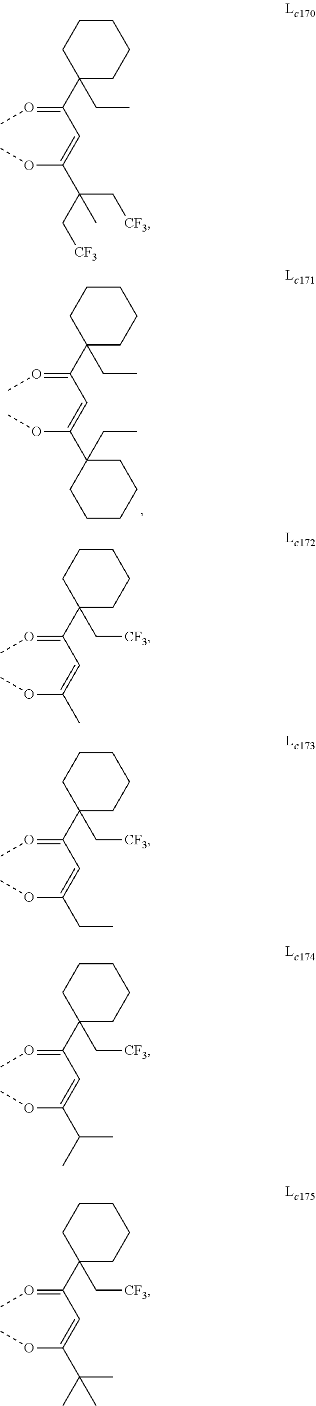

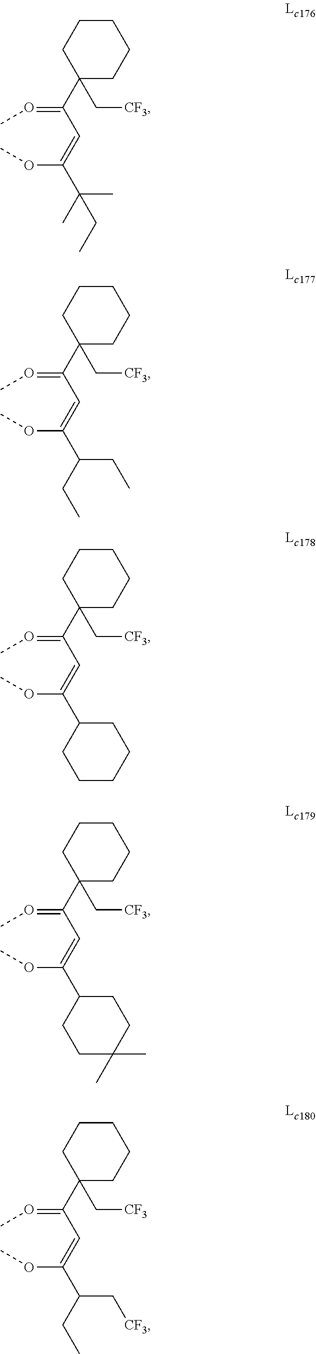

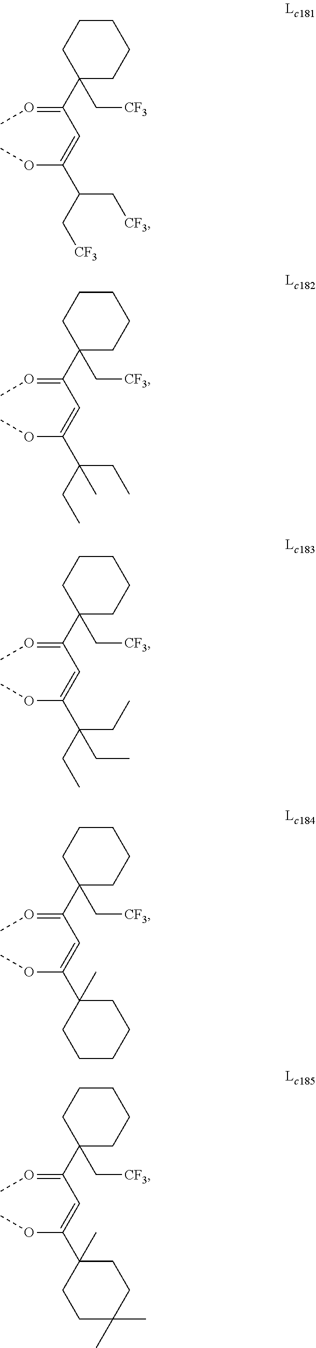

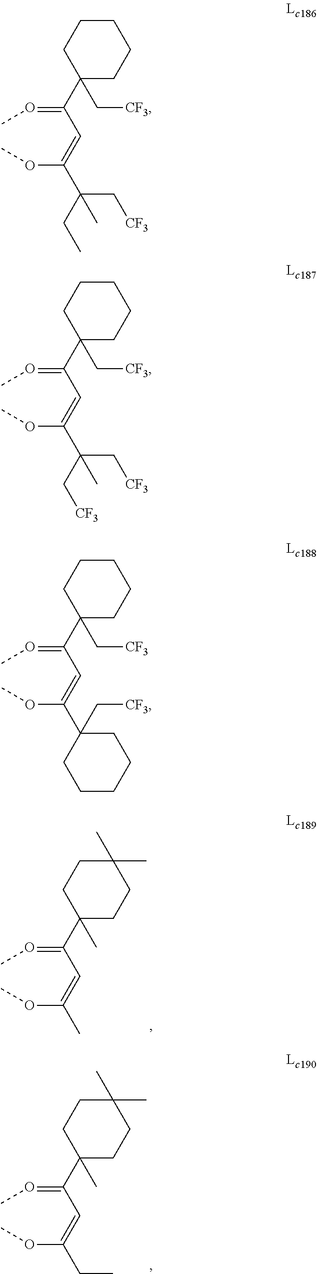

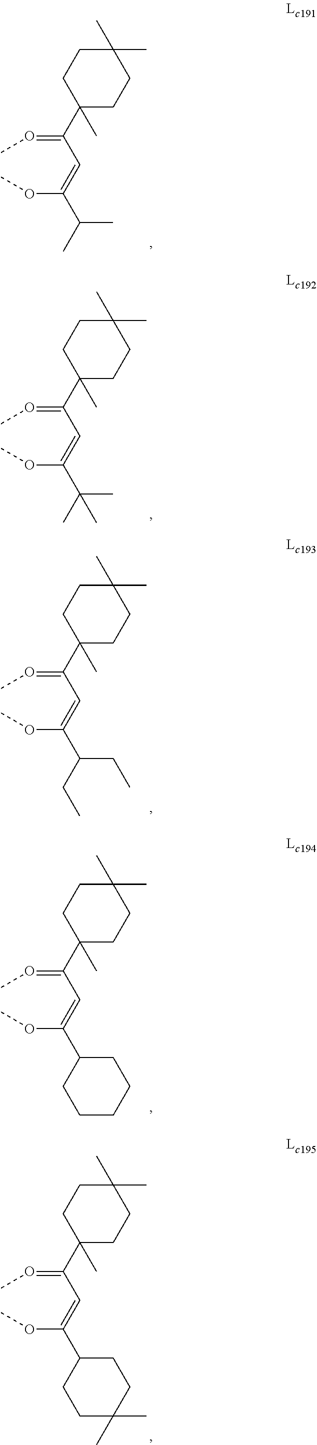

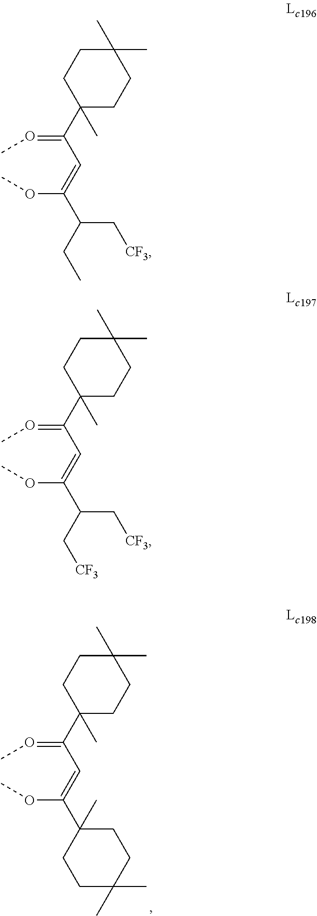

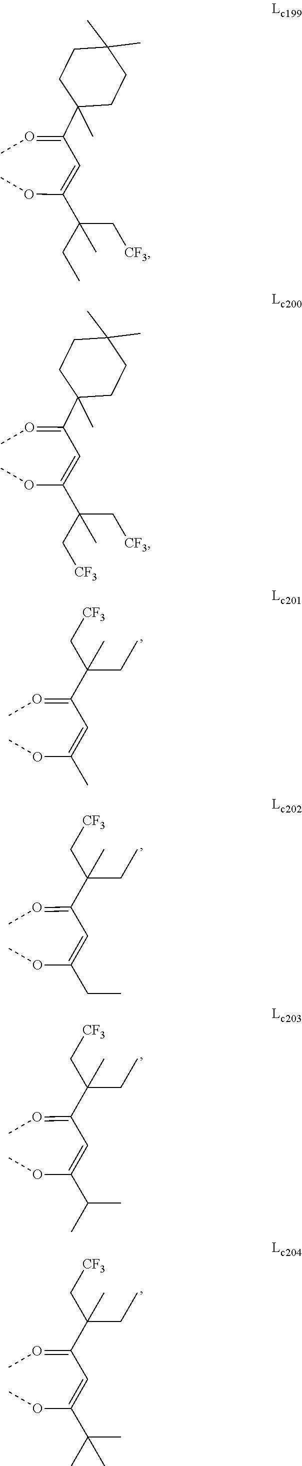

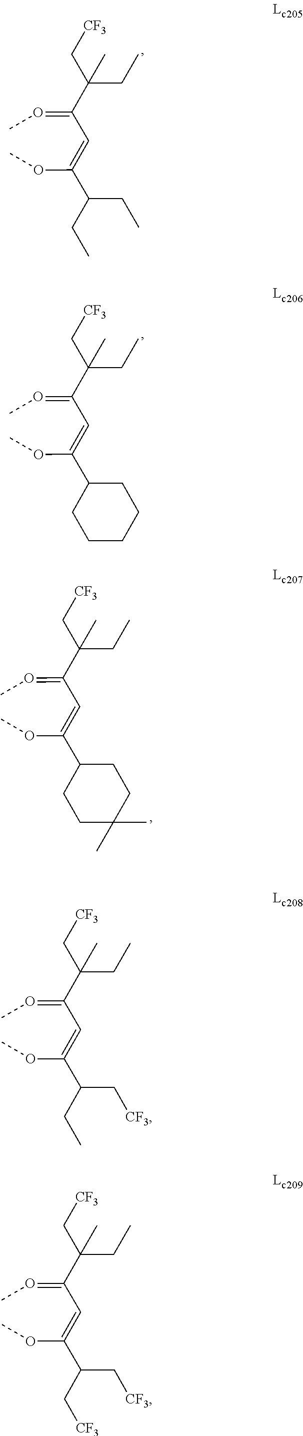

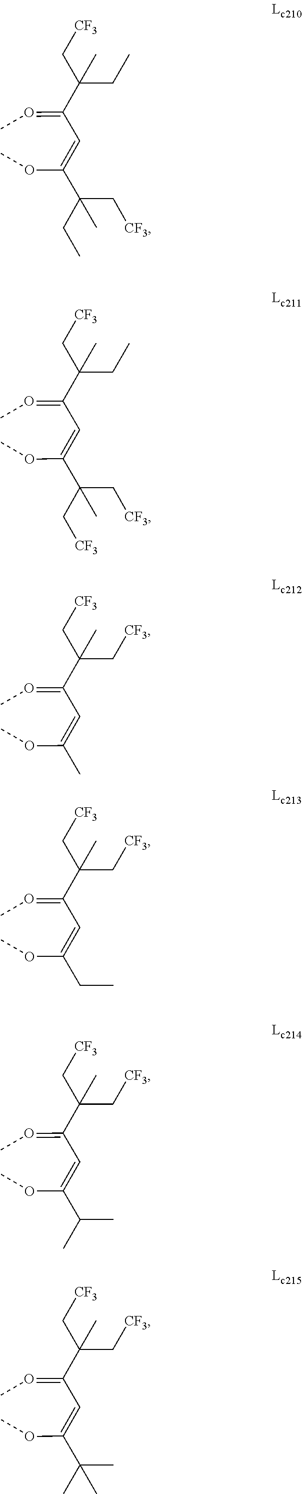

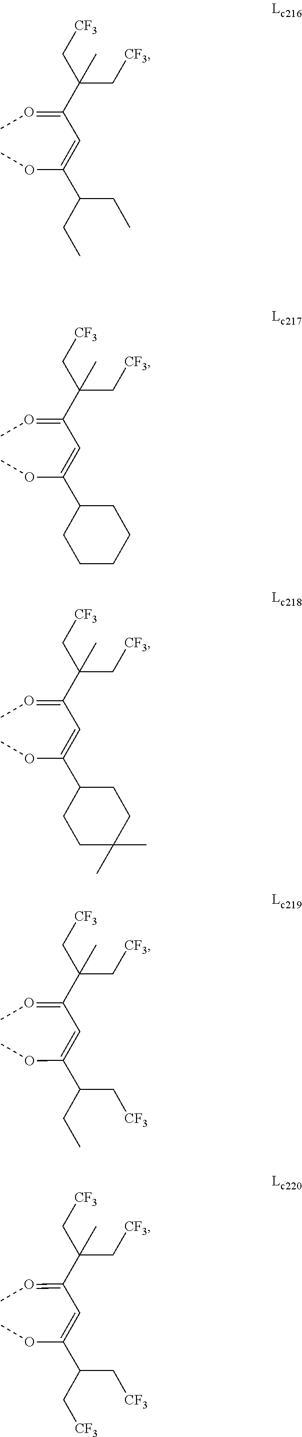

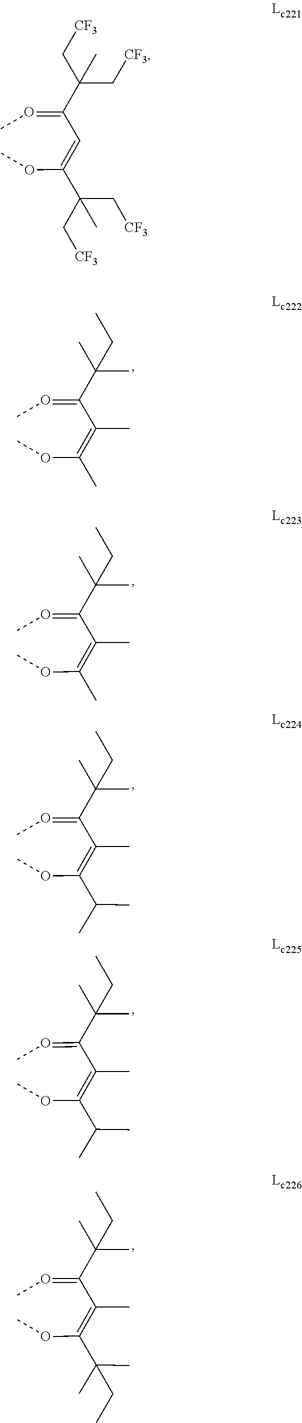

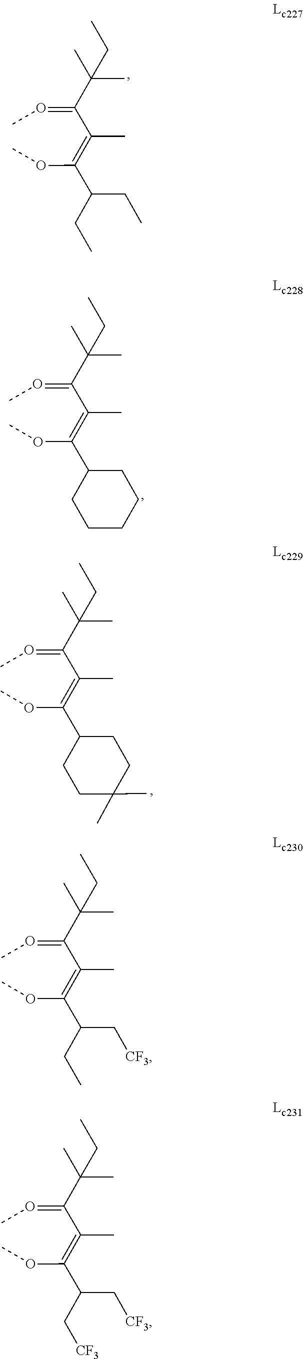

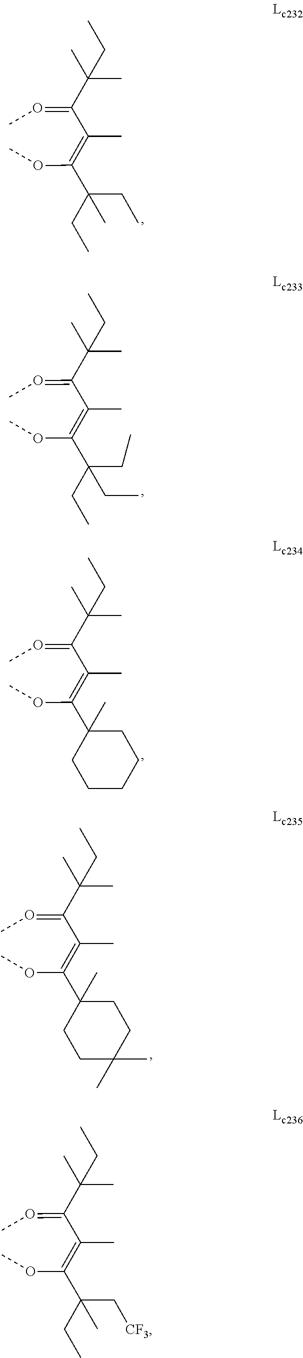

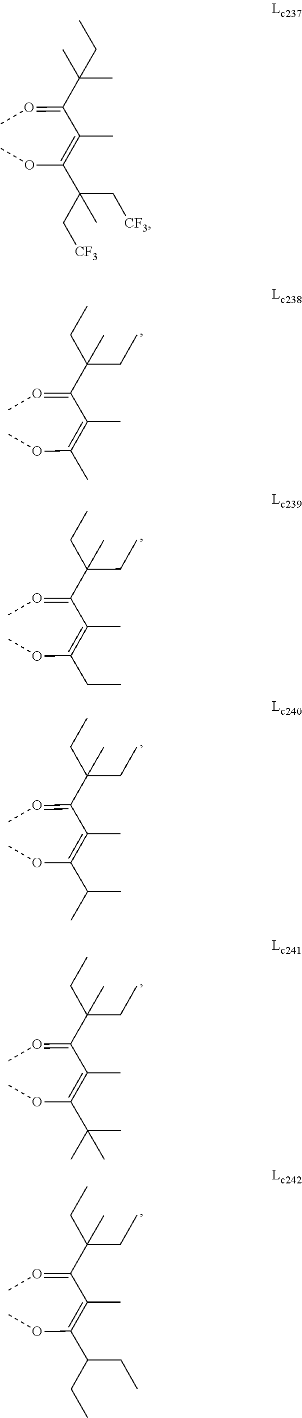

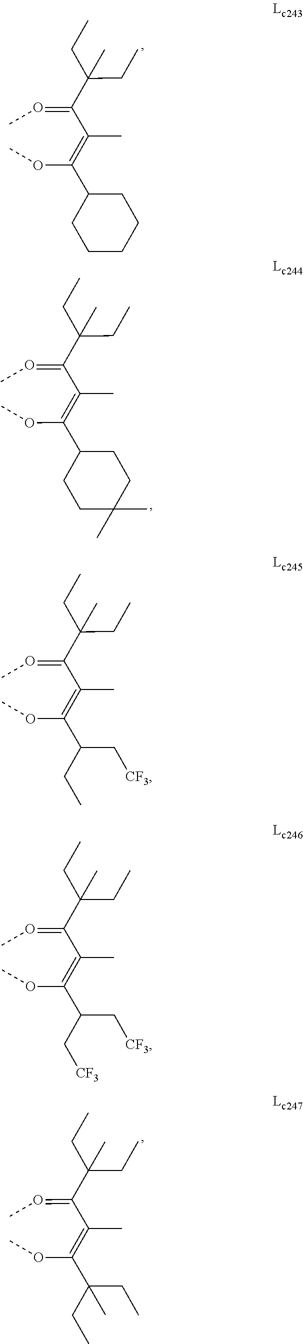

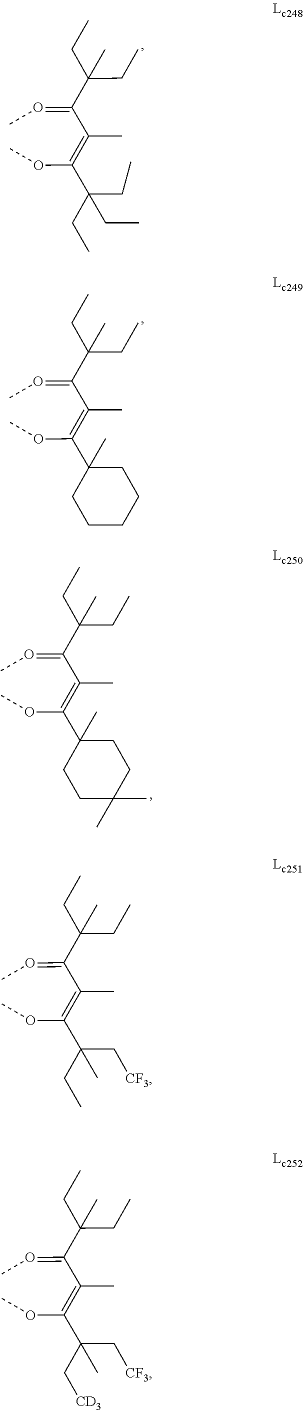

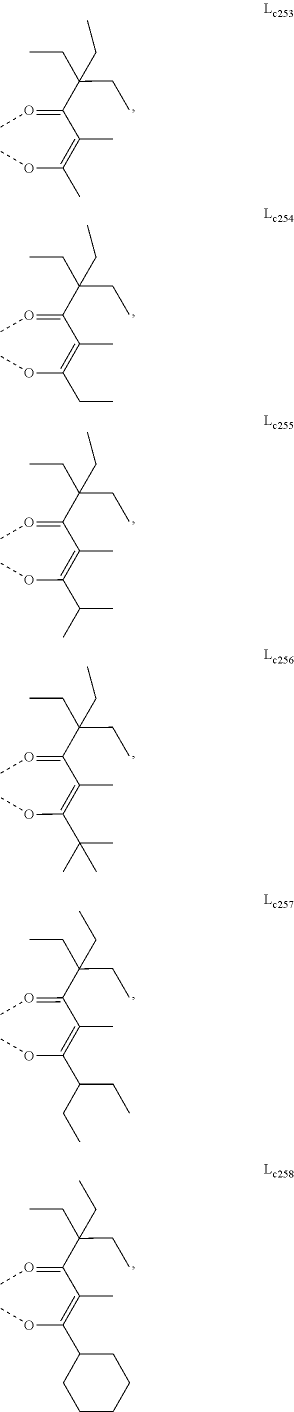

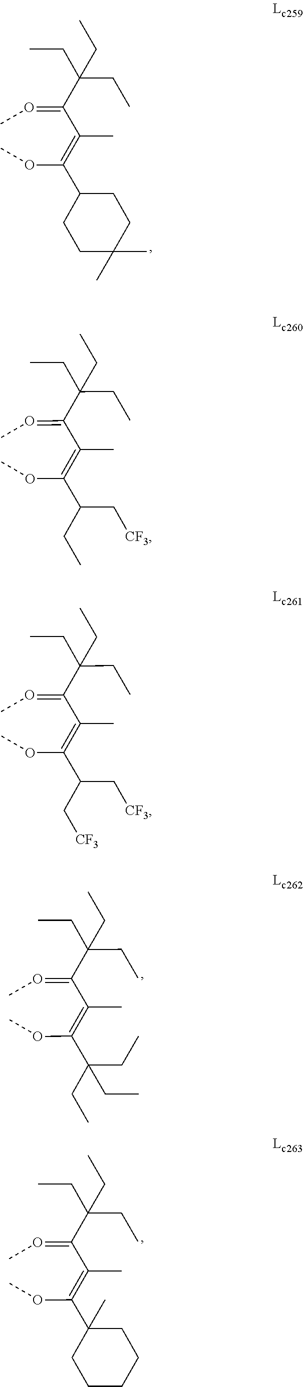

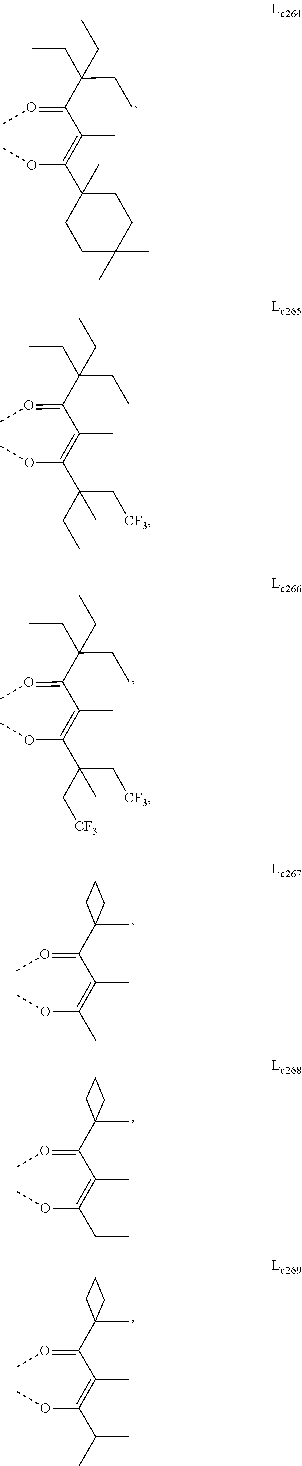

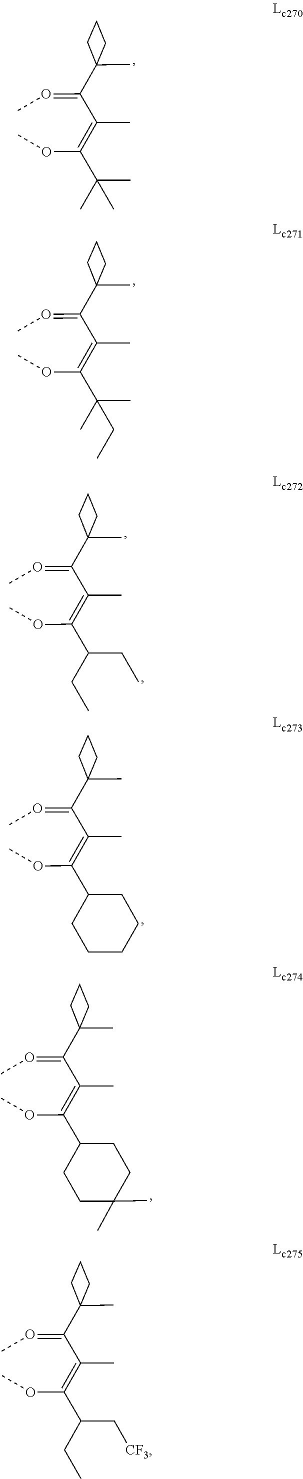

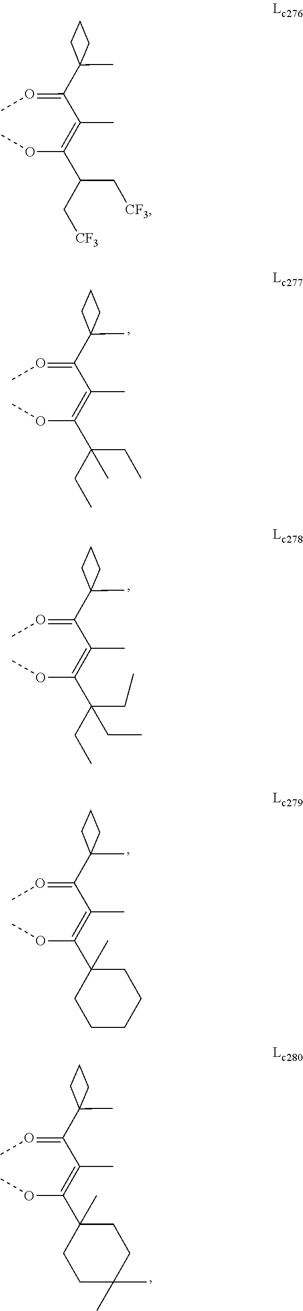

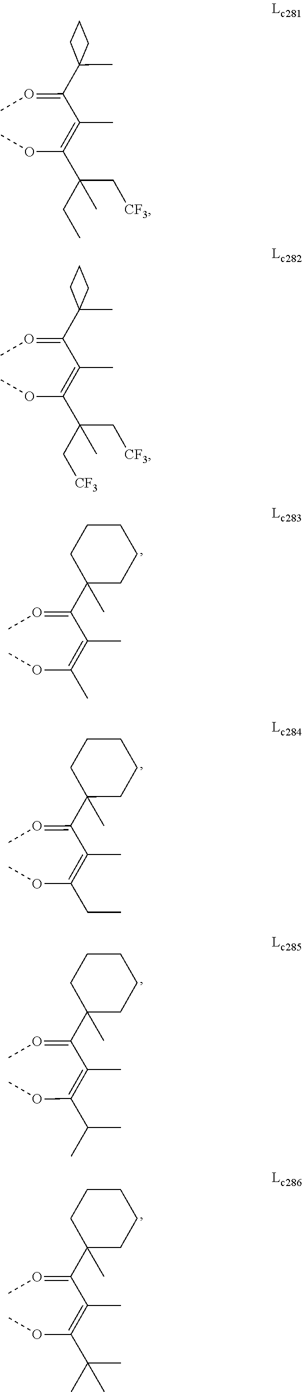

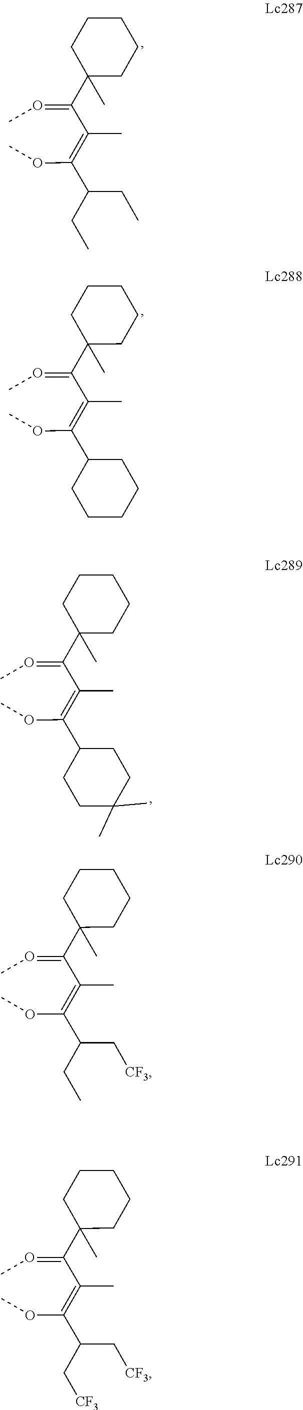

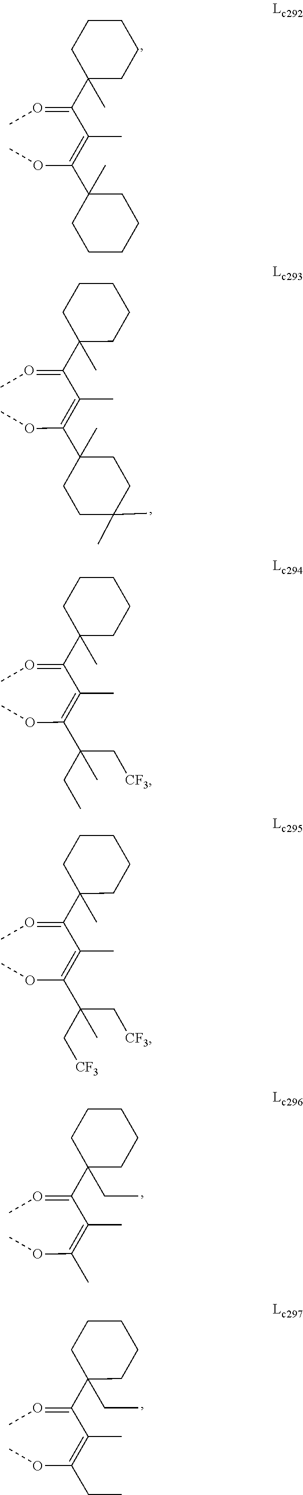

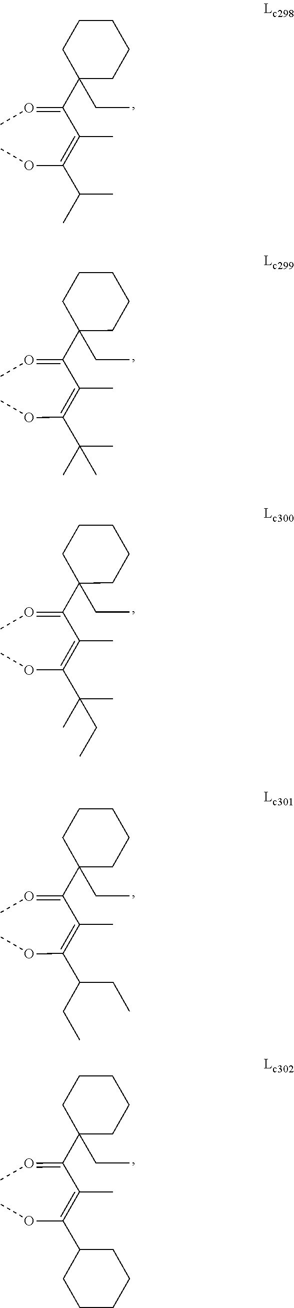

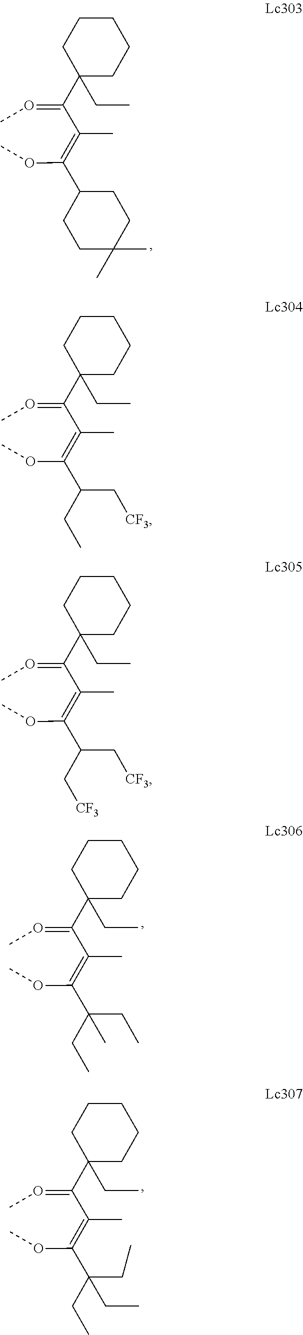

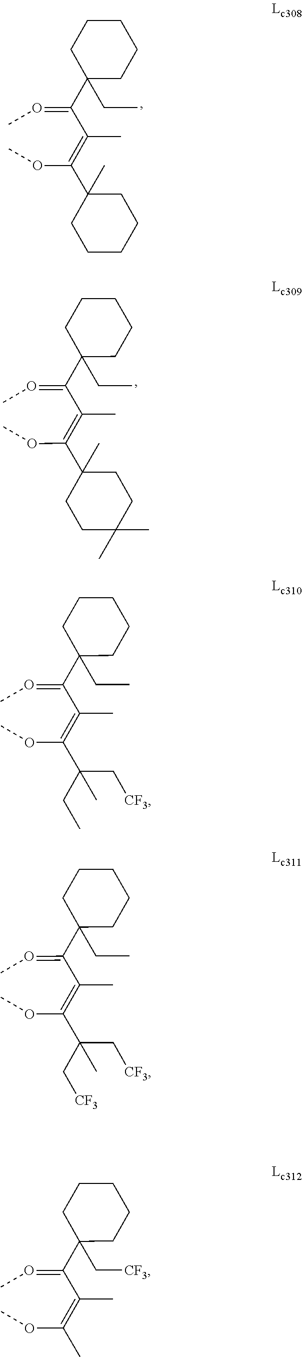

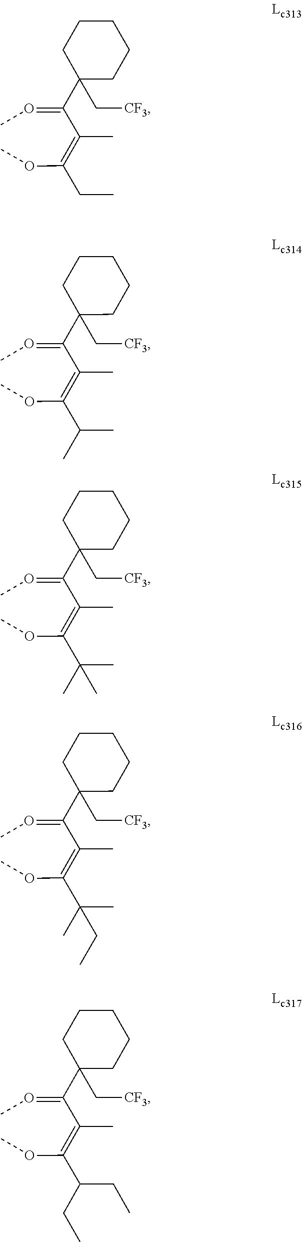

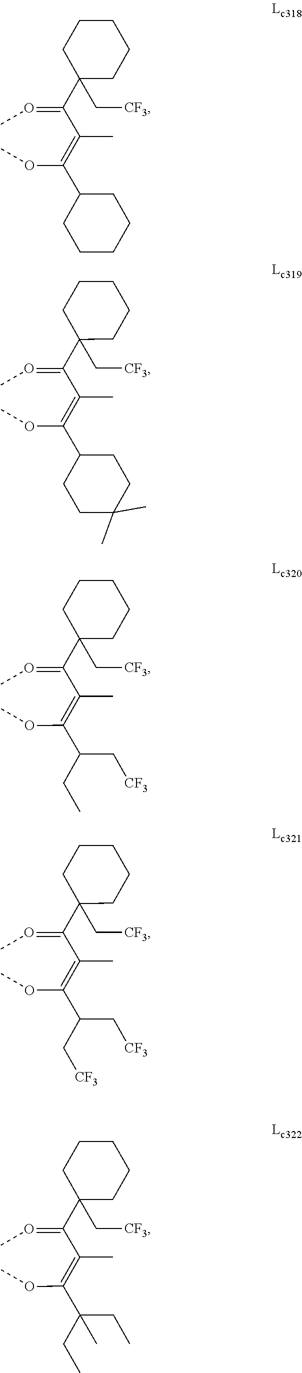

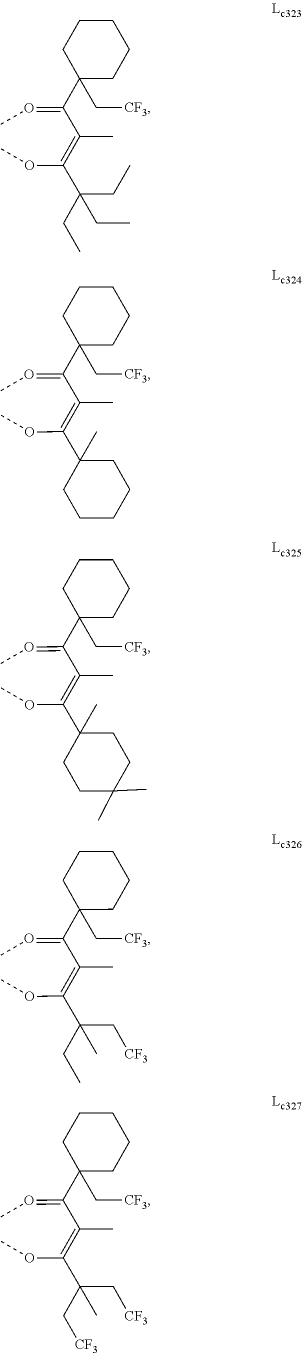

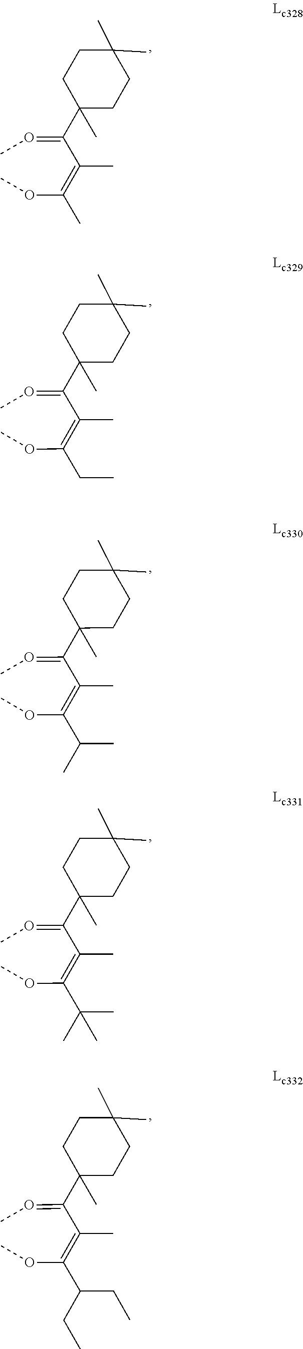

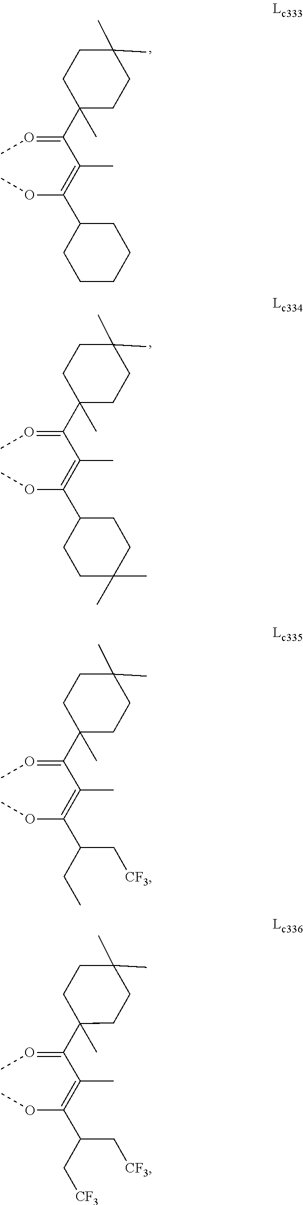

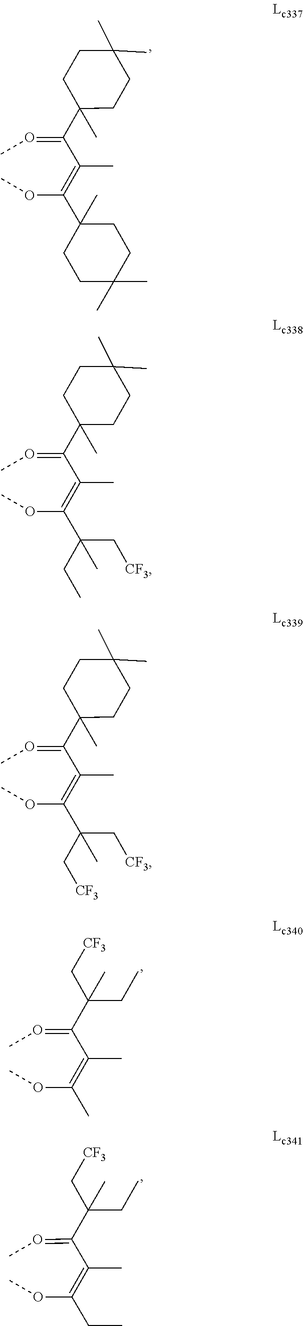

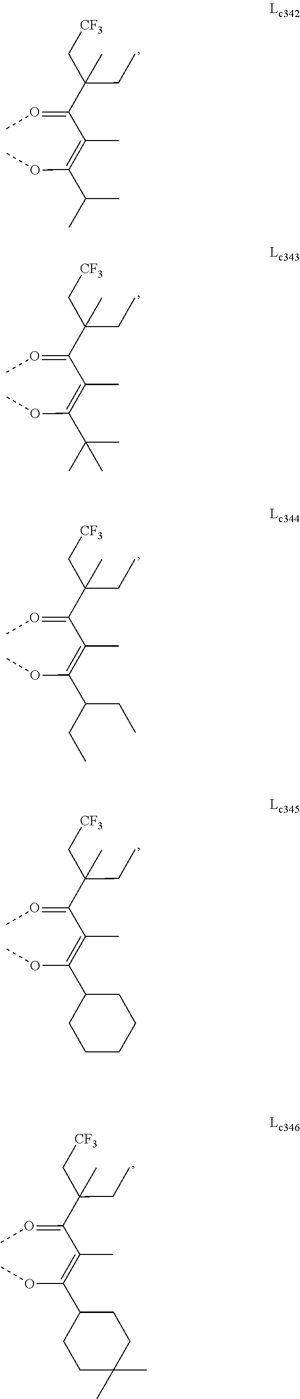

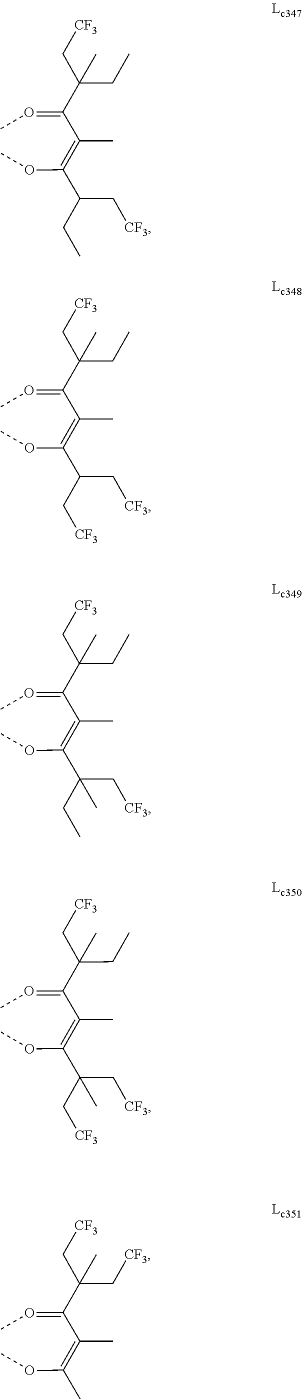

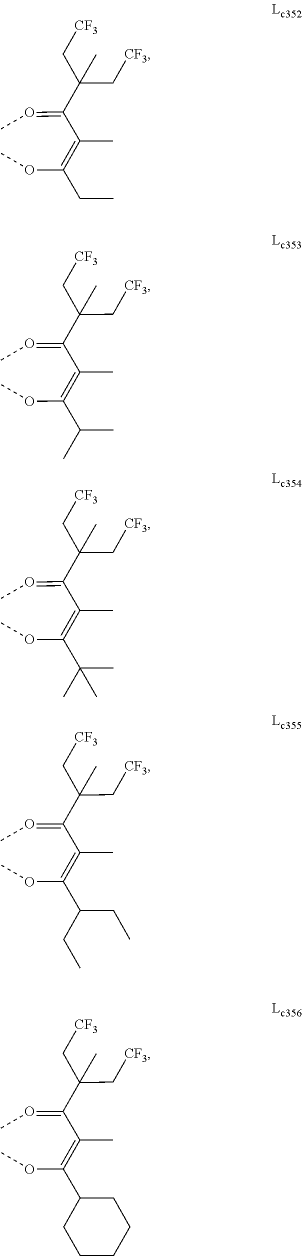

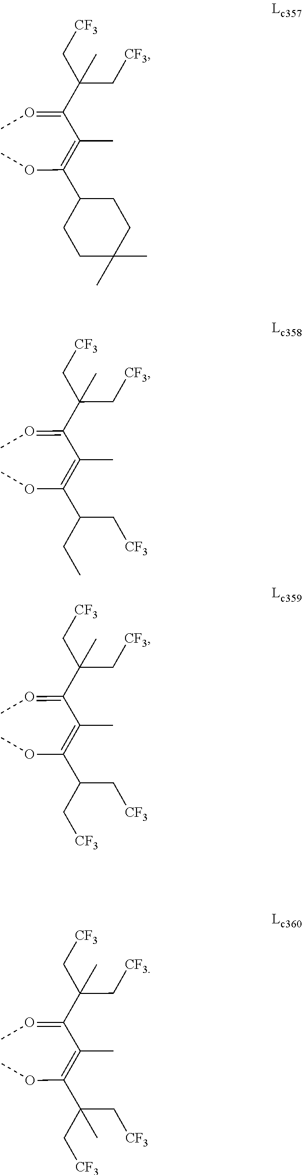

13. The electroluminescent device according to claim 1, wherein the first metal complex is selected from the group consisting of:

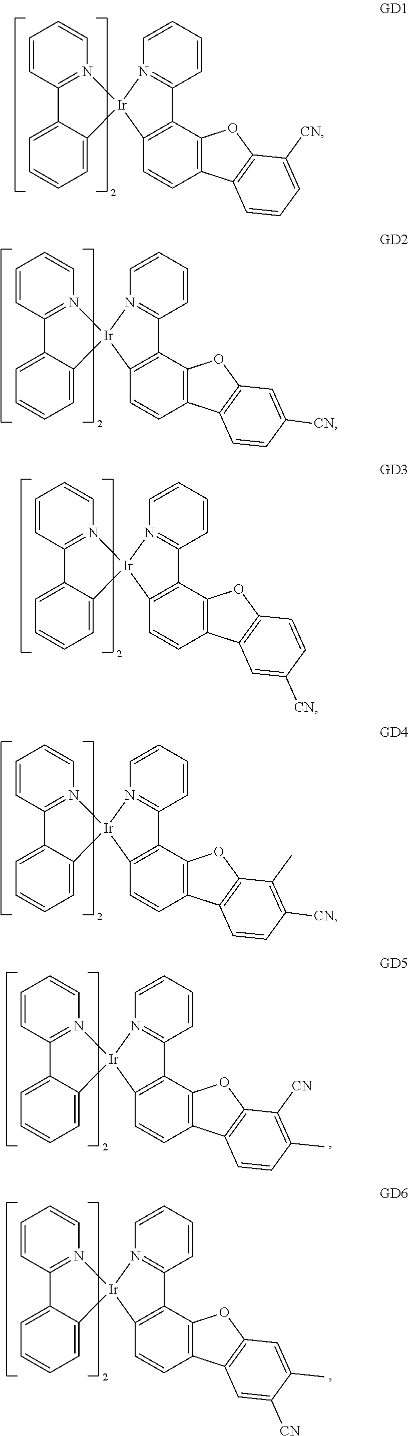

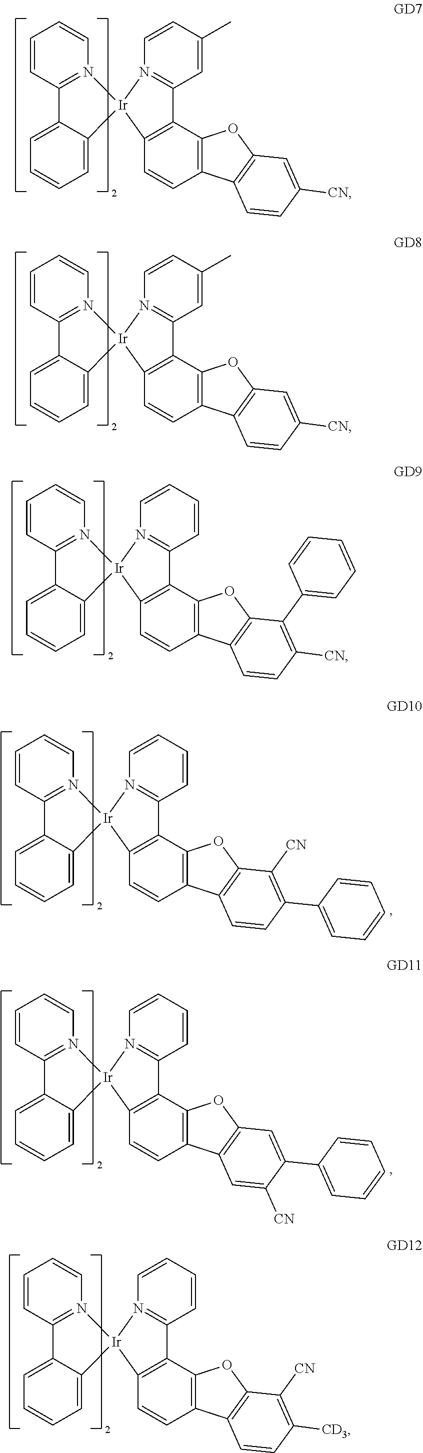

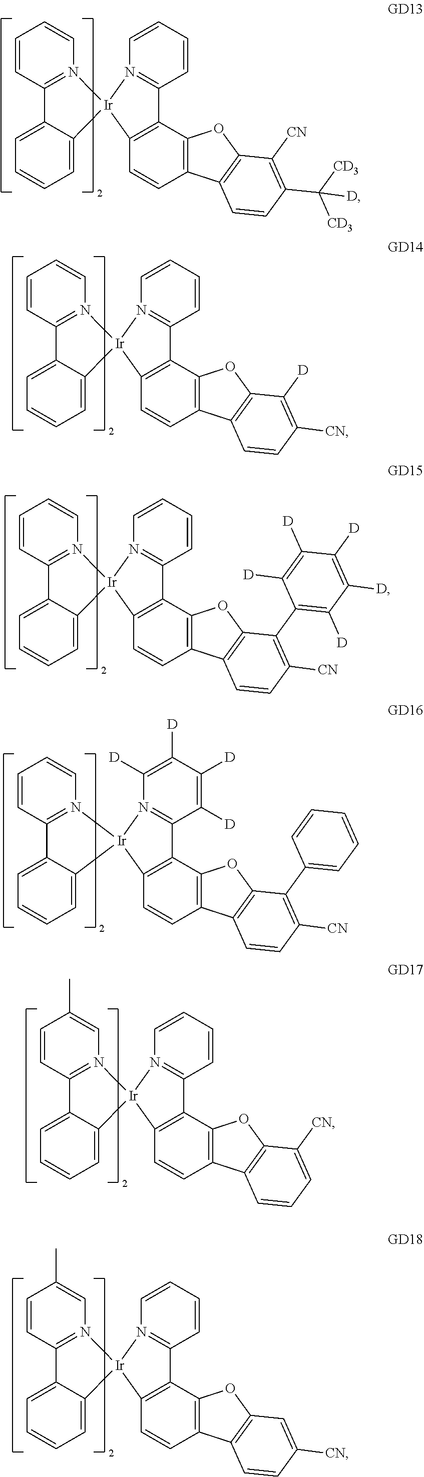

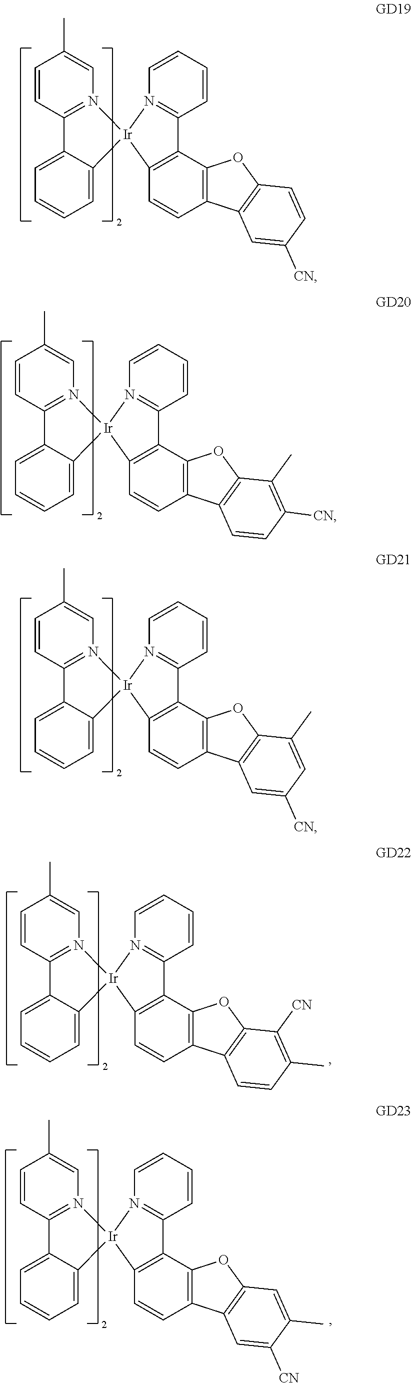

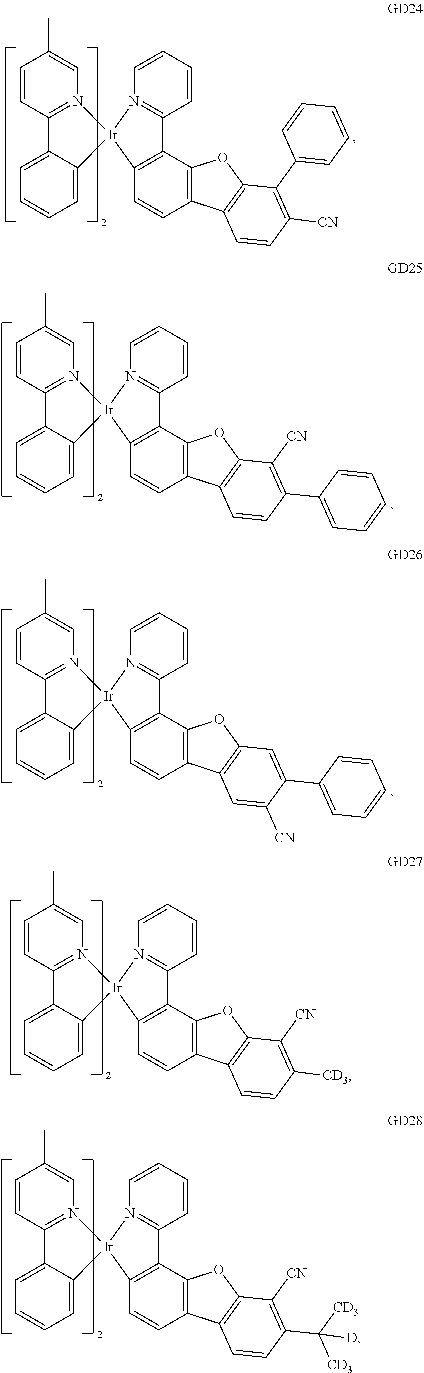

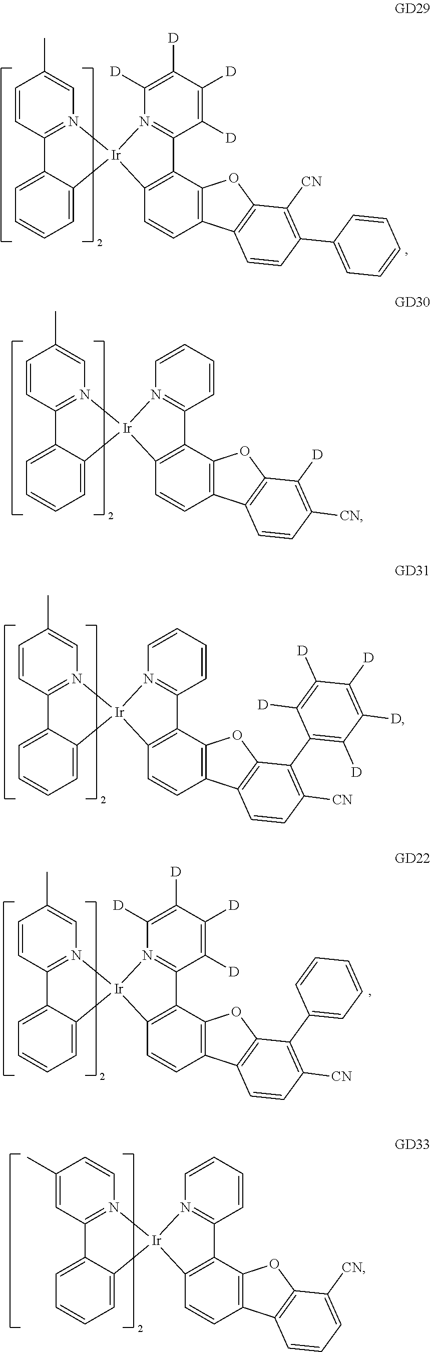

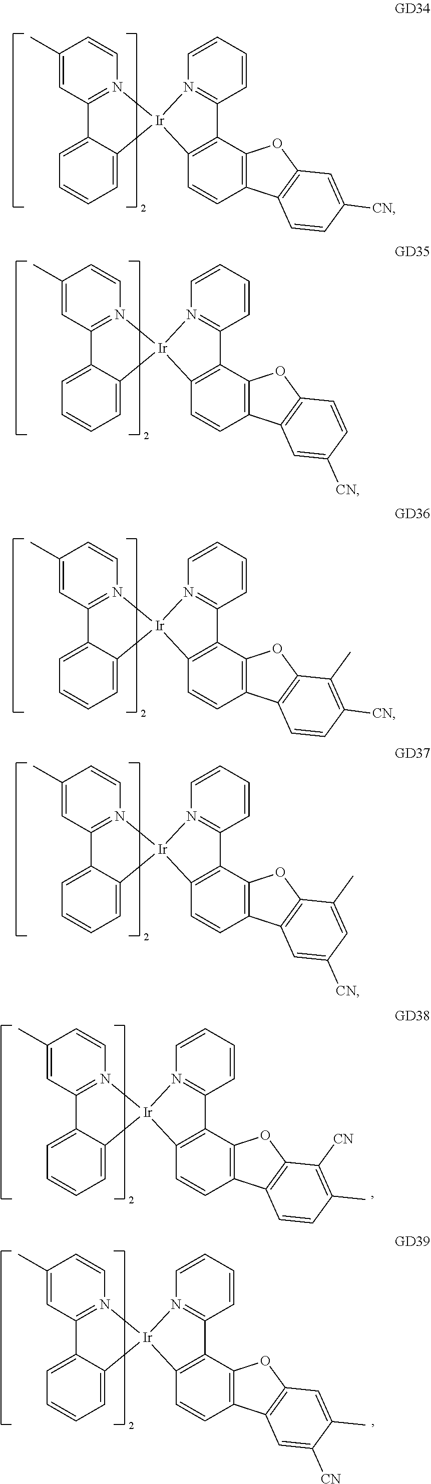

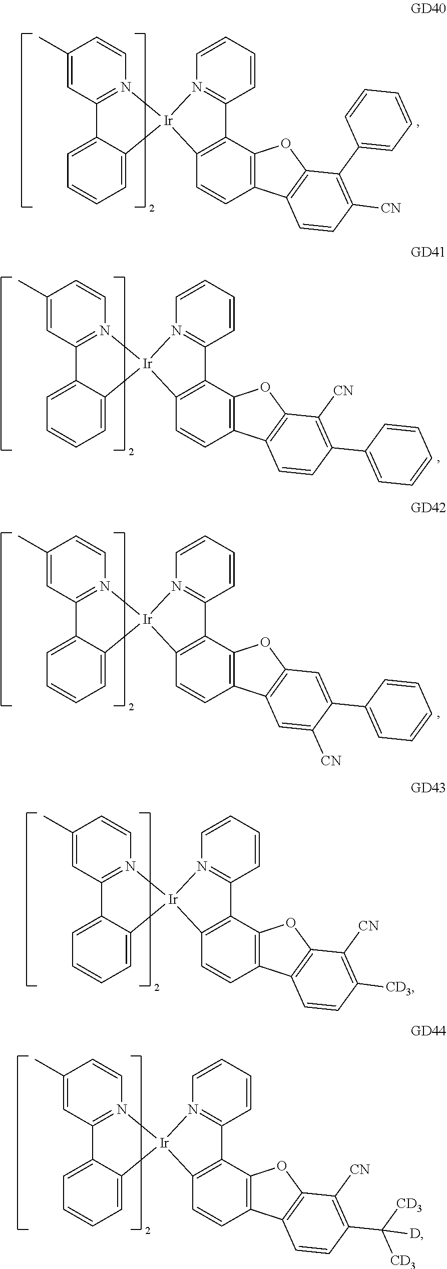

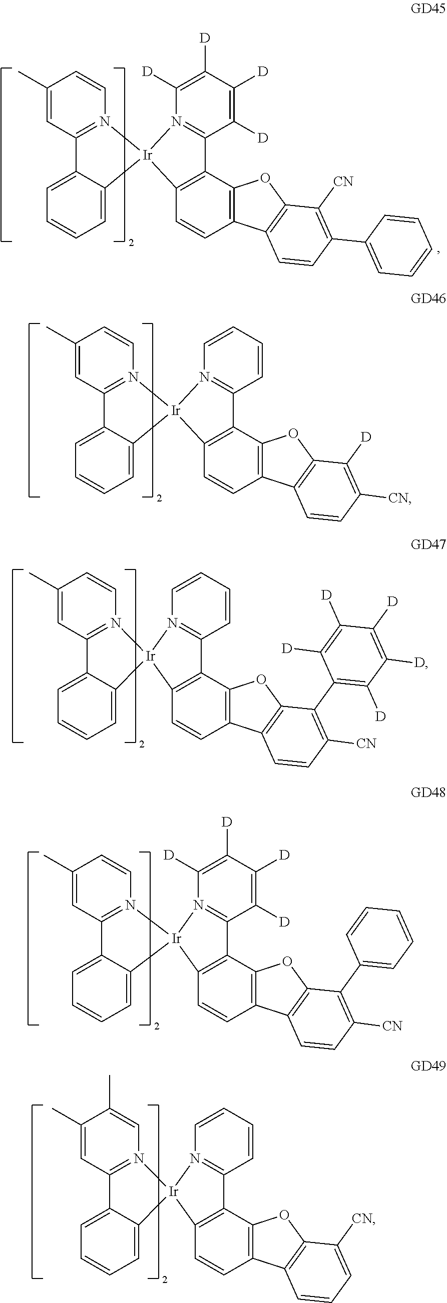

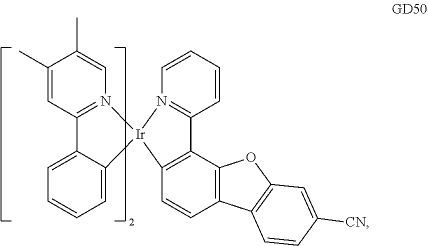

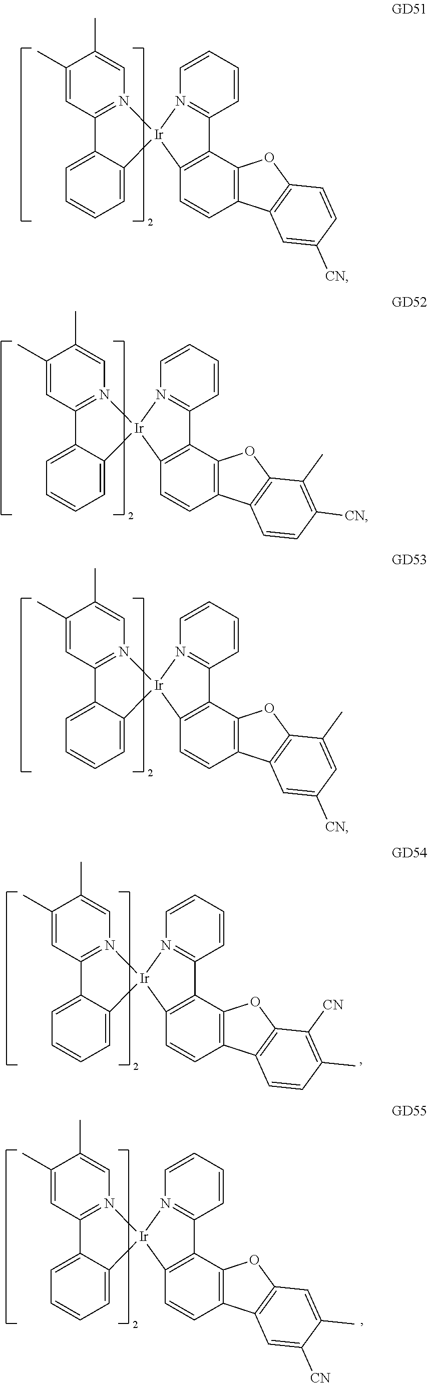

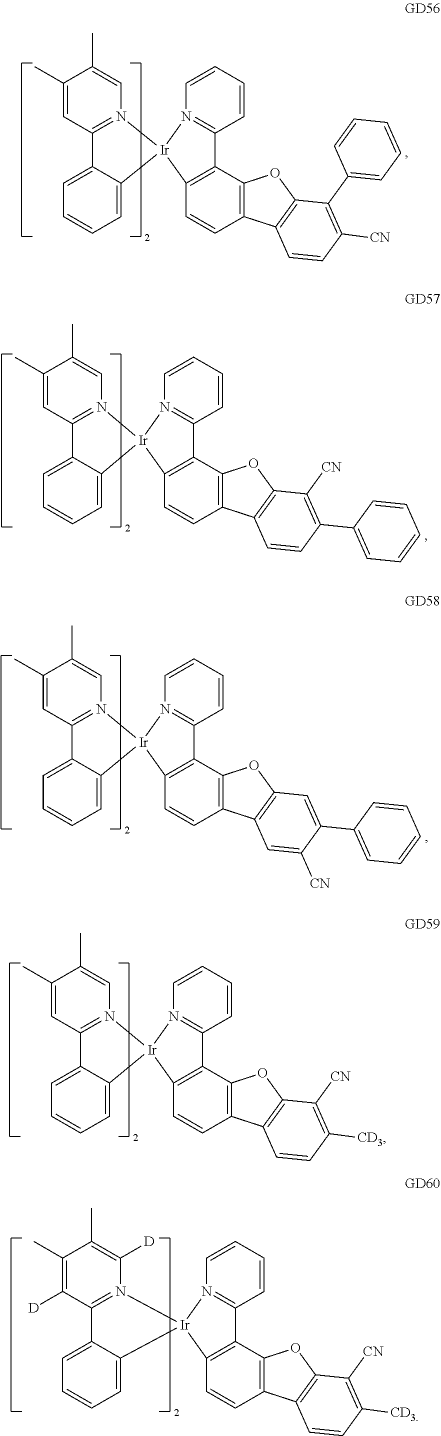

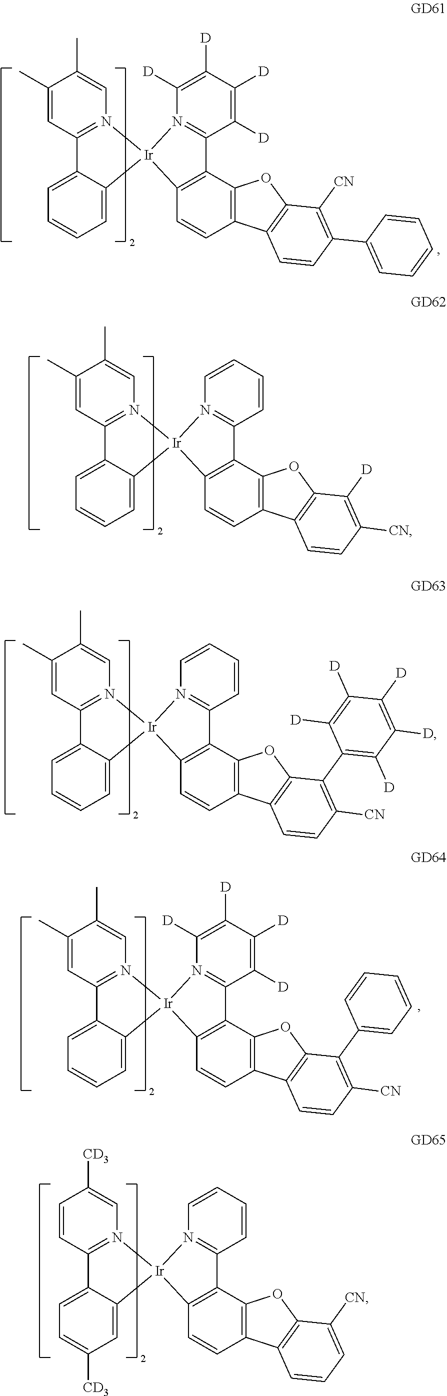

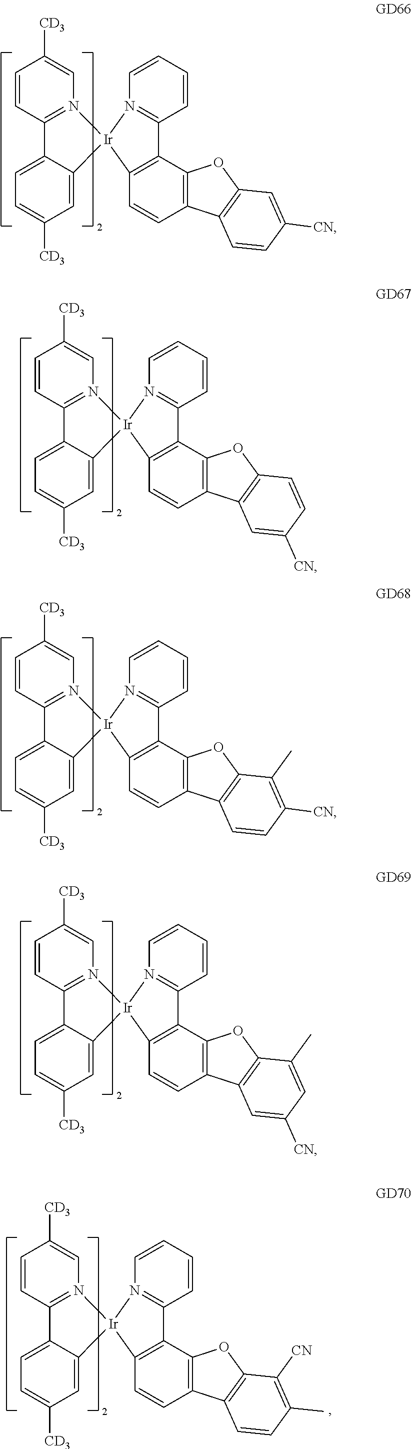

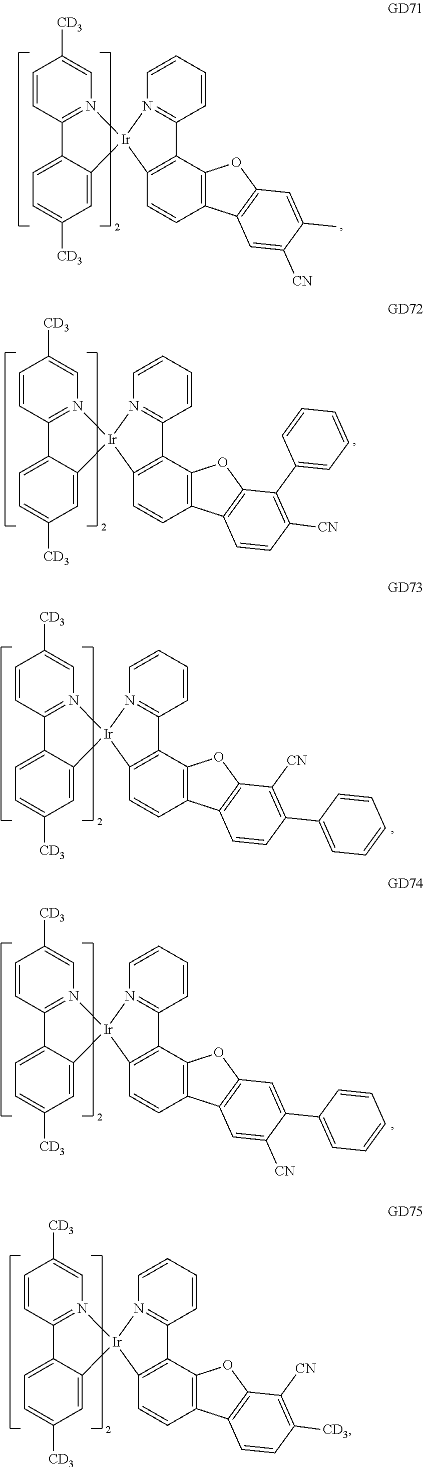

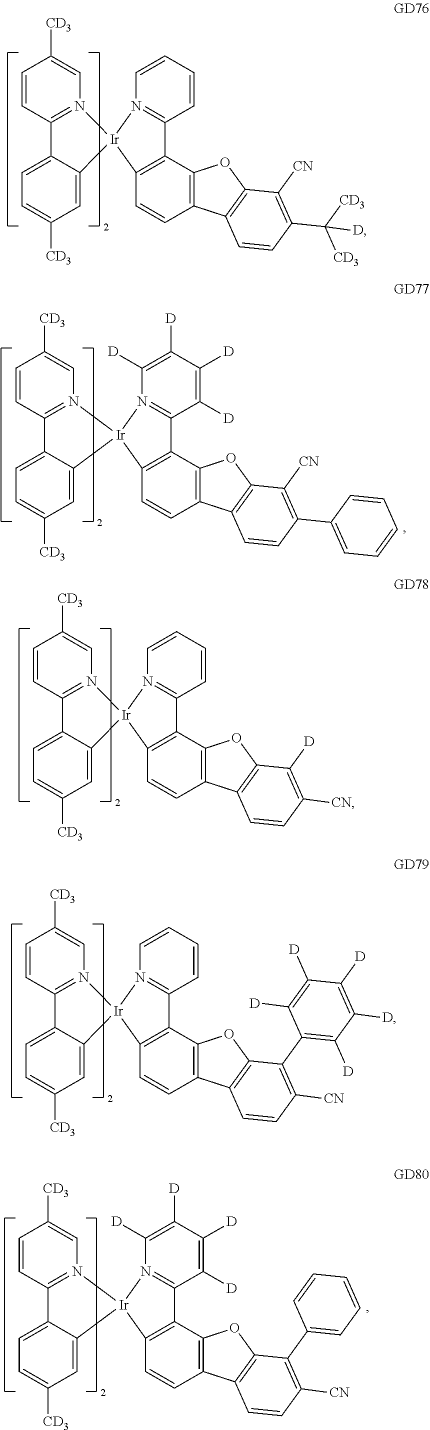

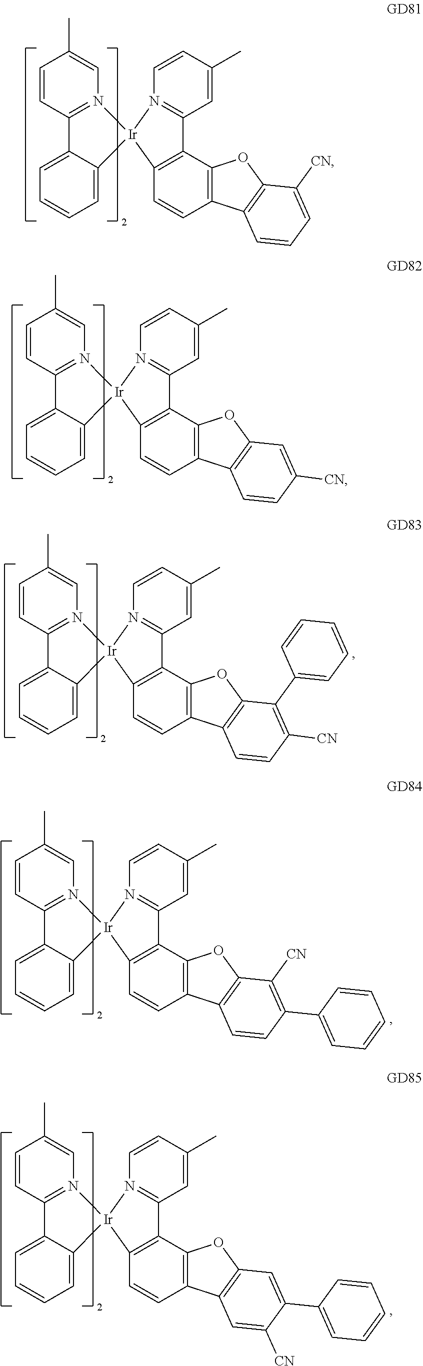

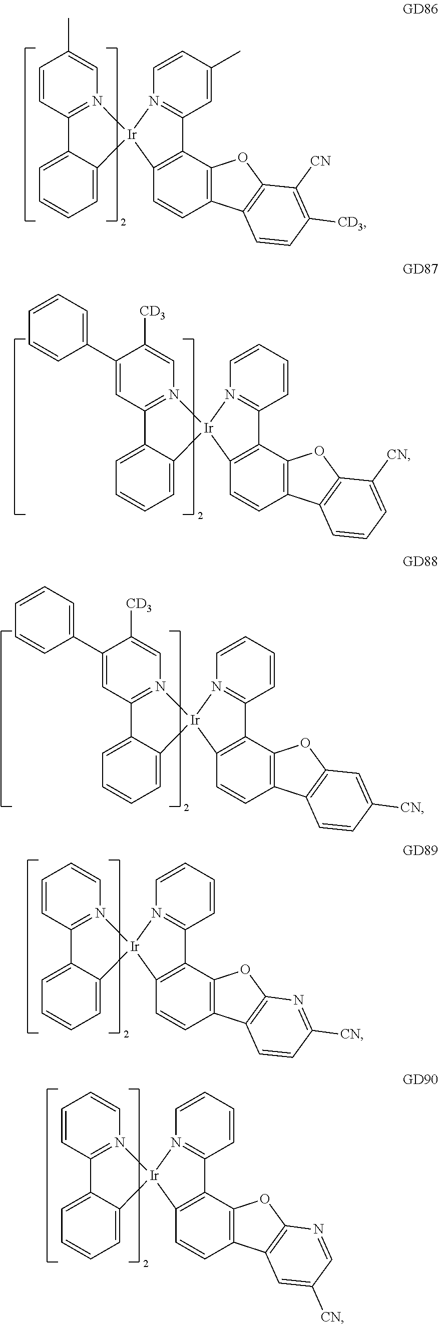

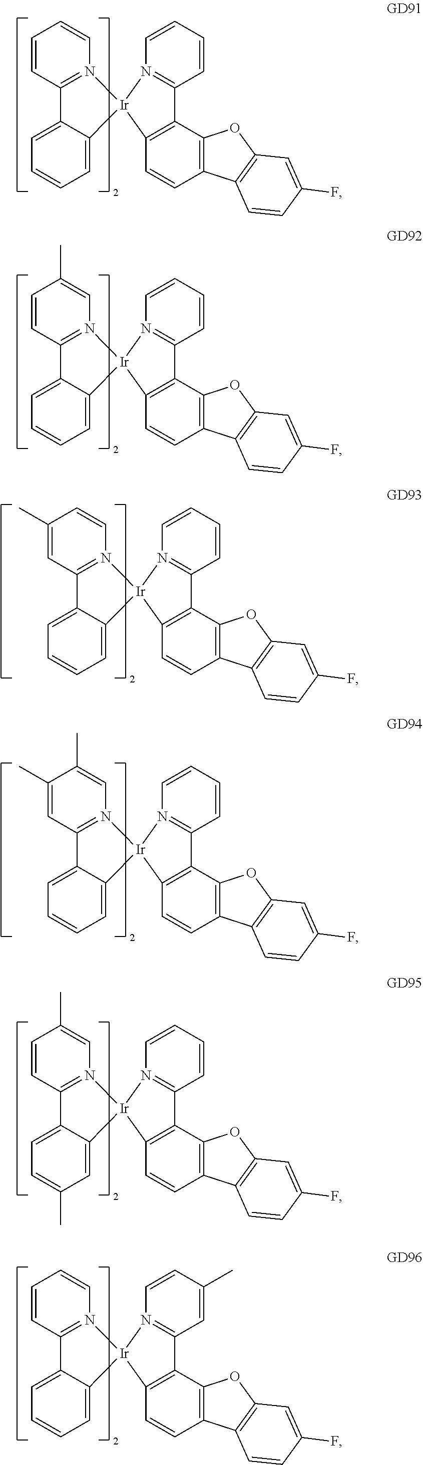

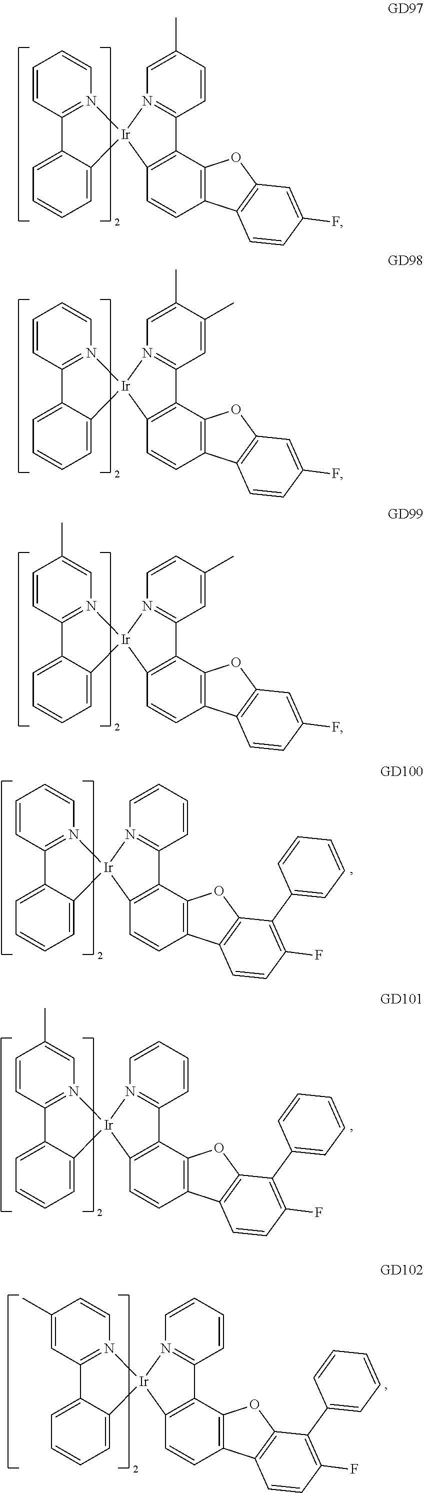

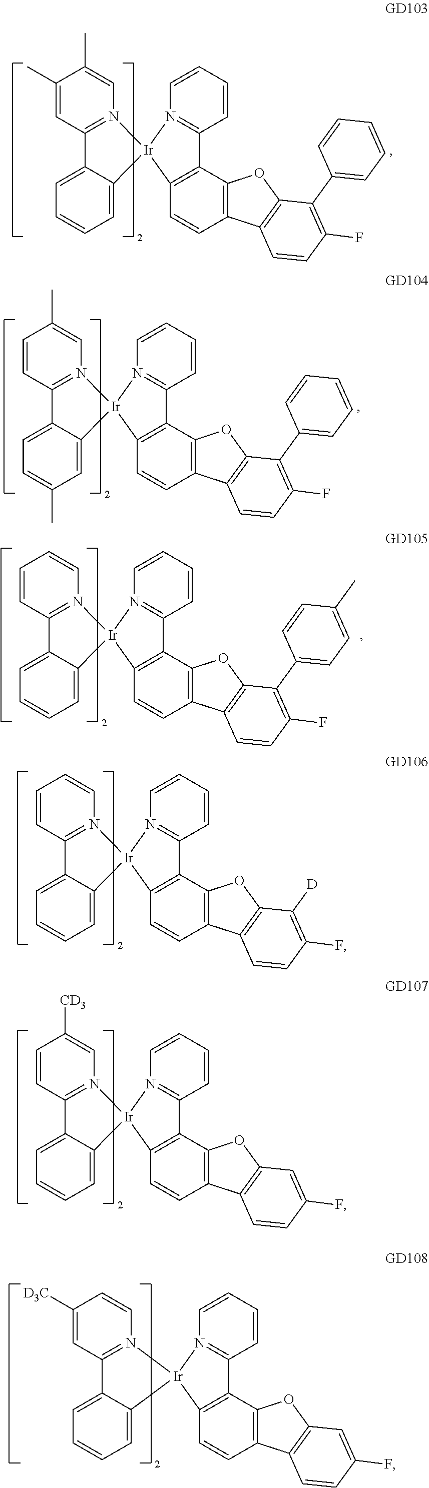

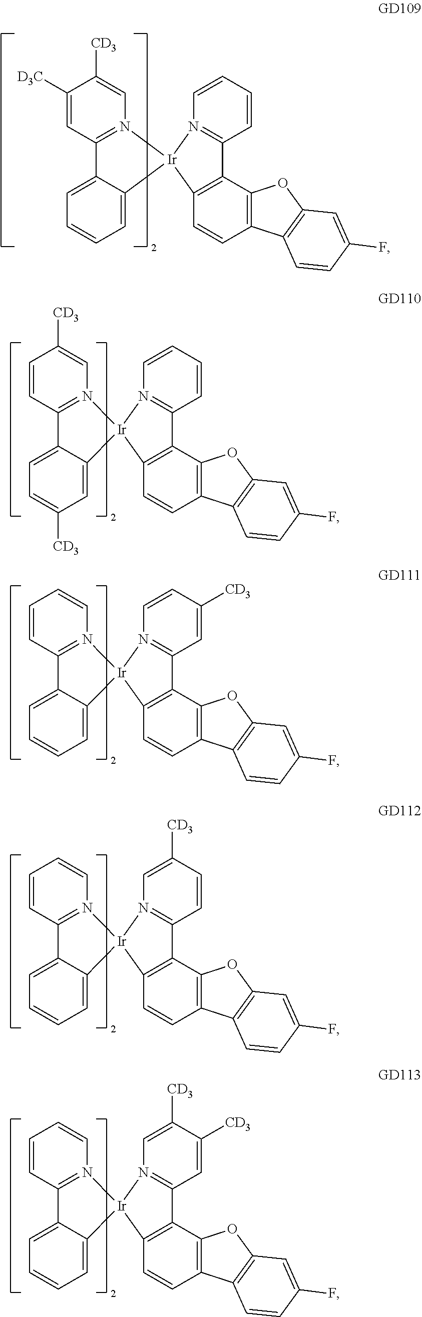

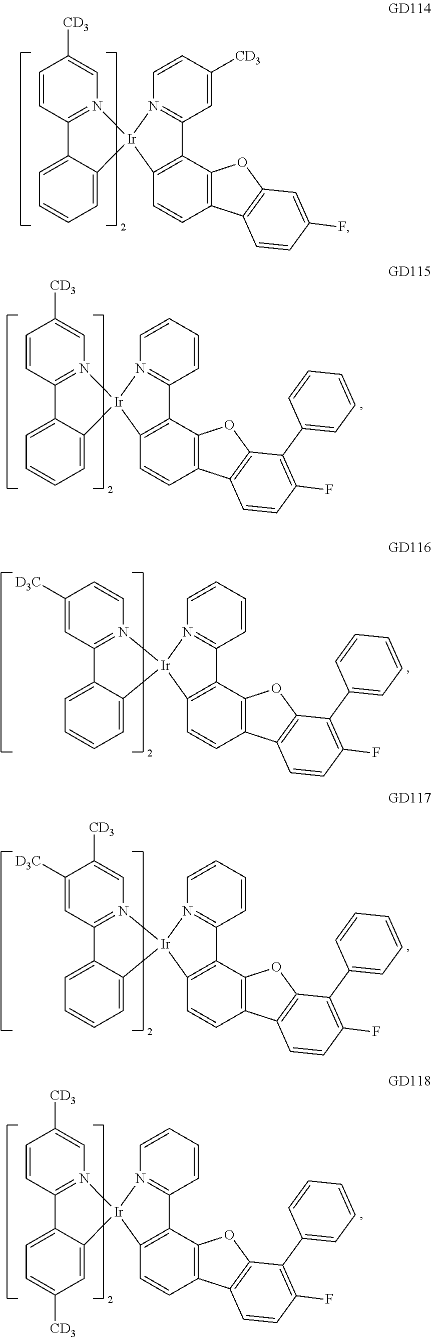

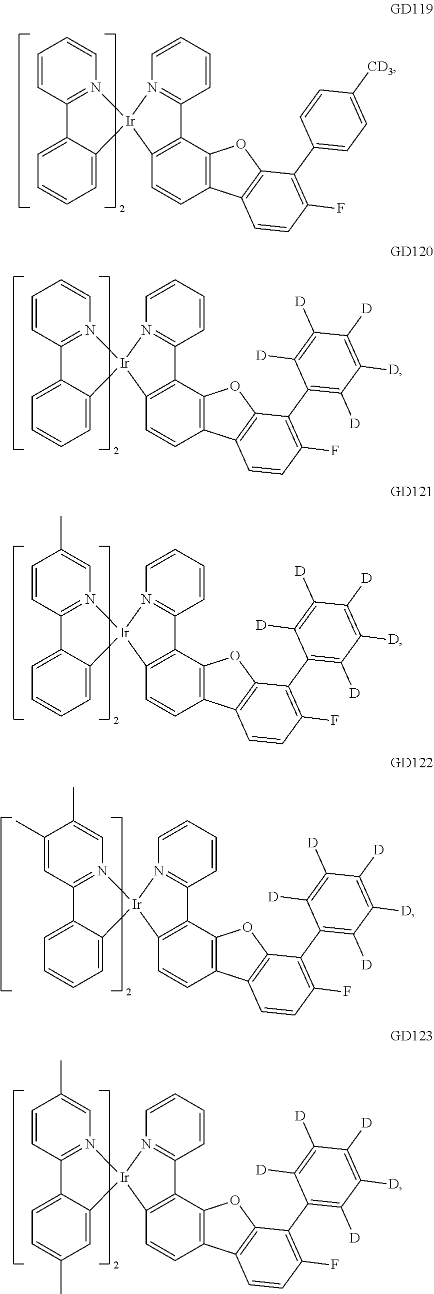

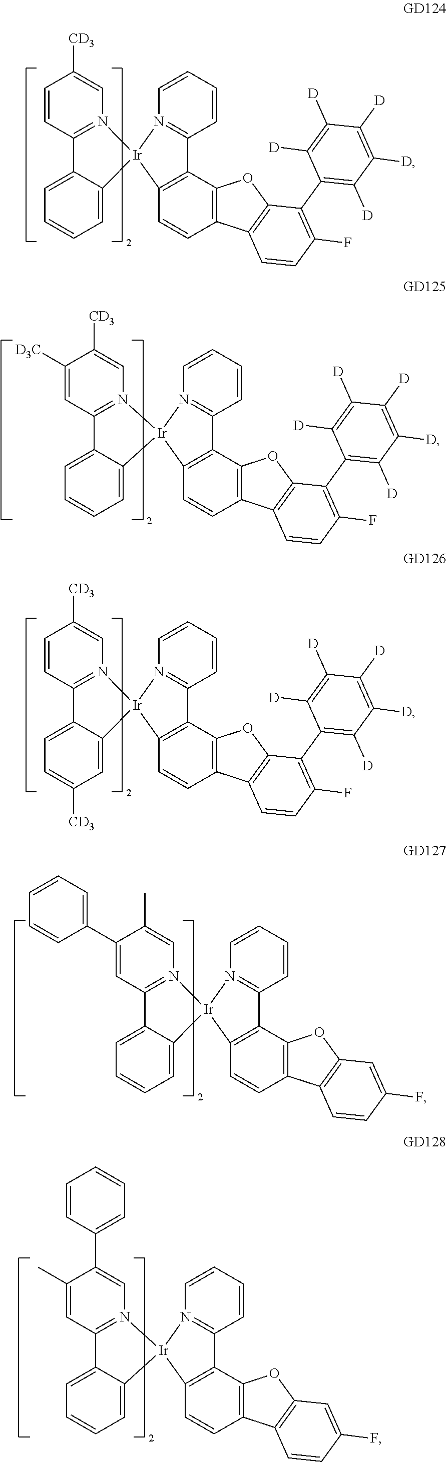

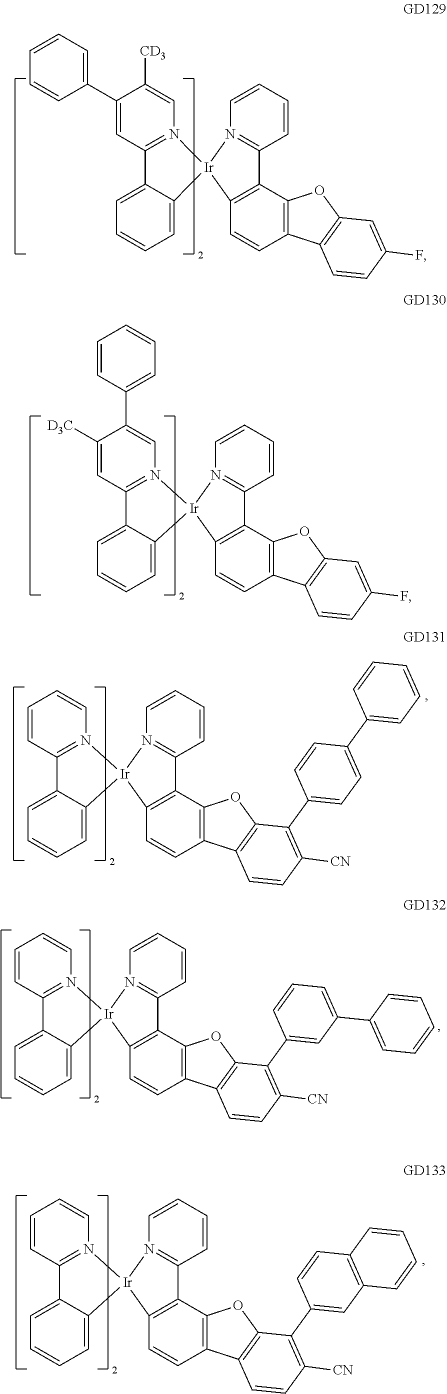

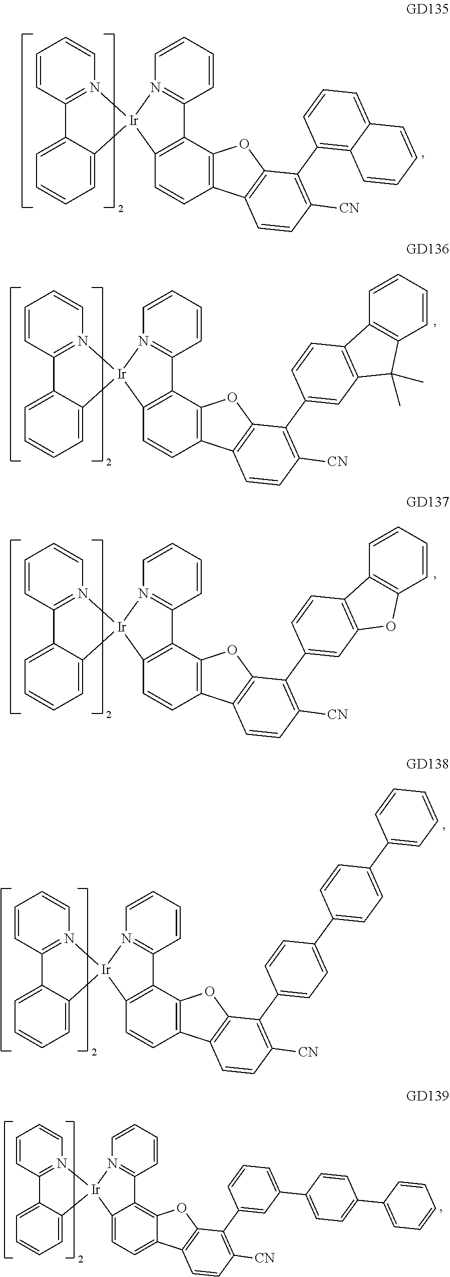

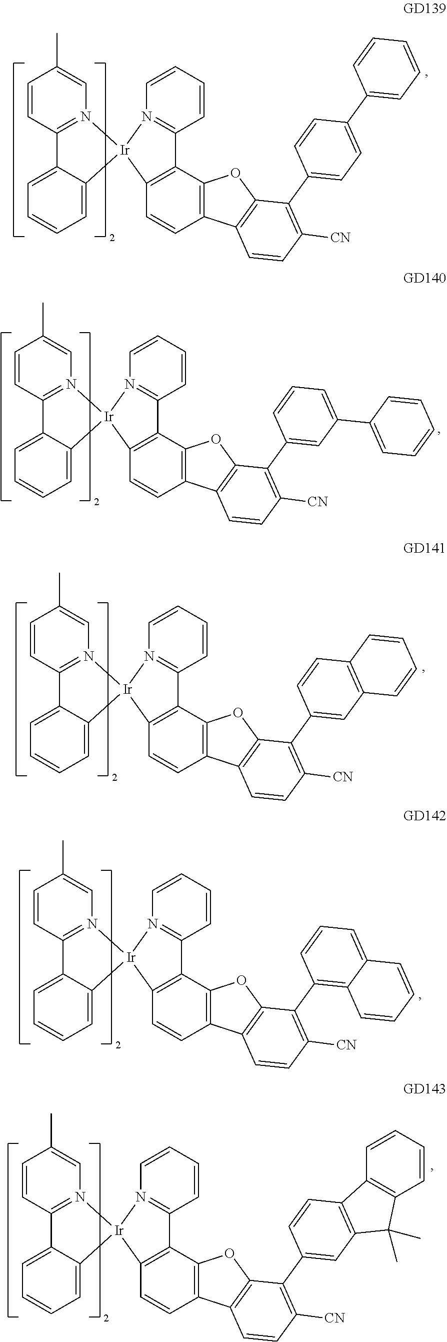

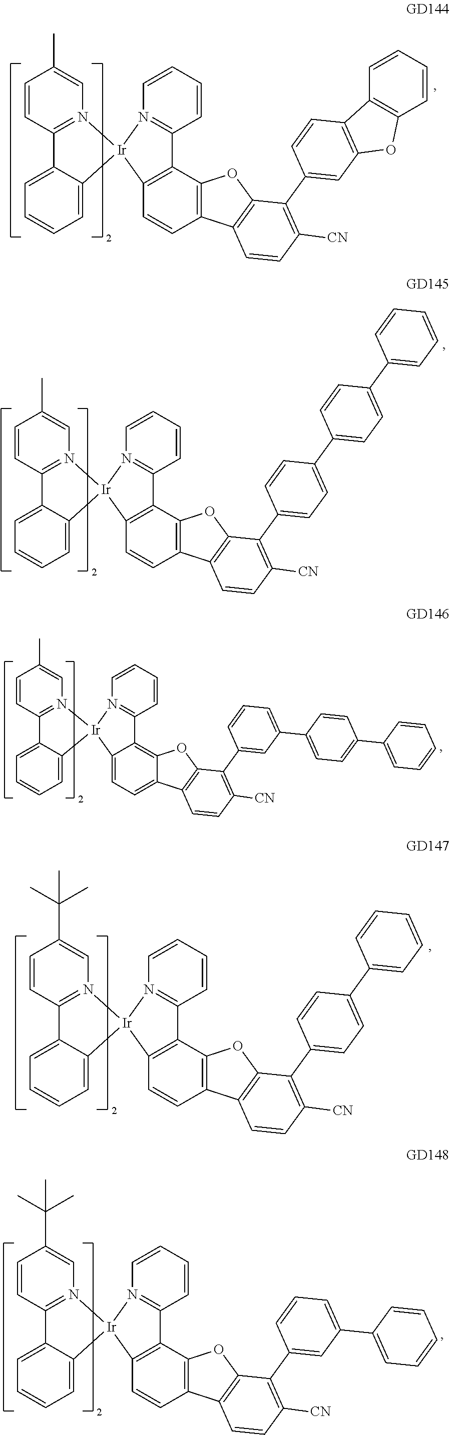

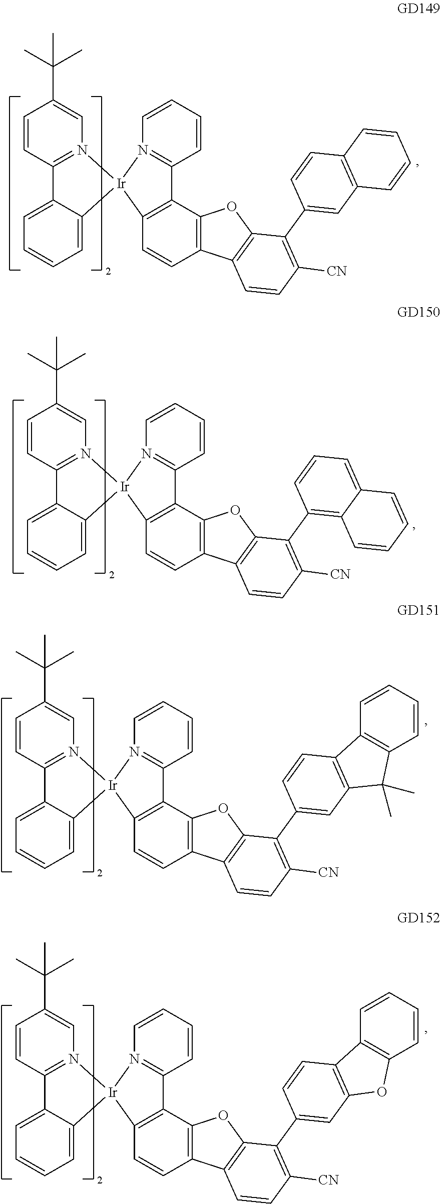

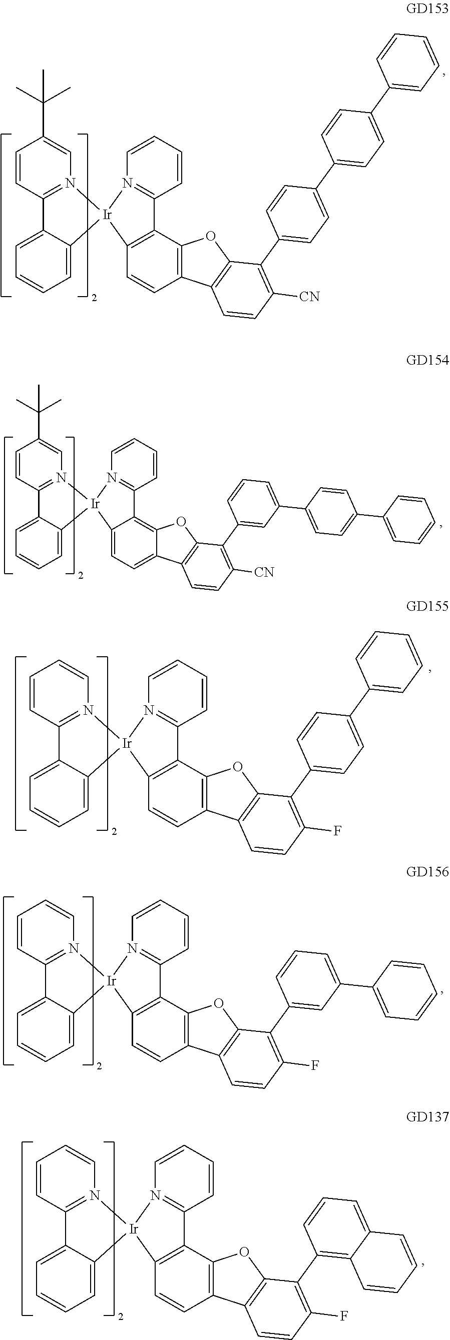

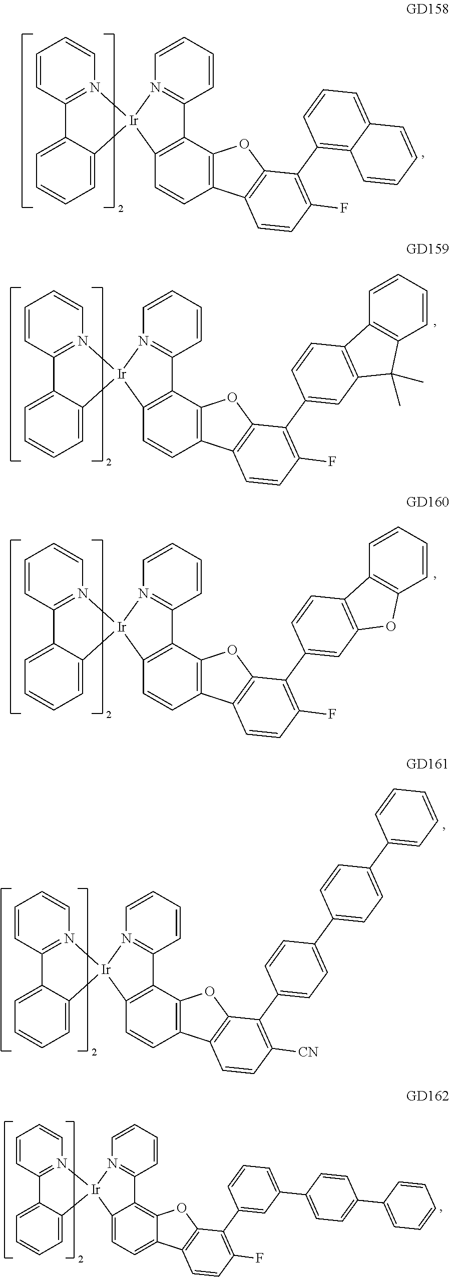

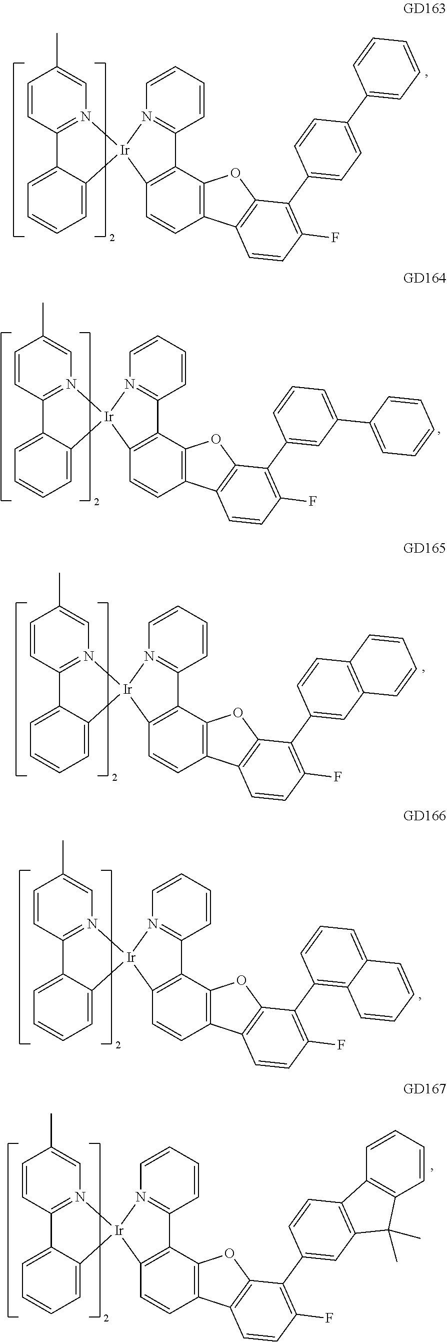

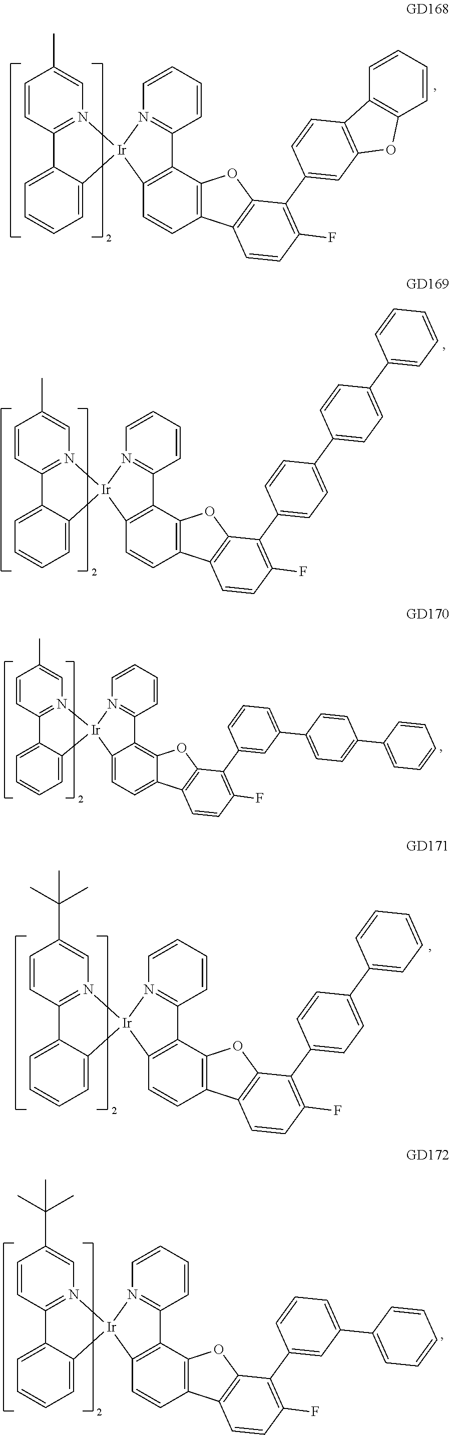

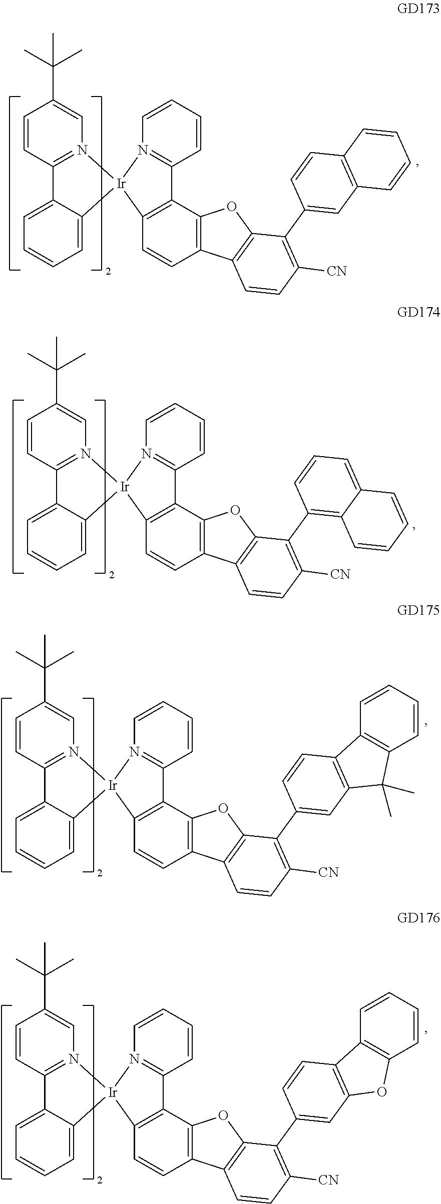

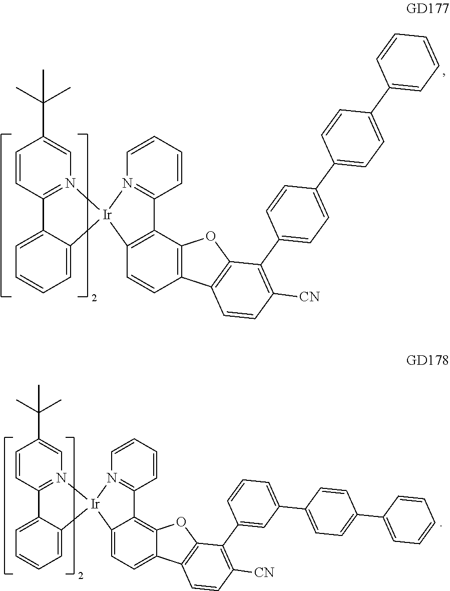

14. The electroluminescent device according to claim 1, wherein the first compound has a structure represented by Formula 2a: 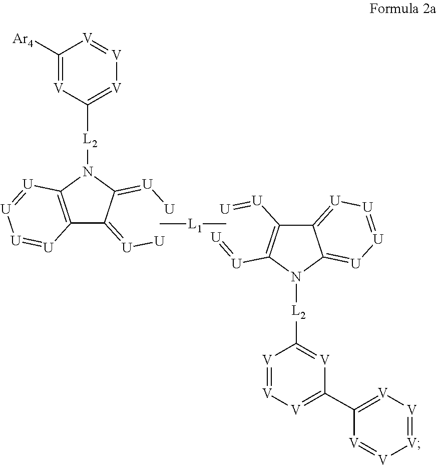
wherein in Formula 2a,
U is, at each occurrence identically or differently, selected from C, CRᵤ or N;
V is, at each occurrence identically or differently, selected from CRᵥ or N;
Ar₄ is, at each occurrence identically or differently, selected from substituted or unsubstituted aryl having 6 to 30 carbon atoms, substituted or unsubstituted heteroaryl having 3 to 30 carbon atoms or a combination thereof;
L₁ and L₂ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;
Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and
adjacent substituents Ar₄, Rᵤ and Rᵥ can be optionally joined to form a ring.

15. The electroluminescent device according to claim 1, wherein the first compound has a structure represented by Formula 2b: 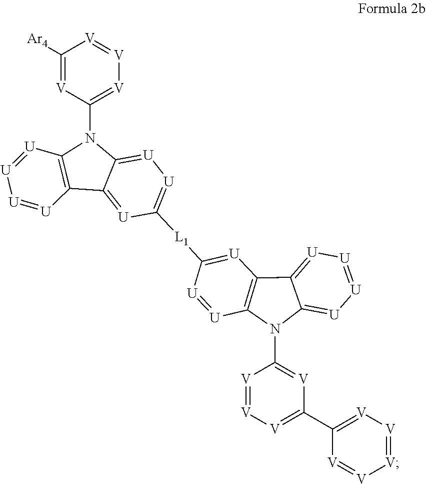
wherein in Formula 2b,
U is, at each occurrence identically or differently, selected from CRᵤ or N;
V is, at each occurrence identically or differently, selected from CRᵥ or N;
Ar₄ is, at each occurrence identically or differently, selected from substituted or unsubstituted aryl having 6 to 30 carbon atoms, substituted or unsubstituted heteroaryl having 3 to 30 carbon atoms or a combination thereof;
L₁ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;
Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and
adjacent substituents Ar₄, Rᵤ and Rᵥ can be optionally joined to form a ring.

16. The electroluminescent device according to claim 1, wherein U is, at each occurrence identically or differently, selected from C or CRᵤ, and/or V is, at each occurrence identically or differently, selected from CRᵥ.

17. The electroluminescent device according to claim 1, wherein Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, cyano, isocyano, hydroxyl, a sulfanyl group and combinations thereof;
preferably, Rᵤ and Rᵥ are, at each occurrence identically or differently, selected from the group consisting of: hydrogen, deuterium, fluorine, substituted or unsubstituted aryl having 6 to 20 carbon atoms, substituted or unsubstituted heteroaryl having 3 to 20 carbon atoms and combinations thereof; and
more preferably, Rᵤ and Rᵥ are, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbozolyl, substituted or unsubstituted dibenzofuranyl, substituted or unsubstituted dibenzothienyl and combinations thereof.

18. The electroluminescent device according to claim 1, wherein Z₁ to Z₈ are, at each occurrence identically or differently, selected from C or CR_{z}.

19. The electroluminescent device according to claim 1, wherein at least two of Z₁ to Z₈ are CRₓ, wherein at least one R_{z} is selected from substituted or unsubstituted aryl having 6 to 30 carbon atoms; and at least another R_{z} is selected from hydrogen, deuterium, halogen, cyano, substituted or unsubstituted aryl having 6 to 30 carbon atoms or a combination thereof.

20. The electroluminescent device according to claim 1, wherein at least one, two or three of Z₁ to Z₈ is(are) selected from CR_{z}, wherein the R_{z} is selected from substituted or unsubstituted aryl having 6 to 30 carbon atoms; and
preferably, at least one, two or three of Z₁ to Z₈ is(are) selected from CR_{z}, wherein the R_{z} is selected from any one of the following groups that are unsubstituted or substituted with one or more of deuterium, halogen and cyano: phenyl, naphthyl, biphenyl and terphenyl.

21. The electroluminescent device according to claim 1, wherein at least one of Z₁ to Z₄ is selected from C and joined to L₃; Z₅ and/or Z₈ are(is) selected from CR_{z}, wherein the R_{z} is substituted or unsubstituted aryl having 6 to 30 carbon atoms.

22. The electroluminescent device according to claim 1, wherein at least one of Z₁ to Z₄ is selected from C and joined to L₃; and at least one of Z₁ to Z₄ is selected from CR_{z}, wherein the R_{z} is substituted or unsubstituted aryl having 6 to 30 carbon atoms.

23. The electroluminescent device according to claim 1, wherein the second compound has a structure represented by one of Formula 3-1 to Formula 3-4: 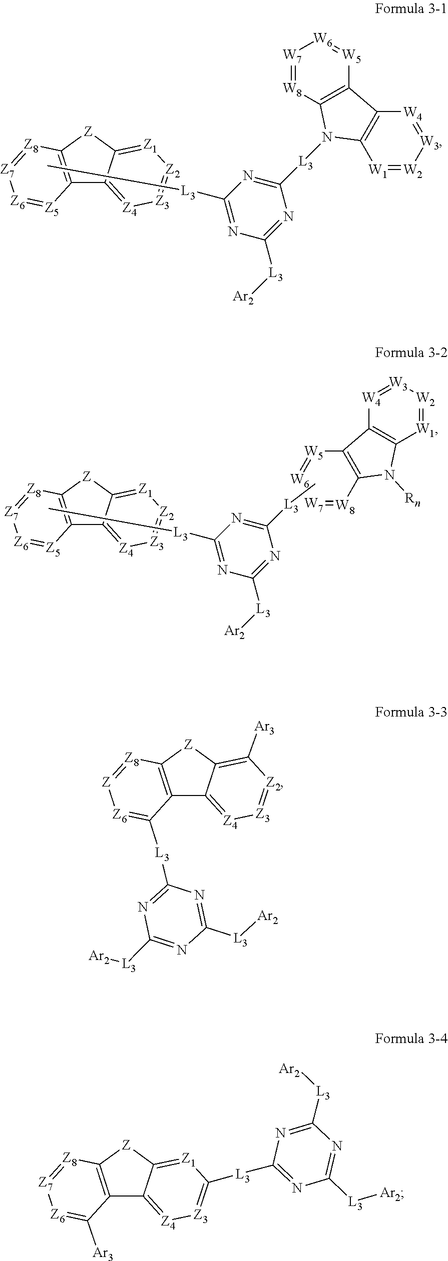
wherein
Z is, at each occurrence identically or differently, selected from the group consisting of O, S and Se;
L₃ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 20 carbon atoms or a combination thereof;
Z₁ to Z₈ are, at each occurrence identically or differently, selected from C, CR_{z} or N;
in Formula 3-1 and Formula 3-2, at least one of Z₁ to Z₈ is C and joined to L₃, and at least one of Z₁ to Z₈ is selected from CR_{z}, wherein the R_{z} is substituted or unsubstituted aryl having 6 to 30 carbon atoms;
W₁ to W₈ are, at each occurrence identically or differently, selected from C, CR_{L} or N;
Are and Ara are, at each occurrence identically or differently, selected from substituted or unsubstituted aryl having 6 to 30 carbon atoms, substituted or unsubstituted heteroaryl having 3 to 30 carbon atoms or a combination thereof;
R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;
R_{w} and Rₙ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and
adjacent substituents R_{z}, R_{w}, Rₙ can be optionally joined to form a ring.

24. The electroluminescent device according to claim 1, wherein L₁ to L₃ are, at each occurrence identically or differently, selected from a single bond, substituted or unsubstituted arylene having 6 to 20 carbon atoms, substituted or unsubstituted heteroarylene having 3 to 20 carbon atoms or a combination thereof;
preferably, L₁ to L₃ are, at each occurrence identically or differently, selected from a single bond, substituted or unsubstituted phenylene, substituted or unsubstituted biphenylene or a combination thereof; and
more preferably, L₁ to L₃ are, at each occurrence identically or differently, selected from a single bond.

25. The electroluminescent device according to claim 1, wherein Ar₂ to Ar₄ are, at each occurrence identically or differently, selected from substituted or unsubstituted aryl having 6 to 20 carbon atoms, substituted or unsubstituted heteroaryl having 3 to 20 carbon atoms or a combination thereof; and
preferably, Ar₂ to Ar₄ are, at each occurrence identically of differently, selected from any one of the following groups that are unsubstituted or substituted with one or more of deuterium, halogen and cyano: phenyl, naphthyl, biphenyl, terphenyl, phenanthryl, fluorenyl, carbazolyl, dibenzofuranyl, dibenzothienyl, pyridyl, pyrimidinyl, pyrazinyl, azafluorenyl, azacarbazolyl, azadibenzofuranyl, azadibenzothienyl, diazafluorenyl, diazacarbazolyl, diazadibenzofuranyl, diazadibenzothienyl and combinations thereof.

26. The electroluminescent device according to claim 1, wherein the first compound is selected from the group consisting of:

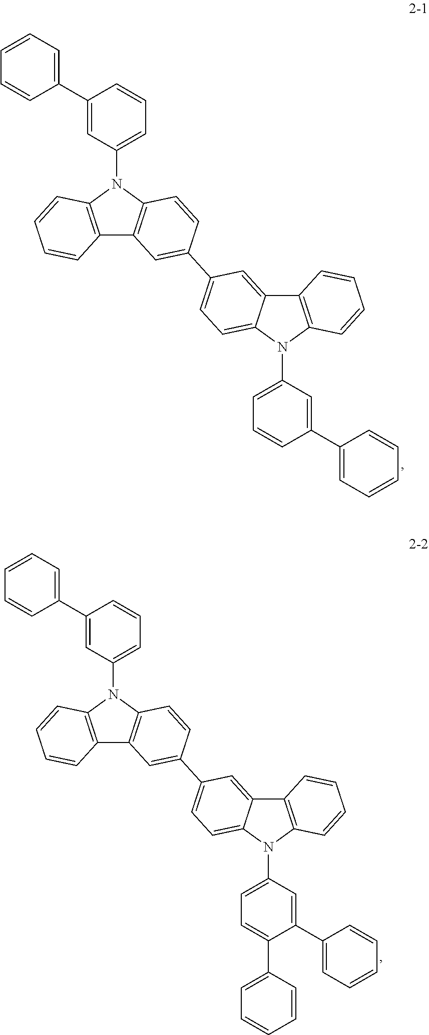 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccc(-c6ccccc6)c(-c6ccccc6)c5)ccc43)c2)cc1]

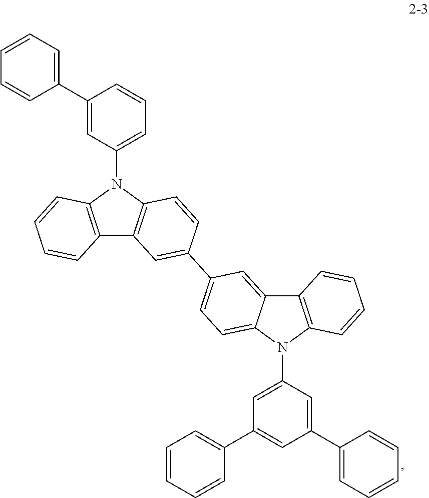 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

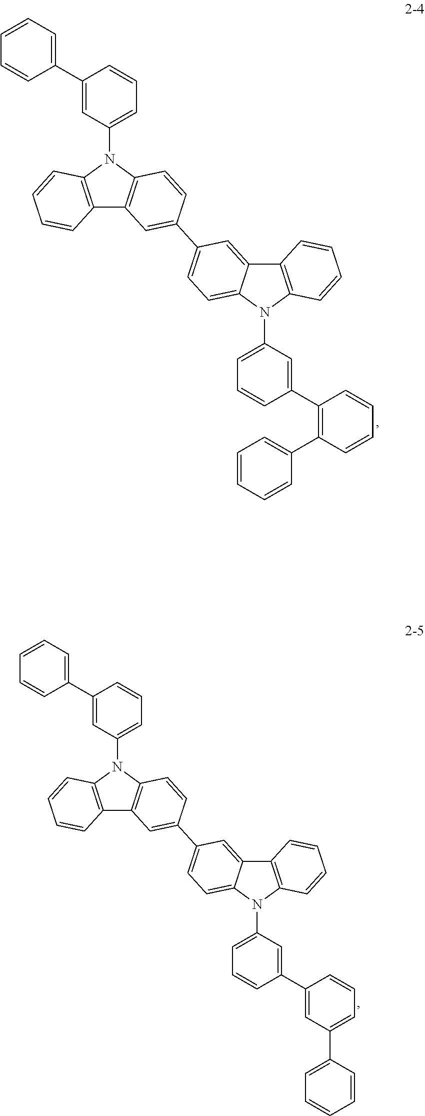 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

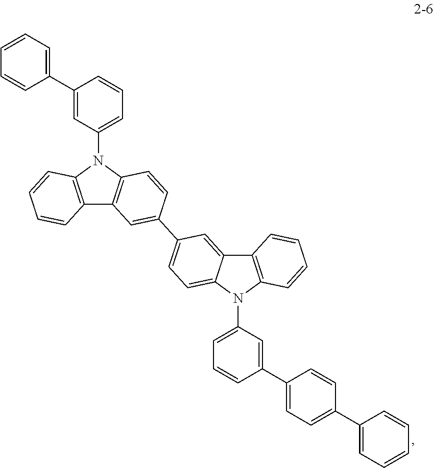 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

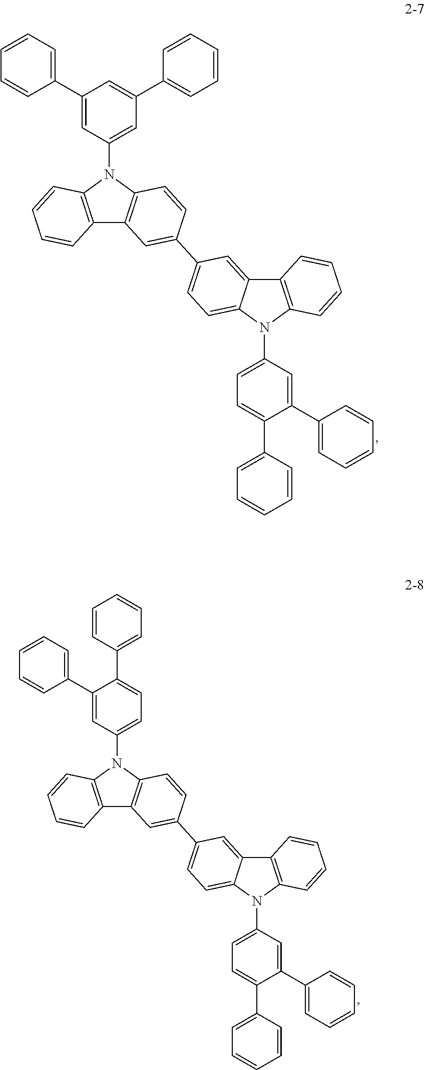 [SMILES: c1ccc(-c2cc(-c3ccccc3)cc(-n3c4ccccc4c4cc(-c5ccc6c(c5)c5ccccc5n6-c5ccc(-c6ccccc6)c(-c6ccccc6)c5)ccc43)c2)cc1 ; c1ccc(-c2ccc(-n3c4ccccc4c4cc(-c5ccc6c(c5)c5ccccc5n6-c5ccc(-c6ccccc6)c(-c6ccccc6)c5)ccc43)cc2-c2ccccc2)cc1]

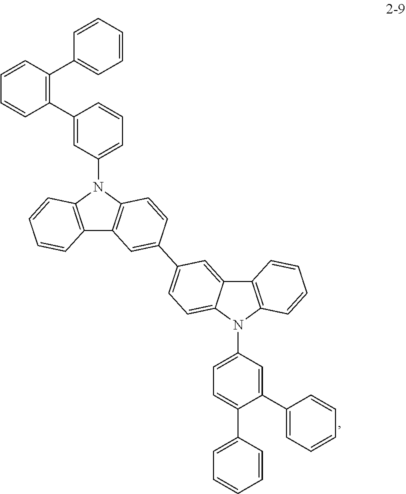 [SMILES: c1ccc(-c2ccccc2-c2cccc(-n3c4ccccc4c4cc(-c5ccc6c(c5)c5ccccc5n6-c5ccc(-c6ccccc6)c(-c6ccccc6)c5)ccc43)c2)cc1]

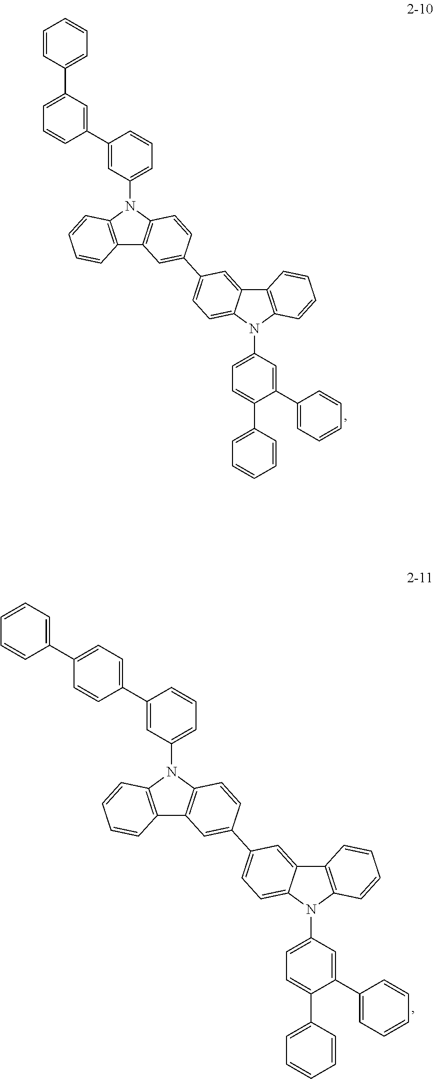 [SMILES: c1ccc(-c2cccc(-c3cccc(-n4c5ccccc5c5cc(-c6ccc7c(c6)c6ccccc6n7-c6ccc(-c7ccccc7)c(-c7ccccc7)c6)ccc54)c3)c2)cc1 ; c1ccc(-c2ccc(-c3cccc(-n4c5ccccc5c5cc(-c6ccc7c(c6)c6ccccc6n7-c6ccc(-c7ccccc7)c(-c7ccccc7)c6)ccc54)c3)cc2)cc1]

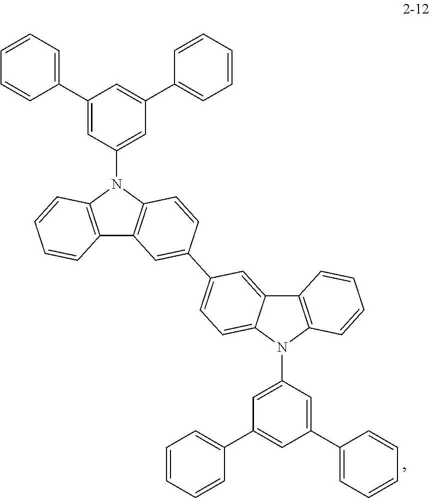 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1]

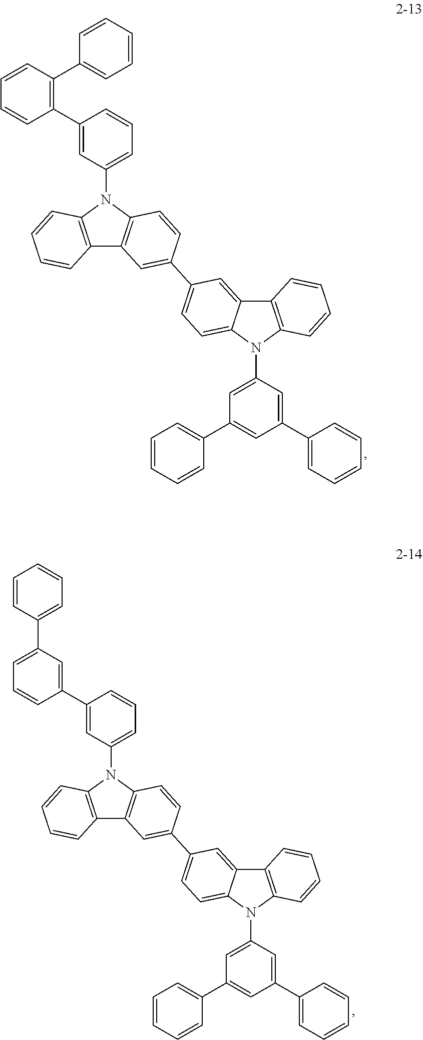 [SMILES: c1ccc(-c2cc(-c3ccccc3)cc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3cccc(-n4c5ccccc5c5cc(-c6ccc7c(c6)c6ccccc6n7-c6cc(-c7ccccc7)cc(-c7ccccc7)c6)ccc54)c3)c2)cc1]

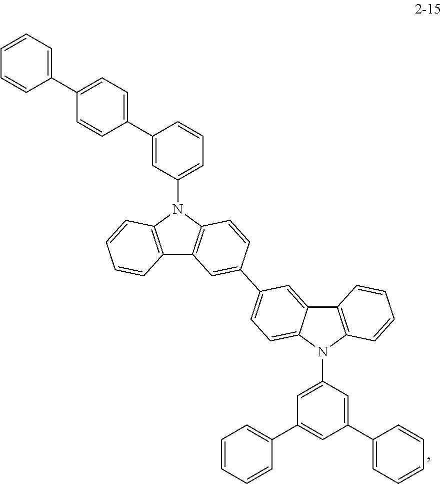 [SMILES: c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1]

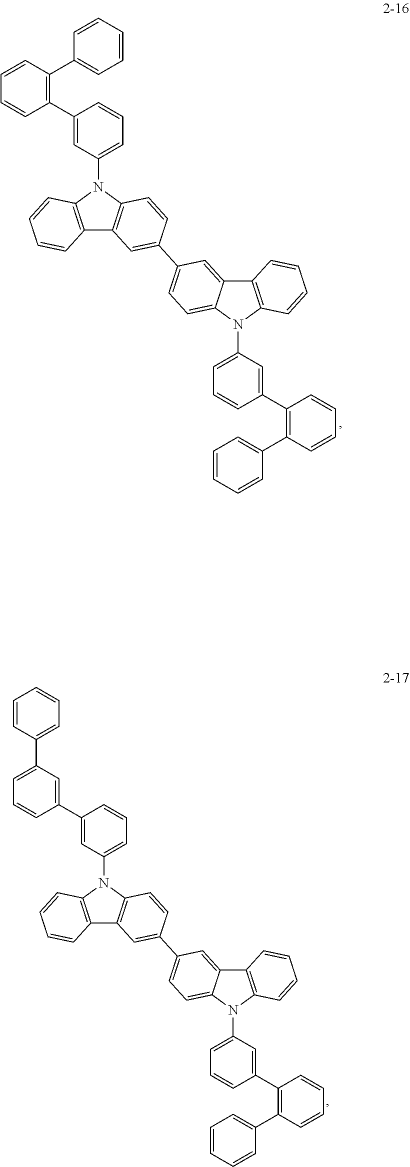 [SMILES: c1ccc(-c2ccccc2-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3cccc(-n4c5ccccc5c5cc(-c6ccc7c(c6)c6ccccc6n7-c6cccc(-c7ccccc7-c7ccccc7)c6)ccc54)c3)c2)cc1]

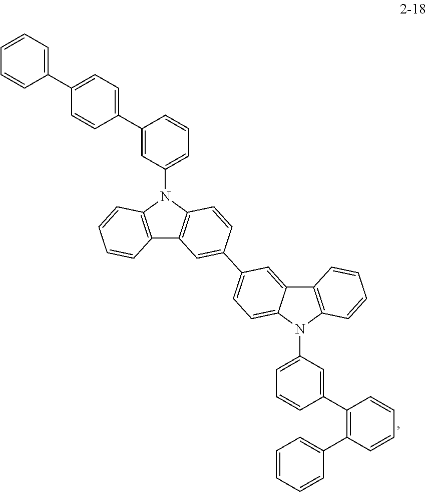 [SMILES: c1ccc(-c2ccc(-c3cccc(-n4c5ccccc5c5cc(-c6ccc7c(c6)c6ccccc6n7-c6cccc(-c7ccccc7-c7ccccc7)c6)ccc54)c3)cc2)cc1]

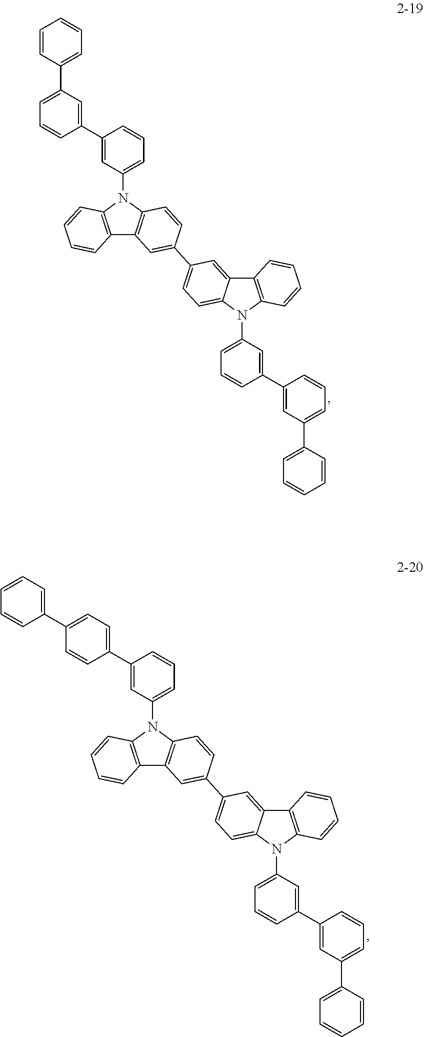 [SMILES: c1ccc(-c2cccc(-c3cccc(-n4c5ccccc5c5cc(-c6ccc7c(c6)c6ccccc6n7-c6cccc(-c7cccc(-c8ccccc8)c7)c6)ccc54)c3)c2)cc1 ; c1ccc(-c2ccc(-c3cccc(-n4c5ccccc5c5cc(-c6ccc7c(c6)c6ccccc6n7-c6cccc(-c7cccc(-c8ccccc8)c7)c6)ccc54)c3)cc2)cc1]

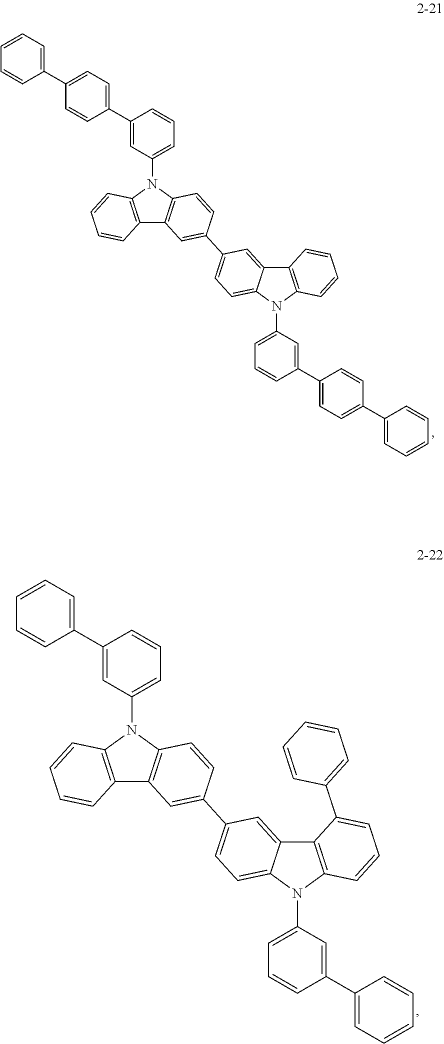 [SMILES: c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2cccc(-n3c4ccccc4c4cc(-c5ccc6c(c5)c5c(-c7ccccc7)cccc5n6-c5cccc(-c6ccccc6)c5)ccc43)c2)cc1]

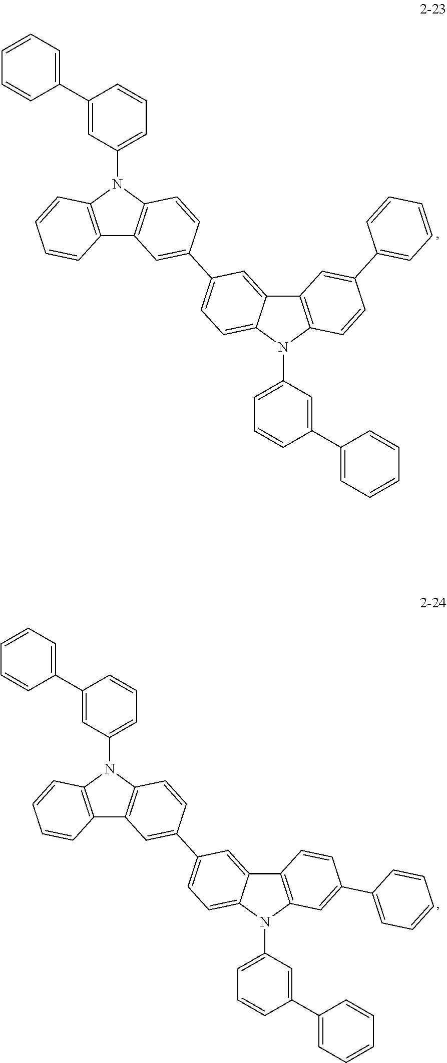 [SMILES: c1ccc(-c2cccc(-n3c4ccccc4c4cc(-c5ccc6c(c5)c5cc(-c7ccccc7)ccc5n6-c5cccc(-c6ccccc6)c5)ccc43)c2)cc1 ; c1ccc(-c2cccc(-n3c4ccccc4c4cc(-c5ccc6c(c5)c5ccc(-c7ccccc7)cc5n6-c5cccc(-c6ccccc6)c5)ccc43)c2)cc1]

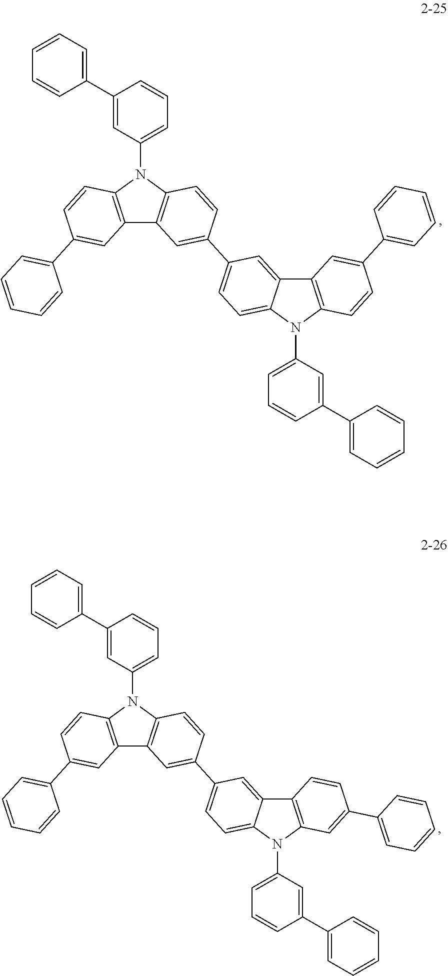 [SMILES: c1ccc(-c2cccc(-n3c4ccc(-c5ccccc5)cc4c4cc(-c5ccc6c(c5)c5cc(-c7ccccc7)ccc5n6-c5cccc(-c6ccccc6)c5)ccc43)c2)cc1 ; c1ccc(-c2cccc(-n3c4ccc(-c5ccccc5)cc4c4cc(-c5ccc6c(c5)c5ccc(-c7ccccc7)cc5n6-c5cccc(-c6ccccc6)c5)ccc43)c2)cc1]

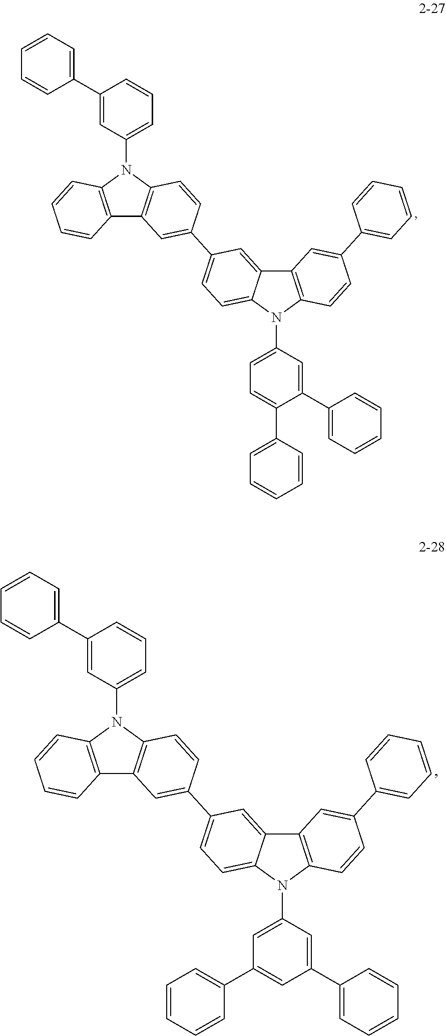 [SMILES: c1ccc(-c2cccc(-n3c4ccccc4c4cc(-c5ccc6c(c5)c5cc(-c7ccccc7)ccc5n6-c5ccc(-c6ccccc6)c(-c6ccccc6)c5)ccc43)c2)cc1 ; c1ccc(-c2cccc(-n3c4ccccc4c4cc(-c5ccc6c(c5)c5cc(-c7ccccc7)ccc5n6-c5cc(-c6ccccc6)cc(-c6ccccc6)c5)ccc43)c2)cc1]

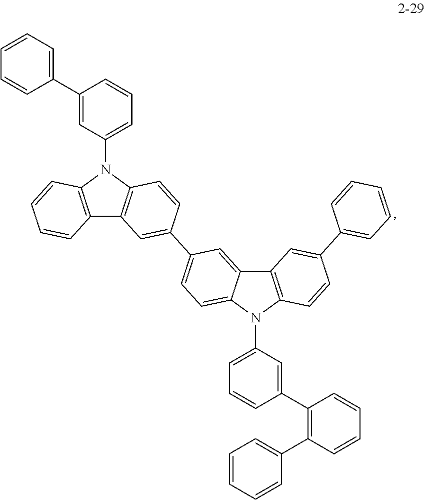 [SMILES: c1ccc(-c2cccc(-n3c4ccccc4c4cc(-c5ccc6c(c5)c5cc(-c7ccccc7)ccc5n6-c5cccc(-c6ccccc6-c6ccccc6)c5)ccc43)c2)cc1]

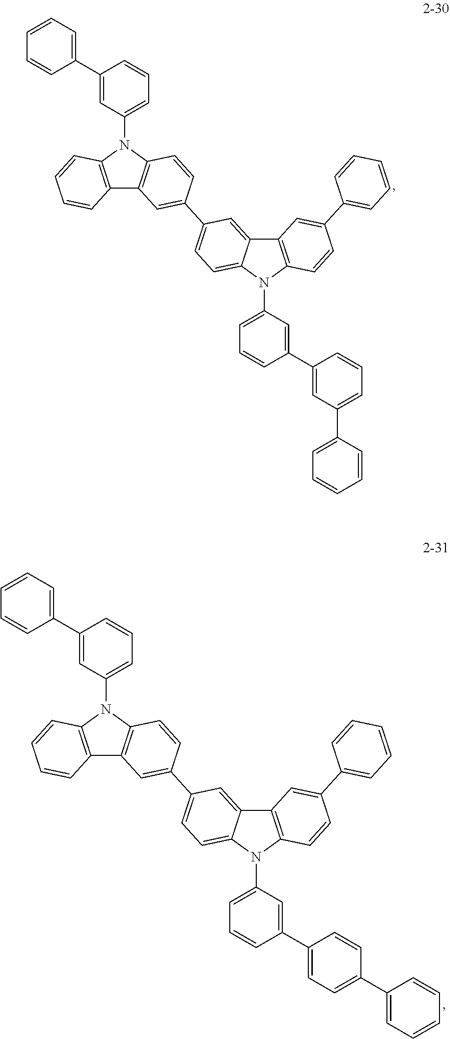 [SMILES: c1ccc(-c2cccc(-c3cccc(-n4c5ccc(-c6ccccc6)cc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccc(-c6ccccc6)cc5c5cc(-c6ccc7c(c6)c6ccccc6n7-c6cccc(-c7ccccc7)c6)ccc54)c3)cc2)cc1]

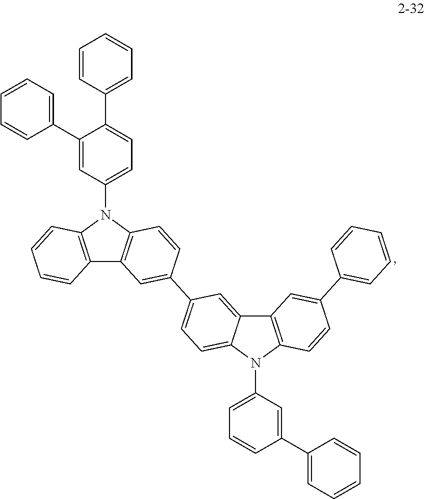 [SMILES: c1ccc(-c2cccc(-n3c4ccc(-c5ccccc5)cc4c4cc(-c5ccc6c(c5)c5ccccc5n6-c5ccc(-c6ccccc6)c(-c6ccccc6)c5)ccc43)c2)cc1]

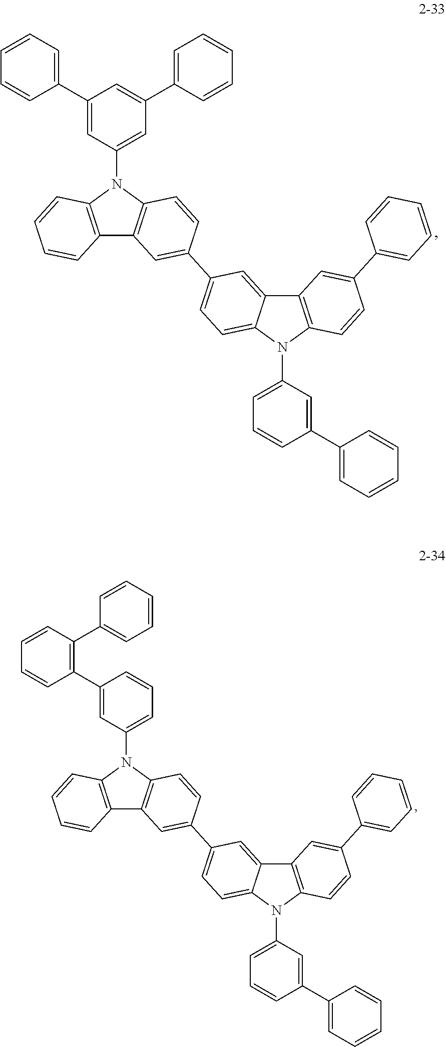 [SMILES: c1ccc(-c2cccc(-n3c4ccc(-c5ccccc5)cc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccc(-c5ccccc5)cc4c4cc(-c5ccc6c(c5)c5ccccc5n6-c5cccc(-c6ccccc6-c6ccccc6)c5)ccc43)c2)cc1]

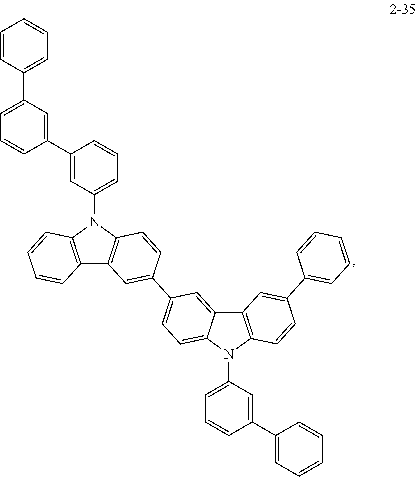 [SMILES: c1ccc(-c2cccc(-c3cccc(-n4c5ccccc5c5cc(-c6ccc7c(c6)c6cc(-c8ccccc8)ccc6n7-c6cccc(-c7ccccc7)c6)ccc54)c3)c2)cc1]

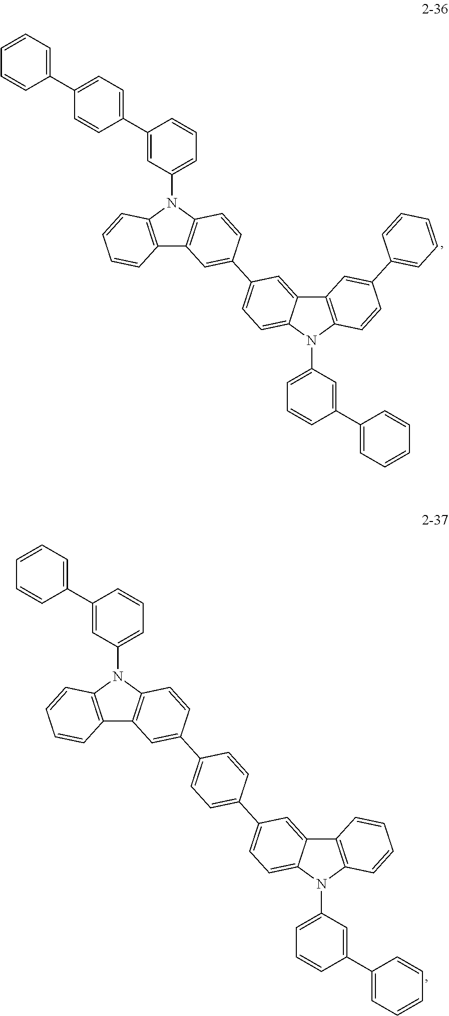 [SMILES: c1ccc(-c2ccc(-c3cccc(-n4c5ccccc5c5cc(-c6ccc7c(c6)c6cc(-c8ccccc8)ccc6n7-c6cccc(-c7ccccc7)c6)ccc54)c3)cc2)cc1 ; c1ccc(-c2cccc(-n3c4ccccc4c4cc(-c5ccc(-c6ccc7c(c6)c6ccccc6n7-c6cccc(-c7ccccc7)c6)cc5)ccc43)c2)cc1]

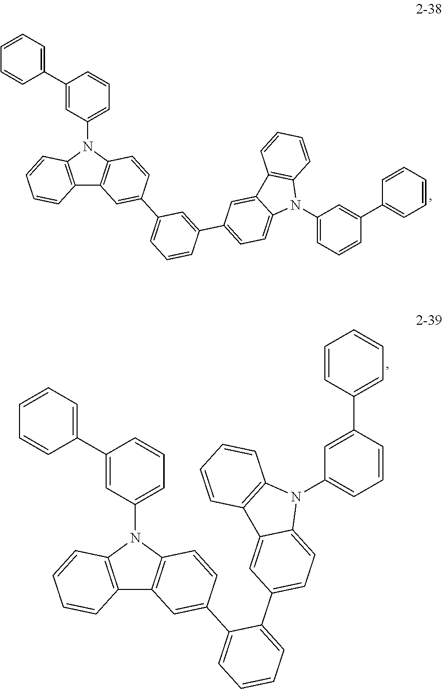 [SMILES: c1ccc(-c2cccc(-n3c4ccccc4c4cc(-c5cccc(-c6ccc7c(c6)c6ccccc6n7-c6cccc(-c7ccccc7)c6)c5)ccc43)c2)cc1 ; c1ccc(-c2cccc(-n3c4ccccc4c4cc(-c5ccccc5-c5ccc6c(c5)c5ccccc5n6-c5cccc(-c6ccccc6)c5)ccc43)c2)cc1]

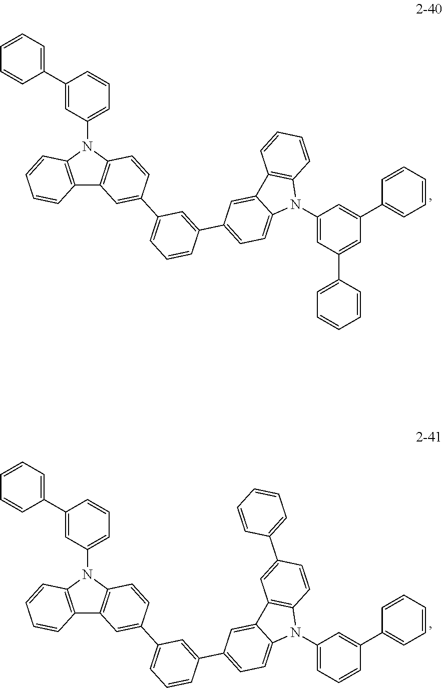 [SMILES: c1ccc(-c2cccc(-n3c4ccccc4c4cc(-c5cccc(-c6ccc7c(c6)c6ccccc6n7-c6cc(-c7ccccc7)cc(-c7ccccc7)c6)c5)ccc43)c2)cc1 ; c1ccc(-c2cccc(-n3c4ccccc4c4cc(-c5cccc(-c6ccc7c(c6)c6cc(-c8ccccc8)ccc6n7-c6cccc(-c7ccccc7)c6)c5)ccc43)c2)cc1]

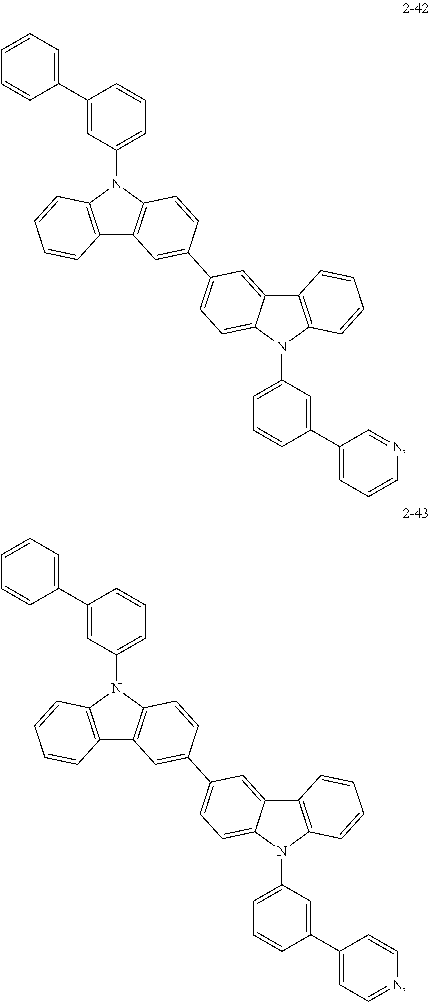 [SMILES: c1ccc(-c2cccc(-n3c4ccccc4c4cc(-c5ccc6c(c5)c5ccccc5n6-c5cccc(-c6cccnc6)c5)ccc43)c2)cc1 ; c1ccc(-c2cccc(-n3c4ccccc4c4cc(-c5ccc6c(c5)c5ccccc5n6-c5cccc(-c6ccncc6)c5)ccc43)c2)cc1]

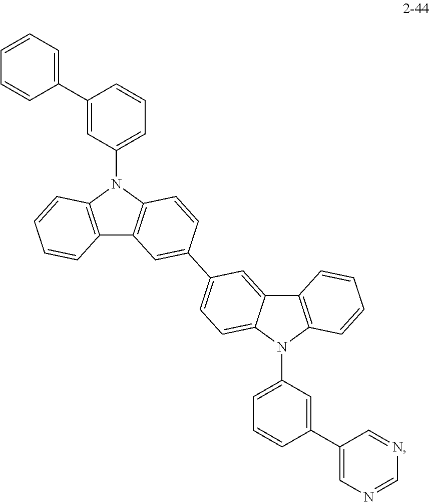 [SMILES: c1ccc(-c2cccc(-n3c4ccccc4c4cc(-c5ccc6c(c5)c5ccccc5n6-c5cccc(-c6cncnc6)c5)ccc43)c2)cc1]

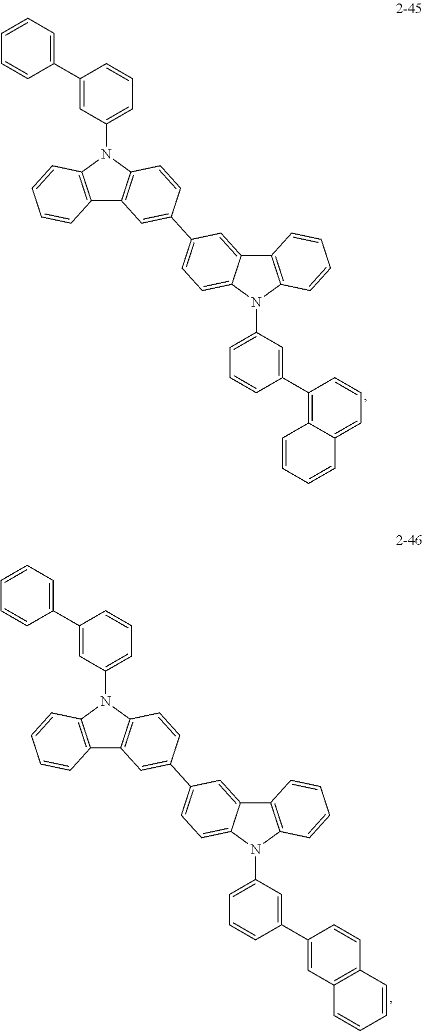 [SMILES: c1ccc(-c2cccc(-n3c4ccccc4c4cc(-c5ccc6c(c5)c5ccccc5n6-c5cccc(-c6cccc7ccccc67)c5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1]

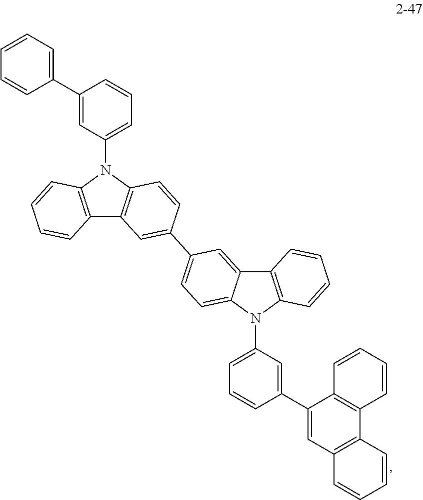 [SMILES: c1ccc(-c2cccc(-n3c4ccccc4c4cc(-c5ccc6c(c5)c5ccccc5n6-c5cccc(-c6cc7ccccc7c7ccccc67)c5)ccc43)c2)cc1]

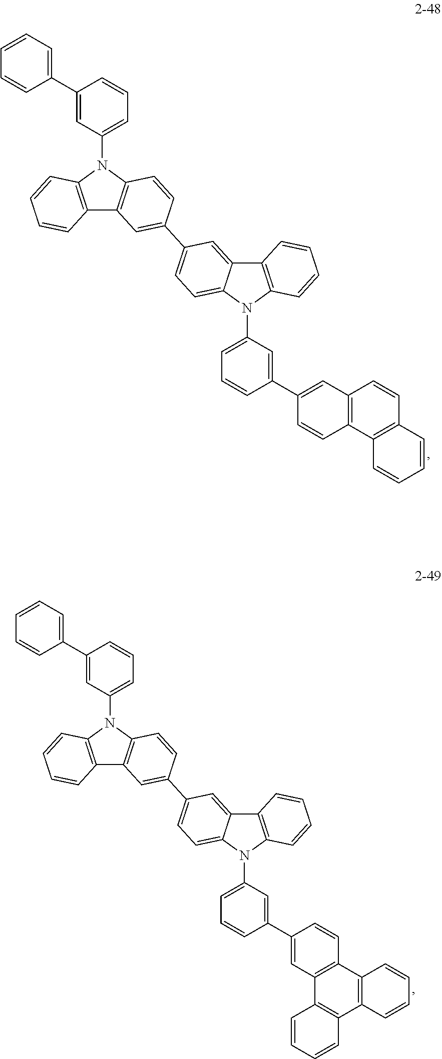 [SMILES: c1ccc(-c2cccc(-n3c4ccccc4c4cc(-c5ccc6c(c5)c5ccccc5n6-c5cccc(-c6ccc7c(ccc8ccccc87)c6)c5)ccc43)c2)cc1 ; c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1]

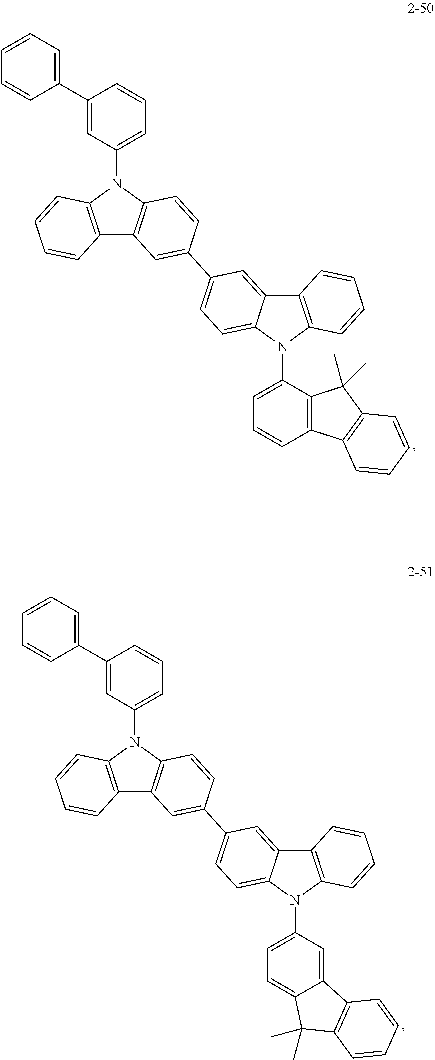 [SMILES: CC1(C)c2ccccc2-c2cccc(-n3c4ccccc4c4cc(-c5ccc6c(c5)c5ccccc5n6-c5cccc(-c6ccccc6)c5)ccc43)c21 ; CC1(C)c2ccccc2-c2cc(-n3c4ccccc4c4cc(-c5ccc6c(c5)c5ccccc5n6-c5cccc(-c6ccccc6)c5)ccc43)ccc21]

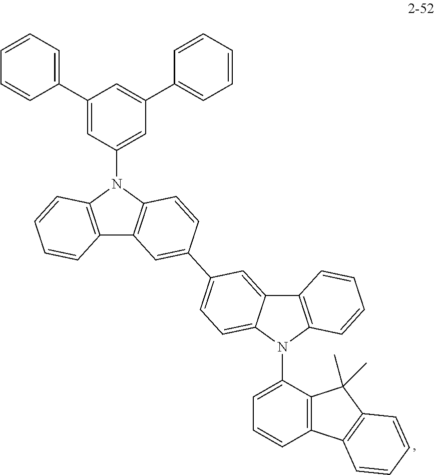 [SMILES: CC1(C)c2ccccc2-c2cccc(-n3c4ccccc4c4cc(-c5ccc6c(c5)c5ccccc5n6-c5cc(-c6ccccc6)cc(-c6ccccc6)c5)ccc43)c21]

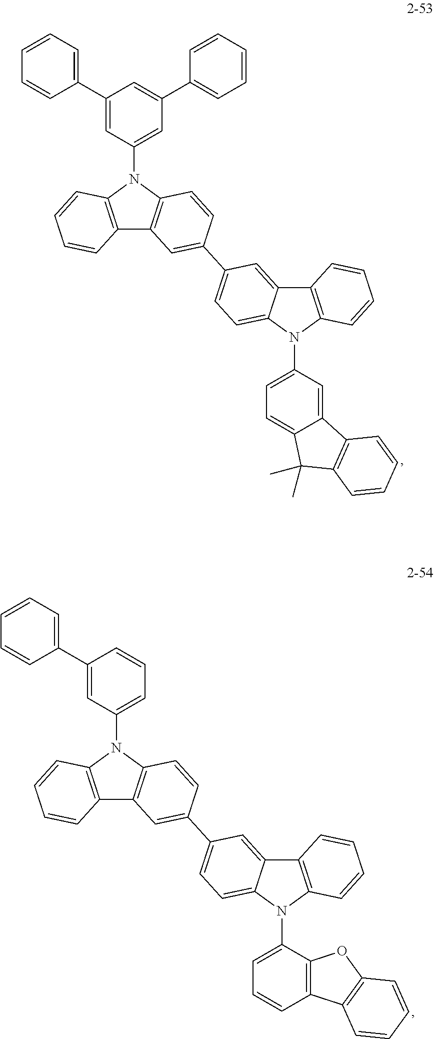 [SMILES: CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5cccc6c5oc5ccccc56)ccc43)c2)cc1]

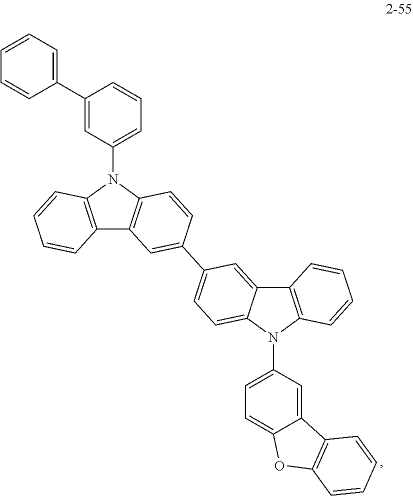 [SMILES: c1ccc(-c2cccc(-n3c4ccccc4c4cc(-c5ccc6c(c5)c5ccccc5n6-c5ccc6oc7ccccc7c6c5)ccc43)c2)cc1]

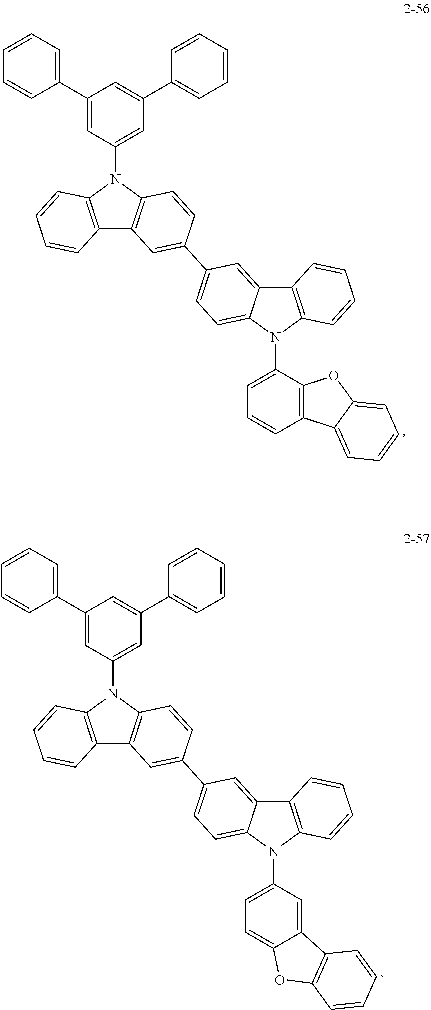 [SMILES: c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

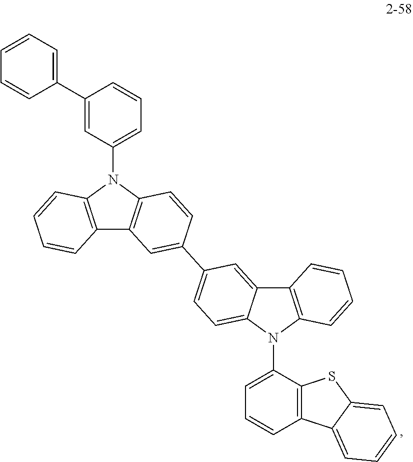 [SMILES: c1ccc(-c2cccc(-n3c4ccccc4c4cc(-c5ccc6c(c5)c5ccccc5n6-c5cccc6c5sc5ccccc56)ccc43)c2)cc1]

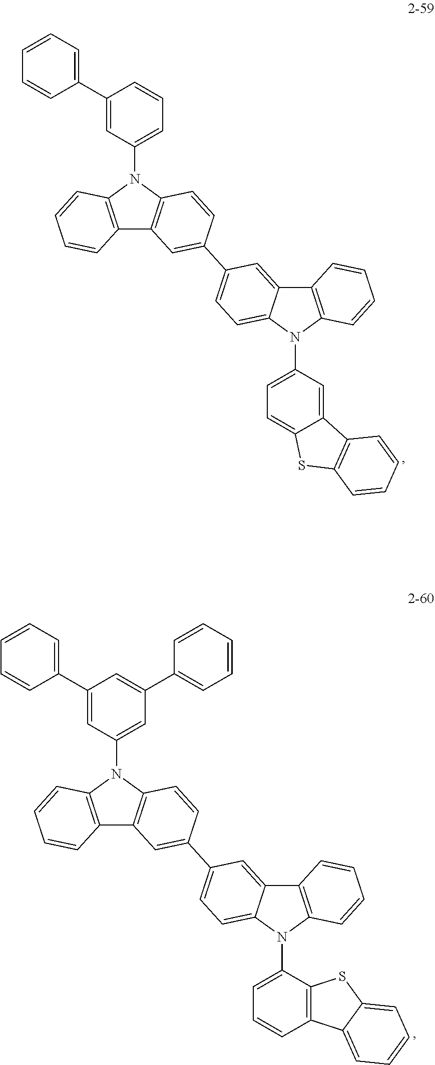 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

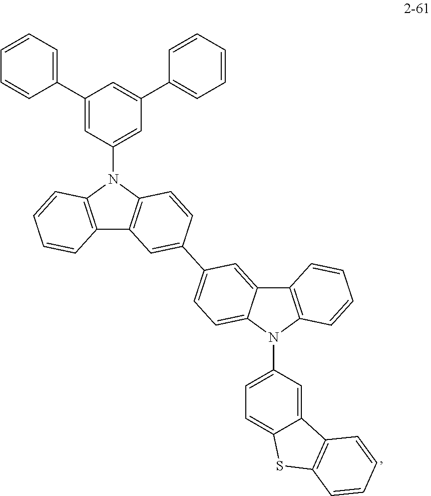 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

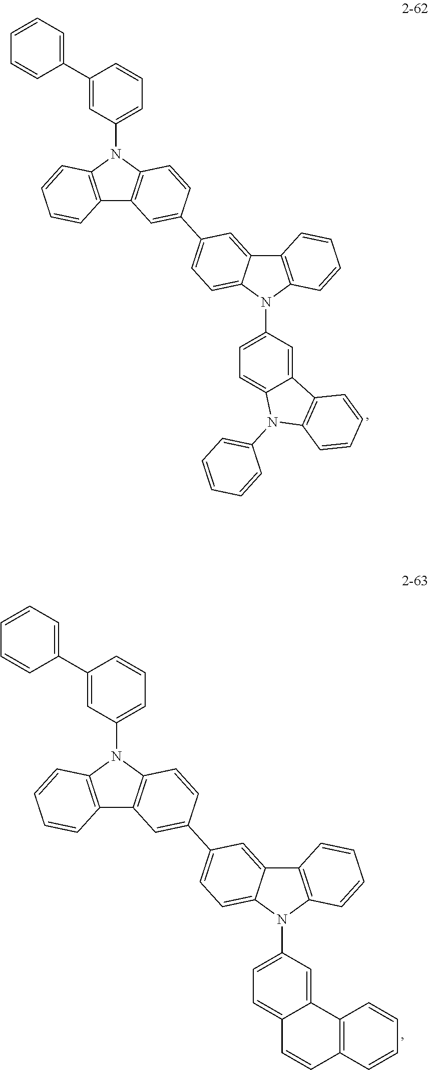 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6ccc7ccccc7c6c5)ccc43)c2)cc1]

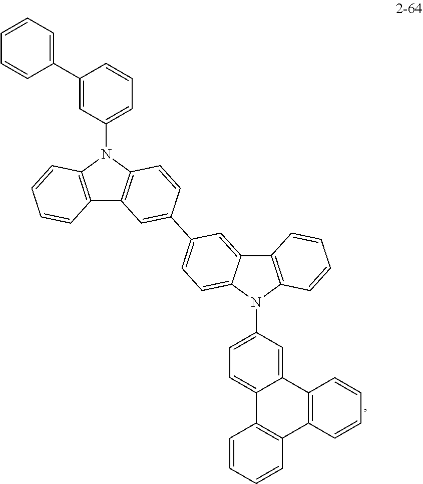 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1]

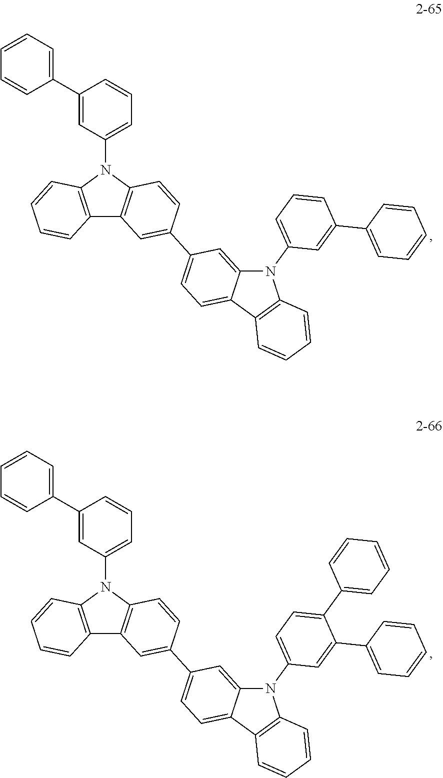 [SMILES: c1ccc(-c2cccc(-n3c4ccccc4c4cc(-c5ccc6c7ccccc7n(-c7cccc(-c8ccccc8)c7)c6c5)ccc43)c2)cc1 ; c1ccc(-c2cccc(-n3c4ccccc4c4cc(-c5ccc6c7ccccc7n(-c7ccc(-c8ccccc8)c(-c8ccccc8)c7)c6c5)ccc43)c2)cc1]

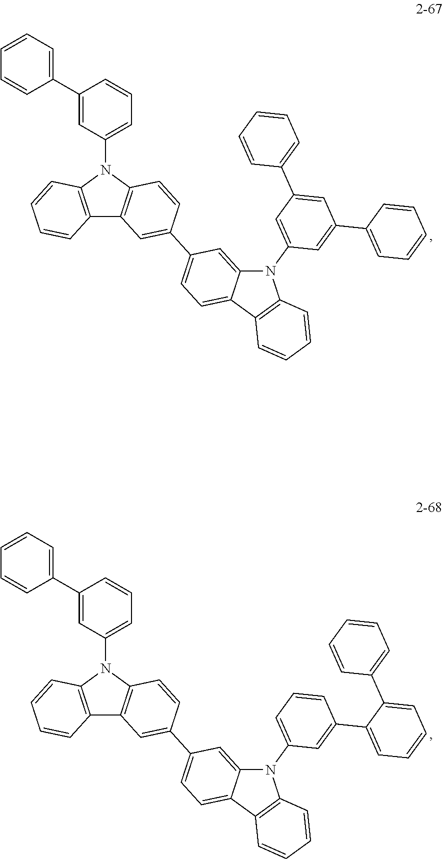 [SMILES: c1ccc(-c2cccc(-n3c4ccccc4c4cc(-c5ccc6c7ccccc7n(-c7cc(-c8ccccc8)cc(-c8ccccc8)c7)c6c5)ccc43)c2)cc1 ; c1ccc(-c2cccc(-n3c4ccccc4c4cc(-c5ccc6c7ccccc7n(-c7cccc(-c8ccccc8-c8ccccc8)c7)c6c5)ccc43)c2)cc1]

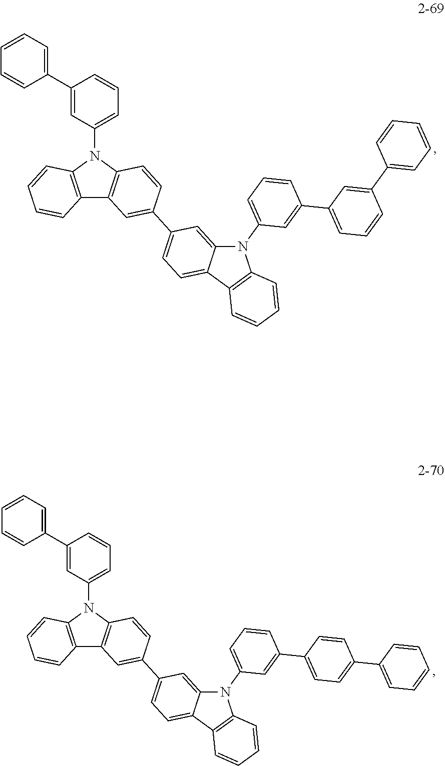 [SMILES: c1ccc(-c2cccc(-c3cccc(-n4c5ccccc5c5ccc(-c6ccc7c(c6)c6ccccc6n7-c6cccc(-c7ccccc7)c6)cc54)c3)c2)cc1 ; c1ccc(-c2ccc(-c3cccc(-n4c5ccccc5c5ccc(-c6ccc7c(c6)c6ccccc6n7-c6cccc(-c7ccccc7)c6)cc54)c3)cc2)cc1]

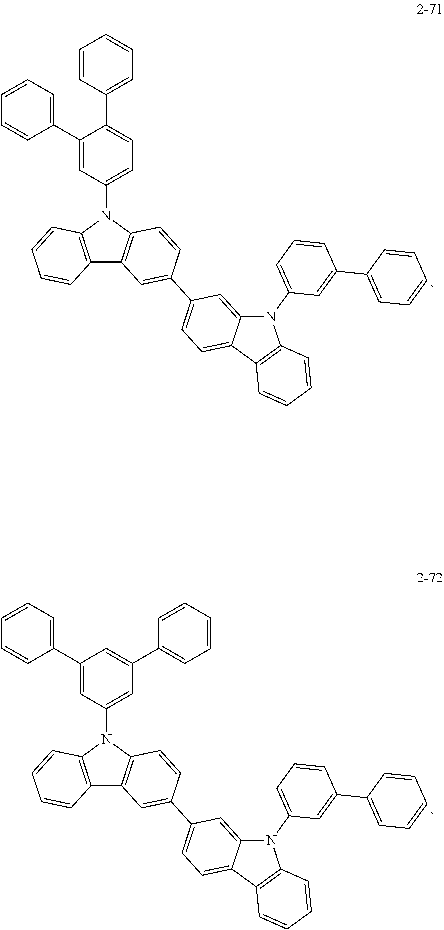 [SMILES: c1ccc(-c2cccc(-n3c4ccccc4c4ccc(-c5ccc6c(c5)c5ccccc5n6-c5ccc(-c6ccccc6)c(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccccc6)c5)cc43)c2)cc1]

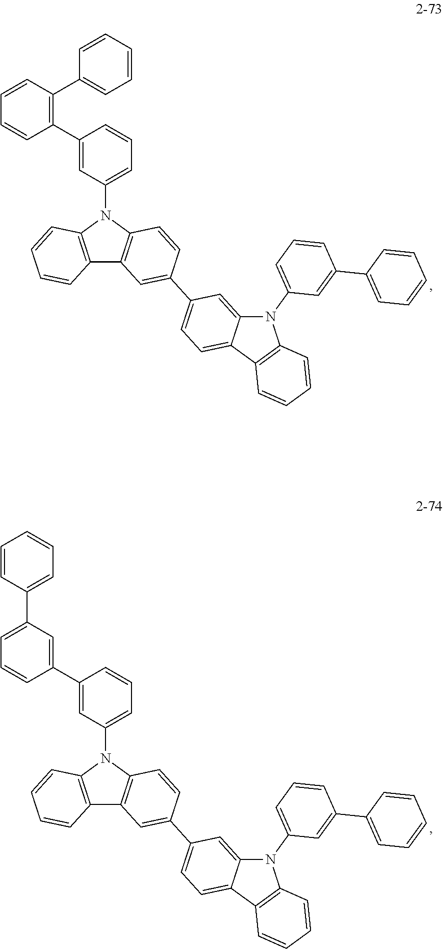 [SMILES: c1ccc(-c2cccc(-n3c4ccccc4c4ccc(-c5ccc6c(c5)c5ccccc5n6-c5cccc(-c6ccccc6-c6ccccc6)c5)cc43)c2)cc1 ; c1ccc(-c2cccc(-c3cccc(-n4c5ccccc5c5cc(-c6ccc7c8ccccc8n(-c8cccc(-c9ccccc9)c8)c7c6)ccc54)c3)c2)cc1]

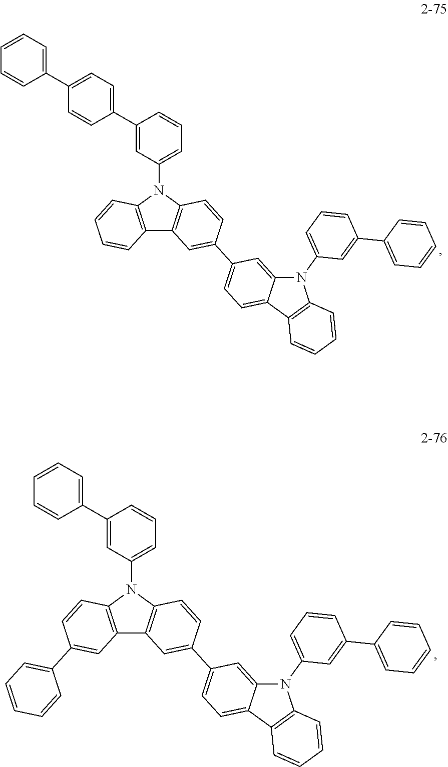 [SMILES: c1ccc(-c2ccc(-c3cccc(-n4c5ccccc5c5cc(-c6ccc7c8ccccc8n(-c8cccc(-c9ccccc9)c8)c7c6)ccc54)c3)cc2)cc1 ; c1ccc(-c2cccc(-n3c4ccc(-c5ccccc5)cc4c4cc(-c5ccc6c7ccccc7n(-c7cccc(-c8ccccc8)c7)c6c5)ccc43)c2)cc1]

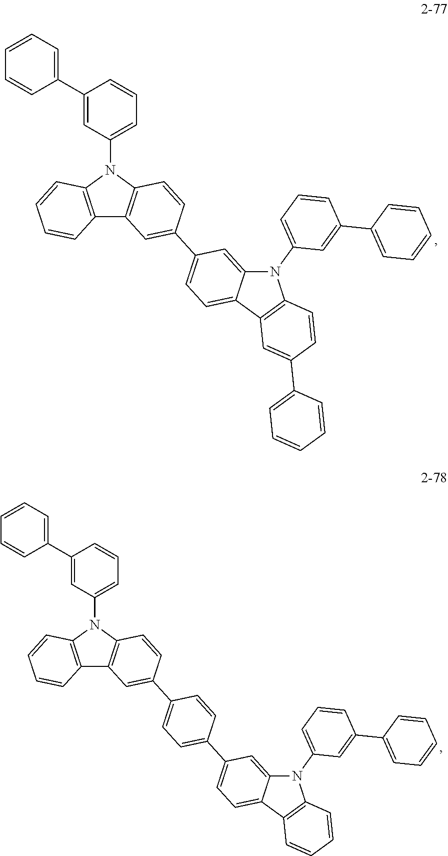 [SMILES: c1ccc(-c2cccc(-n3c4ccccc4c4cc(-c5ccc6c7cc(-c8ccccc8)ccc7n(-c7cccc(-c8ccccc8)c7)c6c5)ccc43)c2)cc1 ; c1ccc(-c2cccc(-n3c4ccccc4c4cc(-c5ccc(-c6ccc7c8ccccc8n(-c8cccc(-c9ccccc9)c8)c7c6)cc5)ccc43)c2)cc1]

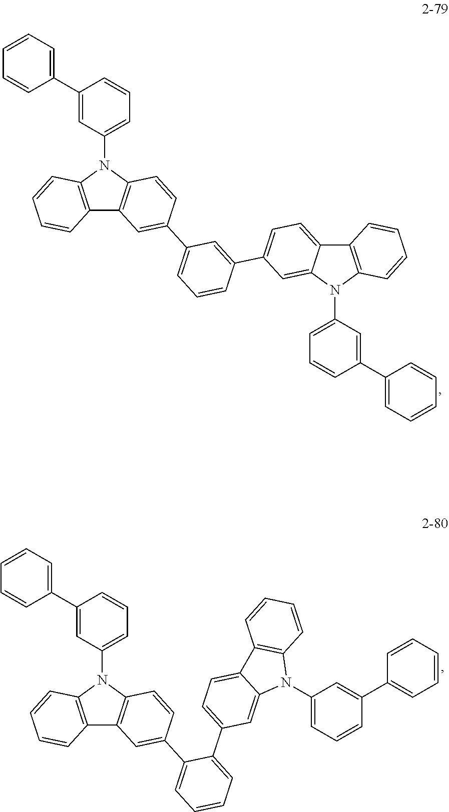 [SMILES: c1ccc(-c2cccc(-n3c4ccccc4c4cc(-c5cccc(-c6ccc7c8ccccc8n(-c8cccc(-c9ccccc9)c8)c7c6)c5)ccc43)c2)cc1 ; c1ccc(-c2cccc(-n3c4ccccc4c4cc(-c5ccccc5-c5ccc6c7ccccc7n(-c7cccc(-c8ccccc8)c7)c6c5)ccc43)c2)cc1]

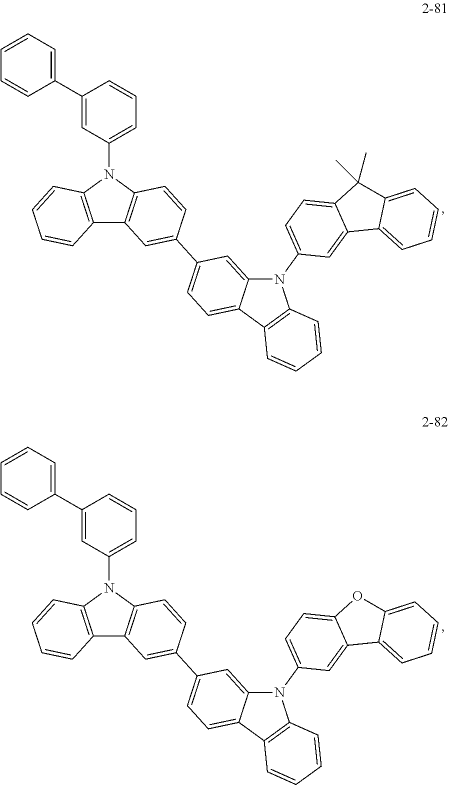 [SMILES: CC1(C)c2ccccc2-c2cc(-n3c4ccccc4c4ccc(-c5ccc6c(c5)c5ccccc5n6-c5cccc(-c6ccccc6)c5)cc43)ccc21 ; c1ccc(-c2cccc(-n3c4ccccc4c4cc(-c5ccc6c7ccccc7n(-c7ccc8oc9ccccc9c8c7)c6c5)ccc43)c2)cc1]

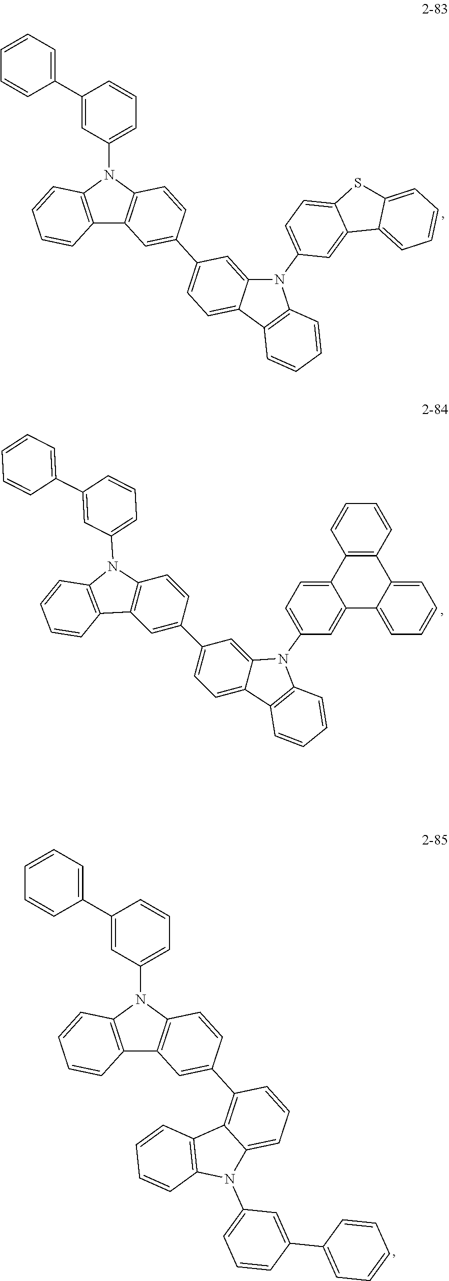 [SMILES: c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8c9ccccc9c9ccccc9c8c7)c6c5)ccc43)c2)cc1 ; c1ccc(-c2cccc(-n3c4ccccc4c4cc(-c5cccc6c5c5ccccc5n6-c5cccc(-c6ccccc6)c5)ccc43)c2)cc1]

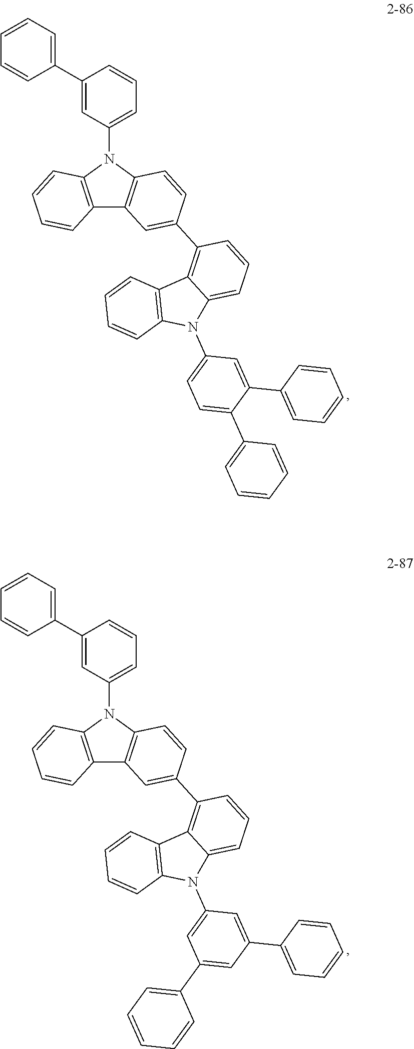 [SMILES: c1ccc(-c2cccc(-n3c4ccccc4c4cc(-c5cccc6c5c5ccccc5n6-c5ccc(-c6ccccc6)c(-c6ccccc6)c5)ccc43)c2)cc1 ; c1ccc(-c2cccc(-n3c4ccccc4c4cc(-c5cccc6c5c5ccccc5n6-c5cc(-c6ccccc6)cc(-c6ccccc6)c5)ccc43)c2)cc1]

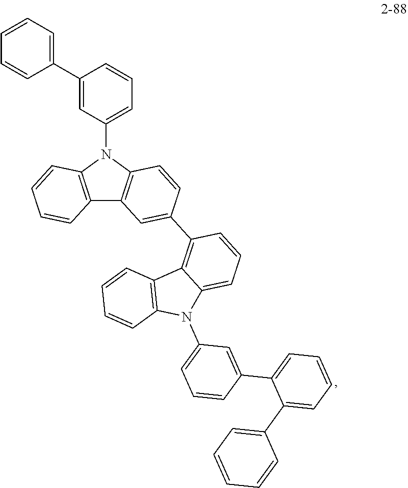 [SMILES: c1ccc(-c2cccc(-n3c4ccccc4c4cc(-c5cccc6c5c5ccccc5n6-c5cccc(-c6ccccc6-c6ccccc6)c5)ccc43)c2)cc1]

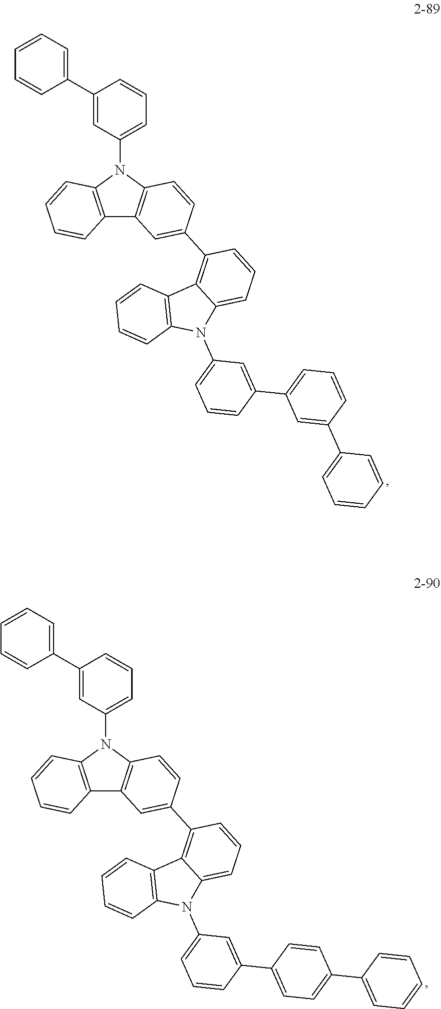 [SMILES: c1ccc(-c2cccc(-c3cccc(-n4c5ccccc5c5c(-c6ccc7c(c6)c6ccccc6n7-c6cccc(-c7ccccc7)c6)cccc54)c3)c2)cc1 ; c1ccc(-c2ccc(-c3cccc(-n4c5ccccc5c5c(-c6ccc7c(c6)c6ccccc6n7-c6cccc(-c7ccccc7)c6)cccc54)c3)cc2)cc1]

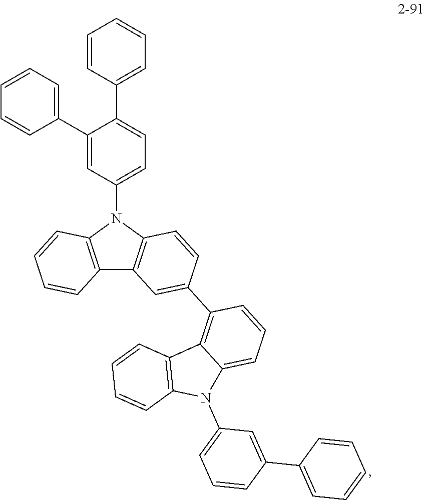 [SMILES: c1ccc(-c2cccc(-n3c4ccccc4c4c(-c5ccc6c(c5)c5ccccc5n6-c5ccc(-c6ccccc6)c(-c6ccccc6)c5)cccc43)c2)cc1]

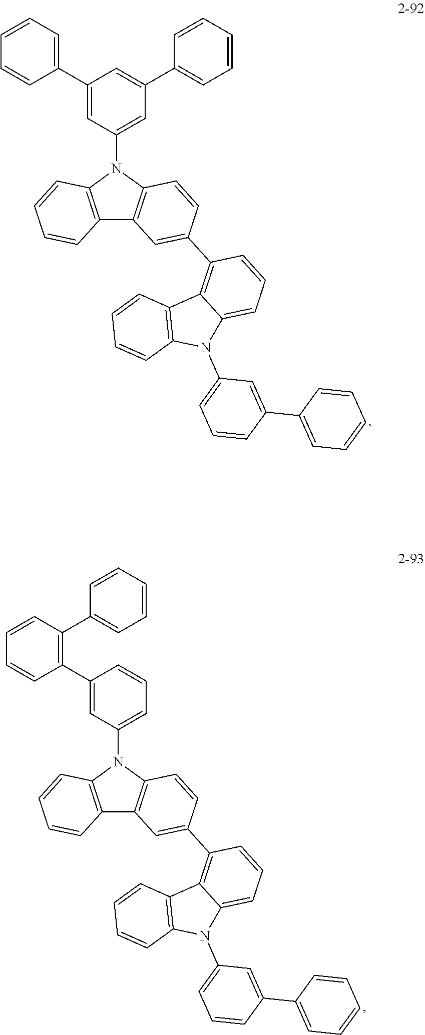 [SMILES: c1ccc(-c2cc(-c3ccccc3)cc(-n3c4ccccc4c4cc(-c5cccc6c5c5ccccc5n6-c5cccc(-c6ccccc6)c5)ccc43)c2)cc1 ; c1ccc(-c2cccc(-n3c4ccccc4c4c(-c5ccc6c(c5)c5ccccc5n6-c5cccc(-c6ccccc6-c6ccccc6)c5)cccc43)c2)cc1]

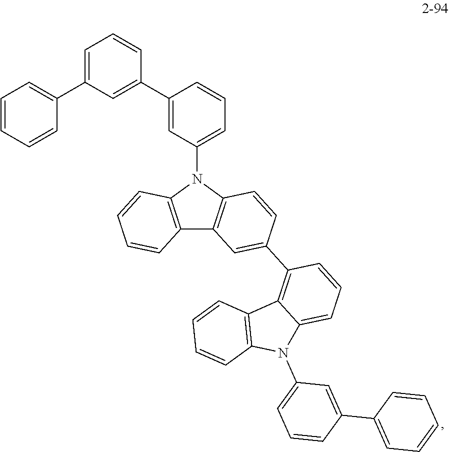 [SMILES: c1ccc(-c2cccc(-c3cccc(-n4c5ccccc5c5cc(-c6cccc7c6c6ccccc6n7-c6cccc(-c7ccccc7)c6)ccc54)c3)c2)cc1]

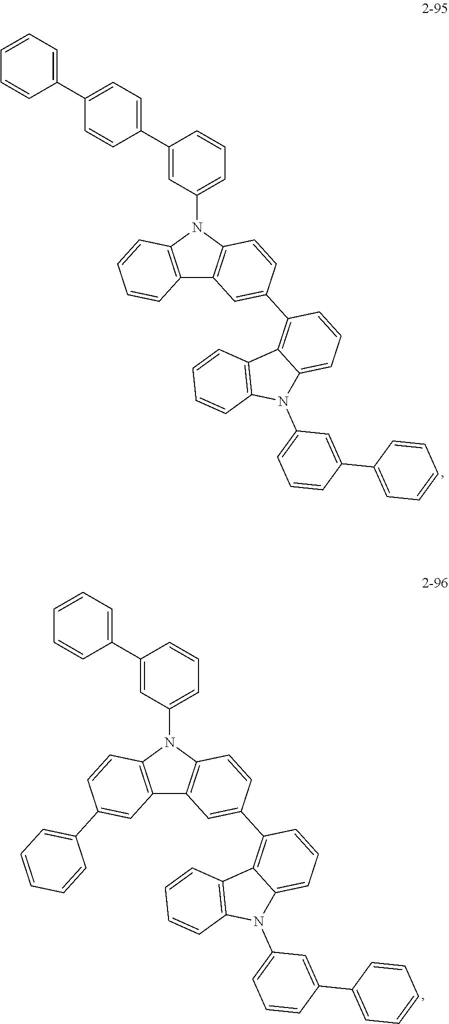 [SMILES: c1ccc(-c2ccc(-c3cccc(-n4c5ccccc5c5cc(-c6cccc7c6c6ccccc6n7-c6cccc(-c7ccccc7)c6)ccc54)c3)cc2)cc1 ; c1ccc(-c2cccc(-n3c4ccc(-c5ccccc5)cc4c4cc(-c5cccc6c5c5ccccc5n6-c5cccc(-c6ccccc6)c5)ccc43)c2)cc1]

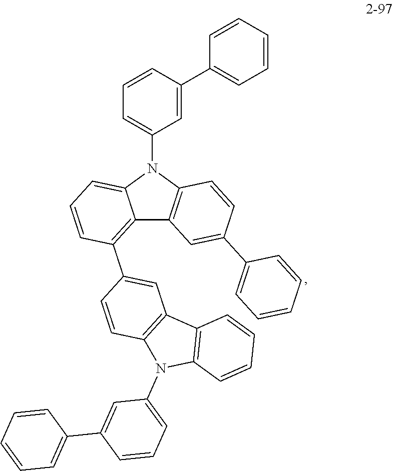 [SMILES: c1ccc(-c2cccc(-n3c4ccccc4c4cc(-c5cccc6c5c5cc(-c7ccccc7)ccc5n6-c5cccc(-c6ccccc6)c5)ccc43)c2)cc1]

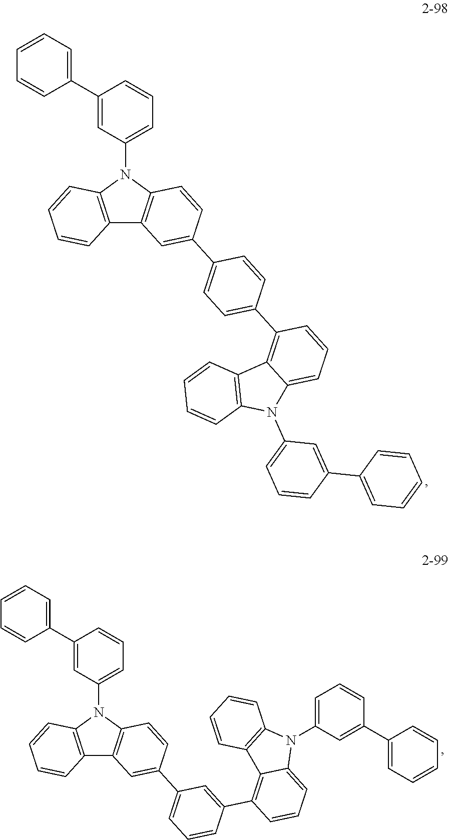 [SMILES: c1ccc(-c2cccc(-n3c4ccccc4c4cc(-c5ccc(-c6cccc7c6c6ccccc6n7-c6cccc(-c7ccccc7)c6)cc5)ccc43)c2)cc1 ; c1ccc(-c2cccc(-n3c4ccccc4c4cc(-c5cccc(-c6cccc7c6c6ccccc6n7-c6cccc(-c7ccccc7)c6)c5)ccc43)c2)cc1]

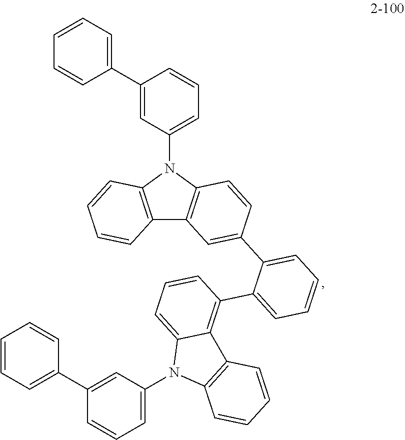 [SMILES: c1ccc(-c2cccc(-n3c4ccccc4c4cc(-c5ccccc5-c5cccc6c5c5ccccc5n6-c5cccc(-c6ccccc6)c5)ccc43)c2)cc1]

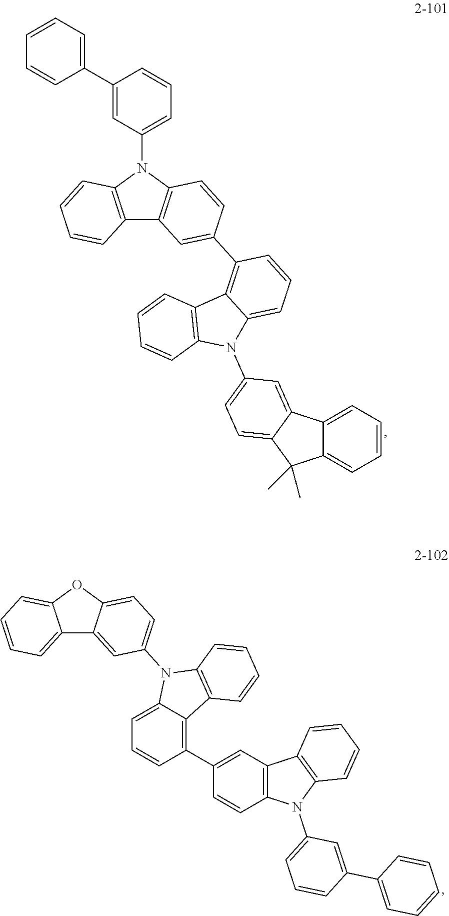 [SMILES: CC1(C)c2ccccc2-c2cc(-n3c4ccccc4c4c(-c5ccc6c(c5)c5ccccc5n6-c5cccc(-c6ccccc6)c5)cccc43)ccc21 ; c1ccc(-c2cccc(-n3c4ccccc4c4cc(-c5cccc6c5c5ccccc5n6-c5ccc6oc7ccccc7c6c5)ccc43)c2)cc1]

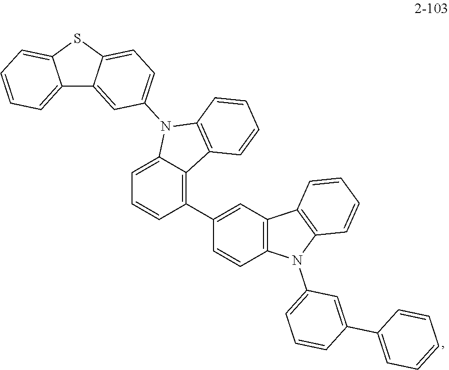 [SMILES: c1ccc(-c2cccc(-n3c4ccccc4c4cc(-c5cccc6c5c5ccccc5n6-c5ccc6sc7ccccc7c6c5)ccc43)c2)cc1]

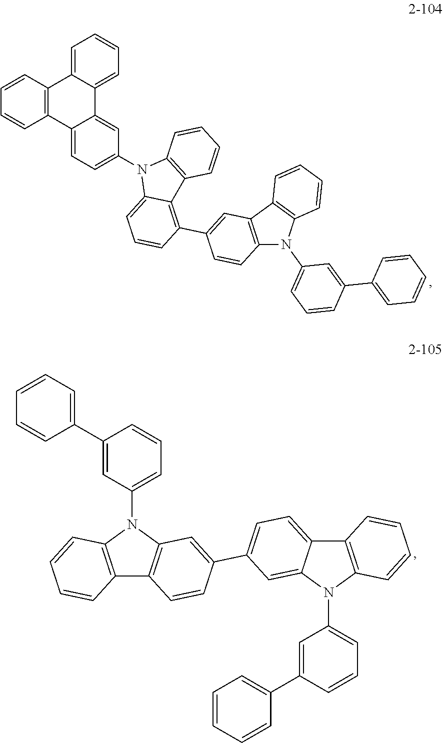 [SMILES: c1ccc(-c2cccc(-n3c4ccccc4c4cc(-c5cccc6c5c5ccccc5n6-c5ccc6c7ccccc7c7ccccc7c6c5)ccc43)c2)cc1 ; c1ccc(-c2cccc(-n3c4ccccc4c4ccc(-c5ccc6c7ccccc7n(-c7cccc(-c8ccccc8)c7)c6c5)cc43)c2)cc1]

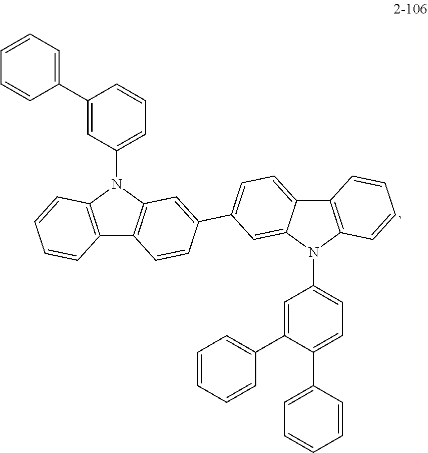 [SMILES: c1ccc(-c2cccc(-n3c4ccccc4c4ccc(-c5ccc6c7ccccc7n(-c7ccc(-c8ccccc8)c(-c8ccccc8)c7)c6c5)cc43)c2)cc1]

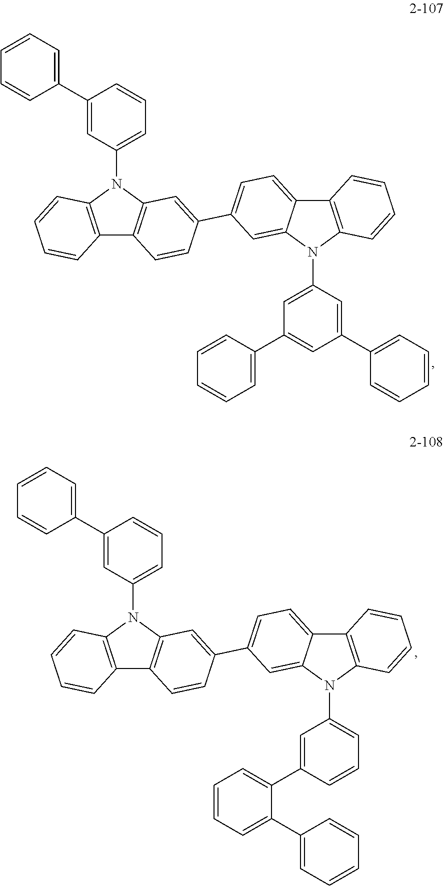 [SMILES: c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1 ; c1ccc(-c2cccc(-n3c4ccccc4c4ccc(-c5ccc6c7ccccc7n(-c7cccc(-c8ccccc8-c8ccccc8)c7)c6c5)cc43)c2)cc1]

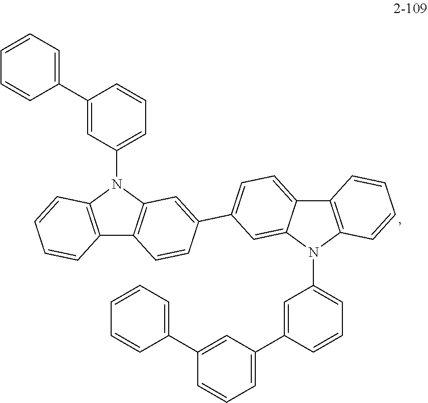 [SMILES: c1ccc(-c2cccc(-c3cccc(-n4c5ccccc5c5ccc(-c6ccc7c8ccccc8n(-c8cccc(-c9ccccc9)c8)c7c6)cc54)c3)c2)cc1]

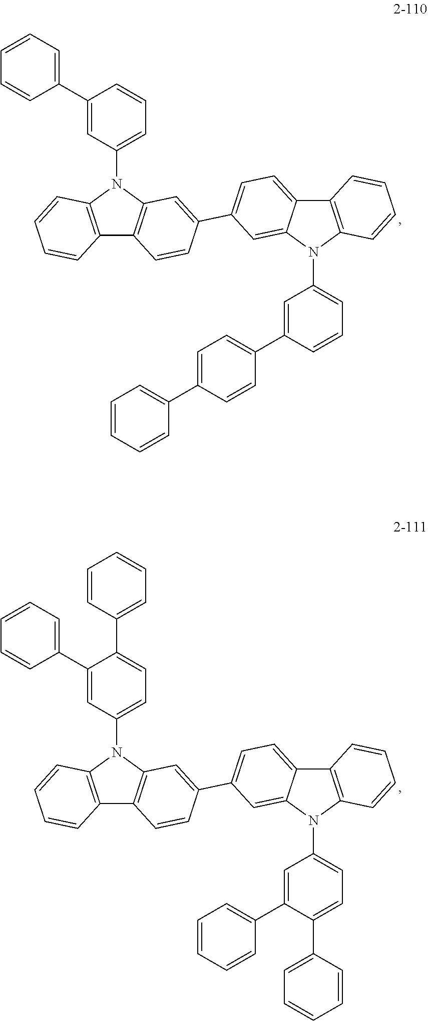 [SMILES: c1ccc(-c2ccc(-c3cccc(-n4c5ccccc5c5ccc(-c6ccc7c8ccccc8n(-c8cccc(-c9ccccc9)c8)c7c6)cc54)c3)cc2)cc1 ; c1ccc(-c2ccc(-n3c4ccccc4c4ccc(-c5ccc6c7ccccc7n(-c7ccc(-c8ccccc8)c(-c8ccccc8)c7)c6c5)cc43)cc2-c2ccccc2)cc1]

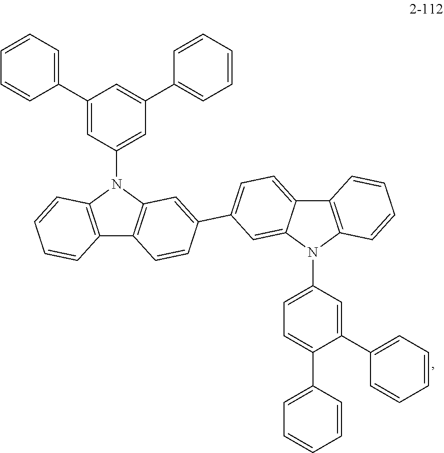 [SMILES: c1ccc(-c2cc(-c3ccccc3)cc(-n3c4ccccc4c4ccc(-c5ccc6c7ccccc7n(-c7ccc(-c8ccccc8)c(-c8ccccc8)c7)c6c5)cc43)c2)cc1]

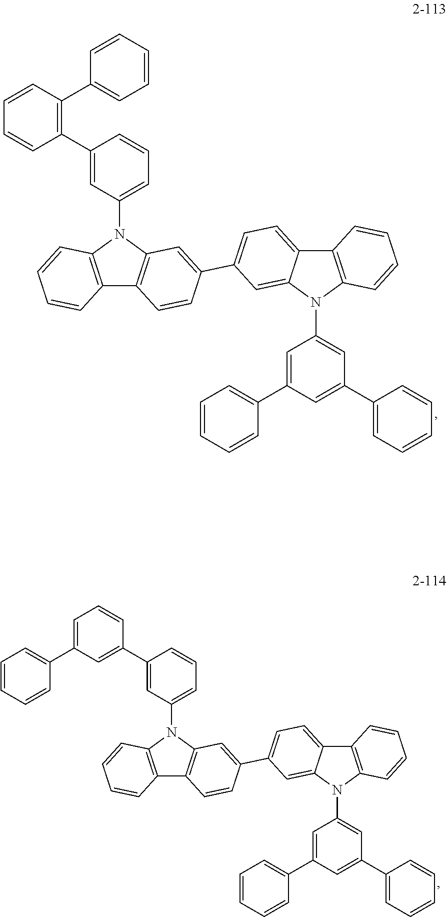 [SMILES: c1ccc(-c2cc(-c3ccccc3)cc(-n3c4ccccc4c4ccc(-c5ccc6c7ccccc7n(-c7cccc(-c8ccccc8-c8ccccc8)c7)c6c5)cc43)c2)cc1 ; c1ccc(-c2cccc(-c3cccc(-n4c5ccccc5c5ccc(-c6ccc7c8ccccc8n(-c8cc(-c9ccccc9)cc(-c9ccccc9)c8)c7c6)cc54)c3)c2)cc1]

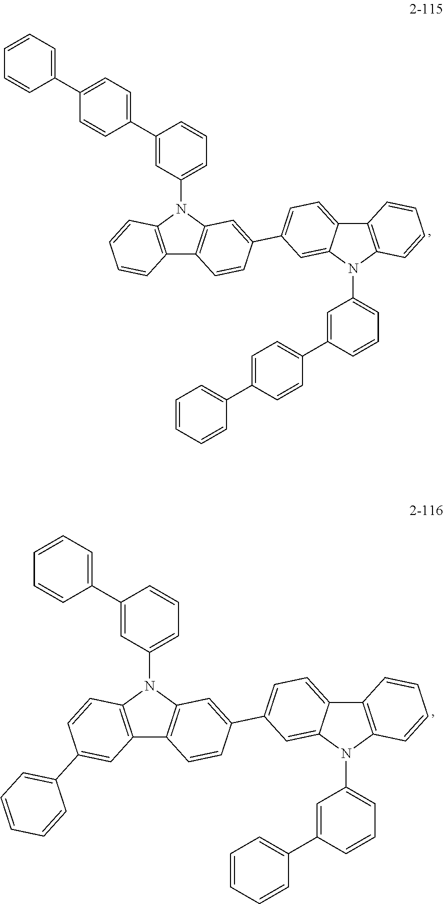 [SMILES: c1ccc(-c2ccc(-c3cccc(-n4c5ccccc5c5ccc(-c6ccc7c8ccccc8n(-c8cccc(-c9ccc(-c%10ccccc%10)cc9)c8)c7c6)cc54)c3)cc2)cc1 ; c1ccc(-c2cccc(-n3c4ccccc4c4ccc(-c5ccc6c7cc(-c8ccccc8)ccc7n(-c7cccc(-c8ccccc8)c7)c6c5)cc43)c2)cc1]

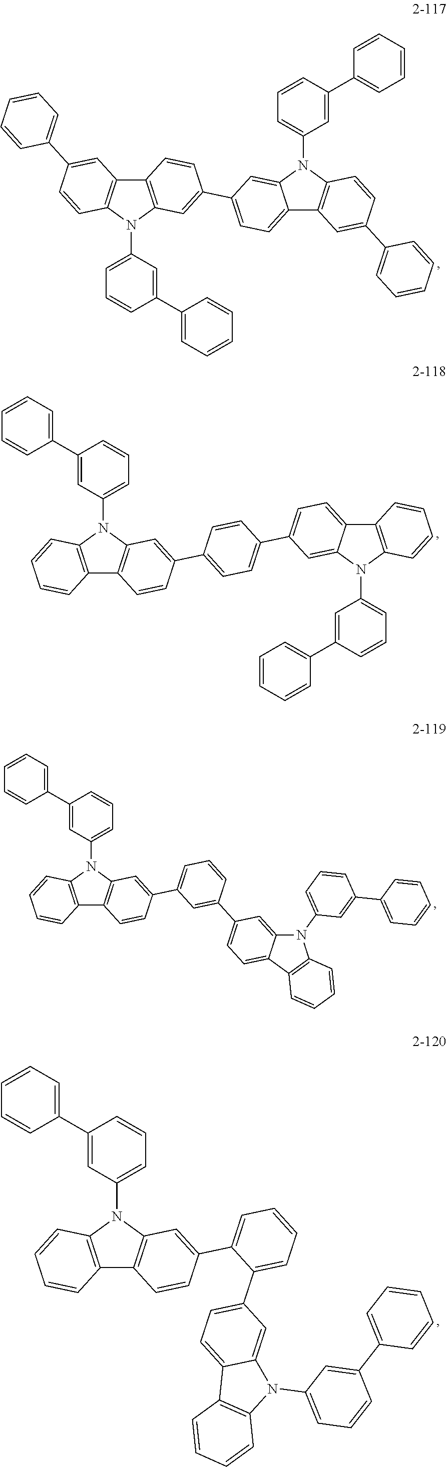 [SMILES: c1ccc(-c2cccc(-n3c4ccc(-c5ccccc5)cc4c4ccc(-c5ccc6c7cc(-c8ccccc8)ccc7n(-c7cccc(-c8ccccc8)c7)c6c5)cc43)c2)cc1 ; c1ccc(-c2cccc(-n3c4ccccc4c4ccc(-c5ccc(-c6ccc7c8ccccc8n(-c8cccc(-c9ccccc9)c8)c7c6)cc5)cc43)c2)cc1 ; c1ccc(-c2cccc(-n3c4ccccc4c4ccc(-c5cccc(-c6ccc7c8ccccc8n(-c8cccc(-c9ccccc9)c8)c7c6)c5)cc43)c2)cc1 ; c1ccc(-c2cccc(-n3c4ccccc4c4ccc(-c5ccccc5-c5ccc6c7ccccc7n(-c7cccc(-c8ccccc8)c7)c6c5)cc43)c2)cc1]

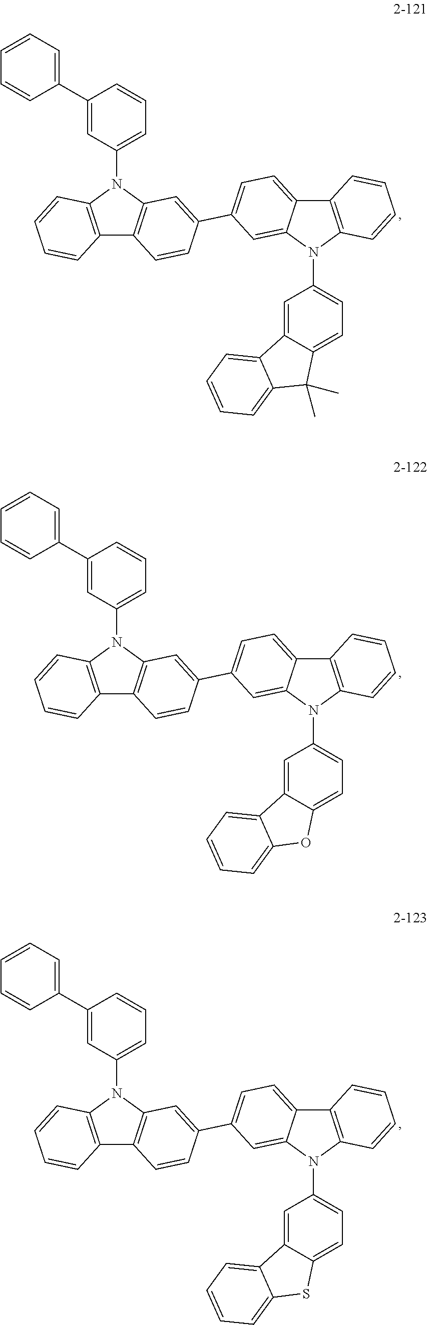 [SMILES: CC1(C)c2ccccc2-c2cc(-n3c4ccccc4c4ccc(-c5ccc6c7ccccc7n(-c7cccc(-c8ccccc8)c7)c6c5)cc43)ccc21 ; c1ccc(-c2cccc(-n3c4ccccc4c4ccc(-c5ccc6c7ccccc7n(-c7ccc8oc9ccccc9c8c7)c6c5)cc43)c2)cc1 ; c1ccc(-c2cccc(-n3c4ccccc4c4ccc(-c5ccc6c7ccccc7n(-c7ccc8sc9ccccc9c8c7)c6c5)cc43)c2)cc1]

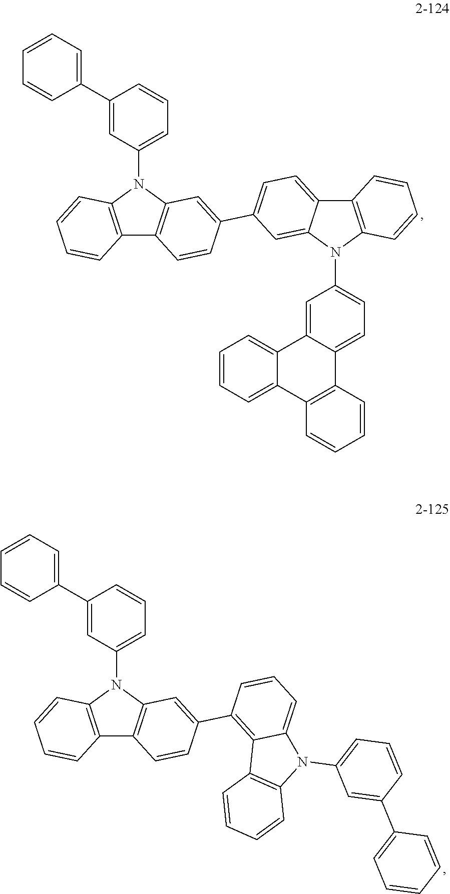 [SMILES: c1ccc(-c2cccc(-n3c4ccccc4c4ccc(-c5ccc6c7ccccc7n(-c7ccc8c9ccccc9c9ccccc9c8c7)c6c5)cc43)c2)cc1 ; c1ccc(-c2cccc(-n3c4ccccc4c4ccc(-c5cccc6c5c5ccccc5n6-c5cccc(-c6ccccc6)c5)cc43)c2)cc1]

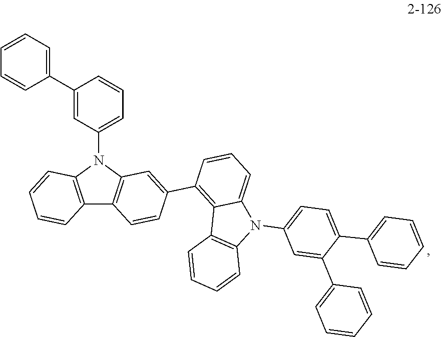 [SMILES: c1ccc(-c2cccc(-n3c4ccccc4c4ccc(-c5cccc6c5c5ccccc5n6-c5ccc(-c6ccccc6)c(-c6ccccc6)c5)cc43)c2)cc1]

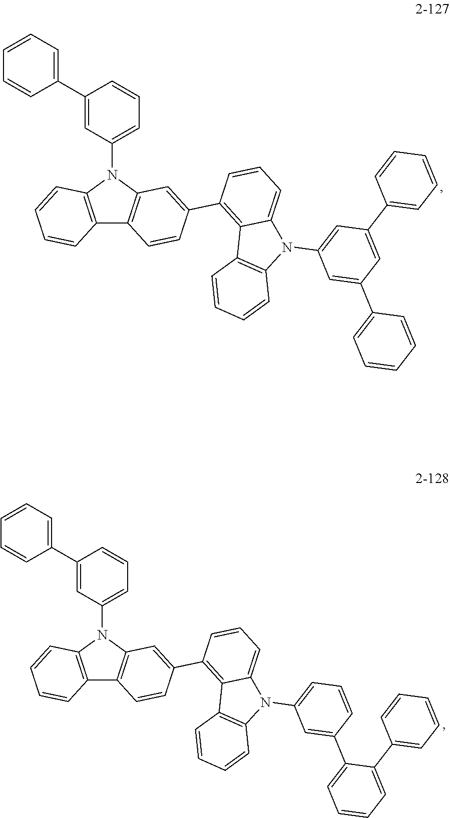 [SMILES: c1ccc(-c2cccc(-n3c4ccccc4c4ccc(-c5cccc6c5c5ccccc5n6-c5cc(-c6ccccc6)cc(-c6ccccc6)c5)cc43)c2)cc1 ; c1ccc(-c2cccc(-n3c4ccccc4c4ccc(-c5cccc6c5c5ccccc5n6-c5cccc(-c6ccccc6-c6ccccc6)c5)cc43)c2)cc1]

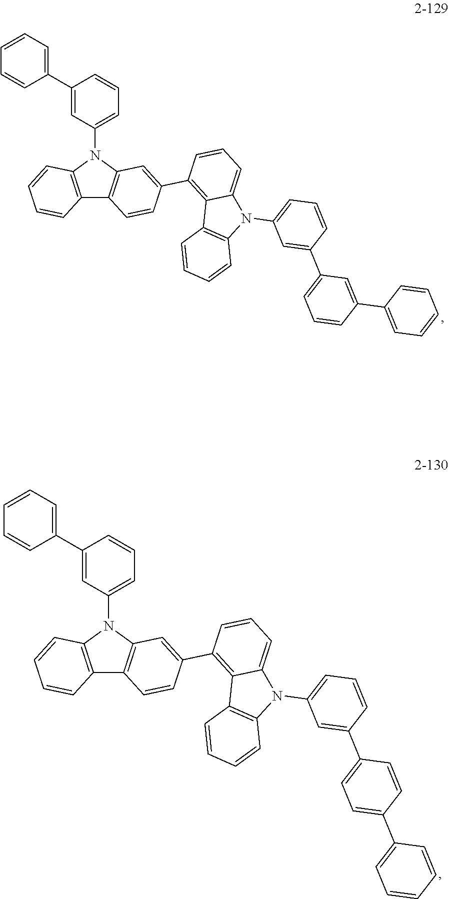 [SMILES: c1ccc(-c2cccc(-c3cccc(-n4c5ccccc5c5c(-c6ccc7c8ccccc8n(-c8cccc(-c9ccccc9)c8)c7c6)cccc54)c3)c2)cc1 ; c1ccc(-c2ccc(-c3cccc(-n4c5ccccc5c5c(-c6ccc7c8ccccc8n(-c8cccc(-c9ccccc9)c8)c7c6)cccc54)c3)cc2)cc1]

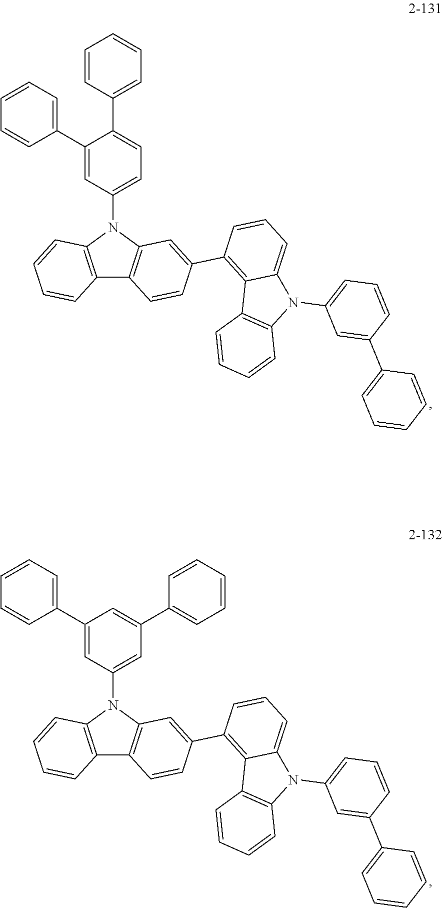 [SMILES: c1ccc(-c2cccc(-n3c4ccccc4c4c(-c5ccc6c7ccccc7n(-c7ccc(-c8ccccc8)c(-c8ccccc8)c7)c6c5)cccc43)c2)cc1 ; c1ccc(-c2cc(-c3ccccc3)cc(-n3c4ccccc4c4ccc(-c5cccc6c5c5ccccc5n6-c5cccc(-c6ccccc6)c5)cc43)c2)cc1]

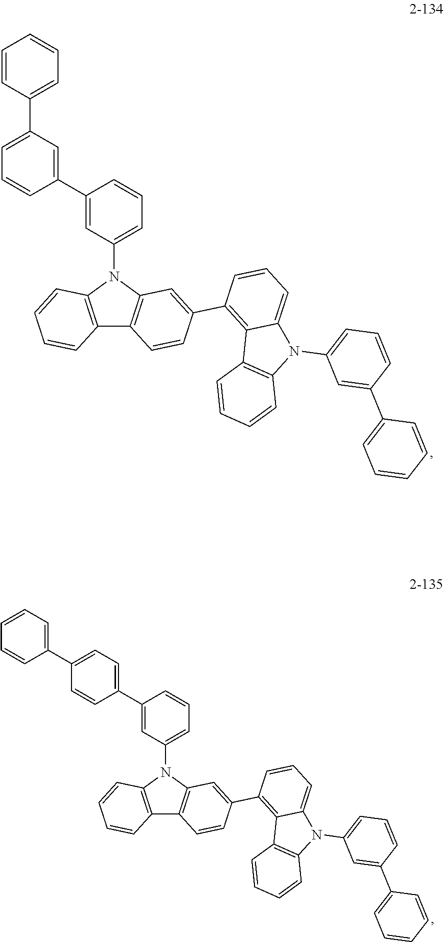 [SMILES: c1ccc(-c2cccc(-c3cccc(-n4c5ccccc5c5ccc(-c6cccc7c6c6ccccc6n7-c6cccc(-c7ccccc7)c6)cc54)c3)c2)cc1 ; c1ccc(-c2ccc(-c3cccc(-n4c5ccccc5c5ccc(-c6cccc7c6c6ccccc6n7-c6cccc(-c7ccccc7)c6)cc54)c3)cc2)cc1]

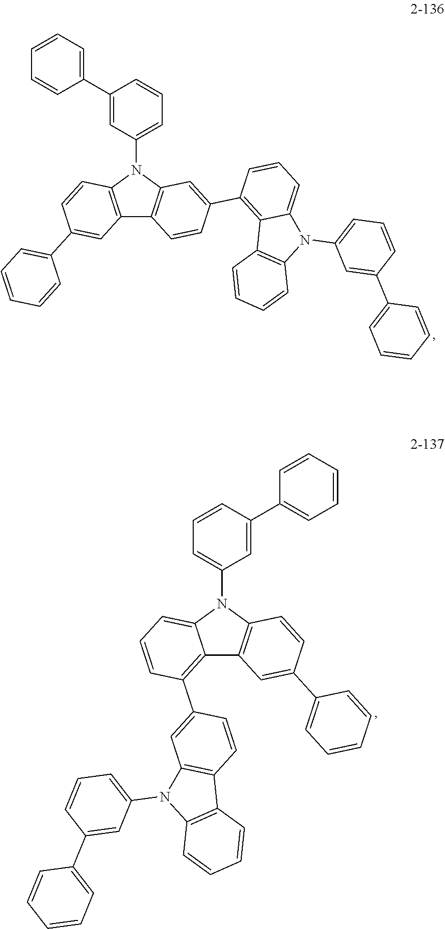 [SMILES: c1ccc(-c2cccc(-n3c4ccc(-c5ccccc5)cc4c4ccc(-c5cccc6c5c5ccccc5n6-c5cccc(-c6ccccc6)c5)cc43)c2)cc1 ; c1ccc(-c2cccc(-n3c4ccccc4c4ccc(-c5cccc6c5c5cc(-c7ccccc7)ccc5n6-c5cccc(-c6ccccc6)c5)cc43)c2)cc1]

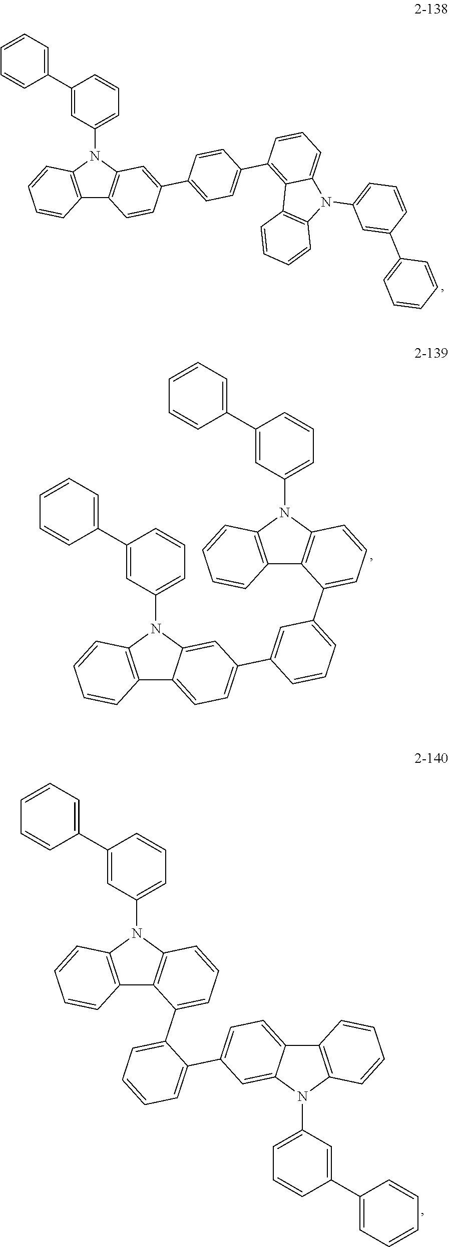 [SMILES: c1ccc(-c2cccc(-n3c4ccccc4c4ccc(-c5ccc(-c6cccc7c6c6ccccc6n7-c6cccc(-c7ccccc7)c6)cc5)cc43)c2)cc1 ; c1ccc(-c2cccc(-n3c4ccccc4c4ccc(-c5cccc(-c6cccc7c6c6ccccc6n7-c6cccc(-c7ccccc7)c6)c5)cc43)c2)cc1 ; c1ccc(-c2cccc(-n3c4ccccc4c4ccc(-c5ccccc5-c5cccc6c5c5ccccc5n6-c5cccc(-c6ccccc6)c5)cc43)c2)cc1]

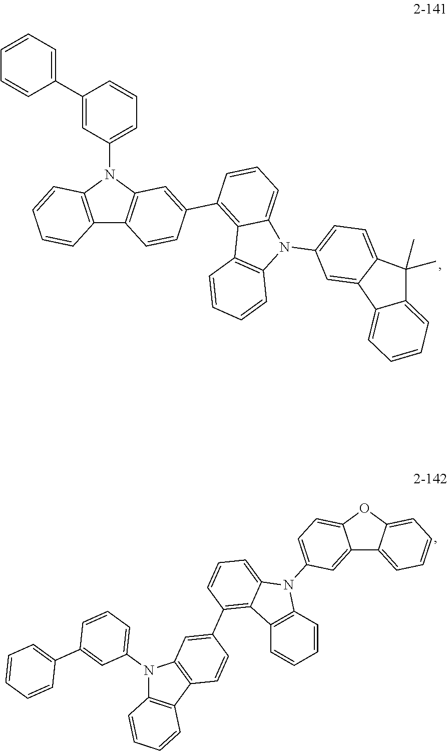 [SMILES: CC1(C)c2ccccc2-c2cc(-n3c4ccccc4c4c(-c5ccc6c7ccccc7n(-c7cccc(-c8ccccc8)c7)c6c5)cccc43)ccc21 ; c1ccc(-c2cccc(-n3c4ccccc4c4ccc(-c5cccc6c5c5ccccc5n6-c5ccc6oc7ccccc7c6c5)cc43)c2)cc1]

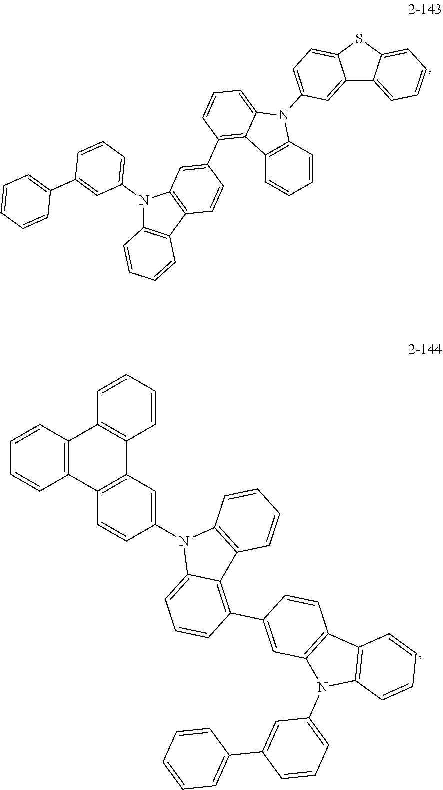 [SMILES: c1ccc(-c2cccc(-n3c4ccccc4c4ccc(-c5cccc6c5c5ccccc5n6-c5ccc6sc7ccccc7c6c5)cc43)c2)cc1 ; c1ccc(-c2cccc(-n3c4ccccc4c4ccc(-c5cccc6c5c5ccccc5n6-c5ccc6c7ccccc7c7ccccc7c6c5)cc43)c2)cc1]

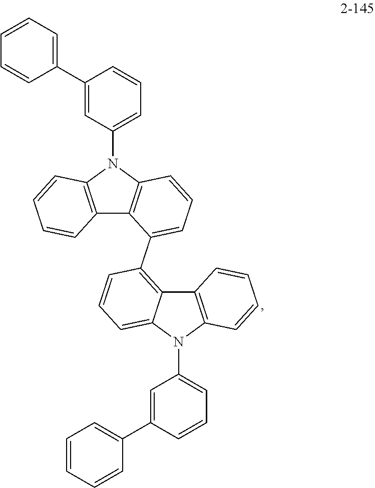 [SMILES: c1ccc(-c2cccc(-n3c4ccccc4c4c(-c5cccc6c5c5ccccc5n6-c5cccc(-c6ccccc6)c5)cccc43)c2)cc1]

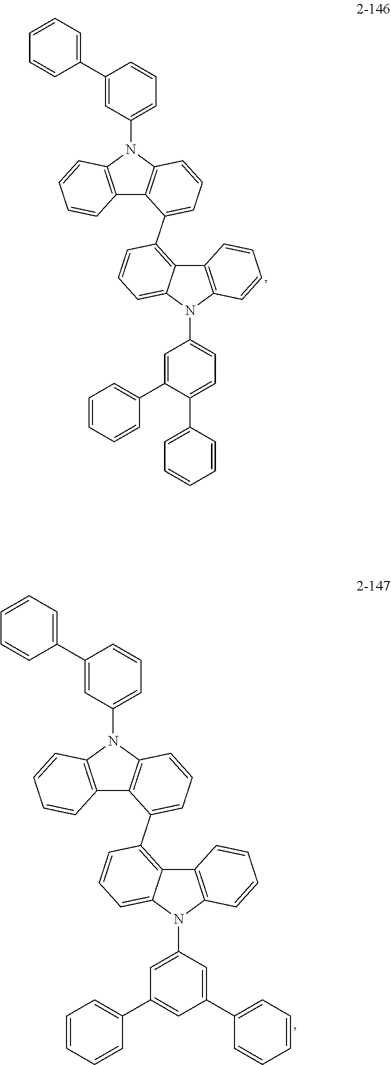 [SMILES: c1ccc(-c2cccc(-n3c4ccccc4c4c(-c5cccc6c5c5ccccc5n6-c5ccc(-c6ccccc6)c(-c6ccccc6)c5)cccc43)c2)cc1 ; c1ccc(-c2cccc(-n3c4ccccc4c4c(-c5cccc6c5c5ccccc5n6-c5cc(-c6ccccc6)cc(-c6ccccc6)c5)cccc43)c2)cc1]

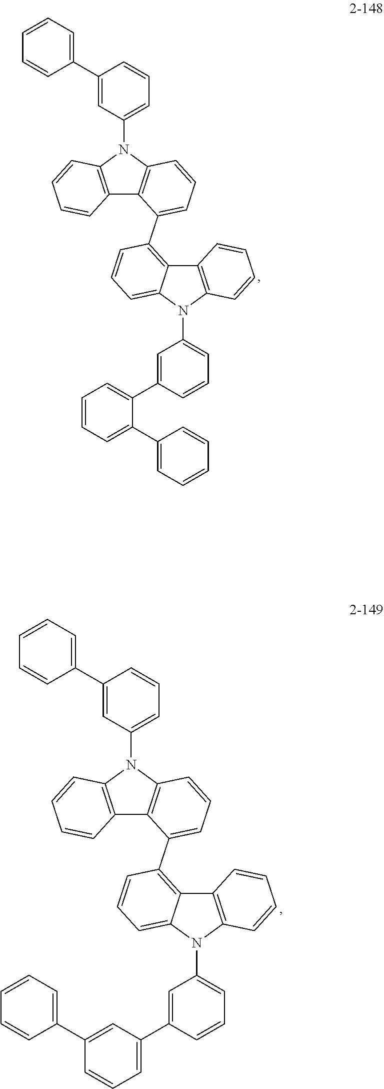 [SMILES: c1ccc(-c2cccc(-n3c4ccccc4c4c(-c5cccc6c5c5ccccc5n6-c5cccc(-c6ccccc6-c6ccccc6)c5)cccc43)c2)cc1 ; c1ccc(-c2cccc(-c3cccc(-n4c5ccccc5c5c(-c6cccc7c6c6ccccc6n7-c6cccc(-c7ccccc7)c6)cccc54)c3)c2)cc1]

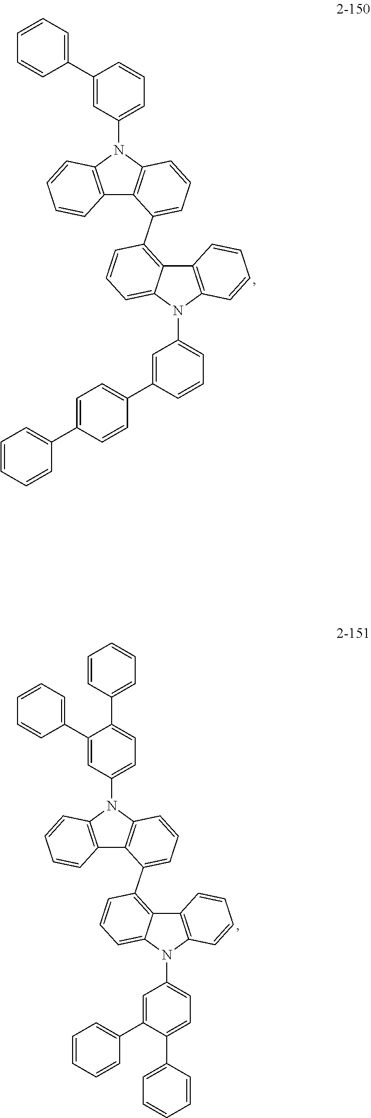 [SMILES: c1ccc(-c2ccc(-c3cccc(-n4c5ccccc5c5c(-c6cccc7c6c6ccccc6n7-c6cccc(-c7ccccc7)c6)cccc54)c3)cc2)cc1 ; c1ccc(-c2ccc(-n3c4ccccc4c4c(-c5cccc6c5c5ccccc5n6-c5ccc(-c6ccccc6)c(-c6ccccc6)c5)cccc43)cc2-c2ccccc2)cc1]

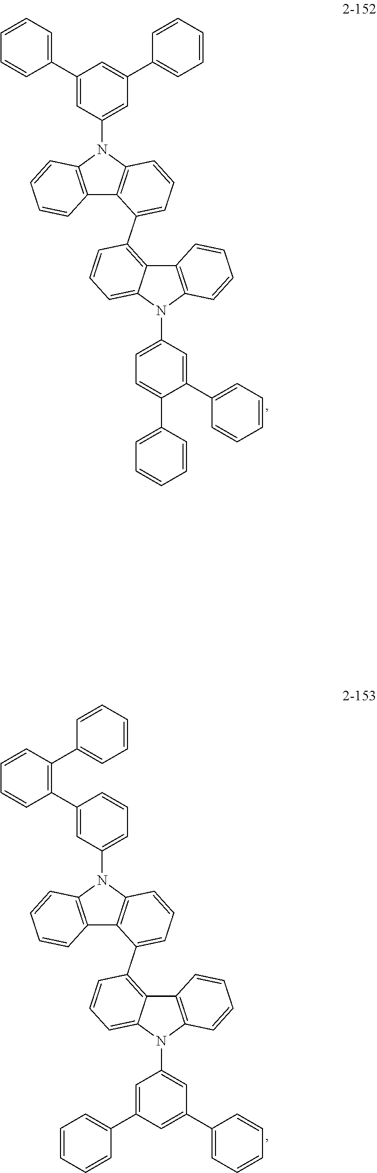 [SMILES: c1ccc(-c2cc(-c3ccccc3)cc(-n3c4ccccc4c4c(-c5cccc6c5c5ccccc5n6-c5ccc(-c6ccccc6)c(-c6ccccc6)c5)cccc43)c2)cc1 ; c1ccc(-c2cc(-c3ccccc3)cc(-n3c4ccccc4c4c(-c5cccc6c5c5ccccc5n6-c5cccc(-c6ccccc6-c6ccccc6)c5)cccc43)c2)cc1]

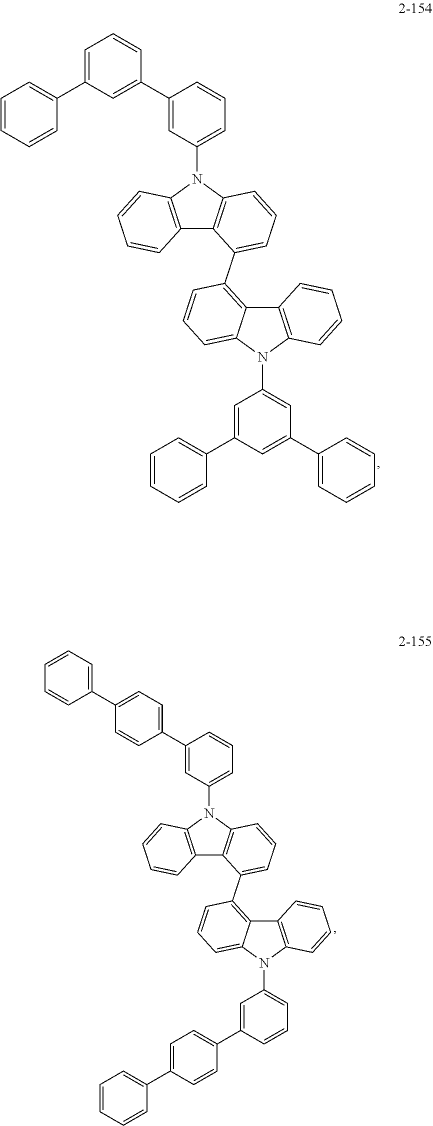 [SMILES: c1ccc(-c2cccc(-c3cccc(-n4c5ccccc5c5c(-c6cccc7c6c6ccccc6n7-c6cc(-c7ccccc7)cc(-c7ccccc7)c6)cccc54)c3)c2)cc1 ; c1ccc(-c2ccc(-c3cccc(-n4c5ccccc5c5c(-c6cccc7c6c6ccccc6n7-c6cccc(-c7ccc(-c8ccccc8)cc7)c6)cccc54)c3)cc2)cc1]

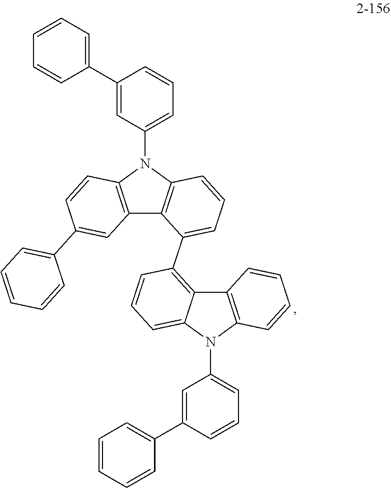 [SMILES: c1ccc(-c2cccc(-n3c4ccccc4c4c(-c5cccc6c5c5cc(-c7ccccc7)ccc5n6-c5cccc(-c6ccccc6)c5)cccc43)c2)cc1]

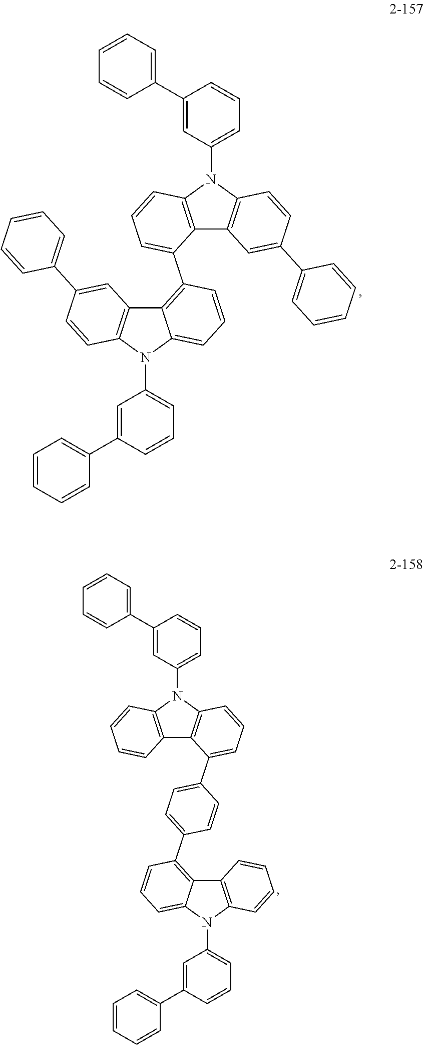 [SMILES: c1ccc(-c2cccc(-n3c4ccc(-c5ccccc5)cc4c4c(-c5cccc6c5c5cc(-c7ccccc7)ccc5n6-c5cccc(-c6ccccc6)c5)cccc43)c2)cc1 ; c1ccc(-c2cccc(-n3c4ccccc4c4c(-c5ccc(-c6cccc7c6c6ccccc6n7-c6cccc(-c7ccccc7)c6)cc5)cccc43)c2)cc1]

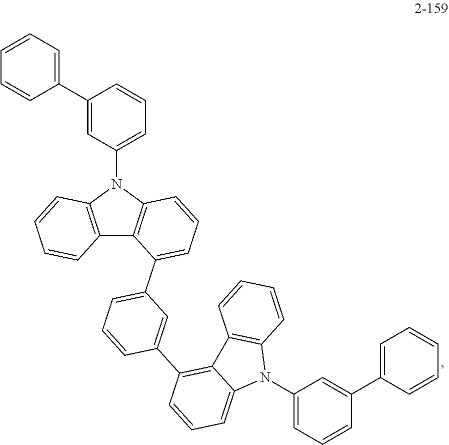 [SMILES: c1ccc(-c2cccc(-n3c4ccccc4c4c(-c5cccc(-c6cccc7c6c6ccccc6n7-c6cccc(-c7ccccc7)c6)c5)cccc43)c2)cc1]

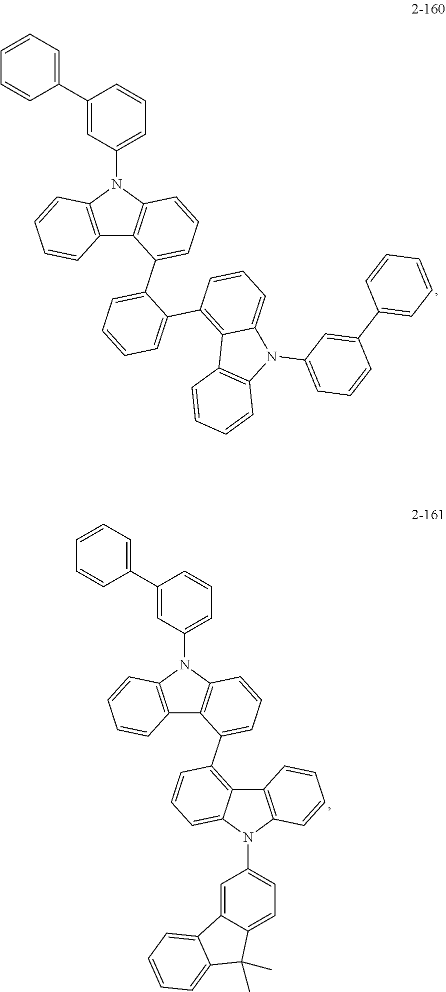 [SMILES: c1ccc(-c2cccc(-n3c4ccccc4c4c(-c5ccccc5-c5cccc6c5c5ccccc5n6-c5cccc(-c6ccccc6)c5)cccc43)c2)cc1 ; CC1(C)c2ccccc2-c2cc(-n3c4ccccc4c4c(-c5cccc6c5c5ccccc5n6-c5cccc(-c6ccccc6)c5)cccc43)ccc21]

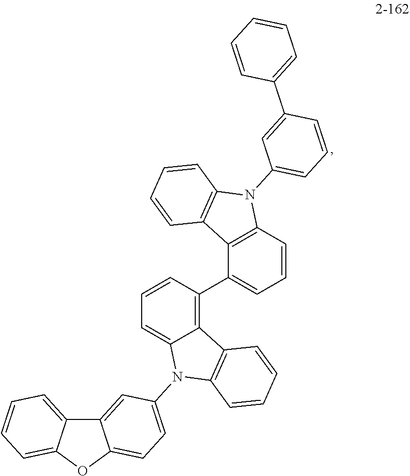 [SMILES: c1ccc(-c2cccc(-n3c4ccccc4c4c(-c5cccc6c5c5ccccc5n6-c5ccc6oc7ccccc7c6c5)cccc43)c2)cc1]

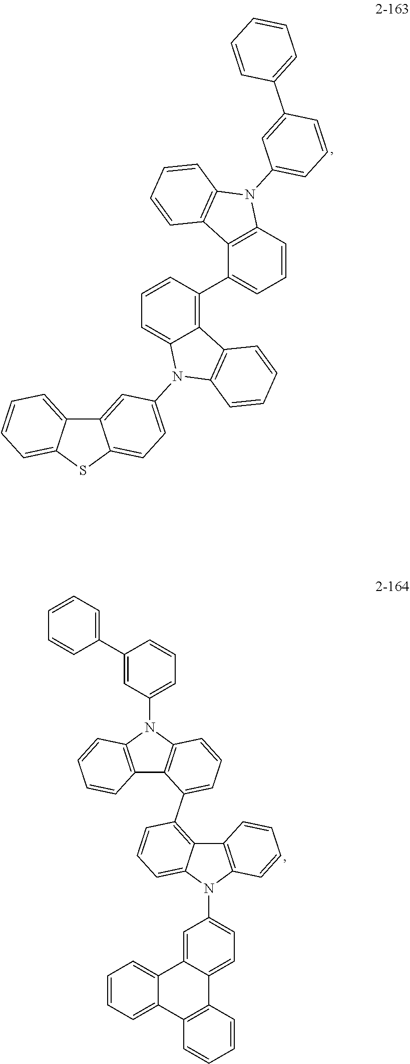 [SMILES: c1ccc(-c2cccc(-n3c4ccccc4c4c(-c5cccc6c5c5ccccc5n6-c5ccc6sc7ccccc7c6c5)cccc43)c2)cc1 ; c1ccc(-c2cccc(-n3c4ccccc4c4c(-c5cccc6c5c5ccccc5n6-c5ccc6c7ccccc7c7ccccc7c6c5)cccc43)c2)cc1]

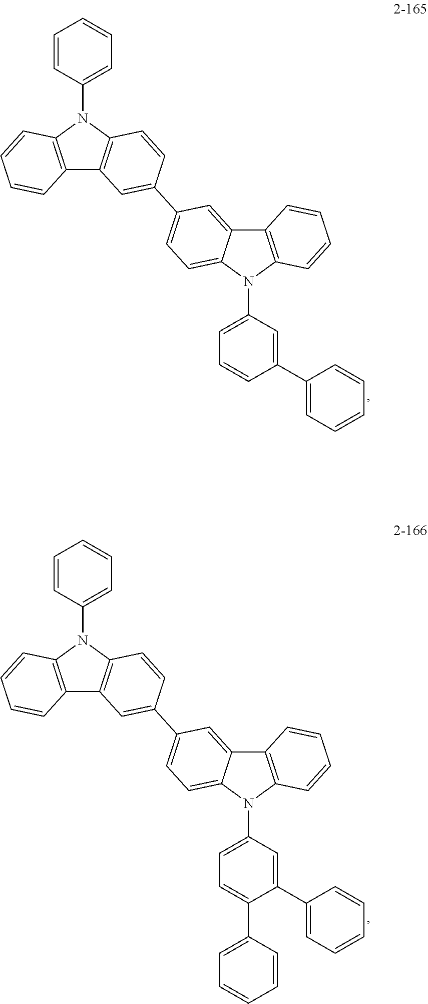 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2-c2ccccc2)cc1]

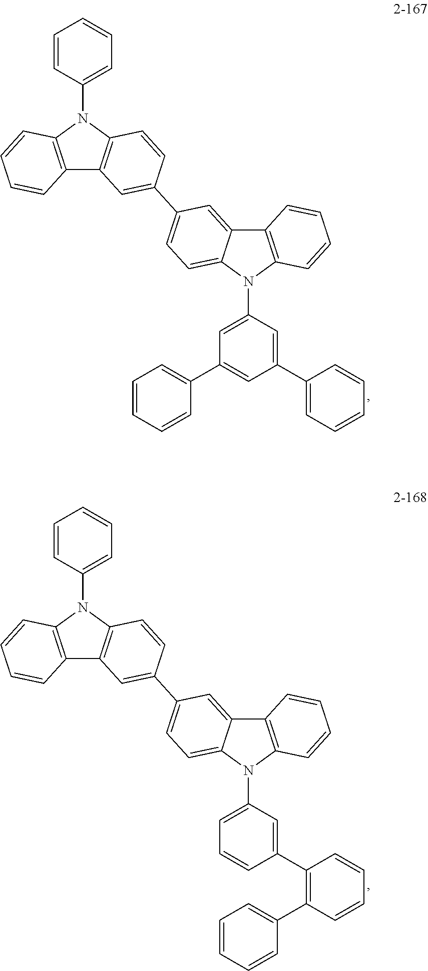 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccccc2-c2cccc(-n3c4ccccc4c4cc(-c5ccc6c(c5)c5ccccc5n6-c5ccccc5)ccc43)c2)cc1]

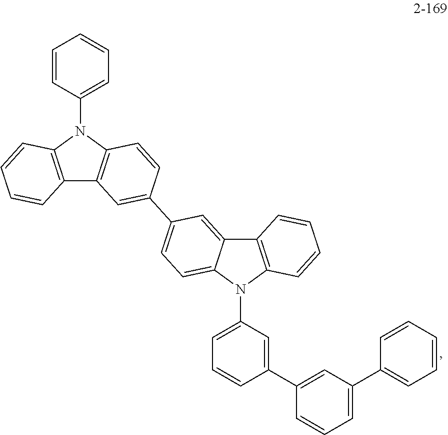 [SMILES: c1ccc(-c2cccc(-c3cccc(-n4c5ccccc5c5cc(-c6ccc7c(c6)c6ccccc6n7-c6ccccc6)ccc54)c3)c2)cc1]

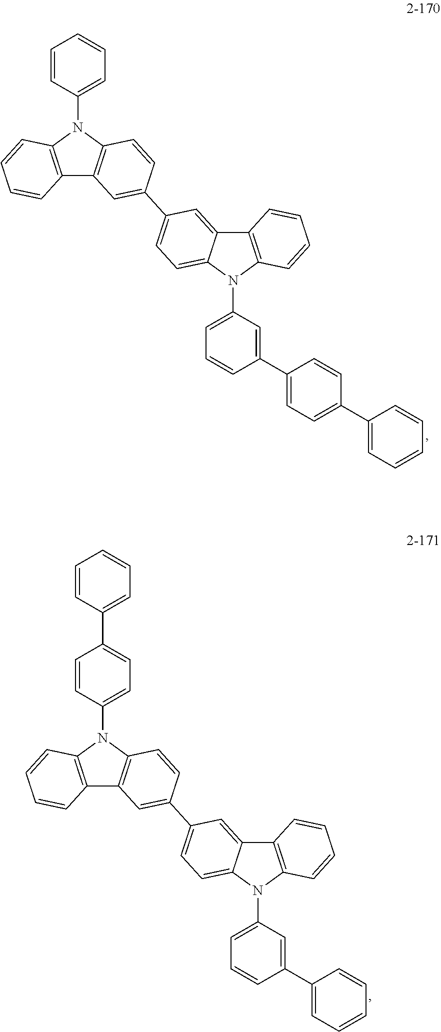 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

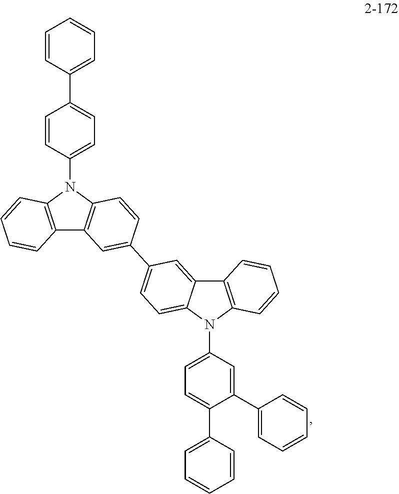 [SMILES: c1ccc(-c2ccc(-n3c4ccccc4c4cc(-c5ccc6c(c5)c5ccccc5n6-c5ccc(-c6ccccc6)c(-c6ccccc6)c5)ccc43)cc2)cc1]

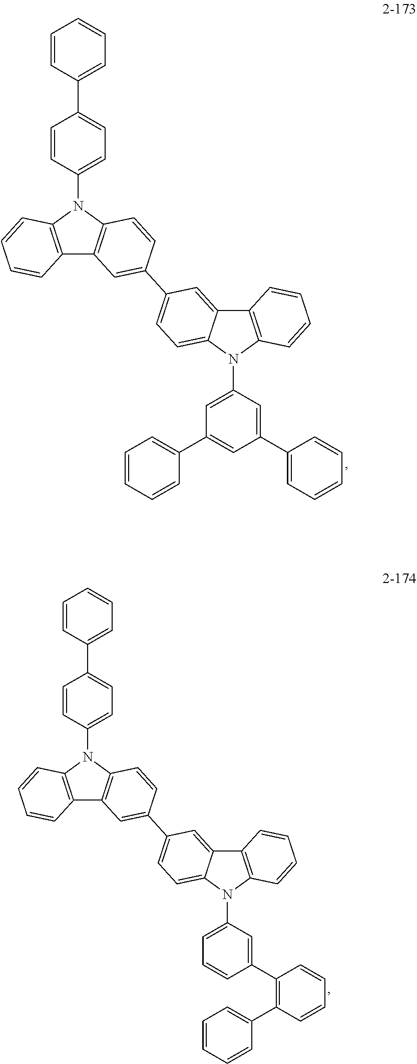 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6-c6ccccc6)c5)ccc43)cc2)cc1]

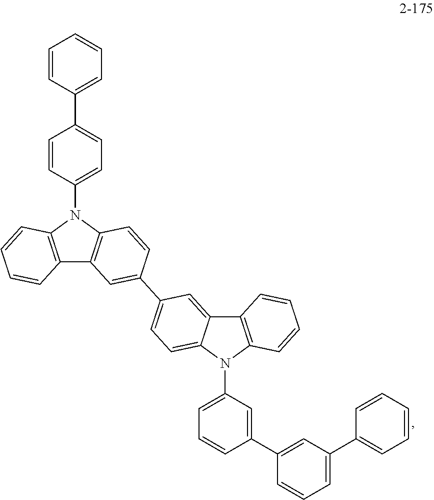 [SMILES: c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1]

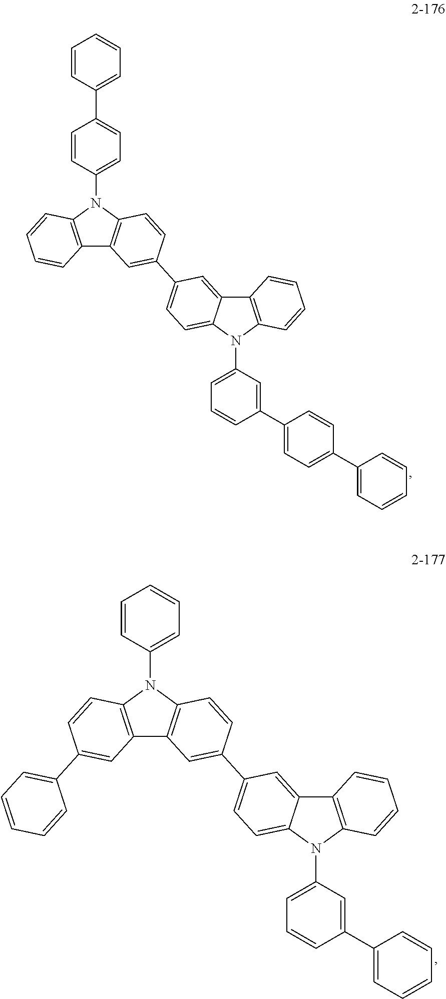 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2cccc(-n3c4ccccc4c4cc(-c5ccc6c(c5)c5cc(-c7ccccc7)ccc5n6-c5ccccc5)ccc43)c2)cc1]

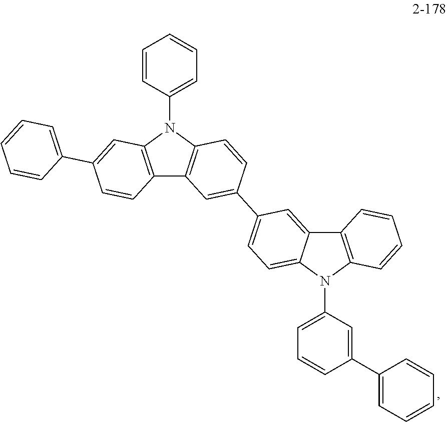 [SMILES: c1ccc(-c2cccc(-n3c4ccccc4c4cc(-c5ccc6c(c5)c5ccc(-c7ccccc7)cc5n6-c5ccccc5)ccc43)c2)cc1]

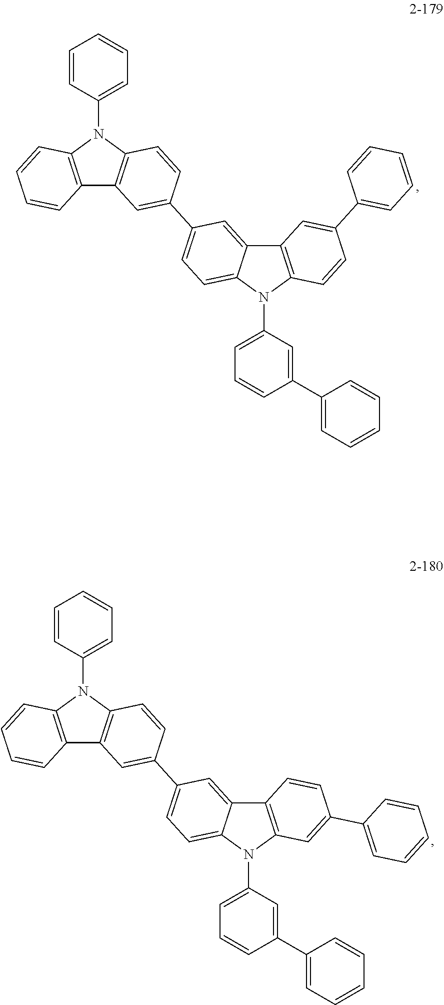 [SMILES: c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2cccc(-n3c4ccc(-c5ccc6c(c5)c5ccccc5n6-c5ccccc5)cc4c4ccc(-c5ccccc5)cc43)c2)cc1]

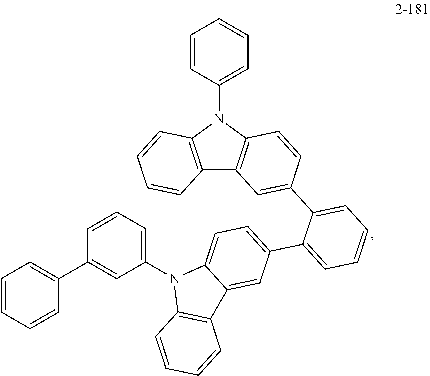 [SMILES: c1ccc(-c2cccc(-n3c4ccccc4c4cc(-c5ccccc5-c5ccc6c(c5)c5ccccc5n6-c5ccccc5)ccc43)c2)cc1]

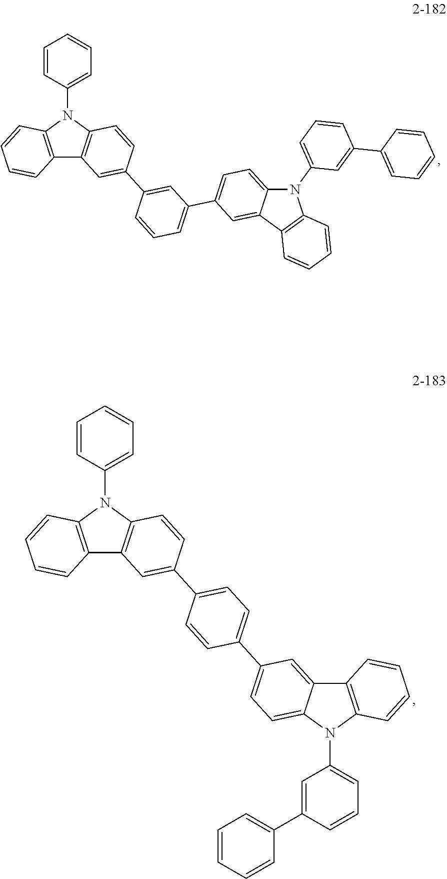 [SMILES: c1ccc(-c2cccc(-n3c4ccccc4c4cc(-c5cccc(-c6ccc7c(c6)c6ccccc6n7-c6ccccc6)c5)ccc43)c2)cc1 ; c1ccc(-c2cccc(-n3c4ccccc4c4cc(-c5ccc(-c6ccc7c(c6)c6ccccc6n7-c6ccccc6)cc5)ccc43)c2)cc1]

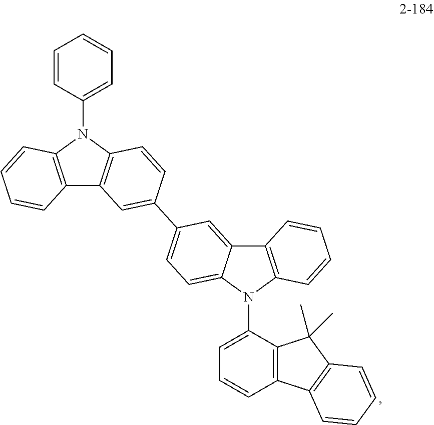 [SMILES: CC1(C)c2ccccc2-c2cccc(-n3c4ccccc4c4cc(-c5ccc6c(c5)c5ccccc5n6-c5ccccc5)ccc43)c21]

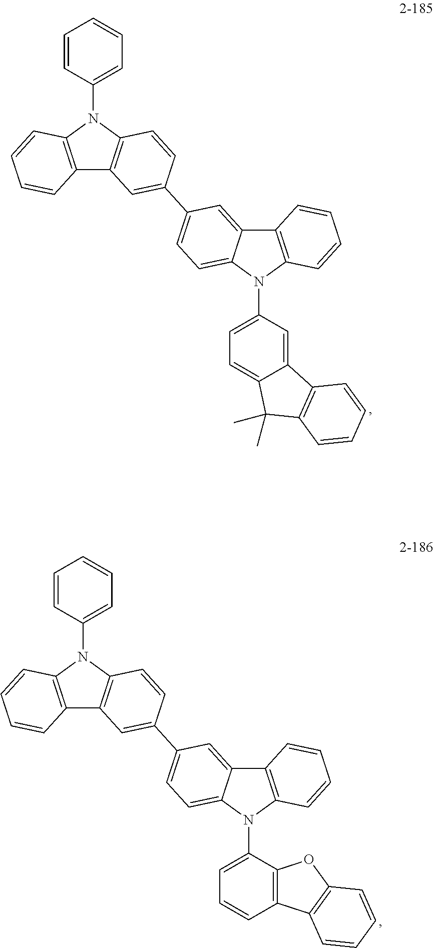 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4cccc5c4oc4ccccc45)ccc32)cc1]

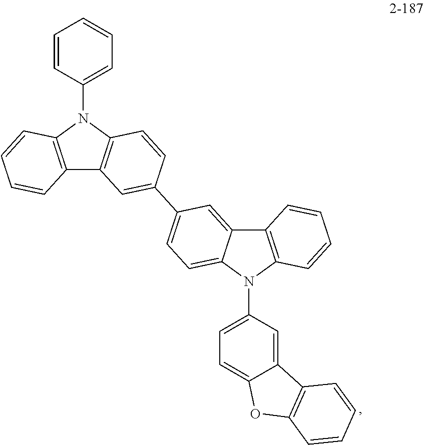 [SMILES: c1ccc(-n2c3ccccc3c3cc(-c4ccc5c(c4)c4ccccc4n5-c4ccc5oc6ccccc6c5c4)ccc32)cc1]

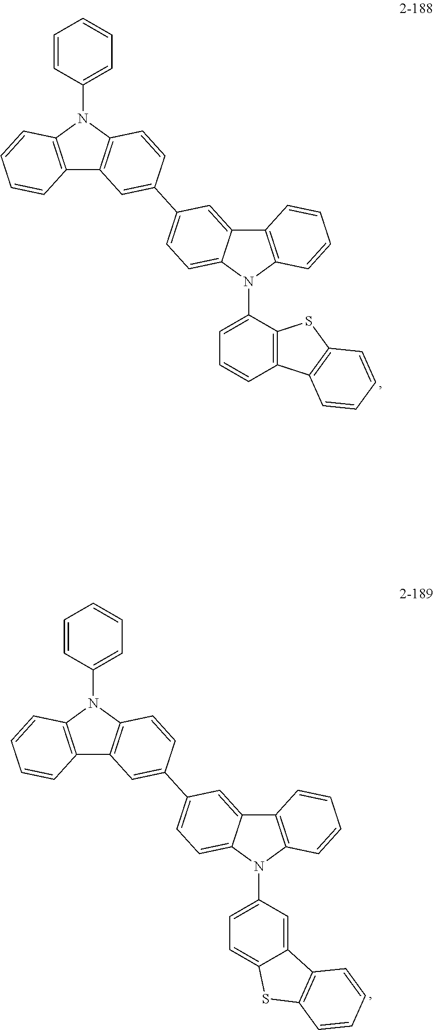 [SMILES: c1ccc(-n2c3ccccc3c3cc(-c4ccc5c(c4)c4ccccc4n5-c4cccc5c4sc4ccccc45)ccc32)cc1 ; c1ccc(-n2c3ccccc3c3cc(-c4ccc5c(c4)c4ccccc4n5-c4ccc5sc6ccccc6c5c4)ccc32)cc1]

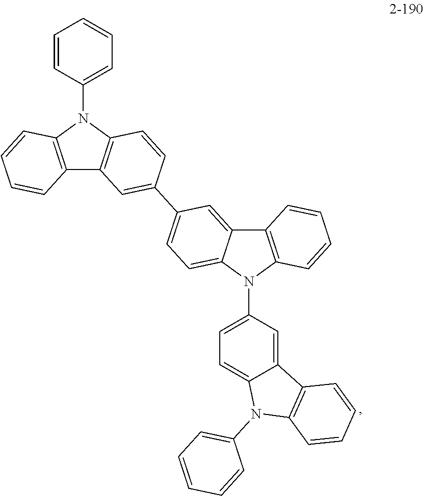 [SMILES: c1ccc(-n2c3ccccc3c3cc(-c4ccc5c(c4)c4ccccc4n5-c4ccc5c(c4)c4ccccc4n5-c4ccccc4)ccc32)cc1]

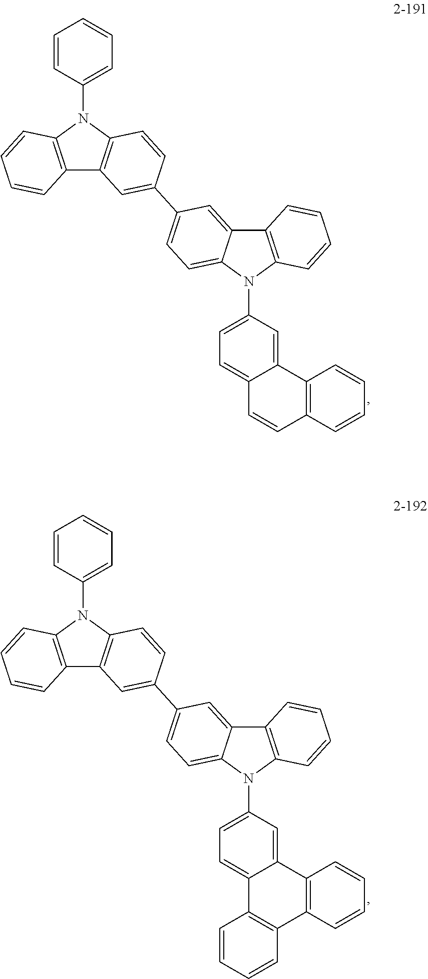 [SMILES: c1ccc(-n2c3ccccc3c3cc(-c4ccc5c(c4)c4ccccc4n5-c4ccc5ccc6ccccc6c5c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

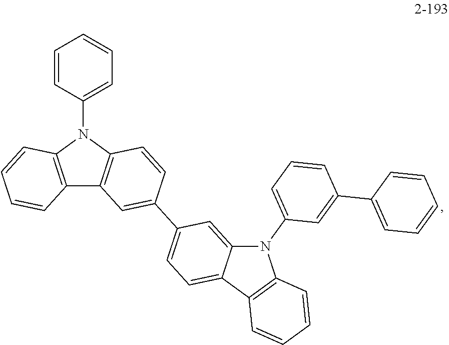 [SMILES: c1ccc(-c2cccc(-n3c4ccccc4c4ccc(-c5ccc6c(c5)c5ccccc5n6-c5ccccc5)cc43)c2)cc1]

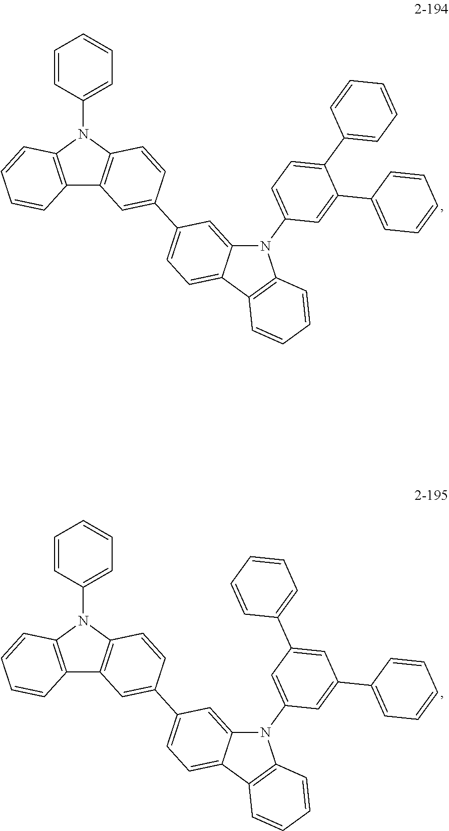 [SMILES: c1ccc(-c2ccc(-n3c4ccccc4c4ccc(-c5ccc6c(c5)c5ccccc5n6-c5ccccc5)cc43)cc2-c2ccccc2)cc1 ; c1ccc(-c2cc(-c3ccccc3)cc(-n3c4ccccc4c4ccc(-c5ccc6c(c5)c5ccccc5n6-c5ccccc5)cc43)c2)cc1]

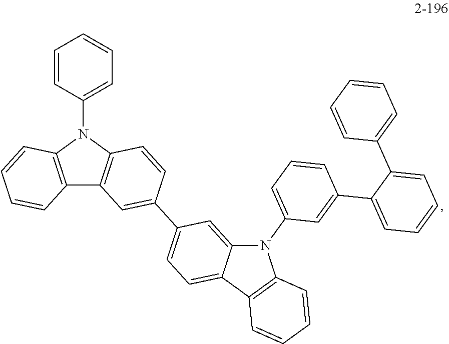 [SMILES: c1ccc(-c2ccccc2-c2cccc(-n3c4ccccc4c4ccc(-c5ccc6c(c5)c5ccccc5n6-c5ccccc5)cc43)c2)cc1]

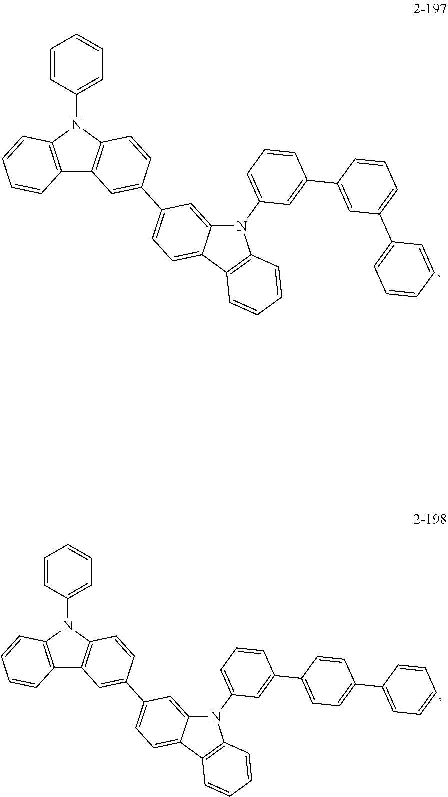 [SMILES: c1ccc(-c2cccc(-c3cccc(-n4c5ccccc5c5ccc(-c6ccc7c(c6)c6ccccc6n7-c6ccccc6)cc54)c3)c2)cc1 ; c1ccc(-c2ccc(-c3cccc(-n4c5ccccc5c5ccc(-c6ccc7c(c6)c6ccccc6n7-c6ccccc6)cc54)c3)cc2)cc1]

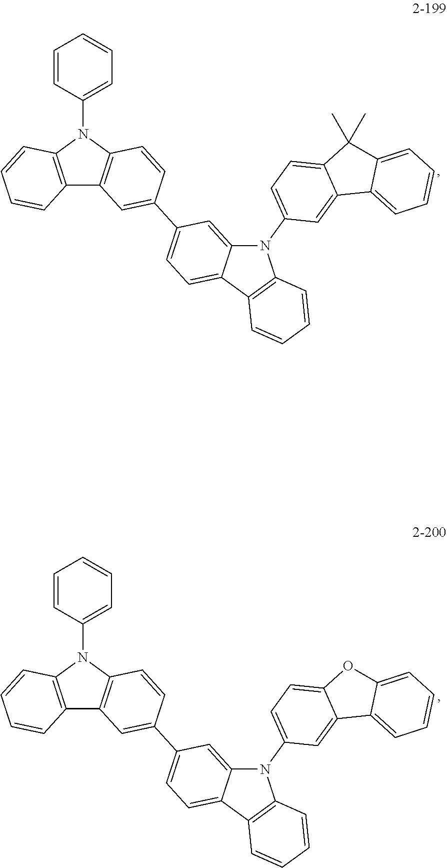 [SMILES: CC1(C)c2ccccc2-c2cc(-n3c4ccccc4c4ccc(-c5ccc6c(c5)c5ccccc5n6-c5ccccc5)cc43)ccc21 ; c1ccc(-n2c3ccccc3c3cc(-c4ccc5c6ccccc6n(-c6ccc7oc8ccccc8c7c6)c5c4)ccc32)cc1]

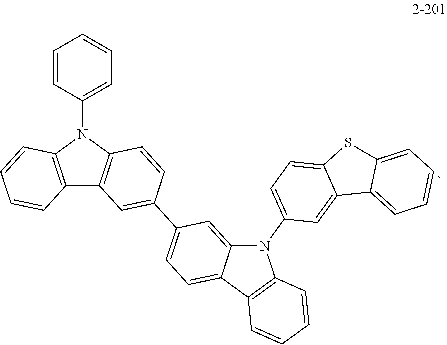 [SMILES: c1ccc(-n2c3ccccc3c3cc(-c4ccc5c6ccccc6n(-c6ccc7sc8ccccc8c7c6)c5c4)ccc32)cc1]

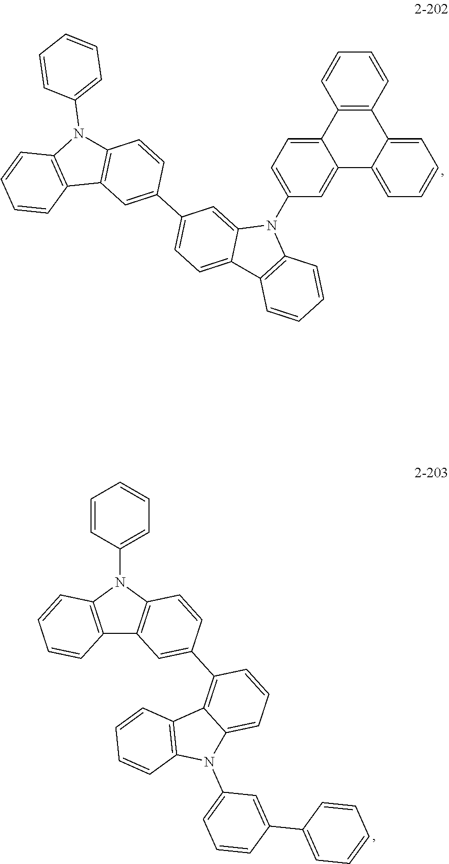 [SMILES: c1ccc(-n2c3ccccc3c3cc(-c4ccc5c6ccccc6n(-c6ccc7c8ccccc8c8ccccc8c7c6)c5c4)ccc32)cc1 ; c1ccc(-c2cccc(-n3c4ccccc4c4c(-c5ccc6c(c5)c5ccccc5n6-c5ccccc5)cccc43)c2)cc1]

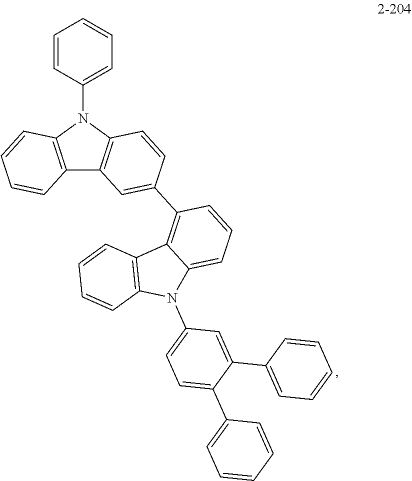 [SMILES: c1ccc(-c2ccc(-n3c4ccccc4c4c(-c5ccc6c(c5)c5ccccc5n6-c5ccccc5)cccc43)cc2-c2ccccc2)cc1]

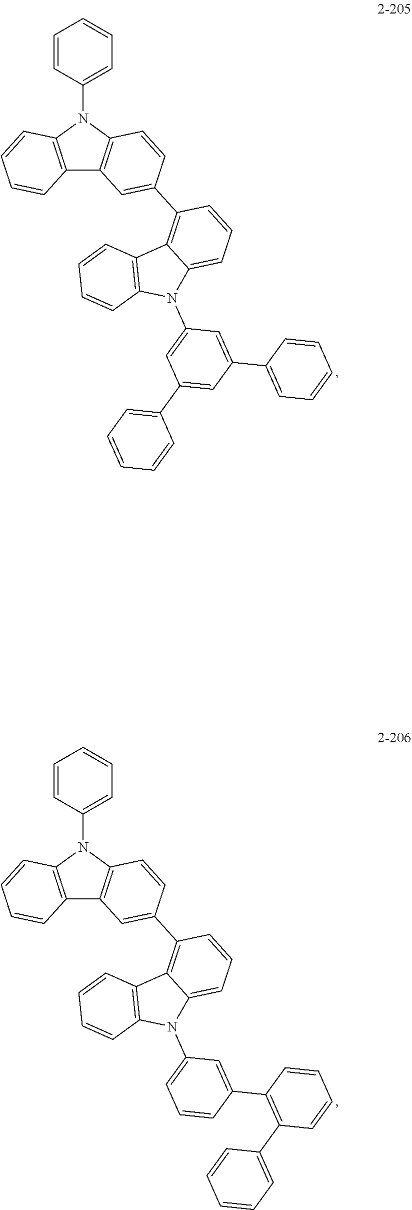 [SMILES: c1ccc(-c2cc(-c3ccccc3)cc(-n3c4ccccc4c4c(-c5ccc6c(c5)c5ccccc5n6-c5ccccc5)cccc43)c2)cc1 ; c1ccc(-c2ccccc2-c2cccc(-n3c4ccccc4c4c(-c5ccc6c(c5)c5ccccc5n6-c5ccccc5)cccc43)c2)cc1]

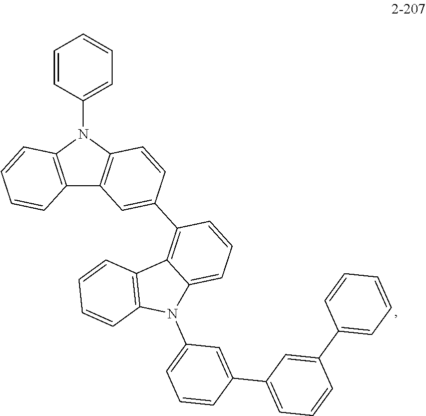 [SMILES: c1ccc(-c2cccc(-c3cccc(-n4c5ccccc5c5c(-c6ccc7c(c6)c6ccccc6n7-c6ccccc6)cccc54)c3)c2)cc1]

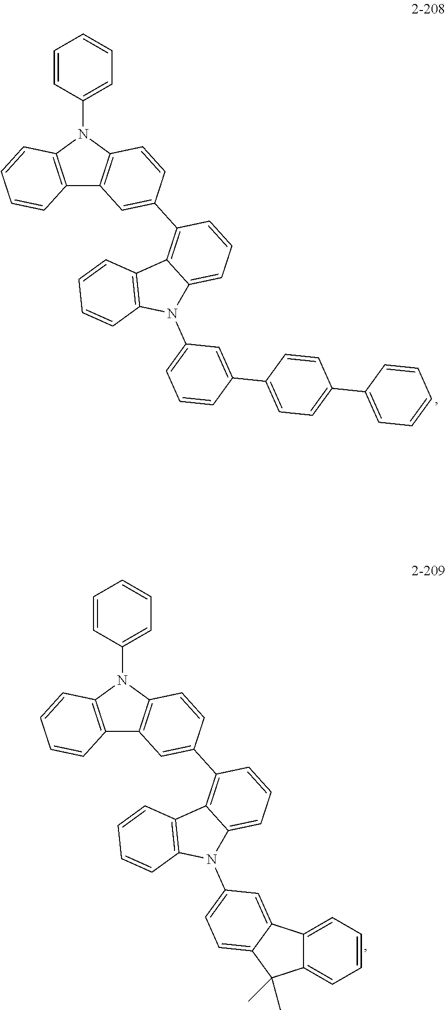 [SMILES: c1ccc(-c2ccc(-c3cccc(-n4c5ccccc5c5c(-c6ccc7c(c6)c6ccccc6n7-c6ccccc6)cccc54)c3)cc2)cc1 ; CC1(C)c2ccccc2-c2cc(-n3c4ccccc4c4c(-c5ccc6c(c5)c5ccccc5n6-c5ccccc5)cccc43)ccc21]

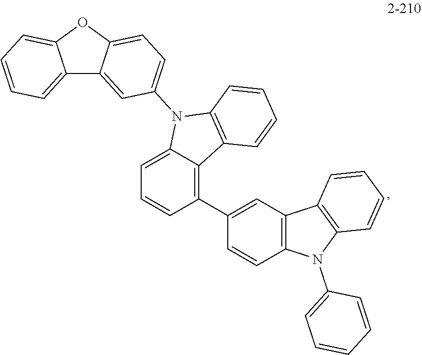 [SMILES: c1ccc(-n2c3ccccc3c3cc(-c4cccc5c4c4ccccc4n5-c4ccc5oc6ccccc6c5c4)ccc32)cc1]

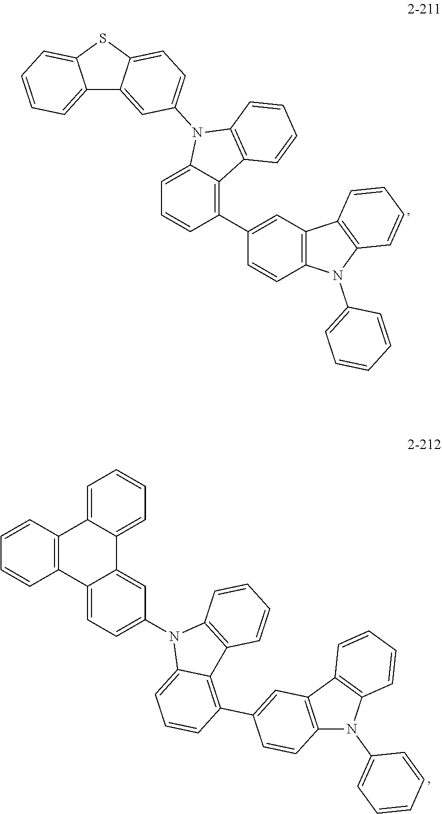 [SMILES: c1ccc(-n2c3ccccc3c3cc(-c4cccc5c4c4ccccc4n5-c4ccc5sc6ccccc6c5c4)ccc32)cc1 ; c1ccc(-n2c3ccccc3c3cc(-c4cccc5c4c4ccccc4n5-c4ccc5c6ccccc6c6ccccc6c5c4)ccc32)cc1]

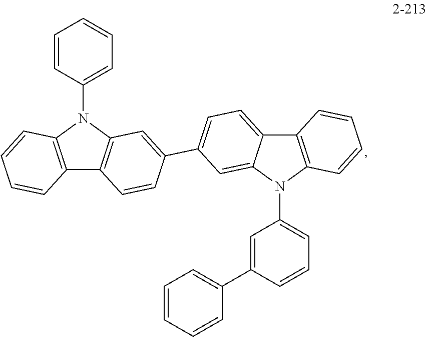 [SMILES: c1ccc(-c2cccc(-n3c4ccccc4c4ccc(-c5ccc6c7ccccc7n(-c7ccccc7)c6c5)cc43)c2)cc1]

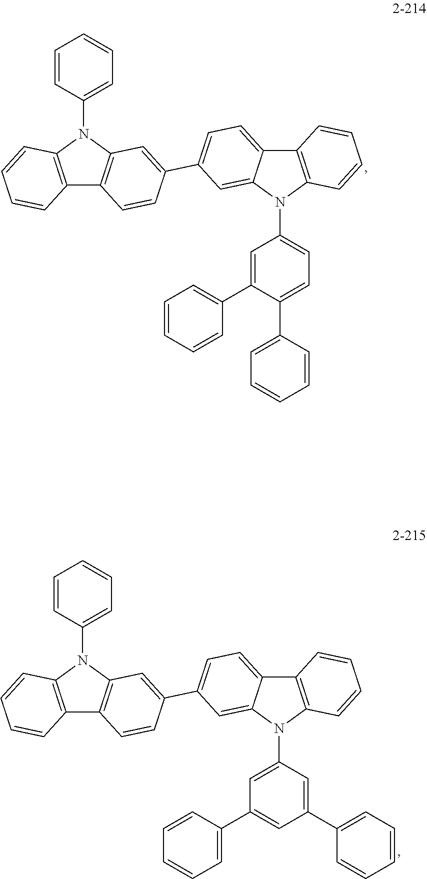 [SMILES: c1ccc(-c2ccc(-n3c4ccccc4c4ccc(-c5ccc6c7ccccc7n(-c7ccccc7)c6c5)cc43)cc2-c2ccccc2)cc1 ; c1ccc(-c2cc(-c3ccccc3)cc(-n3c4ccccc4c4ccc(-c5ccc6c7ccccc7n(-c7ccccc7)c6c5)cc43)c2)cc1]

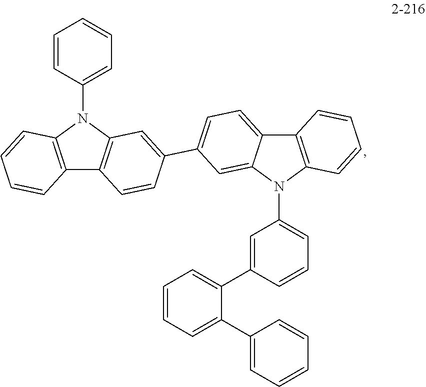 [SMILES: c1ccc(-c2ccccc2-c2cccc(-n3c4ccccc4c4ccc(-c5ccc6c7ccccc7n(-c7ccccc7)c6c5)cc43)c2)cc1]

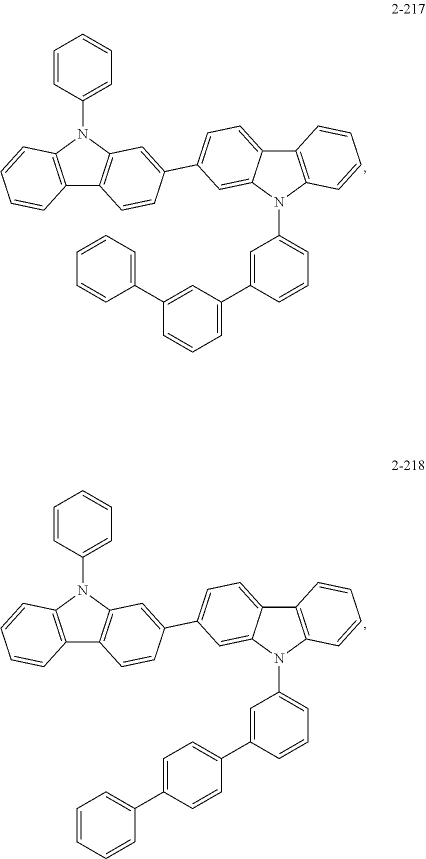 [SMILES: c1ccc(-c2cccc(-c3cccc(-n4c5ccccc5c5ccc(-c6ccc7c8ccccc8n(-c8ccccc8)c7c6)cc54)c3)c2)cc1 ; c1ccc(-c2ccc(-c3cccc(-n4c5ccccc5c5ccc(-c6ccc7c8ccccc8n(-c8ccccc8)c7c6)cc54)c3)cc2)cc1]

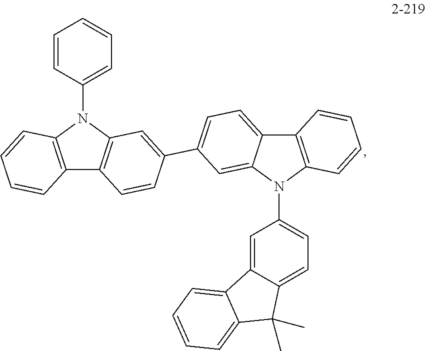 [SMILES: CC1(C)c2ccccc2-c2cc(-n3c4ccccc4c4ccc(-c5ccc6c7ccccc7n(-c7ccccc7)c6c5)cc43)ccc21]

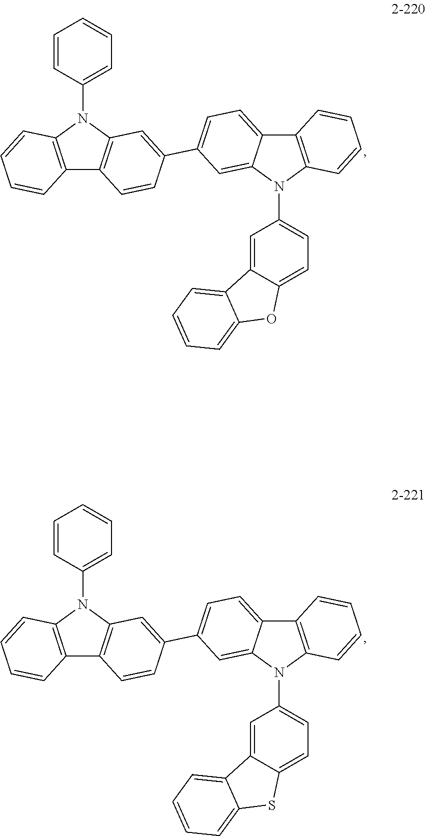 [SMILES: c1ccc(-n2c3ccccc3c3ccc(-c4ccc5c6ccccc6n(-c6ccc7oc8ccccc8c7c6)c5c4)cc32)cc1 ; c1ccc(-n2c3ccccc3c3ccc(-c4ccc5c6ccccc6n(-c6ccc7sc8ccccc8c7c6)c5c4)cc32)cc1]

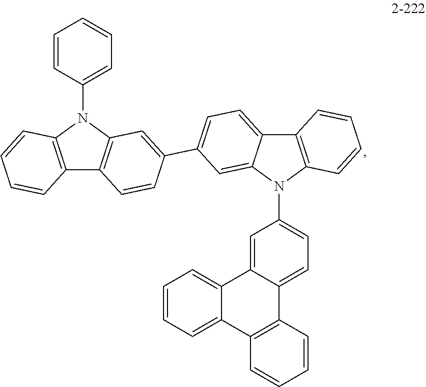 [SMILES: c1ccc(-n2c3ccccc3c3ccc(-c4ccc5c6ccccc6n(-c6ccc7c8ccccc8c8ccccc8c7c6)c5c4)cc32)cc1]

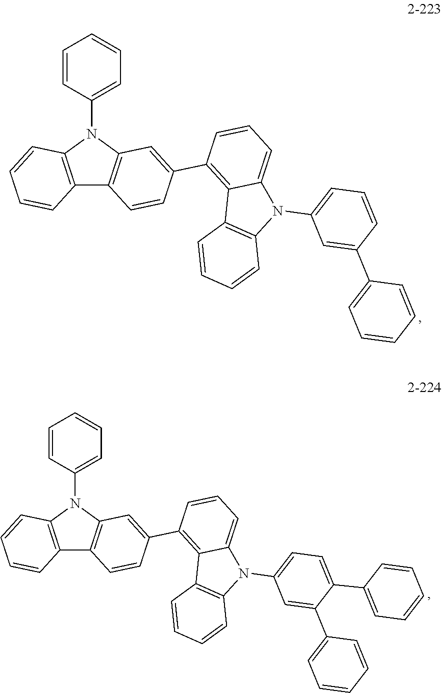 [SMILES: c1ccc(-c2cccc(-n3c4ccccc4c4c(-c5ccc6c7ccccc7n(-c7ccccc7)c6c5)cccc43)c2)cc1 ; c1ccc(-c2ccc(-n3c4ccccc4c4c(-c5ccc6c7ccccc7n(-c7ccccc7)c6c5)cccc43)cc2-c2ccccc2)cc1]

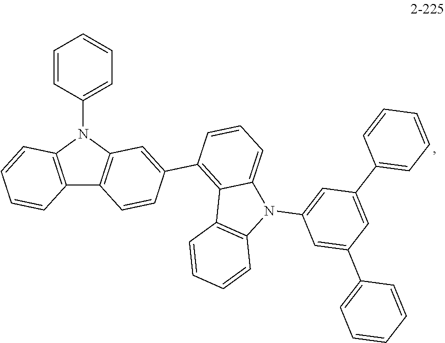 [SMILES: c1ccc(-c2cc(-c3ccccc3)cc(-n3c4ccccc4c4c(-c5ccc6c7ccccc7n(-c7ccccc7)c6c5)cccc43)c2)cc1]

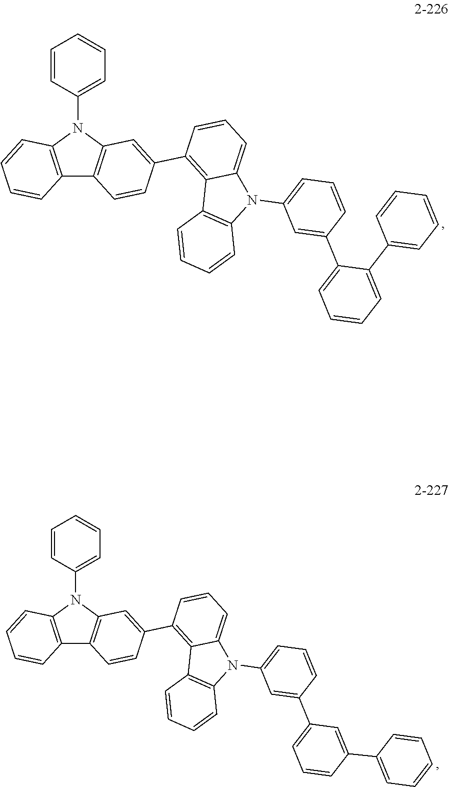 [SMILES: c1ccc(-c2ccccc2-c2cccc(-n3c4ccccc4c4c(-c5ccc6c7ccccc7n(-c7ccccc7)c6c5)cccc43)c2)cc1 ; c1ccc(-c2cccc(-c3cccc(-n4c5ccccc5c5c(-c6ccc7c8ccccc8n(-c8ccccc8)c7c6)cccc54)c3)c2)cc1]

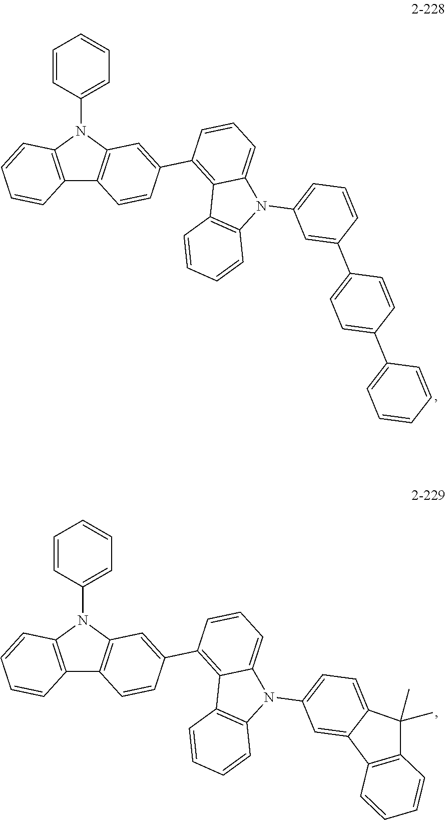 [SMILES: c1ccc(-c2ccc(-c3cccc(-n4c5ccccc5c5c(-c6ccc7c8ccccc8n(-c8ccccc8)c7c6)cccc54)c3)cc2)cc1 ; CC1(C)c2ccccc2-c2cc(-n3c4ccccc4c4c(-c5ccc6c7ccccc7n(-c7ccccc7)c6c5)cccc43)ccc21]

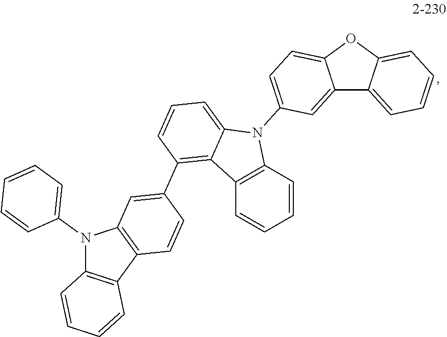 [SMILES: c1ccc(-n2c3ccccc3c3ccc(-c4cccc5c4c4ccccc4n5-c4ccc5oc6ccccc6c5c4)cc32)cc1]

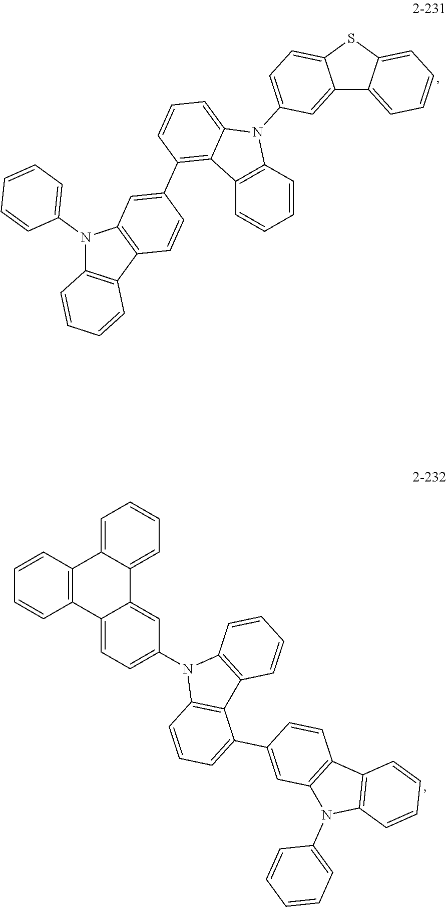 [SMILES: c1ccc(-n2c3ccccc3c3ccc(-c4cccc5c4c4ccccc4n5-c4ccc5sc6ccccc6c5c4)cc32)cc1 ; c1ccc(-n2c3ccccc3c3ccc(-c4cccc5c4c4ccccc4n5-c4ccc5c6ccccc6c6ccccc6c5c4)cc32)cc1]

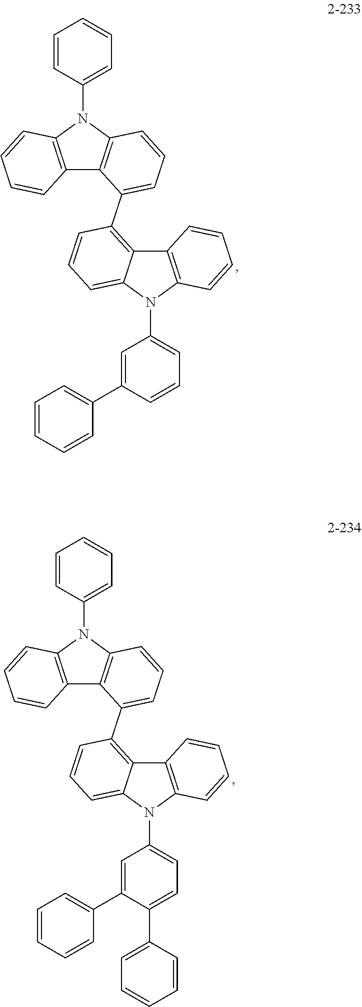 [SMILES: c1ccc(-c2cccc(-n3c4ccccc4c4c(-c5cccc6c5c5ccccc5n6-c5ccccc5)cccc43)c2)cc1 ; c1ccc(-c2ccc(-n3c4ccccc4c4c(-c5cccc6c5c5ccccc5n6-c5ccccc5)cccc43)cc2-c2ccccc2)cc1]

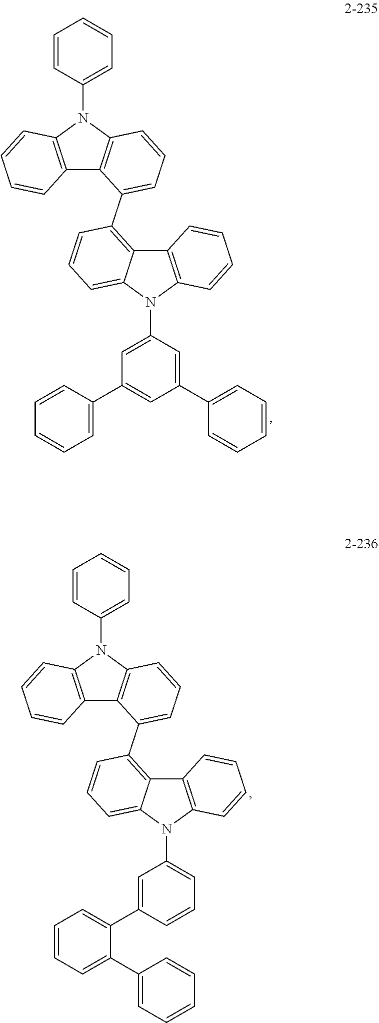 [SMILES: c1ccc(-c2cc(-c3ccccc3)cc(-n3c4ccccc4c4c(-c5cccc6c5c5ccccc5n6-c5ccccc5)cccc43)c2)cc1 ; c1ccc(-c2ccccc2-c2cccc(-n3c4ccccc4c4c(-c5cccc6c5c5ccccc5n6-c5ccccc5)cccc43)c2)cc1]

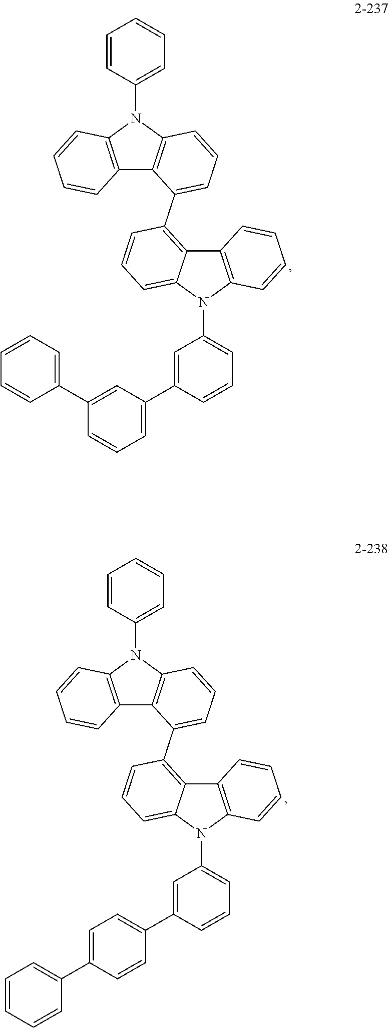 [SMILES: c1ccc(-c2cccc(-c3cccc(-n4c5ccccc5c5c(-c6cccc7c6c6ccccc6n7-c6ccccc6)cccc54)c3)c2)cc1 ; c1ccc(-c2ccc(-c3cccc(-n4c5ccccc5c5c(-c6cccc7c6c6ccccc6n7-c6ccccc6)cccc54)c3)cc2)cc1]

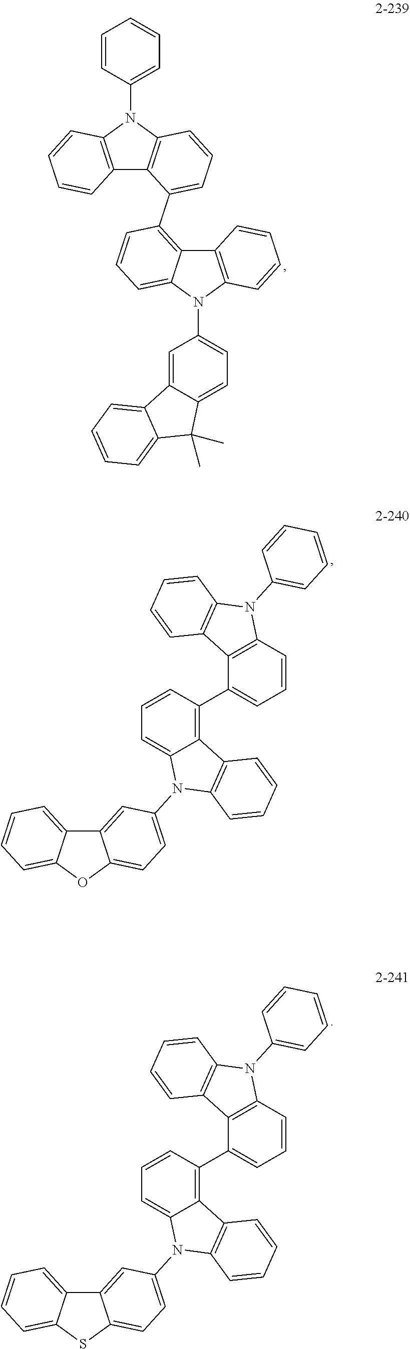 [SMILES: CC1(C)c2ccccc2-c2cc(-n3c4ccccc4c4c(-c5cccc6c5c5ccccc5n6-c5ccccc5)cccc43)ccc21 ; c1ccc(-n2c3ccccc3c3c(-c4cccc5c4c4ccccc4n5-c4ccc5oc6ccccc6c5c4)cccc32)cc1 ; c1ccc(-n2c3ccccc3c3c(-c4cccc5c4c4ccccc4n5-c4ccc5sc6ccccc6c5c4)cccc32)cc1]

27. The electroluminescent device according to claim 1, wherein the second compound is selected from the group consisting of:

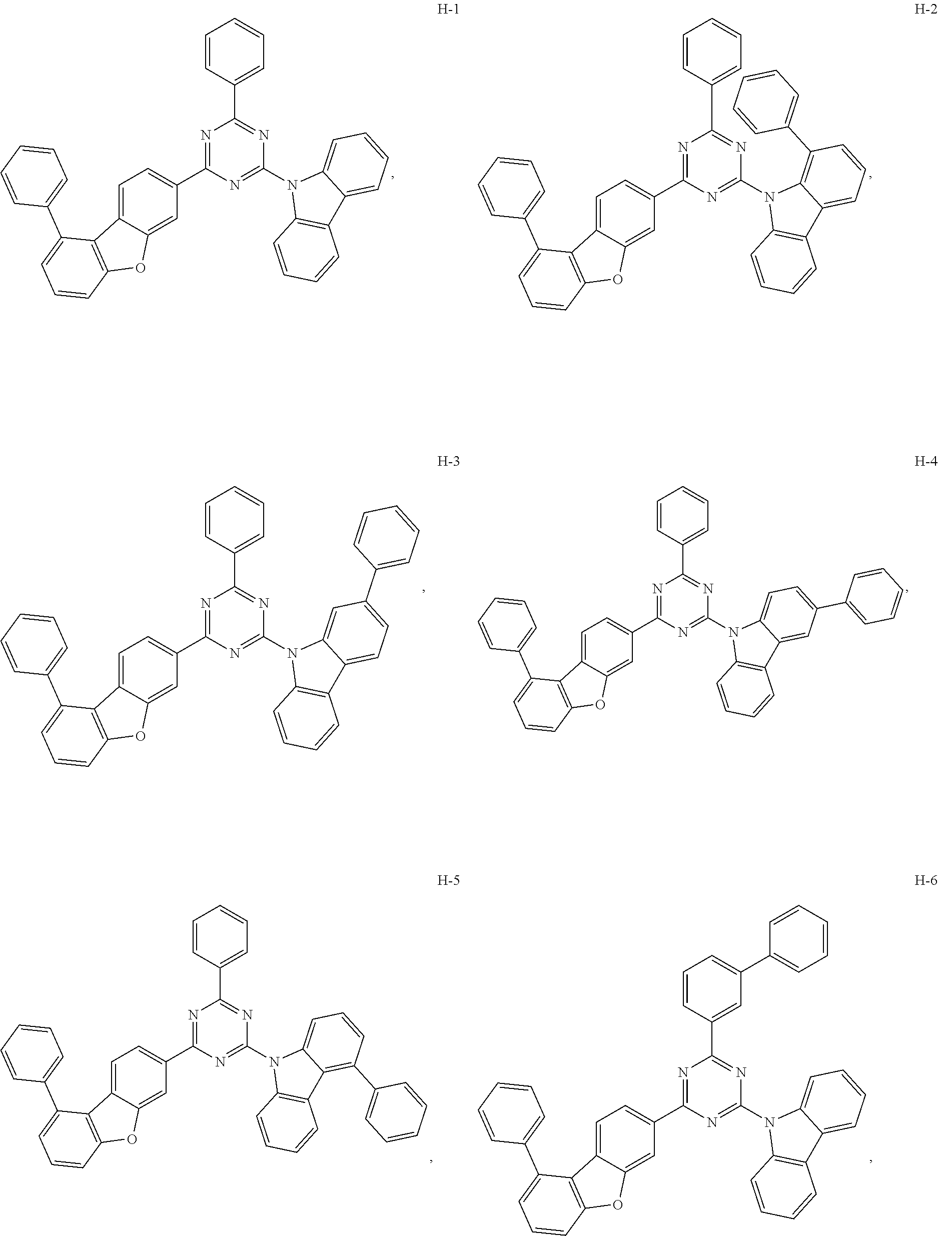

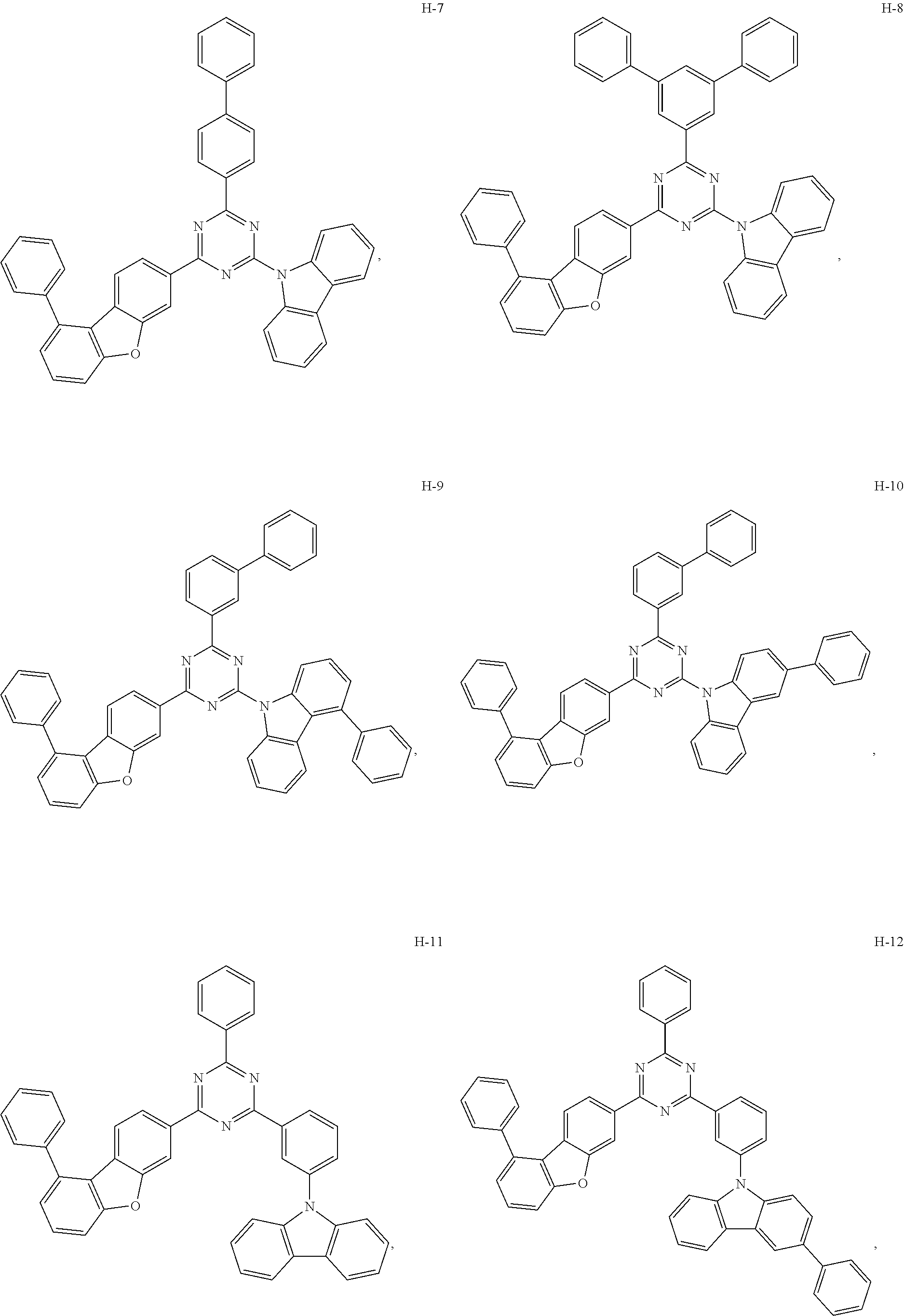

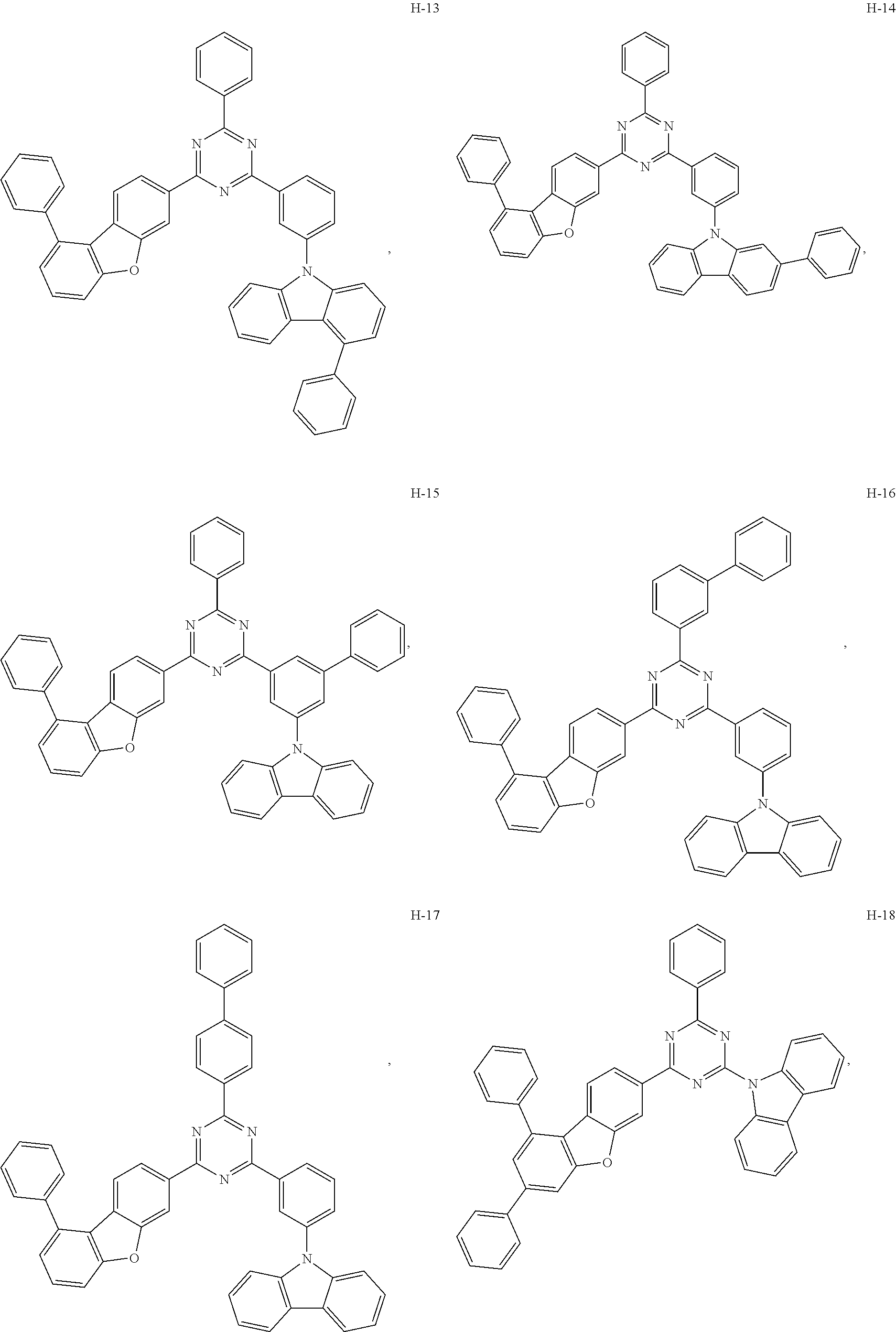

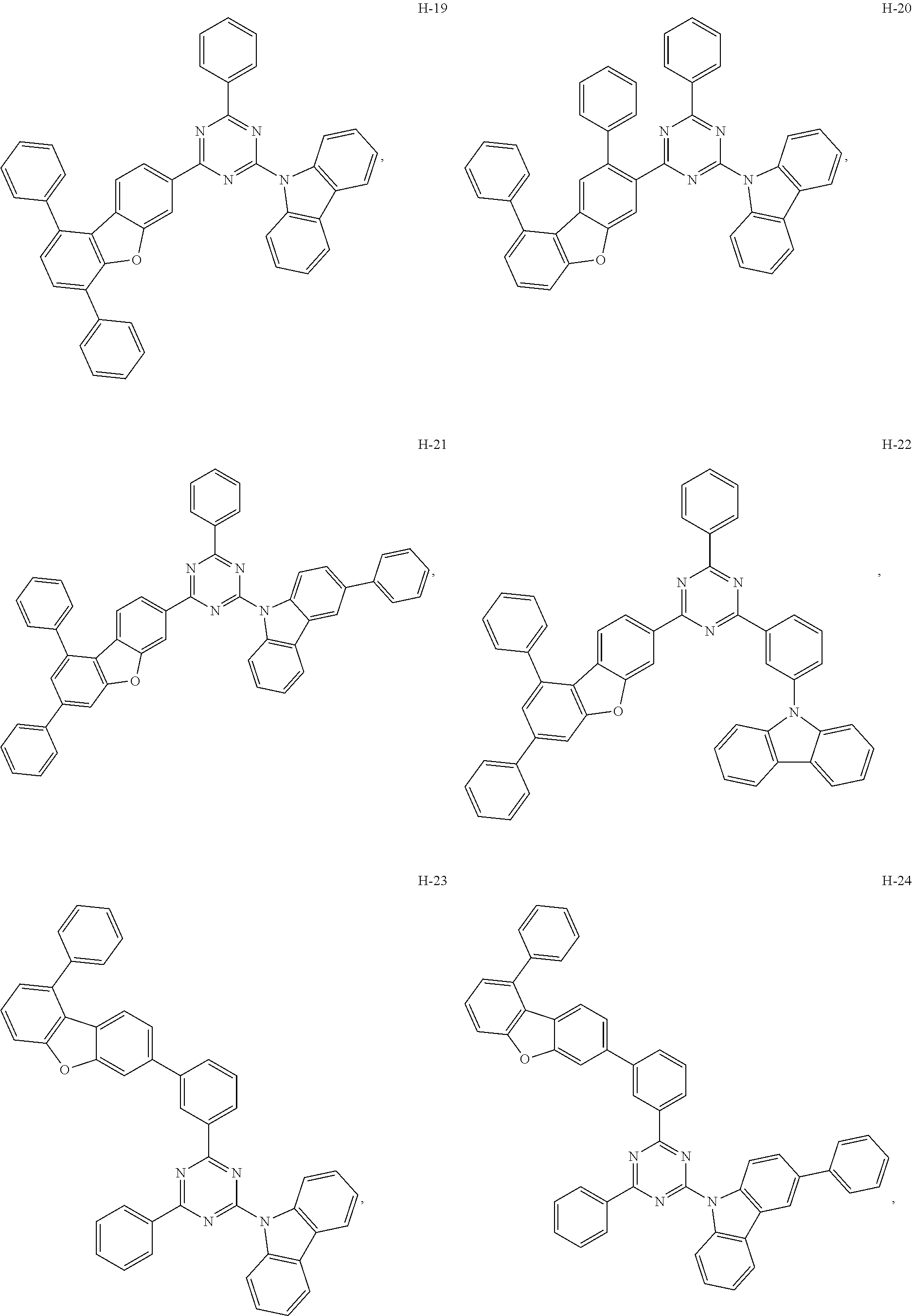

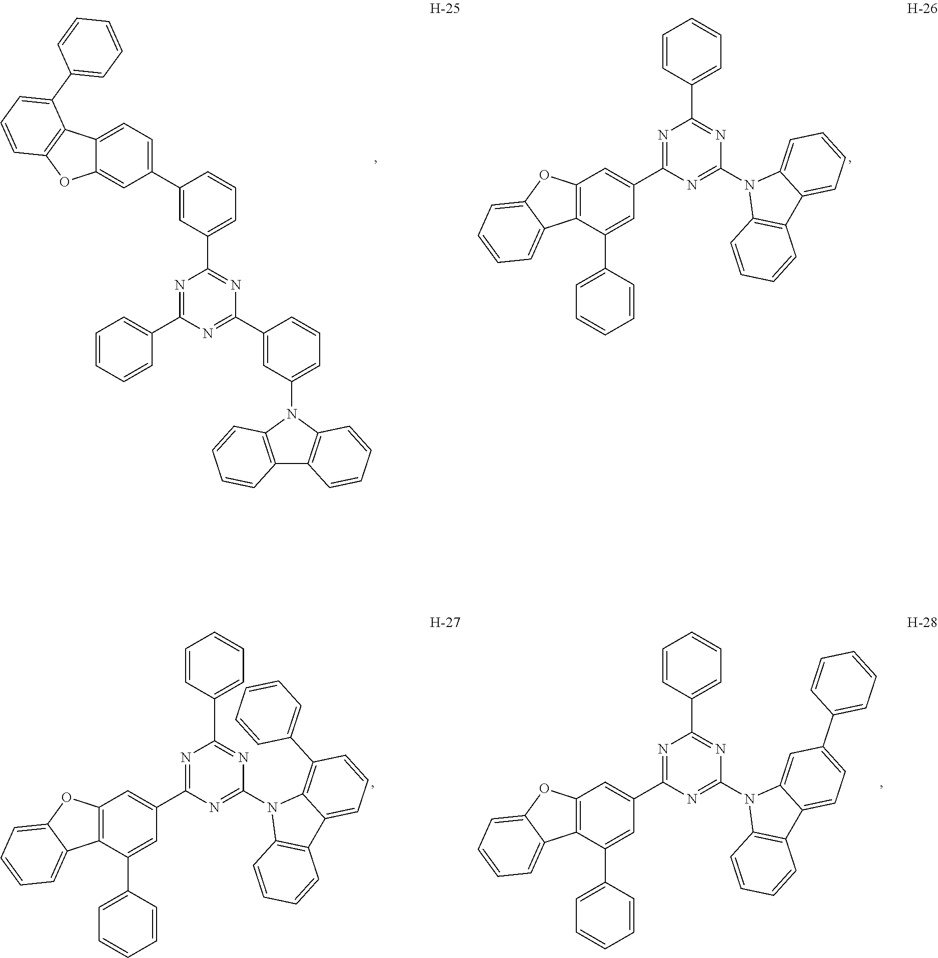 [SMILES: c1ccc(-c2nc(-c3cccc(-c4ccc5c(c4)oc4cccc(-c6ccccc6)c45)c3)nc(-c3cccc(-n4c5ccccc5c5ccccc54)c3)n2)cc1 ; c1ccc(-c2nc(-c3cc(-c4ccccc4)c4c(c3)oc3ccccc34)nc(-n3c4ccccc4c4ccccc43)n2)cc1 ; c1ccc(-c2nc(-c3cc(-c4ccccc4)c4c(c3)oc3ccccc34)nc(-n3c4ccccc4c4cccc(-c5ccccc5)c43)n2)cc1 ; c1ccc(-c2ccc3c4ccccc4n(-c4nc(-c5ccccc5)nc(-c5cc(-c6ccccc6)c6c(c5)oc5ccccc56)n4)c3c2)cc1]

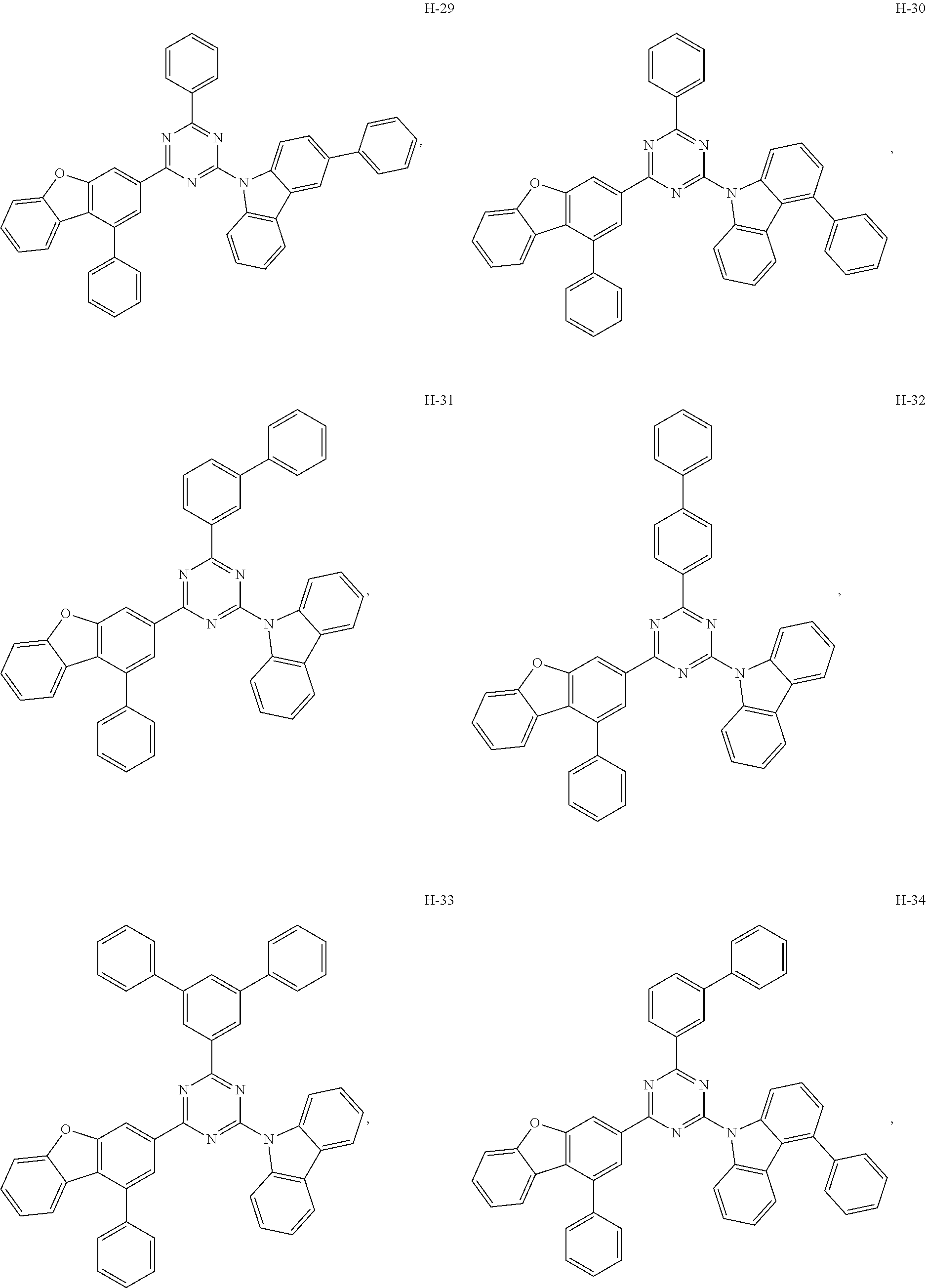

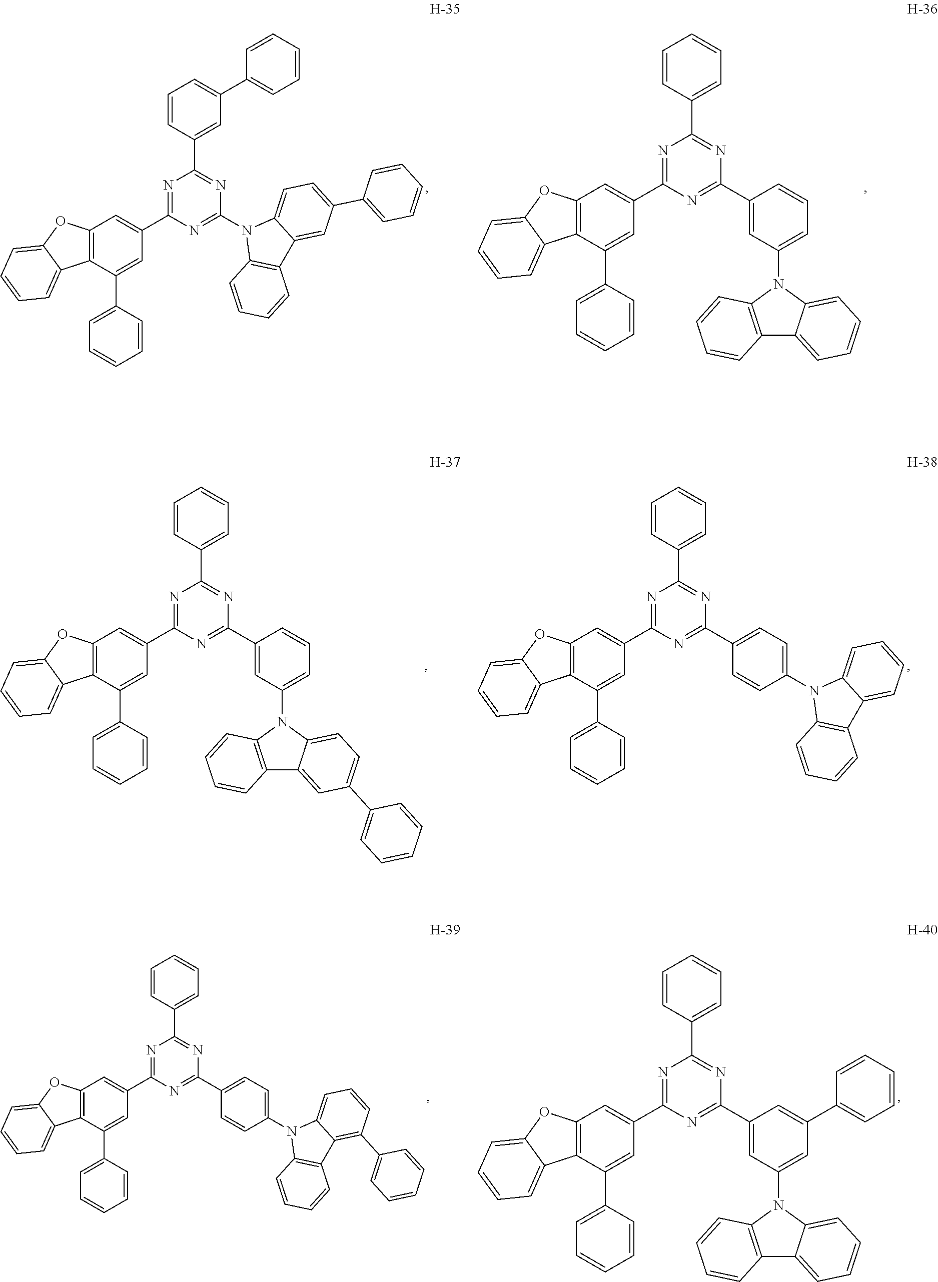

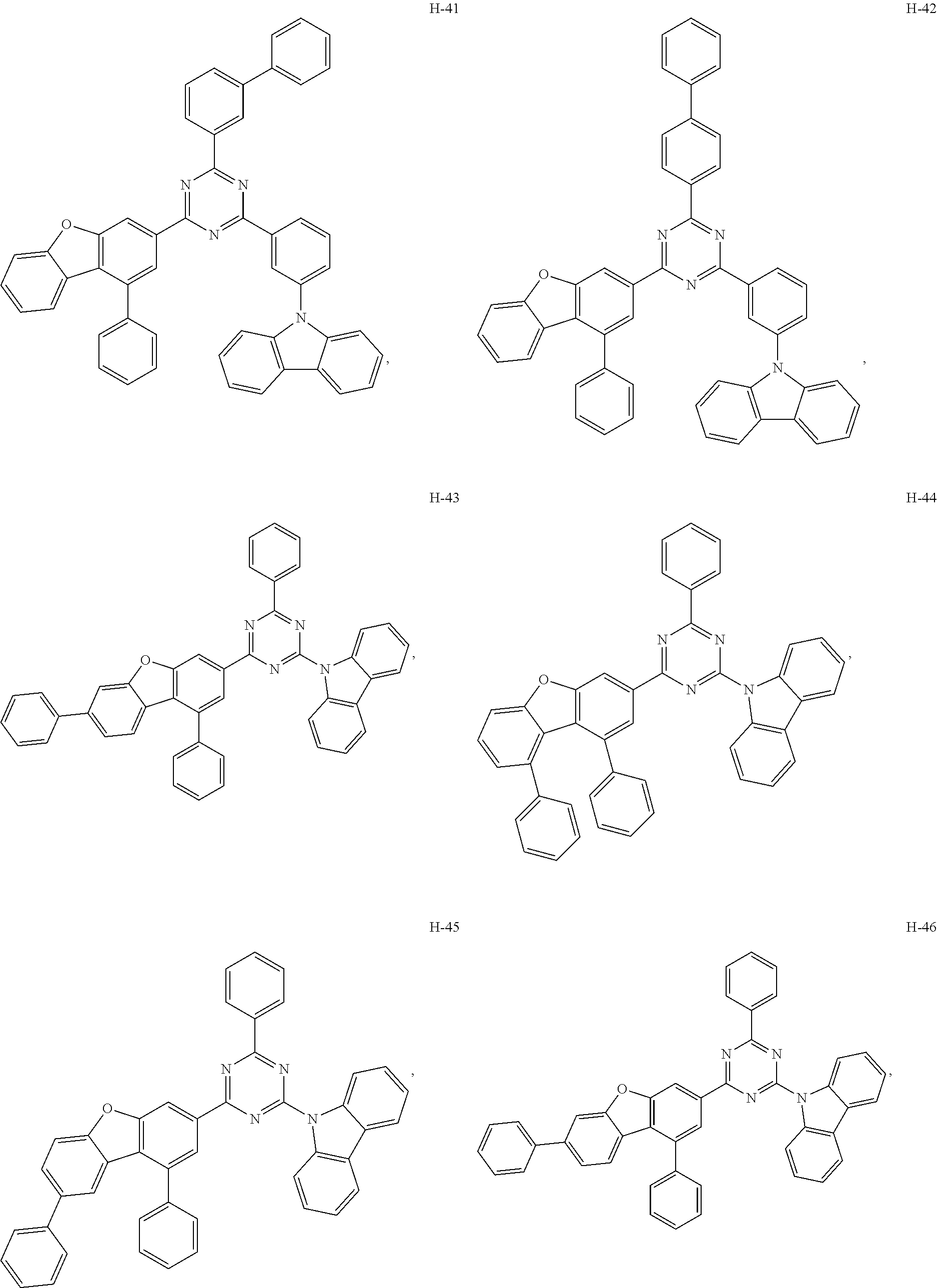

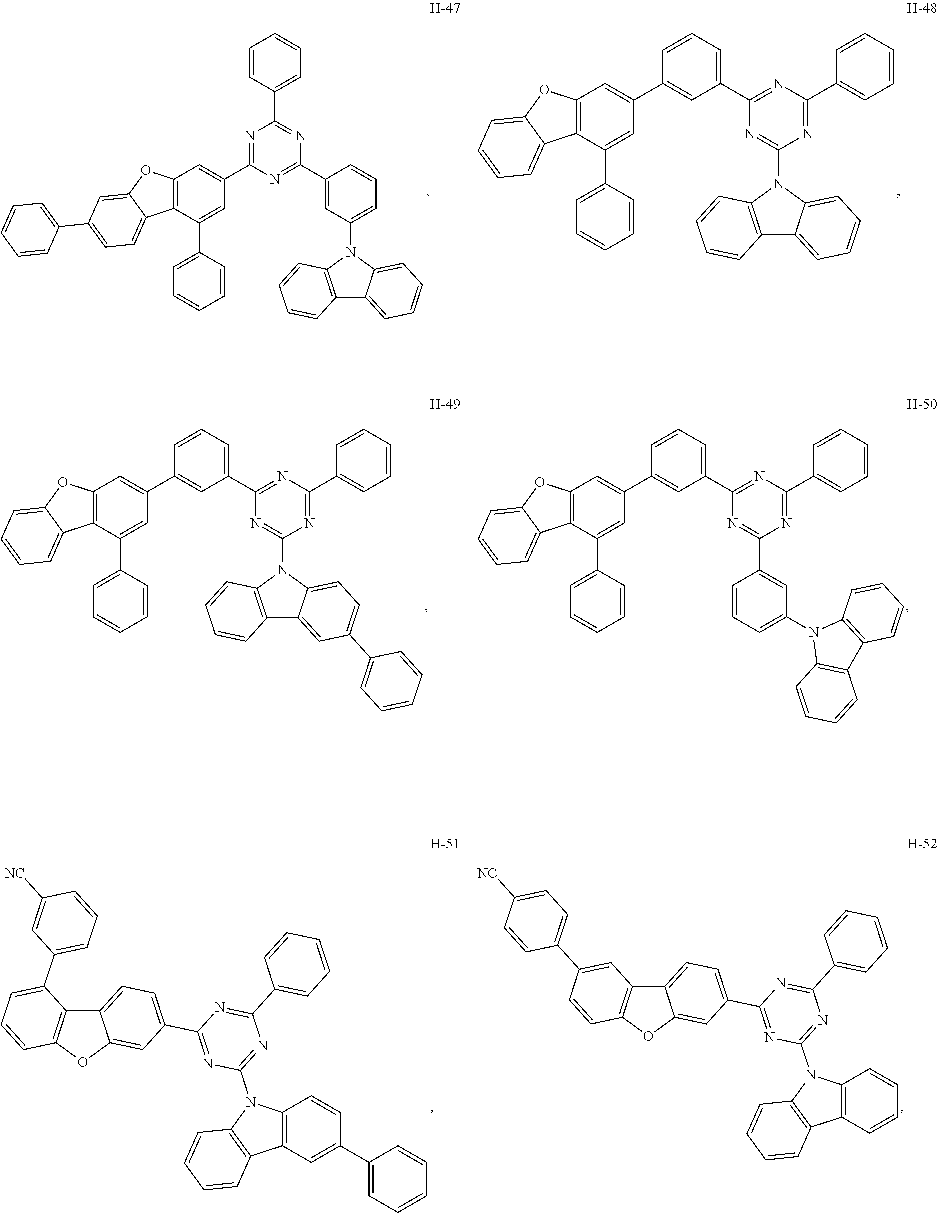

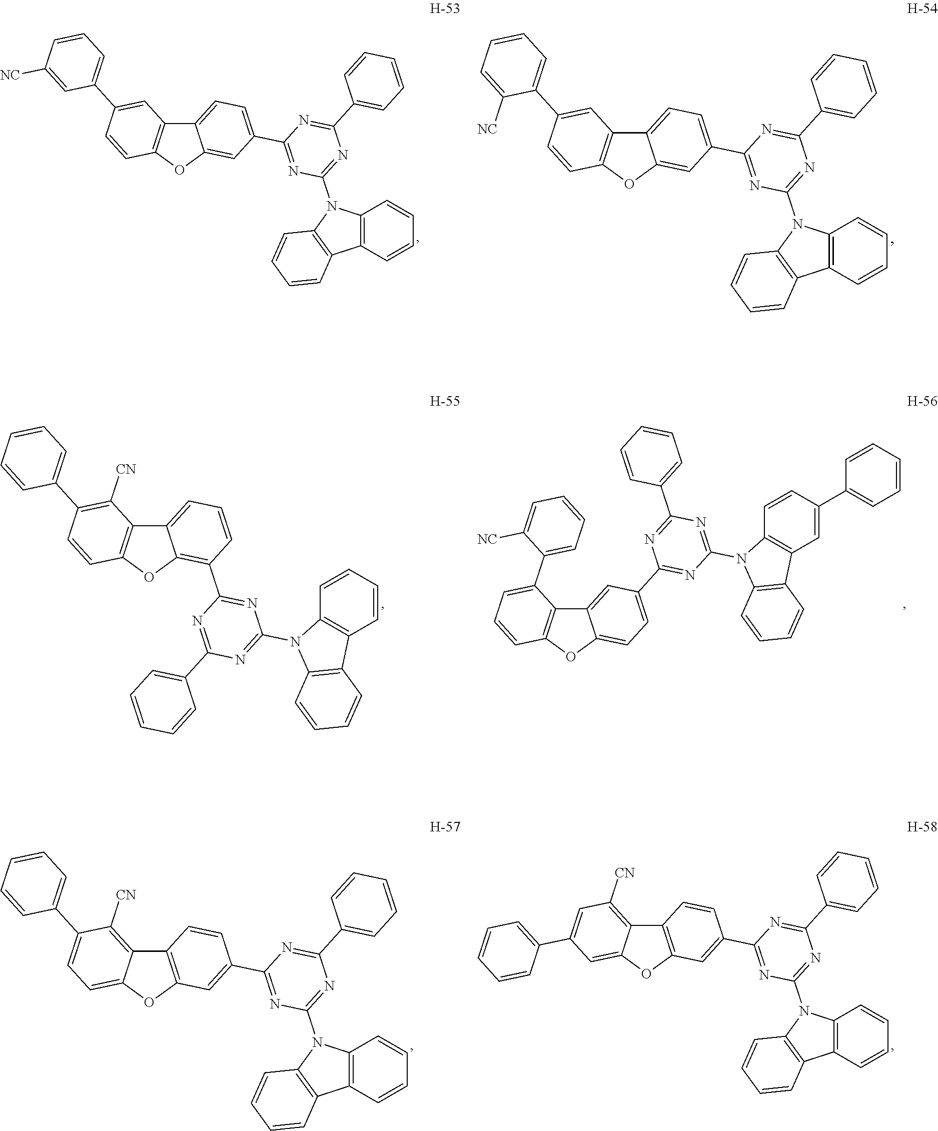 [SMILES: N#Cc1cccc(-c2ccc3oc4cc(-c5nc(-c6ccccc6)nc(-n6c7ccccc7c7ccccc76)n5)ccc4c3c2)c1 ; N#Cc1ccccc1-c1ccc2oc3cc(-c4nc(-c5ccccc5)nc(-n5c6ccccc6c6ccccc65)n4)ccc3c2c1 ; N#Cc1c(-c2ccccc2)ccc2oc3c(-c4nc(-c5ccccc5)nc(-n5c6ccccc6c6ccccc65)n4)cccc3c12 ; N#Cc1ccccc1-c1cccc2oc3ccc(-c4nc(-c5ccccc5)nc(-n5c6ccccc6c6cc(-c7ccccc7)ccc65)n4)cc3c12 ; N#Cc1c(-c2ccccc2)ccc2oc3cc(-c4nc(-c5ccccc5)nc(-n5c6ccccc6c6ccccc65)n4)ccc3c12 ; N#Cc1cc(-c2ccccc2)cc2oc3cc(-c4nc(-c5ccccc5)nc(-n5c6ccccc6c6ccccc65)n4)ccc3c12]

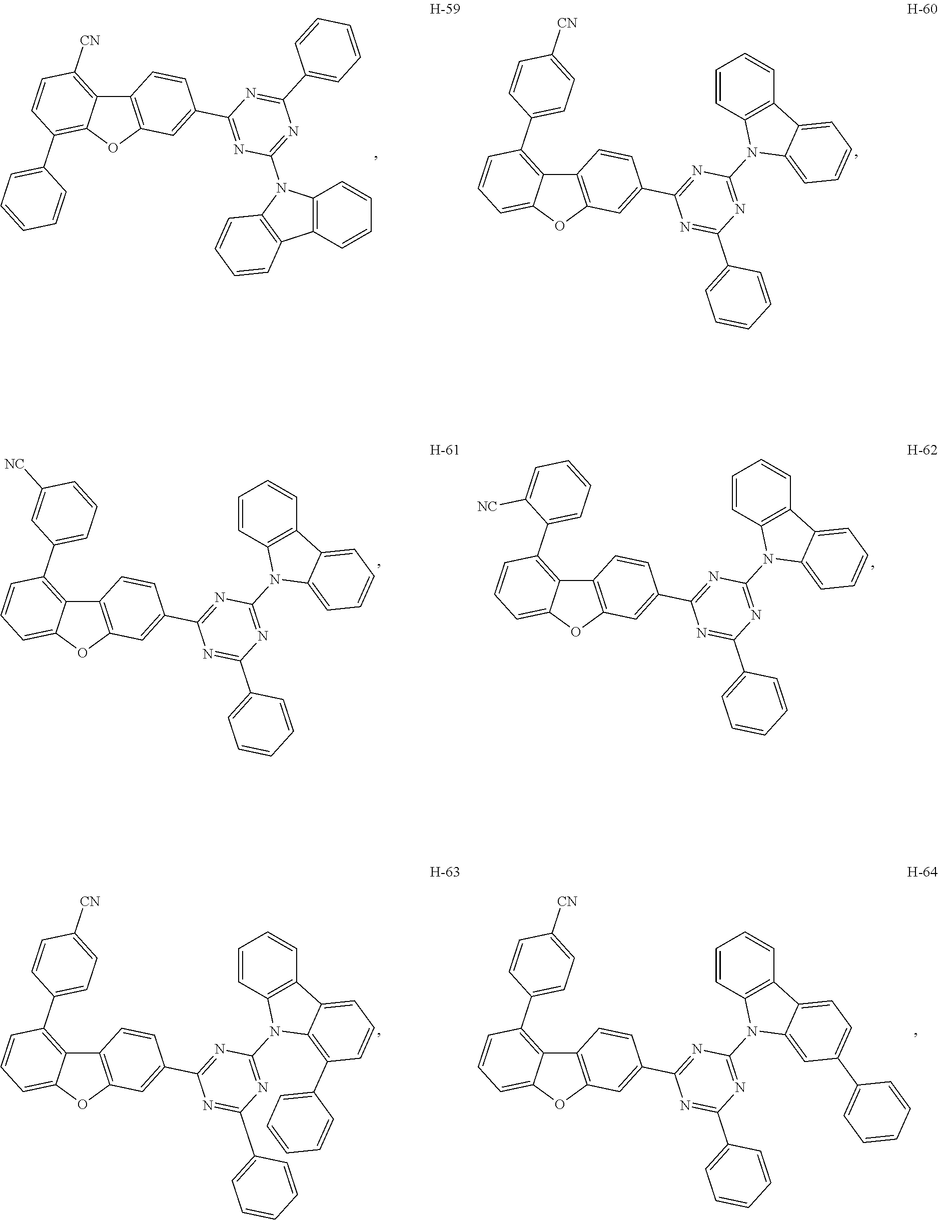 [SMILES: N#Cc1ccc(-c2ccccc2)c2oc3cc(-c4nc(-c5ccccc5)nc(-n5c6ccccc6c6ccccc65)n4)ccc3c12 ; N#Cc1ccc(-c2cccc3oc4cc(-c5nc(-c6ccccc6)nc(-n6c7ccccc7c7ccccc76)n5)ccc4c23)cc1 ; N#Cc1cccc(-c2cccc3oc4cc(-c5nc(-c6ccccc6)nc(-n6c7ccccc7c7ccccc76)n5)ccc4c23)c1 ; N#Cc1ccccc1-c1cccc2oc3cc(-c4nc(-c5ccccc5)nc(-n5c6ccccc6c6ccccc65)n4)ccc3c12 ; N#Cc1ccc(-c2cccc3oc4cc(-c5nc(-c6ccccc6)nc(-n6c7ccccc7c7cccc(-c8ccccc8)c76)n5)ccc4c23)cc1 ; N#Cc1ccc(-c2cccc3oc4cc(-c5nc(-c6ccccc6)nc(-n6c7ccccc7c7ccc(-c8ccccc8)cc76)n5)ccc4c23)cc1]

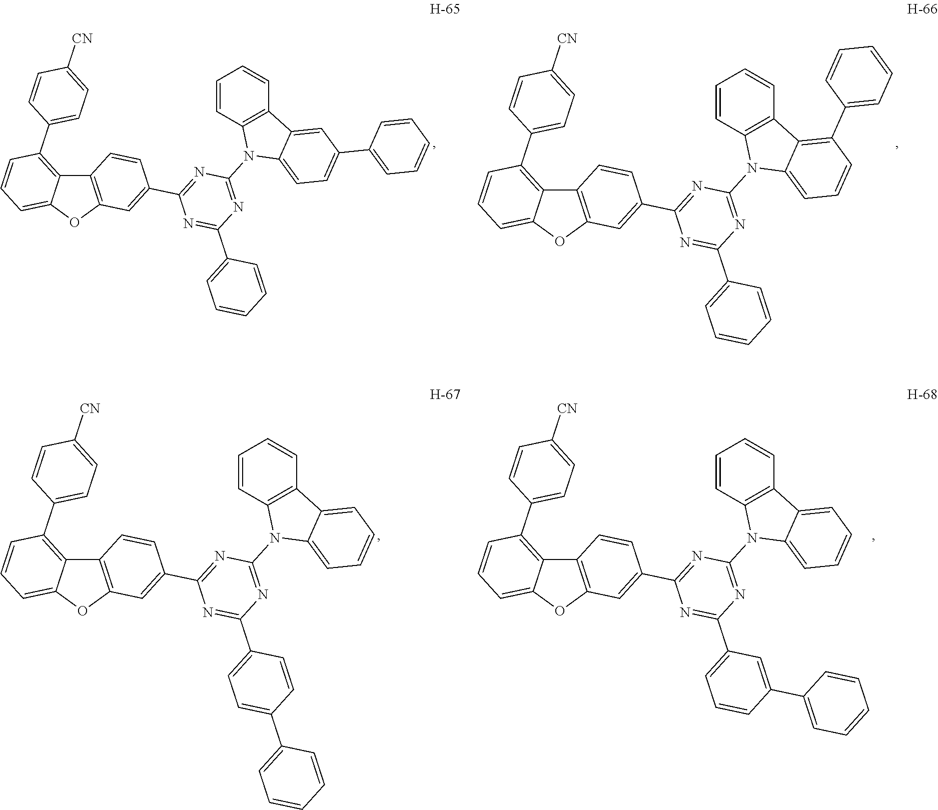 [SMILES: N#Cc1ccc(-c2cccc3oc4cc(-c5nc(-c6ccccc6)nc(-n6c7ccccc7c7cc(-c8ccccc8)ccc76)n5)ccc4c23)cc1 ; N#Cc1ccc(-c2cccc3oc4cc(-c5nc(-c6ccccc6)nc(-n6c7ccccc7c7c(-c8ccccc8)cccc76)n5)ccc4c23)cc1 ; N#Cc1ccc(-c2cccc3oc4cc(-c5nc(-c6ccc(-c7ccccc7)cc6)nc(-n6c7ccccc7c7ccccc76)n5)ccc4c23)cc1 ; N#Cc1ccc(-c2cccc3oc4cc(-c5nc(-c6cccc(-c7ccccc7)c6)nc(-n6c7ccccc7c7ccccc76)n5)ccc4c23)cc1]

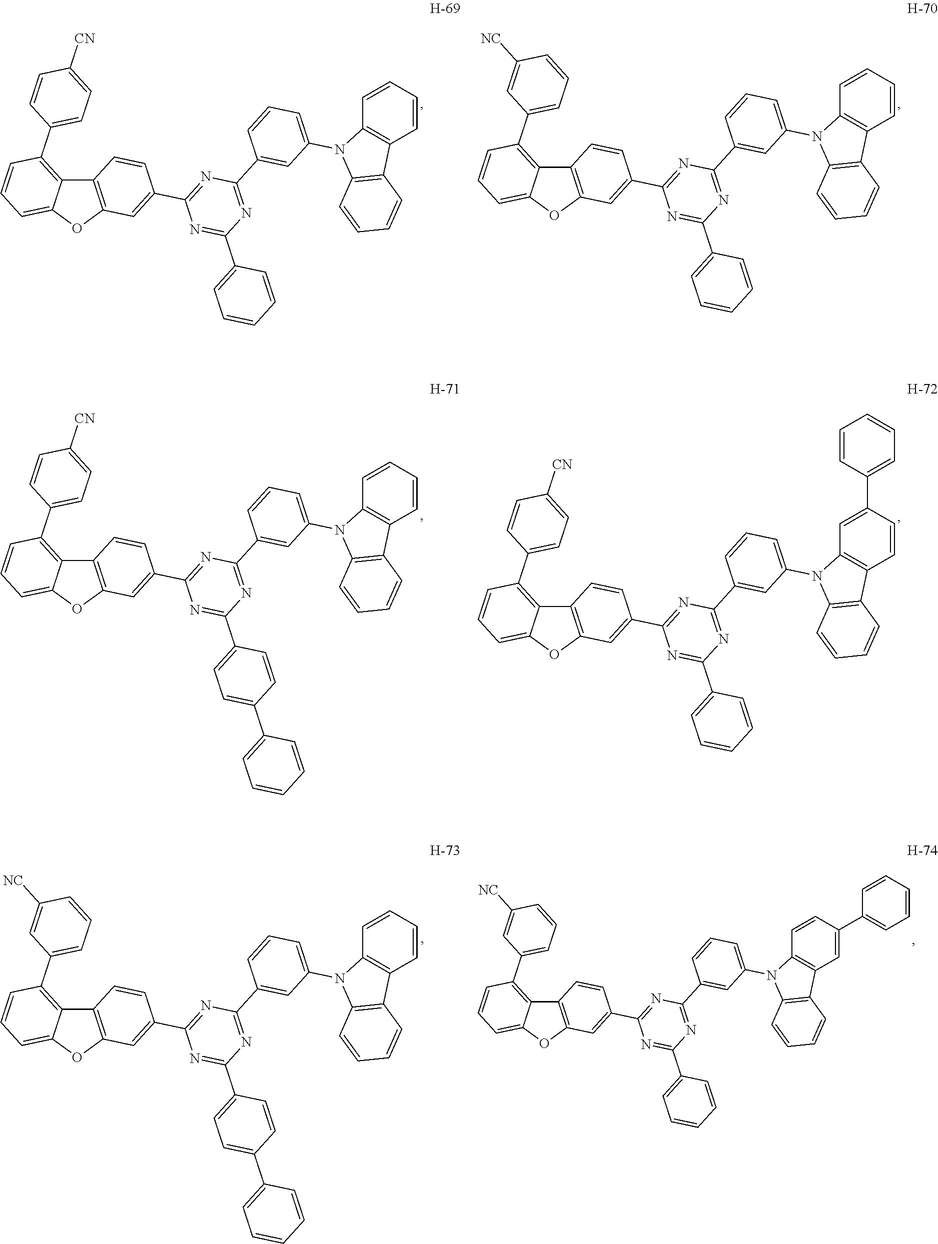

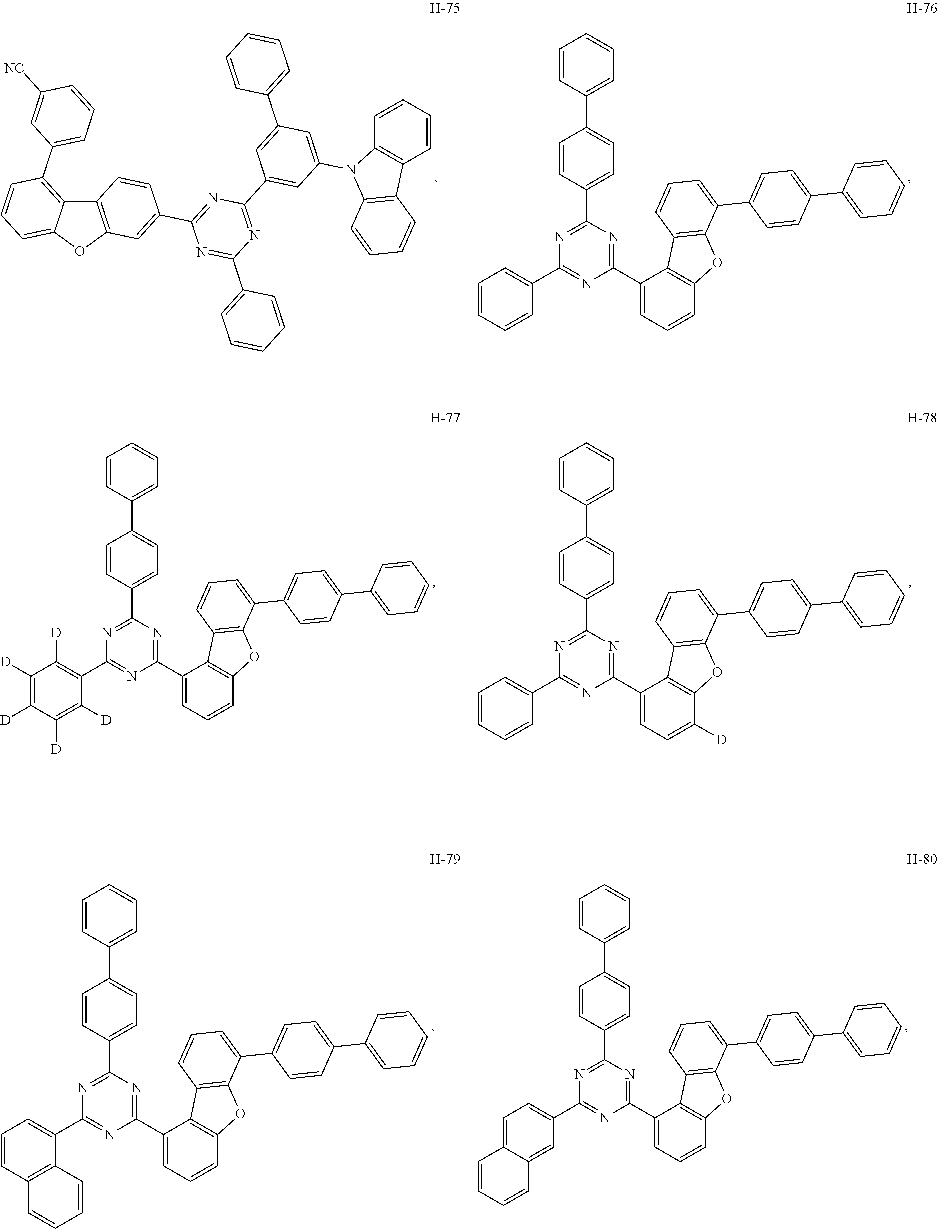

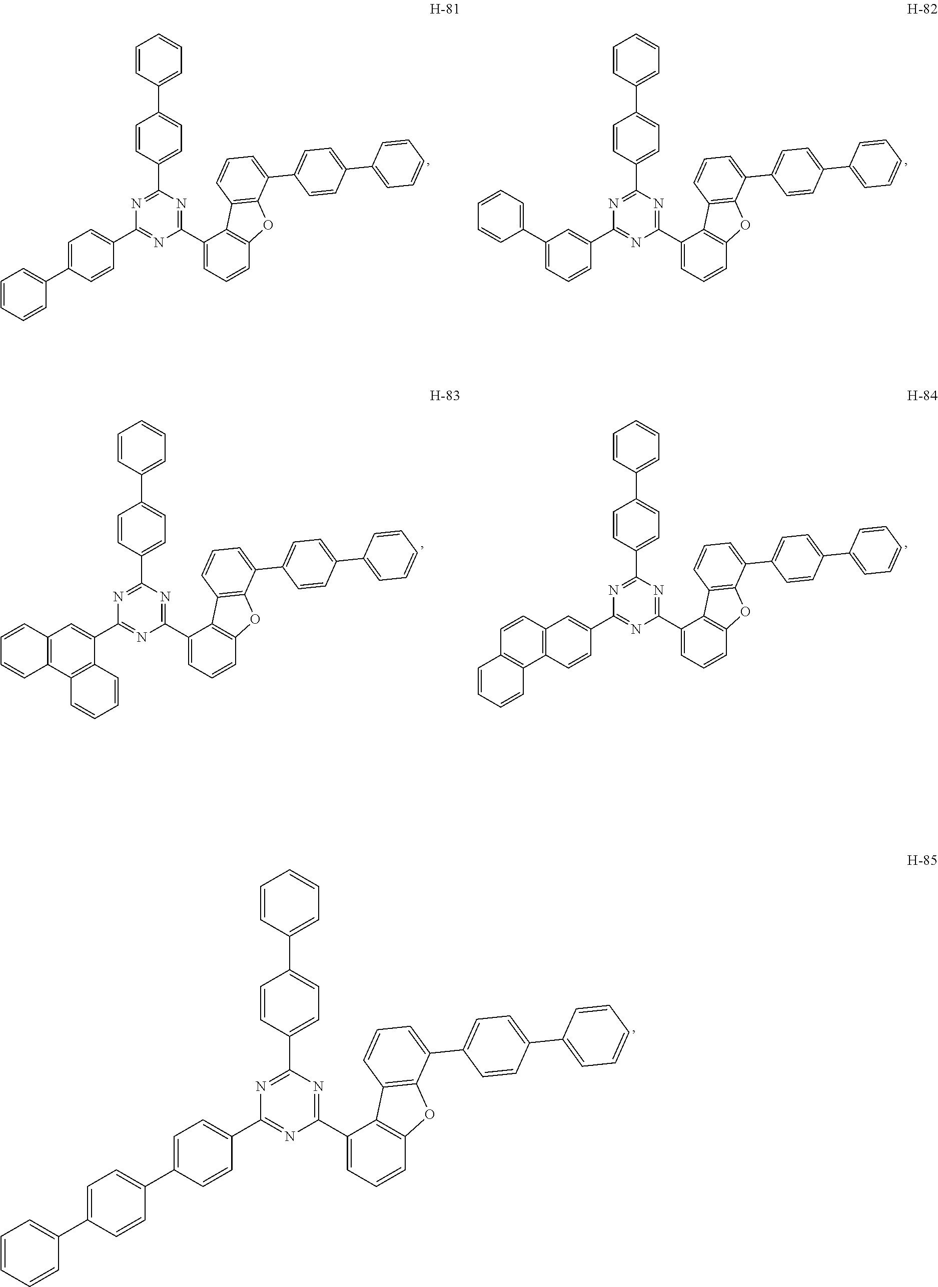

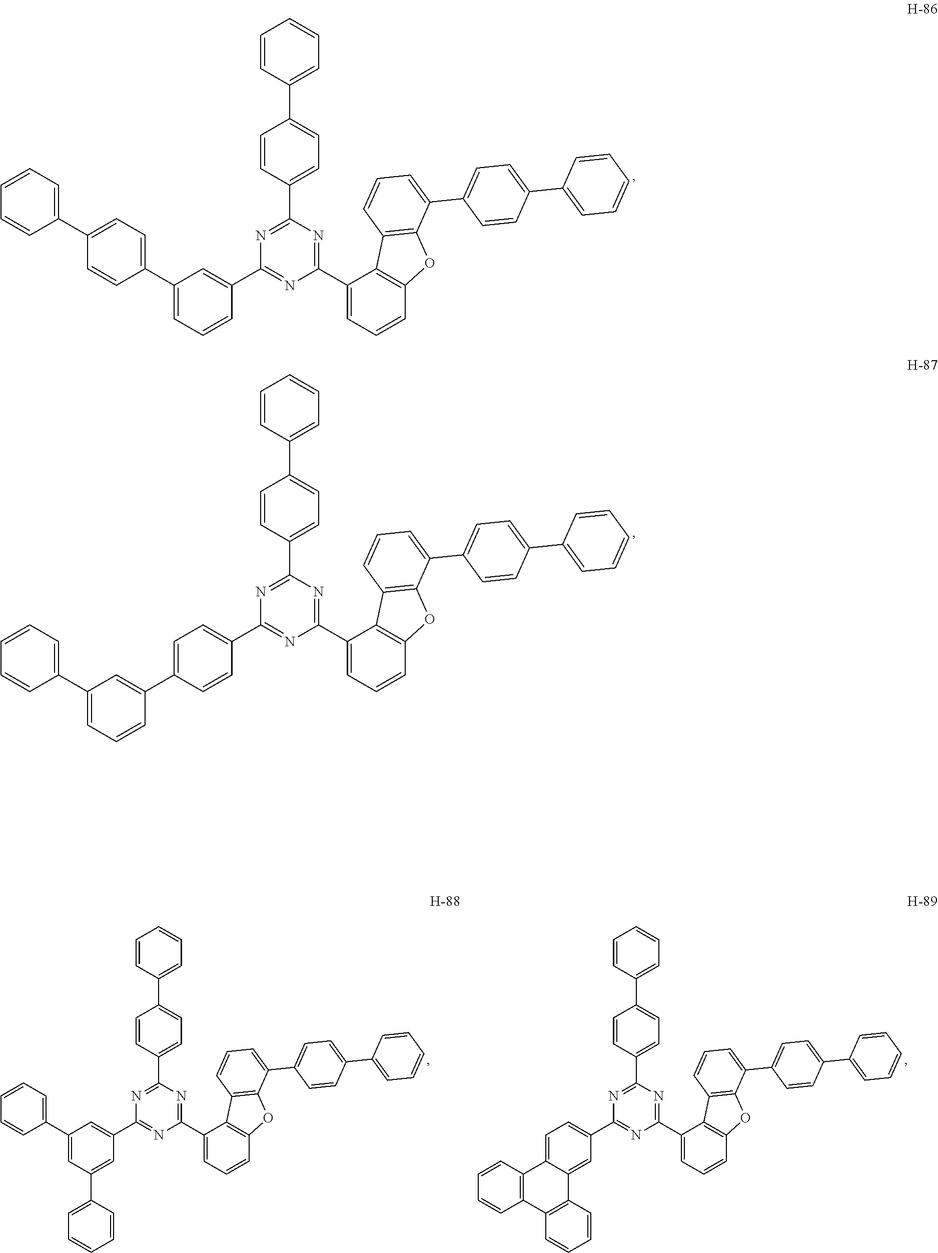 [SMILES: c1ccc(-c2ccc(-c3cccc(-c4nc(-c5ccc(-c6ccccc6)cc5)nc(-c5cccc6oc7c(-c8ccc(-c9ccccc9)cc8)cccc7c56)n4)c3)cc2)cc1 ; c1ccc(-c2ccc(-c3nc(-c4ccc(-c5cccc(-c6ccccc6)c5)cc4)nc(-c4cccc5oc6c(-c7ccc(-c8ccccc8)cc7)cccc6c45)n3)cc2)cc1 ; c1ccc(-c2ccc(-c3nc(-c4cc(-c5ccccc5)cc(-c5ccccc5)c4)nc(-c4cccc5oc6c(-c7ccc(-c8ccccc8)cc7)cccc6c45)n3)cc2)cc1 ; c1ccc(-c2ccc(-c3nc(-c4ccc5c6ccccc6c6ccccc6c5c4)nc(-c4cccc5oc6c(-c7ccc(-c8ccccc8)cc7)cccc6c45)n3)cc2)cc1]

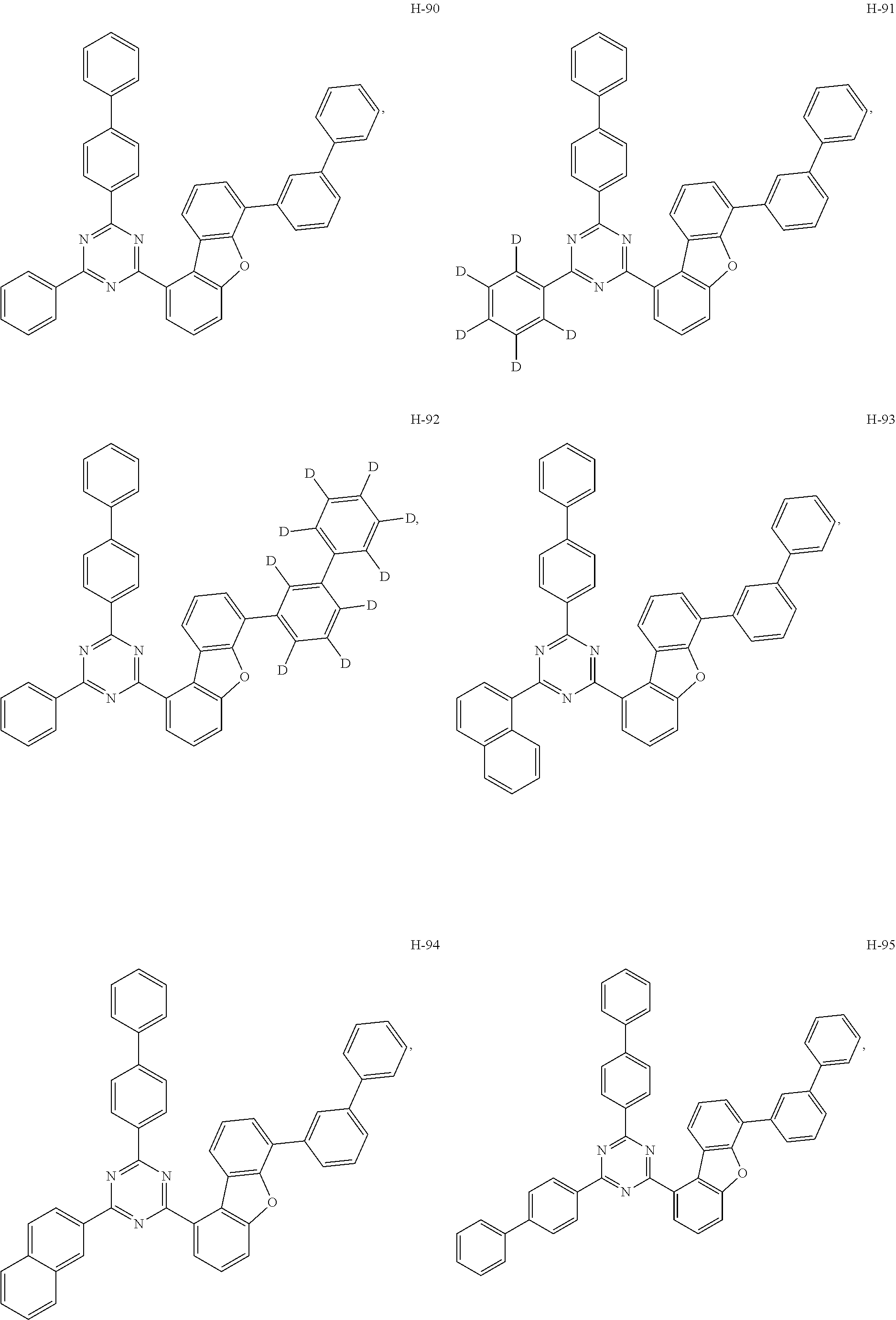

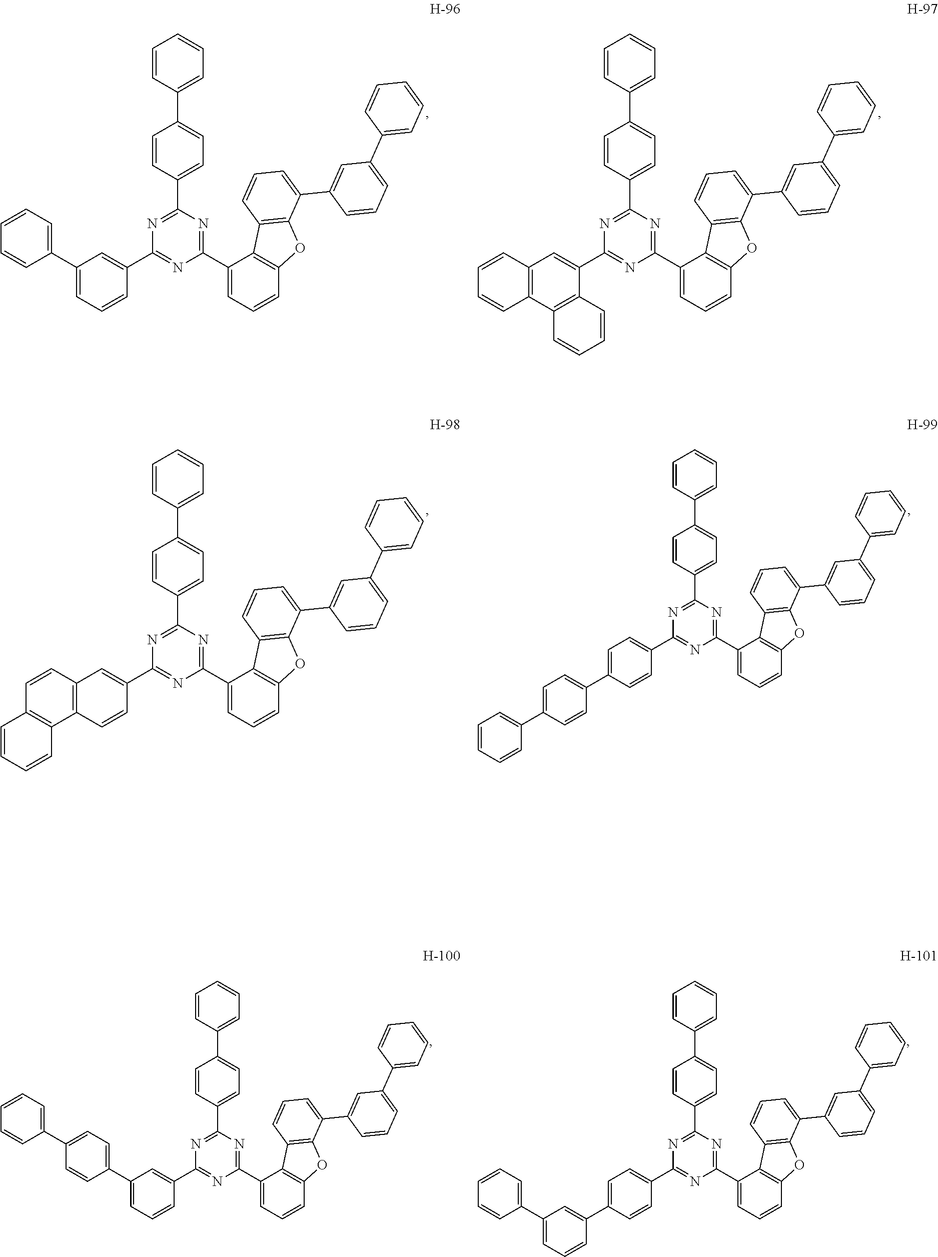

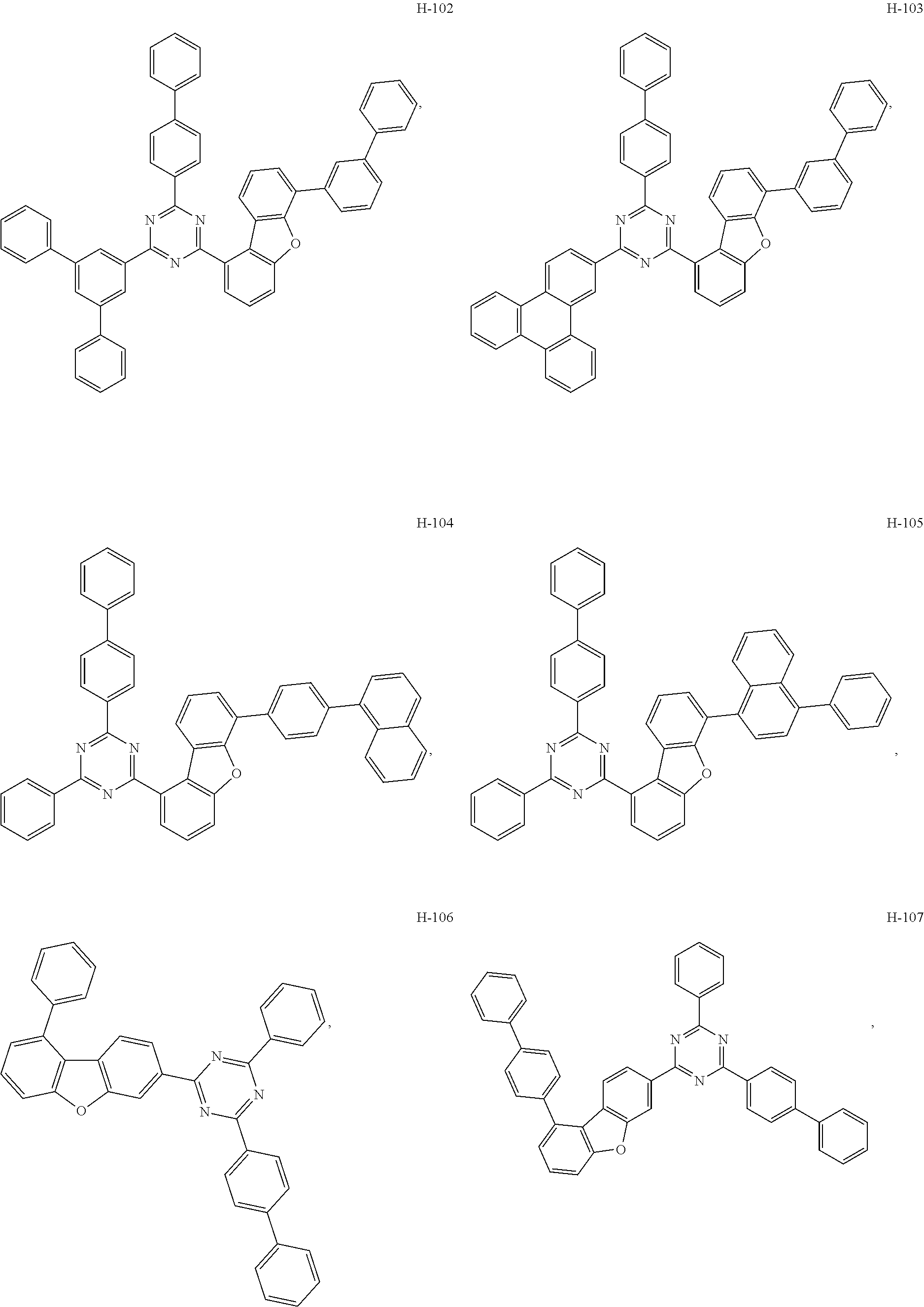

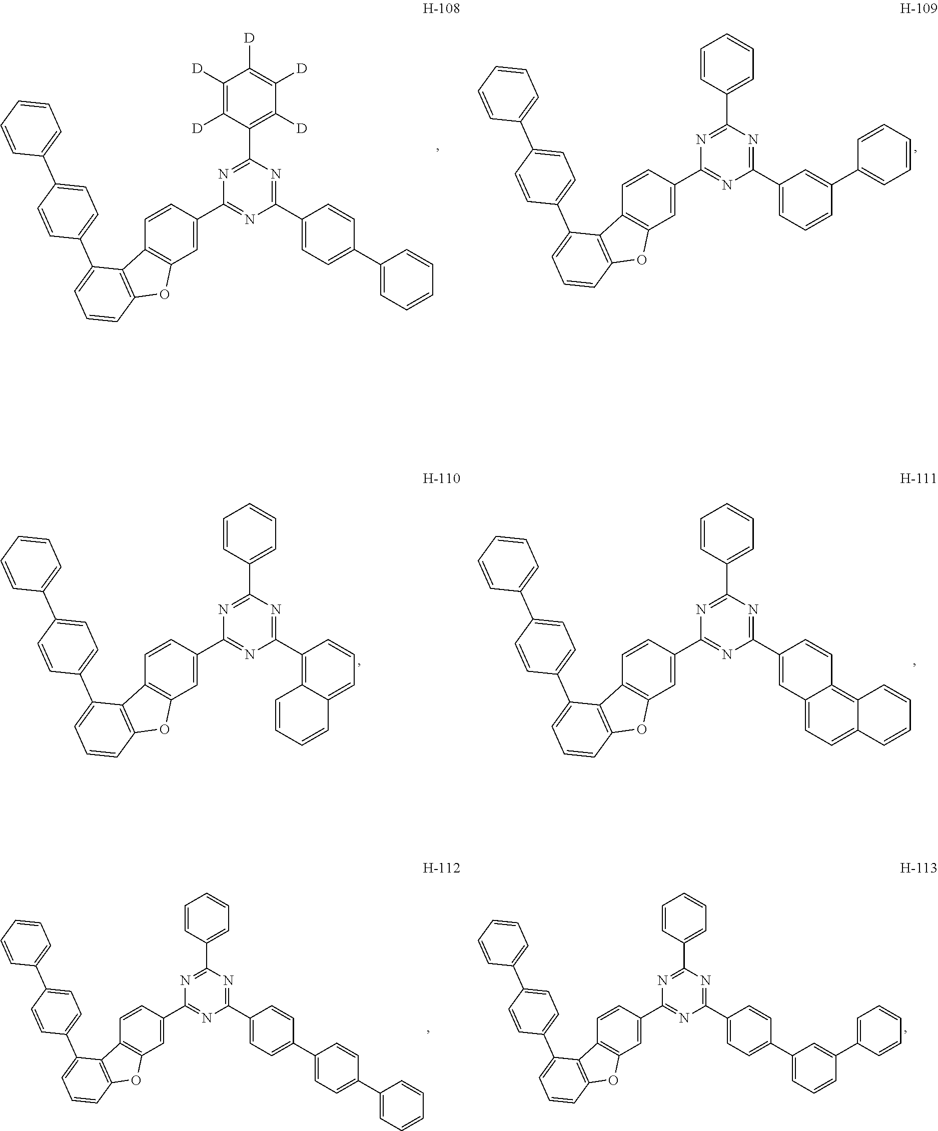

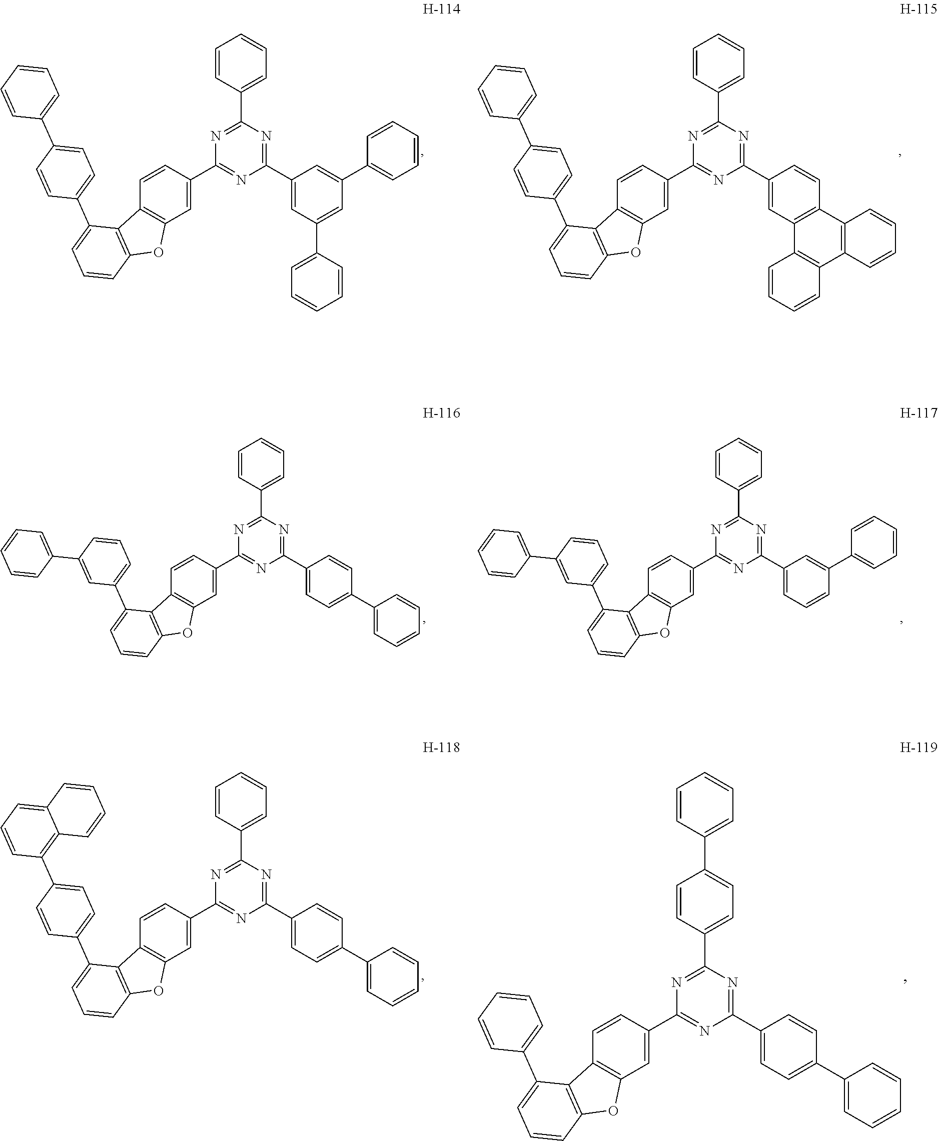

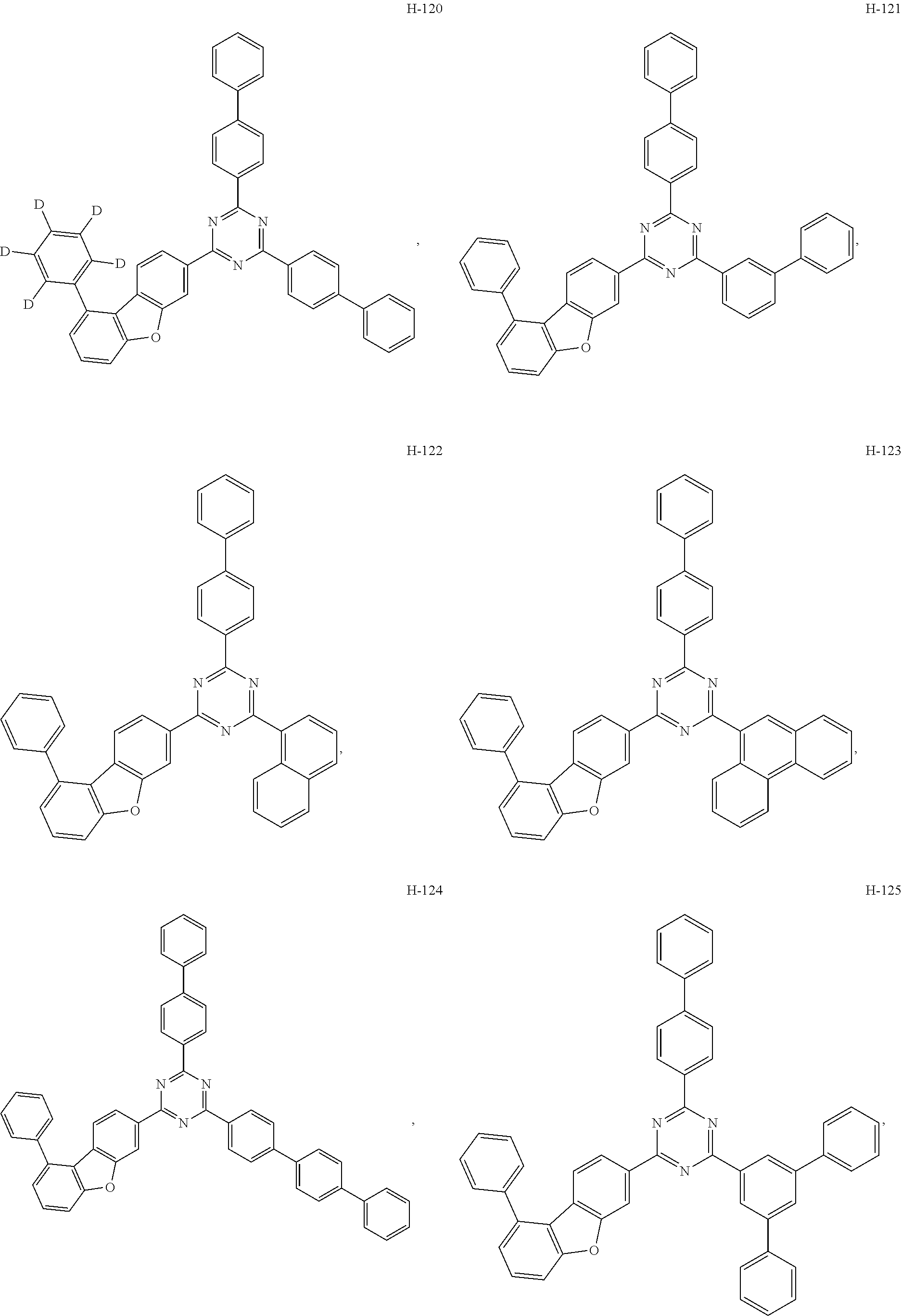

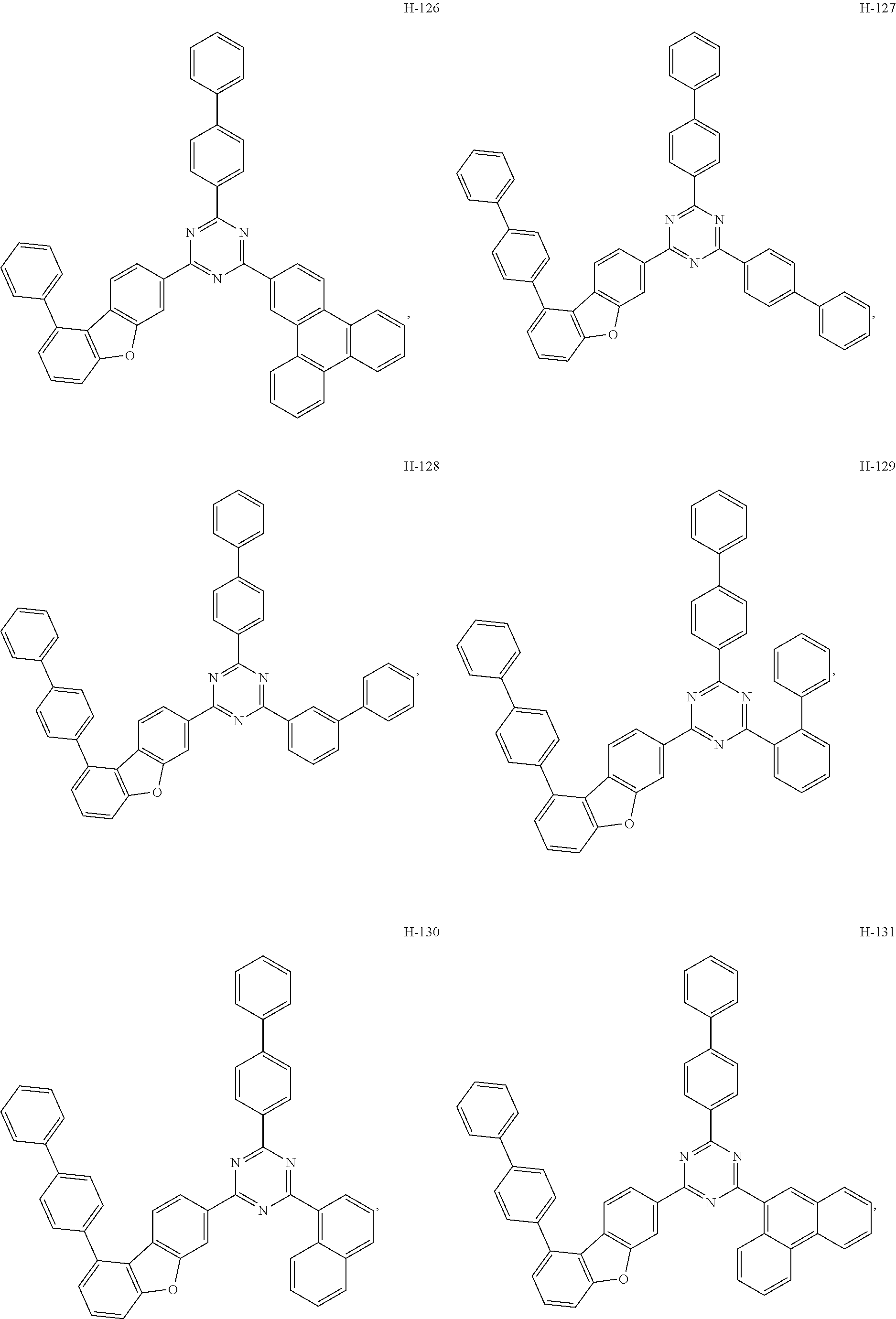

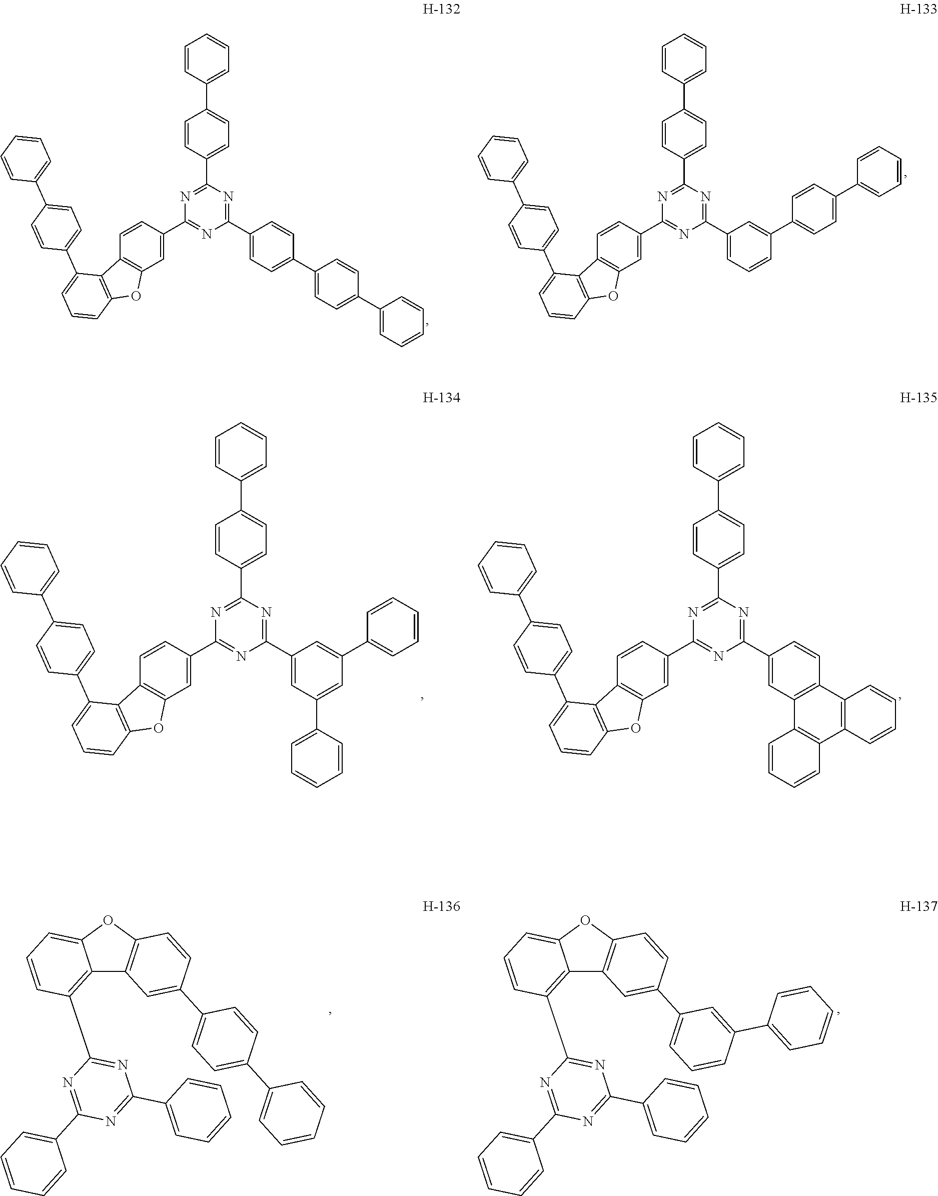

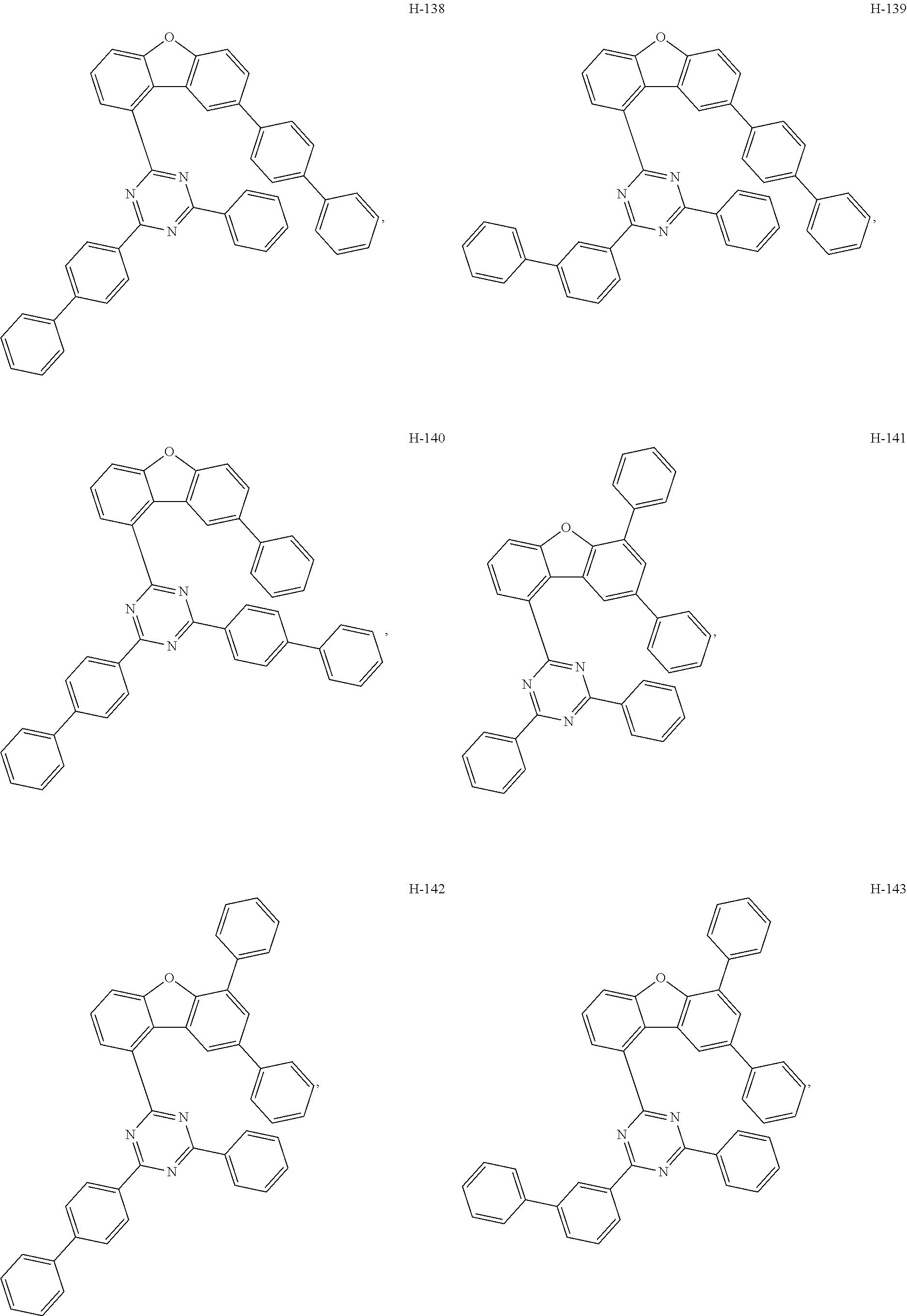

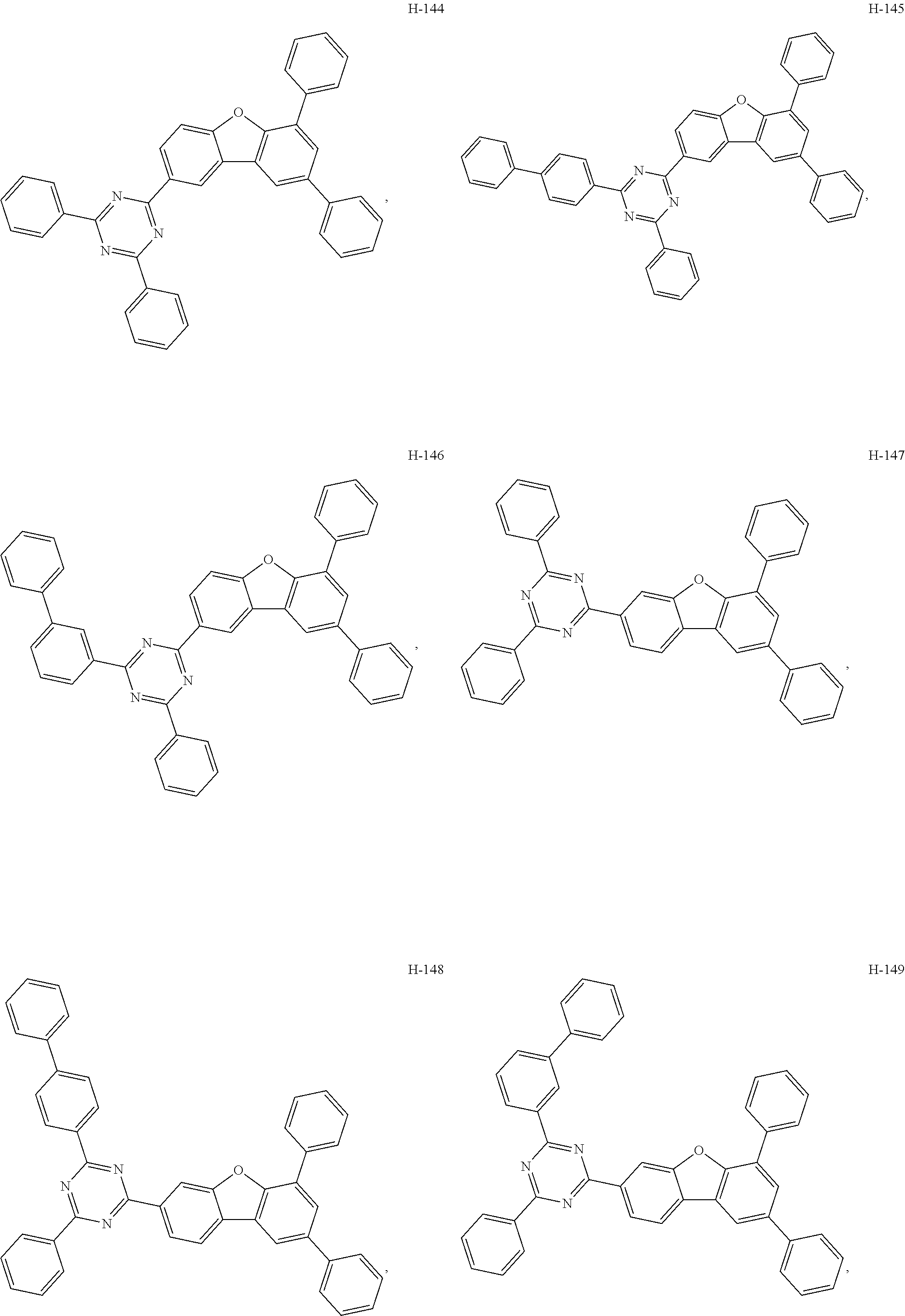

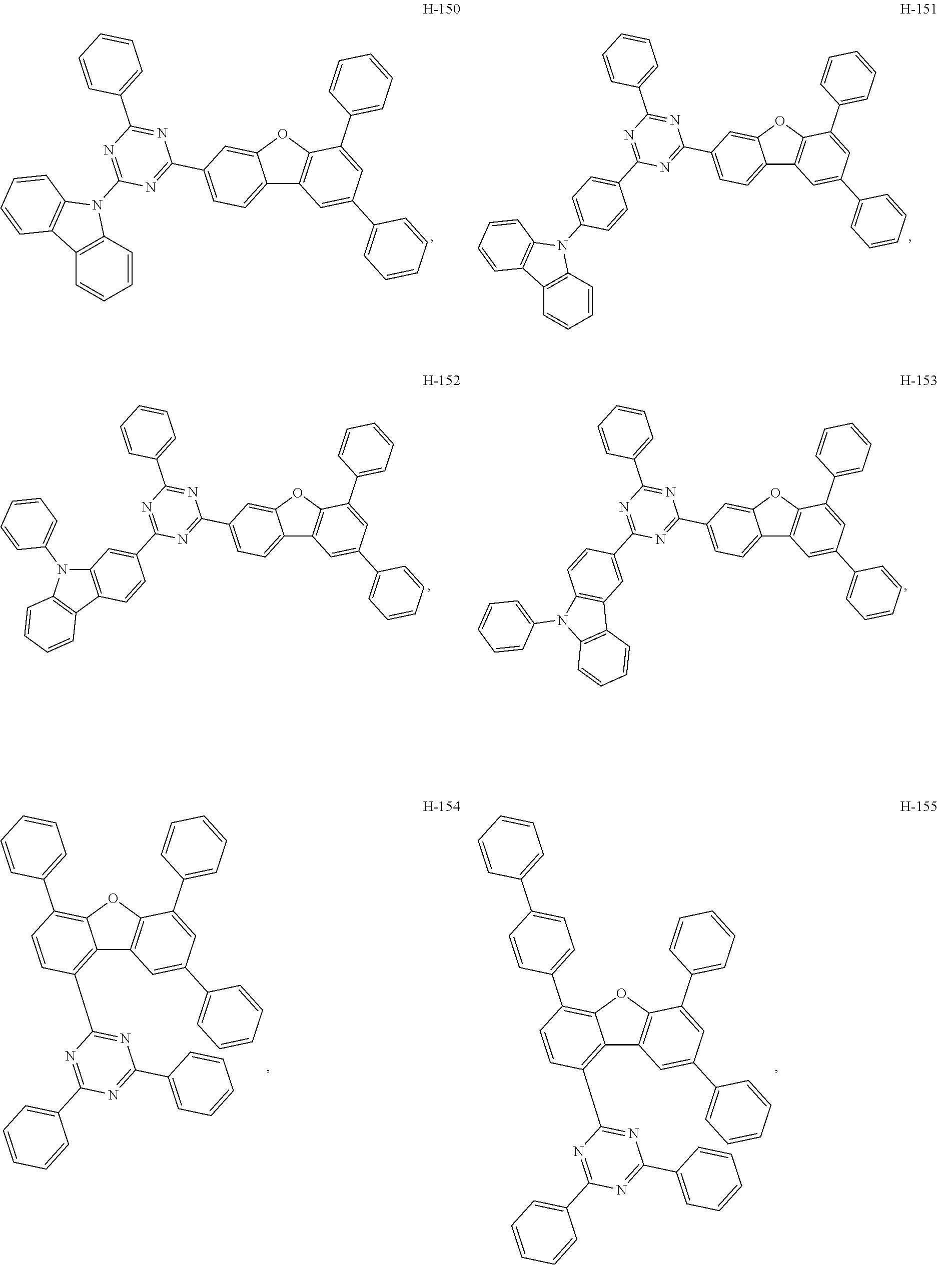

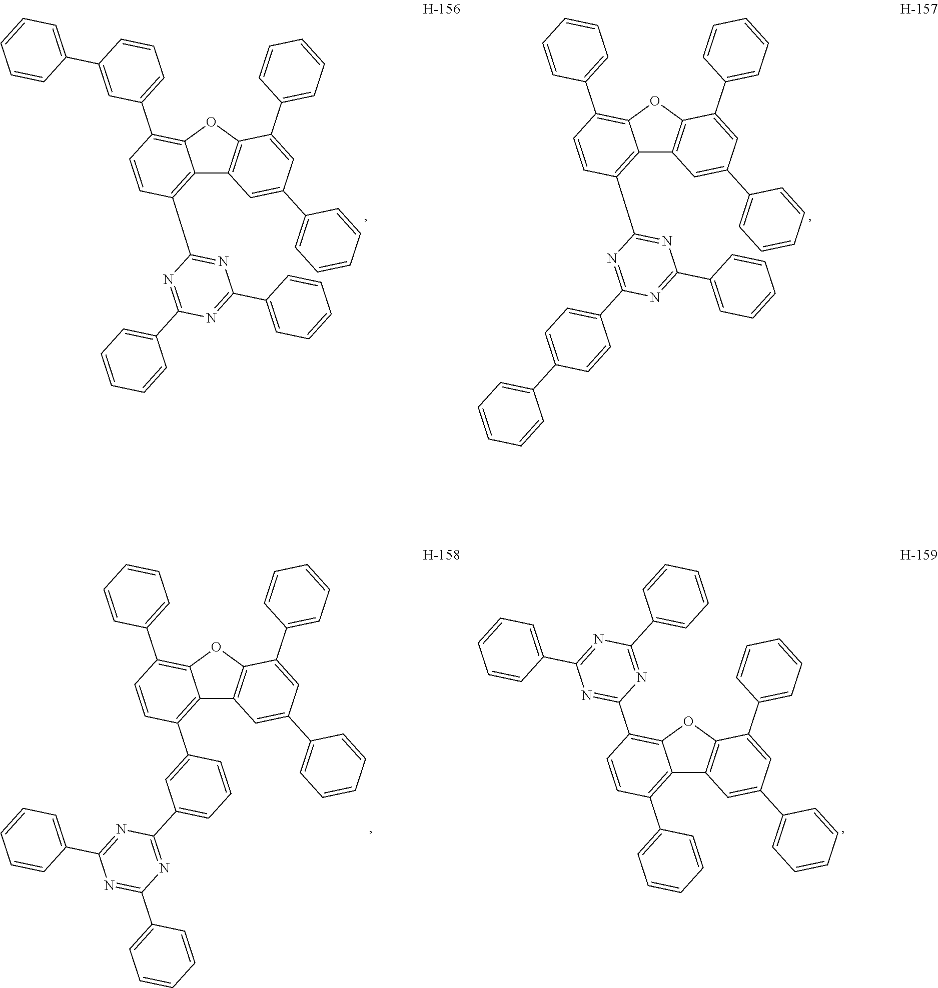 [SMILES: c1ccc(-c2cccc(-c3ccc(-c4nc(-c5ccccc5)nc(-c5ccccc5)n4)c4c3oc3c(-c5ccccc5)cc(-c5ccccc5)cc34)c2)cc1 ; c1ccc(-c2ccc(-c3nc(-c4ccccc4)nc(-c4ccc(-c5ccccc5)c5oc6c(-c7ccccc7)cc(-c7ccccc7)cc6c45)n3)cc2)cc1 ; c1ccc(-c2cc(-c3ccccc3)c3oc4c(-c5ccccc5)ccc(-c5cccc(-c6nc(-c7ccccc7)nc(-c7ccccc7)n6)c5)c4c3c2)cc1 ; c1ccc(-c2cc(-c3ccccc3)c3oc4c(-c5nc(-c6ccccc6)nc(-c6ccccc6)n5)ccc(-c5ccccc5)c4c3c2)cc1]

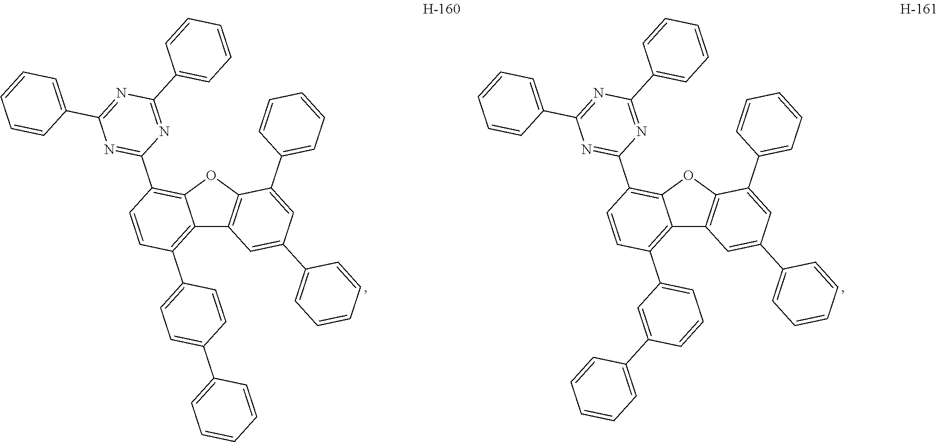 [SMILES: c1ccc(-c2ccc(-c3ccc(-c4nc(-c5ccccc5)nc(-c5ccccc5)n4)c4oc5c(-c6ccccc6)cc(-c6ccccc6)cc5c34)cc2)cc1 ; c1ccc(-c2cccc(-c3ccc(-c4nc(-c5ccccc5)nc(-c5ccccc5)n4)c4oc5c(-c6ccccc6)cc(-c6ccccc6)cc5c34)c2)cc1]

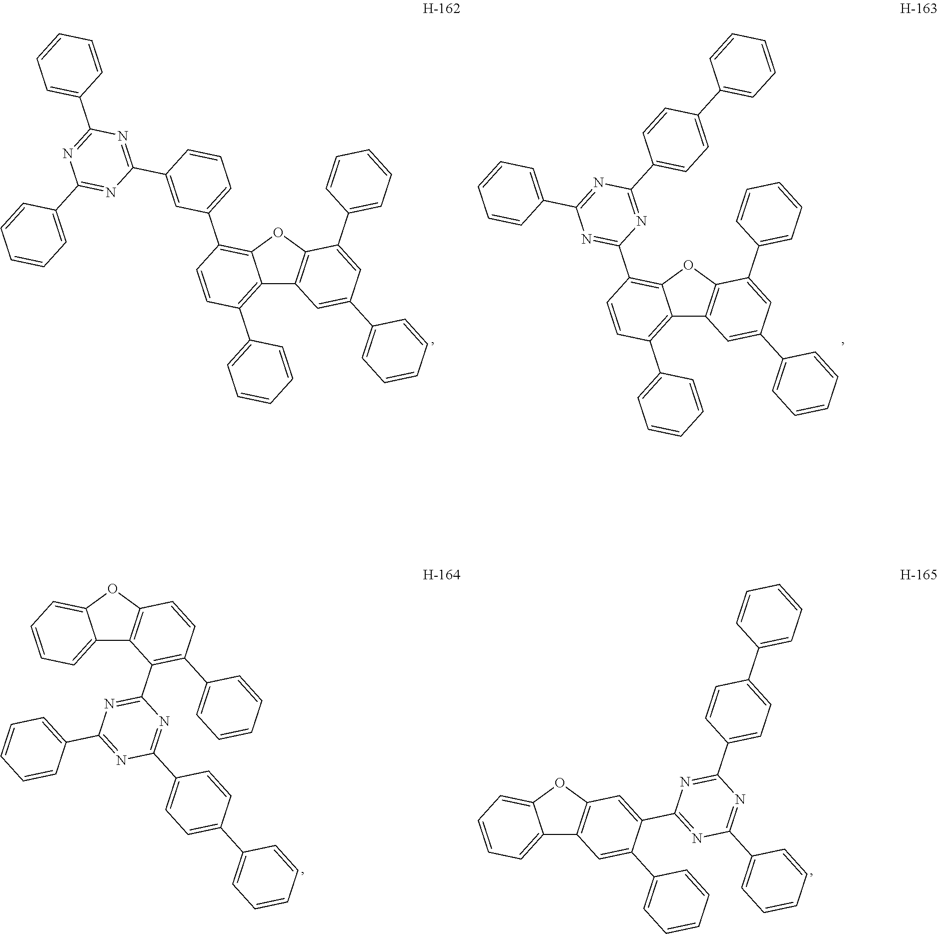 [SMILES: c1ccc(-c2cc(-c3ccccc3)c3oc4c(-c5cccc(-c6nc(-c7ccccc7)nc(-c7ccccc7)n6)c5)ccc(-c5ccccc5)c4c3c2)cc1 ; c1ccc(-c2ccc(-c3nc(-c4ccccc4)nc(-c4ccc(-c5ccccc5)c5c4oc4c(-c6ccccc6)cc(-c6ccccc6)cc45)n3)cc2)cc1 ; c1ccc(-c2ccc(-c3nc(-c4ccccc4)nc(-c4c(-c5ccccc5)ccc5oc6ccccc6c45)n3)cc2)cc1 ; c1ccc(-c2ccc(-c3nc(-c4ccccc4)nc(-c4cc5oc6ccccc6c5cc4-c4ccccc4)n3)cc2)cc1]

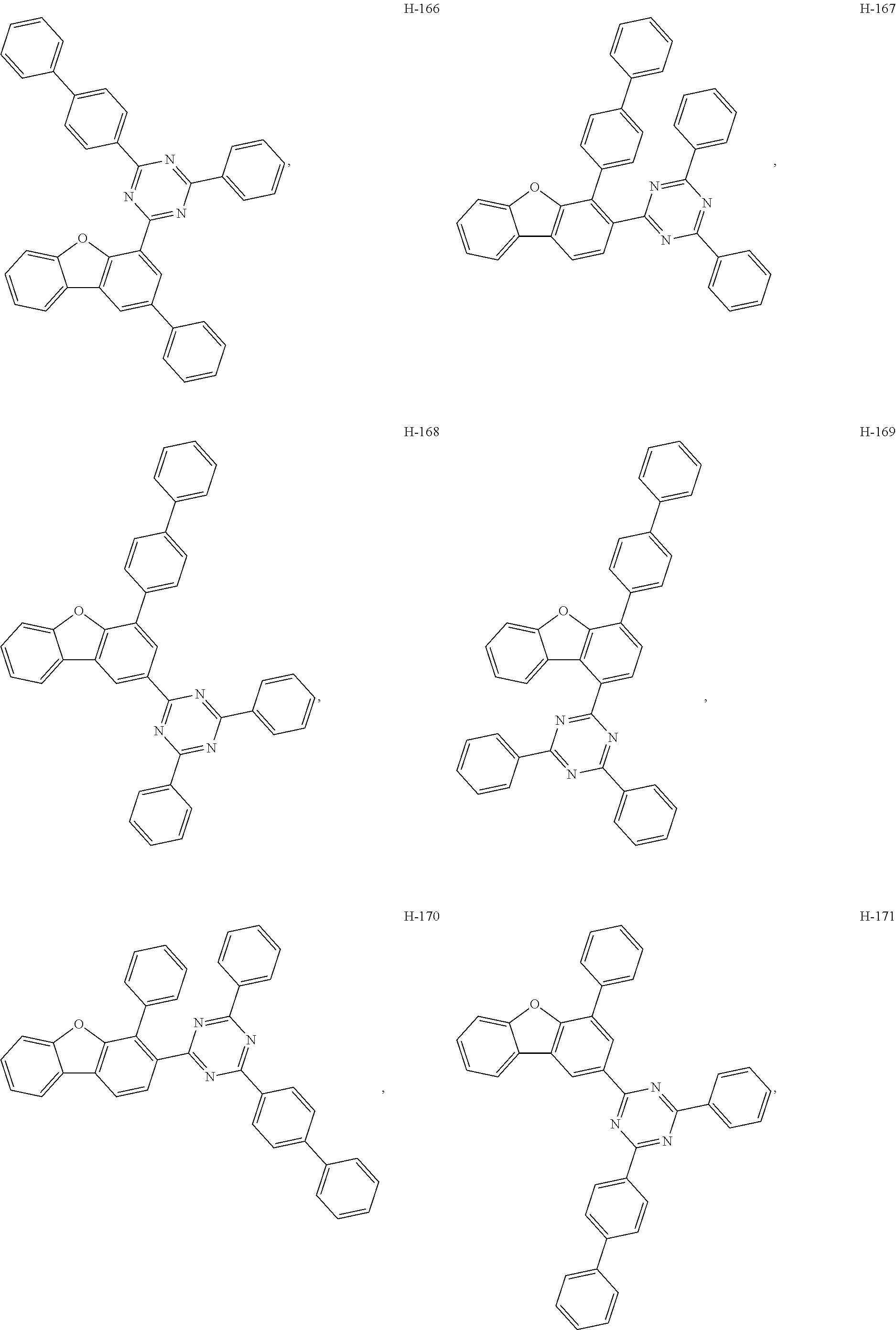 [SMILES: c1ccc(-c2ccc(-c3nc(-c4ccccc4)nc(-c4cc(-c5ccccc5)cc5c4oc4ccccc45)n3)cc2)cc1 ; c1ccc(-c2ccc(-c3c(-c4nc(-c5ccccc5)nc(-c5ccccc5)n4)ccc4c3oc3ccccc34)cc2)cc1 ; c1ccc(-c2ccc(-c3cc(-c4nc(-c5ccccc5)nc(-c5ccccc5)n4)cc4c3oc3ccccc34)cc2)cc1 ; c1ccc(-c2ccc(-c3ccc(-c4nc(-c5ccccc5)nc(-c5ccccc5)n4)c4c3oc3ccccc34)cc2)cc1 ; c1ccc(-c2ccc(-c3nc(-c4ccccc4)nc(-c4ccc5c(oc6ccccc65)c4-c4ccccc4)n3)cc2)cc1 ; c1ccc(-c2ccc(-c3nc(-c4ccccc4)nc(-c4cc(-c5ccccc5)c5oc6ccccc6c5c4)n3)cc2)cc1]

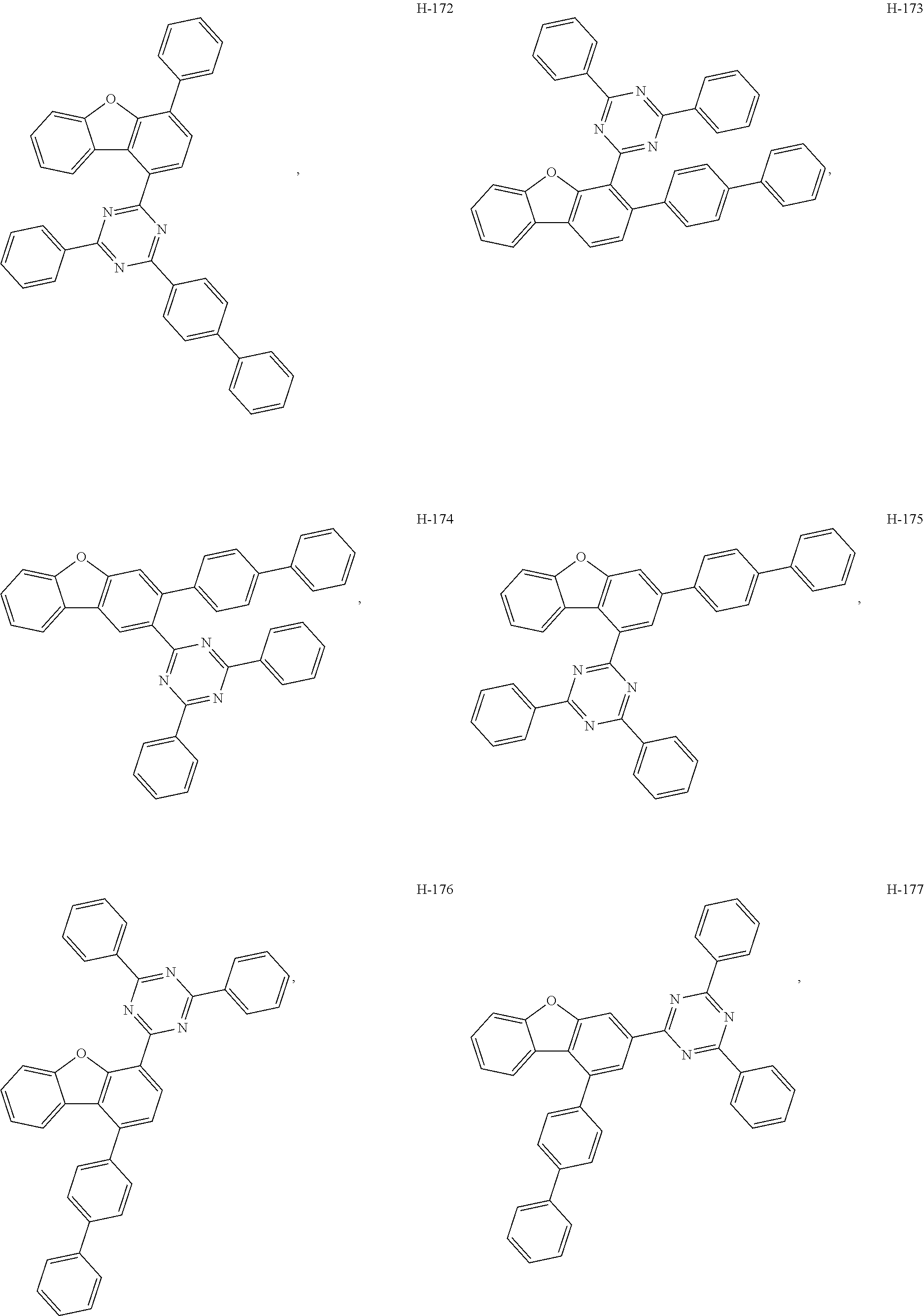 [SMILES: c1ccc(-c2ccc(-c3nc(-c4ccccc4)nc(-c4ccc(-c5ccccc5)c5oc6ccccc6c45)n3)cc2)cc1 ; c1ccc(-c2ccc(-c3ccc4c(oc5ccccc54)c3-c3nc(-c4ccccc4)nc(-c4ccccc4)n3)cc2)cc1 ; c1ccc(-c2ccc(-c3cc4oc5ccccc5c4cc3-c3nc(-c4ccccc4)nc(-c4ccccc4)n3)cc2)cc1 ; c1ccc(-c2ccc(-c3cc(-c4nc(-c5ccccc5)nc(-c5ccccc5)n4)c4c(c3)oc3ccccc34)cc2)cc1 ; c1ccc(-c2ccc(-c3ccc(-c4nc(-c5ccccc5)nc(-c5ccccc5)n4)c4oc5ccccc5c34)cc2)cc1 ; c1ccc(-c2ccc(-c3cc(-c4nc(-c5ccccc5)nc(-c5ccccc5)n4)cc4oc5ccccc5c34)cc2)cc1]

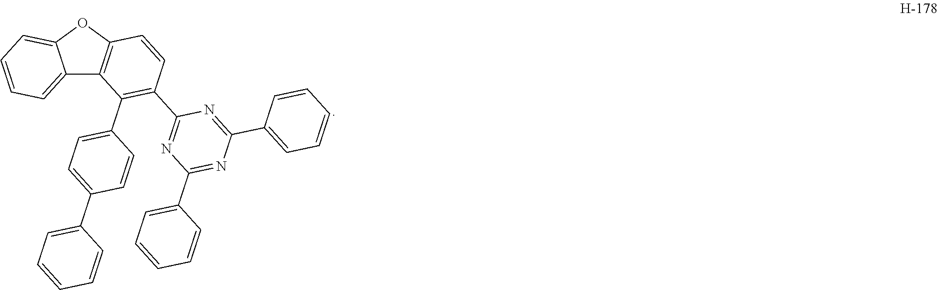 [SMILES: c1ccc(-c2ccc(-c3c(-c4nc(-c5ccccc5)nc(-c5ccccc5)n4)ccc4oc5ccccc5c34)cc2)cc1]

28. The electroluminescent device according to claim 1, wherein the organic layer is a light-emitting layer, the first metal complex is doped in the first compound and the second compound, and the weight of the first metal complex accounts for 1% to 30% of the total weight of the light-emitting layer; and
preferably, the weight of the first metal complex accounts for 3% to 13% of the total weight of the light-emitting layer.

29. An electronic device, comprising the electroluminescent device according to claim 1.

30. A compound composition, comprising a first metal complex, a first compound and a second compound;
wherein the first metal complex comprises a metal M and a ligand Lₐ coordinated to the metal M, wherein the ligand Lₐ has a structure represented by Formula 1: 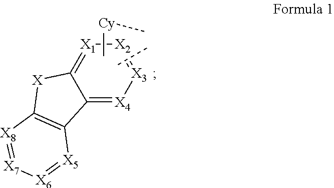
wherein
the metal M is selected from a metal with a relative atomic mass greater than 40;
Cy is, at each occurrence identically or differently, selected from substituted or unsubstituted aryl having 5 to 24 ring atoms or substituted or unsubstituted heteroaryl having 5 to 24 ring atoms; and Cy is joined to the metal M by a metal-carbon bond or a metal-nitrogen bond;
X is, at each occurrence identically or differently, selected from the group consisting of O, S, Se, NR′, CR′R′ and SiR′R′; wherein when two R′ are present, the two R′ are identical or different;
X₁ to X₈ are, at each occurrence identically or differently, selected from C, CRₓ or N, wherein at least one of X₁ to X₄ is C and joined to Cy;
X₁, X₂, X₃ or X₄ is joined to the metal M by a metal-carbon bond or a metal-nitrogen bond;
at least one of X₁ to X₈ is CRₓ, wherein the Rₓ is cyano or fluorine; and
adjacent substituents R′, Rₓ can be optionally joined to form a ring;
wherein the first compound has a structure represented by Formula 2: 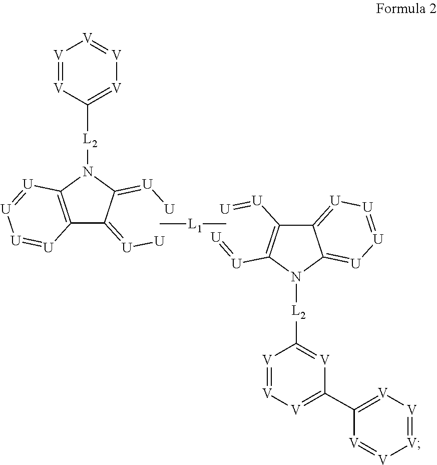
wherein
U is, at each occurrence identically or differently, selected from C, CRᵤ or N;
V is, at each occurrence identically or differently, selected from CRᵥ or N;
L₁ and L₂ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof; and
adjacent substituents Rᵤ and Rᵥ can be optionally joined to form a ring;
wherein the second compound has a structure represented by Formula 3: 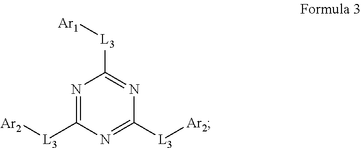
wherein
Ar₁ has a structure represented by Formula A: 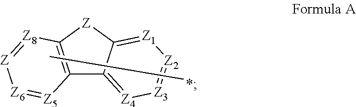
wherein
Z is, at each occurrence identically or differently, selected from the group consisting of O, S and Se;
L₃ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms or a combination thereof;
Z₁ to Z₈ are, at each occurrence identically or differently, selected from C, CR_{z} or N, and at least one of Z₁ to Z₈ is C and joined to L₃;
at least one of Z₁ to Z₈ is CR_{z}, wherein the R_{z} is substituted or unsubstituted aryl having 6 to 30 carbon atoms;
Ar₂ is, at each occurrence identically or differently, selected from substituted or unsubstituted aryl having 6 to 30 carbon atoms, substituted or unsubstituted heteroaryl having 3 to 30 carbon atoms or a combination thereof;
“*” represents a position where Ar₁ is joined to L₃;
adjacent substituents R_{z} can be optionally joined to form a ring;
R_{z} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and
R′, Rₓ, Rᵤ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, a substituted or unsubstituted alkylgermanyl having 3 to 20 carbon atoms, a substituted or unsubstituted arylgermanyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof.
